SEQUENCE LISTING (243 sequences)

SEQ ID NO: 1 - DNA, 553 bp, Homo sapiens
ttcctattgattcttgtaggagttattaatcctgattgcaacacacaagtgaacagacaagagagataaattttgtggcttgtggraaatgaaggaaaatgggcctcatttagtctgtgagcatactaattgaaatagatgtatgaagacttcaccagtgttaaaataacattgtttttataaatcatatgatataaactatataacaataaaatagaatgttraacatgtatttaatcatcatcataattttgattcagaaatctataatttattagttatcttaataatgtttagaatttgttgaacattttaccttatgtgaattaaggacaaaatattaaagctattcagcacaaaaaaagggtctttctgatgtgtatttttcatagaatagggtatgaatttgttattttgttattttgattaatgtctaaaacaaaagataaacacattatgatataacattactattgattttaatrgccccttttgaaatagaattatgtcattgtcagaaaacataagcatttatggtatatcattaatgagt

SEQ ID NO: 2 - DNA, 456 bp, Homo sapiens
atgagctatgctcctgagcacagacggctgttctctttcaaggttacaagcctgatgaagggaaacgaggggatgcctgtgaaggtgacagtgggggaccctttgtcatgaaggtaagcttctctaaagcccagggcctggtgaacacatcttctgggggtggggagaaactctagtatctagaaacagttgcctggcagrggaatactgatgtgaccttgaacttgactctattggaaacctcatctttcttcttcagagcccctttaacaaccgctggtatcaaatgggcatcgtctcatggggtgaaggctgtgaccgggatgggaaatatggcttctacacacatgtgttccgcctgaagaagtggatacagaaggtcattgatcagtttggagagtagggggccactcatattctgggctcctggaaccaatcccgtgaaagaattatttt

SEQ ID NO: 3 - DNA, 456 bp, Homo sapiens
caaacttataacatgtattacctcaaatacttatcattttttgtggtgagaccacttaaagtctactctcttagcaattgtcaagtatacaacacattgttattaactatagtcaccgtattatacaagagatttttctaactaattcctccatccaaccccagcctctactaaccaccattctcttctctgcttccatgrgttcaactgttttcgattccacatataagtgaaatcatgctatatttgtctttcggtgcctgccatatttcacttaacattatgccctctcggctcatccatgttgttgcaaatgacagaatttccttcttttttaaggctgaatagtattccactgtgtatatacatcatattttttattcattcattcattcatcagtggatacctagtttgattccataactgggctattataaataattctgcaatgaaca

SEQ ID NO: 4 - DNA, 456 bp, Homo sapiens
gcactctatattgctcccacactcaaaaaaaagtgtagacacatcaagattaagaggtgacaaagacatagcatgttctcgcctctctgtctttgttcagggtgagttttgagatgcttttgggaaaactaagagctccagactggggcccagtgtttagcagtaactagcctgcctgcagataagtgagcattgttgccsaaagtgtttgagagaacaccgagaactcctgaaaaattgtttgcgatgagatatgatttcacataccattatgtaatttgcacaatgtagtttgaggacacgctcttgagaatccagtgttttgtttgagatttggatcatggggtggagaacagagcttatagaaatgctgcaccctttctcacagtggcctcccagcaaggtgtgtagcctcattagggagtgaagtcaaagcgtactggtttctgccaagct

SEQ ID NO: 5 - DNA, 450 bp, Homo sapiens
cgctggaattctcccagaggcaaactgccagatgtgaggctgctcttcctcagtcactatctctggtcgtaccgggcgatgcctgagccaactgaccctcagacctgtgagccgagccggtcacaccgtggctgacaccggcattcccaccgcctttctcctgtgcgacccgctaagggccccgcgaggtgggcaggccargtattcttgaccttcgtggggtagaagaagccaccgtggctgggagagggccctgctcacagccacacgtttacttcgctgcaggtcccgagcttctgccccaggtgggcaaagcatccgggaaatgccctccgctgcccgaggggagcccagagcccgtgctttctattaaatgttgtaaatgccgcctctcccactttatcaccaaatggaagggaagaattcttccaaggcgccctccctttcctg

SEQ ID NO: 6 - DNA, 456 bp, Homo sapiens
aaaccatatgcacaaaaataaaaataaattgatctgagcttagagtttacgaatttatagttcccaaaagaataactgggggtaaatgggacaaggtagggaaagaccatcagtagaaactaagaatagtaaacatttgtaaaaaccctctgccttataaagcagaataaattgaatacatatgataaatgctaacacagrtatgttaattgctgaactcaatataagcatttctttggcatggataaacgcttccaccaaagcctcctagtgatgcaaggaatcctggctttgttgcaatggtctcctaaaaaaaacaagcctttgtgtgggtaaggaactgattctcagccccattatctagtatctagtgattatgtatctgagatgtaaaacagaaacctaaaagccaaggatggagtctccccacagagcaaatgagcattttcccagtga

SEQ ID NO: 7 - DNA, 456 bp, Homo sapiens
aaaagagaatattgcctcccatagcttcatggaaaatttagaataattaagattcttatatccctatgtacttgtttacgtttttaaaaagagcaaatggtcactgaaaatgtagtgaatgcttacacaggtatagtagattatattttaaaatctagaatagcttactttagaatcagggttctttctgggtttttgaaygtgagcggttagcaaaaatggcggaagtaaactttgttataaaagcaagttataatcgtgggtctggacacaagattttgaaagaaattaccagaactaggaagacactgaagaaacttgctcacttttggagggcatggagacatctttactttccttactcattttatttaacttcattttatttaatttttaggaaaacacctgaagatatttctaaattactattactactagcactgctactgccaccac

SEQ ID NO: 8 - DNA, 475 bp, Homo sapiens
aaagtagattttgggcagaattccaaggagtctgtatttttaacaagcagccttccttctattttgccttctattaaaagtaatggcaaaaaccgcaattatagtttgcaccaacctaacacatgctgcctgaggagttagtgaaggcagcccctcgacagcactttgggtgacgttgtgtgaatctgcctcagatgcagrcacagaagtccaaatggactggtttgattaagagcagggaaaaaaagagggttcttattggtttttcacatgccagtaactcactaatacatctagagagtattaattgtattatattaatatcatattaattaattaatataataattaatagataatttattgtattaaaatcagagacagaagagattcagtcaaatttactcatcttttcatcaagtattagaagatcagtcatccttcctatcagcctgcagacagactgagaagctag

SEQ ID NO: 9 - DNA, 456 bp, Homo sapiens
acttaatgtaggtgattacattttttcacttctcatctgaatattttggccttatgtagagacttccttgagtatatgataaacacctgaaacaaactataacggctatagtttgtatatttaataaatcacaaaaatgtggatcccttttccaaagagacttgcaagtcctgtgcaatgaaatcagctctttataaaacrtcagcattgattcgcccattgcctacattatagcttctaaaagaatattctgaatctttgaatgtagtgaagcattcactaagtttattaacttaggagaggaaaagagttgttcacaaaaataagtaacaagggaagagttactgagtgagcaccctaatgagtaaccaaaaaggctctttatgtaacttcactttcccaaagcttgtatgcaccatttttcatttttaaaaaatactgagagcctttggcaag

SEQ ID NO: 10 - DNA, 456 bp, Homo sapiens
tgcaggtgcgtccggggaggttttctccataaacttggtggaagggcagtgggcaaatccaggagccagcccgggcttcccaaaccccgcccttgctccggacacccccatccaccaggagggttttctggcggctcctgttcaatttctttccttctagaaaccagcatccaggcacaggaggggaggcccttcttggtrgcccaggctttggcgggattatttttcaaagaactttaggagtgggtggtgctttcctggcccccatgggcccctgcctgtgaggtcggacaagcgcagggagtctggggcctctcagagtgcaggaagtgcgcacagggtgctcccaggctggggagcacaggtaggggacggtgcgtgggggatggcgcctggggcatgggggatggggtgtgggaaacggcatgtggggcgtaagggatggggtgtggagga

SEQ ID NO: 11 - DNA, 386 bp, Homo sapiens
cacttaattatggttgttattggtataaaatgtctctgttttccctaatatatttttaaatctcttttttccttttagaatgaattctggaataaatacaaaggtcagtattttttctgttttaaccttcagtgagaggggttcatcaggatatttgaattttgaaaatagttcctgaatttcctttctgcttttgttctmattttactcatttaagactttttccctcagggtgtttccataatagttattgtaaaagagtttttagagtaattttatactaatcctagttttgttattgagttagagatatatatttaaatcagttcattctcatttgaggataccaaattccatgataacttttcttaaataaaagtgtattc

SEQ ID NO: 12 - DNA, 456 bp, Homo sapiens
ccctctcatctctgcagcctggacggtgggtgccttgagtgctgccagaggctgggctcggatggctgggcttggcctttccagccaacggcatcctcaaggccagctgtggctccctggggctgagagtcagacgggcggatcagaggtcacagagacaaaaacacaaggacagagtcagagagagaaagggagagggarggagaaacggagacacagtgagatgggaggccaagaggcagagacagaggtagaaagacggagacagagagagagggaggggttggggcaggcagagacaggacagttagccatctgccaccacagggaggcacaggacgaggggcacagcagaggagctcccagggaggaggaggctgagccgagccagtgccaccactctcggactggctccgtcggggaaggagctgcctaatgcacagctggacaggtggg

SEQ ID NO: 13 - DNA, 456 bp, Homo sapiens
ctgctgttggtgcacacaccgcattggtctctccatacaaacatgcctagaggcgatgtcagagggtggagaccaggagaggcaggagtcagacatctggtgccaccaggaaggcccttctcagaggaccaggctgtgcgtggtgcccgccgtgggaggccagcctggcgttggcatccagcatcatcagtttgtgcagtygggtggggctcagtgagtgcctcctgtgtgccaggcacaatgacgcacaatgtgtgcacaccaggctcatgtgcaggtggctgcgagacagggcgacccatcaaggcagatgcaccatgaggcagtggccagtgctgtgggtgttaggggcattgctccccggccactacggcatagcaggcagtgatcgccacactggccaagctttagaccatttattccagagaccccagaggcaaaaagcccggctgcacc

SEQ ID NO: 14 - DNA, 451 bp, Homo sapiens
  misc_feature (201)..(201): n = absent or A or G
tgccccaagtcctagcgggcagctcgaggaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtggggnagtcagccgtgtatcatcggaggcggccgggcacatggcagggatgagggaaagaccaagagtcctctgttgggcccaagtcctagacagacaaaacctagacaatcacgtggctggctgcatgccctgtggctgttgggctgggcccaggaggagggaggggcgctctttcctggaggtggtccagagcaccgggtggacagccctgggggaaaacttccacgttttgatggaggttatctttgataac

SEQ ID NO: 15 - DNA, 456 bp, Homo sapiens
cttttatttttataggaatagaggaagaaatgtcagatgcgtgcccagctcttcaccccccaatctcttggtggggaggggtgtacctaaatatttatcatatccttgcccttgagtgcttgttagagagaaagagaactactaaggaaaataatattatttaaactcgctcctagtgtttctttgtggtctgtgtcaccrtatctcaggaagtccagccacttgactggcacacacccctccggacatccagcgtgacggagcccacactgccaccttgtggccgcctgagaccctcgcgccccccgcgccccccgcgcccctctttttccccttgatggaaattgaccatacaatttcatcctccttcaggggatcaaaaggacggagtggggggacagagactcagatgaggacagagtggtttccaatgtgttcaatagatttaggagcaga

SEQ ID NO: 16 - DNA, 401 bp, Homo sapiens
agacagagcagagcagagggaaccctctccctccatatcccatcctccaaaatgtgtcccttgatgtggatgggtagacaggattcctgccctggcagccagacccctgccttgggtctgcacctcctctccctccttcctctccccgtcatccctaaatcttgtcctcgagccactgccaccctgtgtaaaccctcatgyccagtcttgggggtgccatcccttctctttaaagctgaatggaccaaacatacccattgagtgttgggtggggacatctctggaaagtcagcacctggaccagctccacccctctctgaggacaccttctttccctttcagaacaaagaacagccaccatgcagctcttcctcctcttgtgcctggtgcttctcagccct

SEQ ID NO: 17 - DNA, 401 bp, Homo sapiens
aaccttccaaagatggctgaaaaagatggatgcttccaatctggattcaatgaggtaccaacttgtcgcactcacttttcactattccttaggcaaaacttctccctcttgcatgcagtgcctgtatacatatagatccaggcagcaacaaaaagtgggtaaatgtaaagaatgttatgtaaatttcatgaggaggccaasttcaagcttttttaaaggcagtttattcttggacaggtatggccagagatggtgccactgtggtgagattttaacaactgtcaaatgtttaaaactcccacaggtttaattagttcatcctgggaaaggtactctcagggccttttccctctctggctgcccctggcagggtccaggtctgccctccctccctgcccagc

SEQ ID NO: 18 - DNA, 401 bp, Homo sapiens
caaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgcsatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgccctcgagcccaccgggaacg

SEQ ID NO: 19 - DNA, 502 bp, Homo sapiens
taaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcctggaacacatcctgtgaccccgcctgtmctgtaggaagccagtctctggaaagtaaaatggaagggctgcttgggaactttgaggatatttagcccaccccctcatttttacttggggaaactaaggcccagagacctaaggtgactgcctaagttagcaaggagaagtcttgggtattcatcccaggttggggggacccaattatttctcaatcccattgtattctggaatgggcaatttgtccacgtcactgtgacctaggaacacgcgaatgagaacccacagctgagggcctctgcgcacagaacagctgttctccccaggaaat

SEQ ID NO: 20 - DNA, 456 bp, Homo sapiens
aatcattccaatgctccccattggtctcctcttctgaaaaaggaaggtaatactagaatctacctaaaaggatcagagaaagggtaaaatggaacaactcgtgcaaagggctagcgttgcacctggcacatagtaagtgcacaataaatgtaagcacattttgaaatgtattattagtctttgggctaagcacctgcaccraatttgttacctcctctttgctgctatttcctcattgatgaaattcagaaaacggtgggacctaattaactgtgttattgtgaagattaaatgacacaatacagtgccagcacctagttattactcaacataaatttgtcacagttctcacaagacatcagaacacccgctgatgtgctgtcccccatggcactcagcatattaagtgtggtcggcacaagcggctgcctggtgtgaagtatgagggcaaaaggc

SEQ ID NO: 21 - DNA, 456 bp, Homo sapiens
ttcagatcatttccatgaccatggaaatgctgtttggagccaggccctggagatggagaggaaggttcacacacttgtgcgtgcaagttaaagcctgaatgaagatttaaaaagtgtgtaggacggatgggagcaggagagaggctagaagacacttgcaataacccaggtgtgaggcaacccaggaatgcggagaggacygagagatcacagggggaggcctcgcaagatgaactgacacatgggatggcggcagggatagggatggggccctggggagagagcgtggcaagttctcagcattcgtccgggaagtcgatggtgtgtcatttgtctaggtgaggagatggatgaattccgtctggggcatgttaagggtcagggaaatggtcatgtggaagggtgcgcctaccaagctggaggagaggtgctgcaacttctttctgcctttgtatc

SEQ ID NO: 22 - DNA, 396 bp, Homo sapiens
acagttaccaggcttccagctggacagcttaccactgcggctcctgcagggaccccctggattctgcacctcagccccctcacccattcccatgacaccccctgccttccccctgacaatattctcccgggaccctccactcctcctgggccccaaggaggaaaggggacggaaatttcataccccttccaaggccagggrgcacaggaggggcggttctaggcaggcaggggccaggtgtccttctctgggggcctctgaaggtcacactgtggccaggcagccactcctccccctcctccctacttggaggcctgtagccaaggcctttgtgccagggtctgaggaacttgcggtgttagcagcgacccctgtccatggctttcctcttgcctc

SEQ ID NO: 23 - DNA, 101 bp, Homo sapiens
gctgcctgtgctggggtggggaggagtagagggcgagaagttggtggggargggaagcggcgccaaaagaatacccacaacatcttgcacctggaaggcaa

SEQ ID NO: 24 - DNA, 101 bp, Homo sapiens
atacaagctggtgccttctgtggttgtgcatggggtcttcatgcttcctgyctgagttcccagaagcttgtctctgcttttctaggcagctgccacagcct

SEQ ID NO: 25 - DNA, 101 bp, Homo sapiens
actataatatctctgggcaaaaatgtccccatctgaaaaacagggacaacrttcctccctcagccagccactatggggctaaaatgagaccacatctgtca

SEQ ID NO: 26 - DNA, 400 bp, Homo sapiens
ttataattaatggtattttagatttgacgaaatatggaatattacctgttgtgctgatcttgggcaaactataatatctctgggcaaaaatgtccccatctgaaaaacagggacaacgttcctccctcagccagccactatggggctaaaatgagaccacatctgtcaagggttttgccctcacctccctccctgctggayggcatccttggtgggcagaggtgggcttcgggcagaacaagccgtgctgagctaggaccaggagtgctagtgccactgtttgtctatggagagggaggcctcagtgctgagggccaagcaaatatttgtggttatggattaactcgaactccaggctgtcatggcggcaggacggcgaacttgcagtatctccacgacc

SEQ ID NO: 27 - DNA, 400 bp, Homo sapiens
ctgccagggcaggcatgcgtgatggcagggagccccgcgatgacctcctaaagctccctcctccacacggggatggtcacagagtcccctgggccttccctctccacccactcactccctcaactgtgaagaccccaggcccaggctaccgtccacactatccagcacagcctcccctactcaaatgcacactggcctcayggctgccctgccccaacccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccc

SEQ ID NO: 28 - DNA, 400 bp, Homo sapiens
tctaggatgccttttcccccatcccttcttgctcacacccccaacttgatctctccctcctaactgtgccctgcaccaagacagacacttcacagagcccaggacacacctggggacccttcctgggtgataggtctgtctatcctccaggtgtccctgcccaaggggagaagcatggggaatacttggttgggggaggaraggaagactggggggatgtgtcaagatggggctgcatgtggtgtactggcagaagagtgagaggatttaacttggcagcctttacagcagcagccagggcttgagtacttatctctgggccaggctgtattggatgttttacatgacggtctcatccccatgtttttggatgagtaaattgaaccttagaaaggtaaag

SEQ ID NO: 29 - DNA, 450 bp, Homo sapiens
ccccaacttgatctctccctcctaactgtgccctgcacccaagacagacacttcacagagcccaggagacacctggggacccttcctgggtgataggtctgtctatcctccaggtgtccctgcccaaggggagaagcatggggaatacttggttgggggaggagaggaagactggggggatgtgtcaagatggggctgcaygtggtgtactggcagaagagtgagaggatttaacttggcagcctttacagcagcagccagggcttgagtacttatctctgggccagggactgtattggatgttttacatgacggtctcatccccatgtttttggatgagtaaattgaaccttagaaaggtaaagacactggctcaaggtcacacagagatcggggtggggttcacagggaggcctgtccatctcagagcaaggcttcgtcctccaactg

SEQ ID NO: 30 - DNA, 400 bp, Homo sapiens
ggagttgtgggggtggctgagtggagcgattaggatgctggccctatgatgtcggccaggcacatgtgactgcaagaaacagaattcaggaagaagctccaggaaagagtgtggggtgaccctaggtggggactcccaccagccacagtgtaggtggttcagtccaccctccagccactgctgagcaccactgcctccccrtcccacctcacaaagaggggacctaaagaccaccctgcttccacccatgcctctgctgatcagggtgtgtgtgtgaccgaaactcacttctgtccacataaaatcgctcactctgtgcctcacatcaaagggagaaaatctgattgttcagggggtcggaagacagggtctgtgtcctatttgtctaagggtcagagtc

SEQ ID NO: 31 - DNA, 400 bp, Homo sapiens
agaccaccctgcttccacccatgcctctgctgatcagggtgtgtgtgtgaccgaaactcacttctgtccacataaaatcgctcactctgtgcctcacatcaaagggagaaaatctgattgttcagggggtcggaagacagggtctgtgtcctatttgtctaagggtcagagtcctttggagcccccagagtcctgtggacrtggccctaggtagtagggtgagcttggtaacggggctggcttcctgagacaaggctcagacccgctctgtccctggggatcgcttcagccactaggacctgaaaattgtgcacggcctgggcccccttccaaggcatccagggatgctttccagtggaggctttcagggcaggagaccctctggcctgcaccctctctt

SEQ ID NO: 32 - DNA, 400 bp, Homo sapiens
tcacatcaaagggagaaaatctgattgttcagggggtcggaagacagggtctgtgtcctatttgtctaagggtcagagtcctttggagcccccagagtcctgtggacgtggccctaggtagtagggtgagcttggtaacggggctggcttcctgagacaaggctcagacccgctctgtccctggggatcgcttcagccacyaggacctgaaaattgtgcacgcctgggcccccttccaaggcatccagggatgctttccagtggaggctttcagggcaggagaccctctggcctgcaccctctcttgccctcagcctccacctccttgactggacccccatctggacctccatccccaccacctctttccccagtggcctccctggcagacaccacagtg

SEQ ID NO: 33 - DNA, 573 bp, Homo sapiens
cccctcagagcagggtggggcaggggagctggtgcctgtgcaggctgtggacatttgcatgactccctgtggtcagctaagagcaccactccttcctgaagcggggcctgaagtccctagtcagagcctctggttcaccttctgcaggcagggagaggggagtcaagtcagtgaggagggctttcgcagtttctcttacaaactctcaacatgccctcccacctgcactgccttcctggaagccccacagcctcctatggttccgtggtccagtccttcagcttctgggcgcccccatcacgggctgagatttttgctttccagtctgccaagtcagttactgtgtccatccatctgctgtcagcttctggaattgttgctgttgtgccctttccattcttttgttatgatgcagctcccctgctgacgacgtcccattgctcttttaagtctagatatctggactgggcattcaaggcccattttgagcagagtcgggcygacctttcagccctcagttctccatggagtatgcgctctcttcttggcagggaggcctcacaaacatgccat

SEQ ID NO: 34 - DNA, 400 bp, Homo sapiens
ctatgcccatatgaccagggaacccaggaaagtgcatatgaaacccaggtgccctggactggaggctgtcaggaggcagccctgtgatgtcatcatcccaccccattccaggtggtcctgctggactcaaagaagaagctggcctgcggggcagtgctcatccacccctcctgggtgctgacagcggcccactgcatggaygagtccaagaagctccttgtcaggcttggtatgggctggagccaggcagaagggggctgccagaggcctgggtagggggaccaggcaggctgttcaggtttgggggaccccgctccccaggtgcttaagcaagaggcttcttgagctccacagaaggtgtttggggggaagaggcctatgtgcccccaccctgcccacc

SEQ ID NO: 35 - DNA, 400 bp, Homo sapiens
agcataatctatggccagtgcccccgtgggcttggcttagaattcccaggtgctcttcccagggaaccatcagtctggactgagaggaccttctctctcaggtgggacccggccctgtcctccctggcagtgccgtgttctgggggtcctcctctctgggtctcactgcccctggggtctctccagctacctttgctccaygttcctttgtggctctggtctgtgtctggggtttccaggggtctcgggcttccctgctgcccattccttctctggtctcacggctccgtgactcctgaaaaccaaccagcatcctacctctttgggattgacacctgttggccactccttctggcaggaaaagtcaccgttgatagggttccacggcatagacaggtgg

SEQ ID NO: 36 - DNA, 450 bp, Homo sapiens
ttccctccttatttcatcttcattcctggaaagtatttttgctaaatttaacaaaattctaggtttgcagttagtagattctattgtttctgttgtgaagtcagctgttagtctaatcattacttttctgaacgtattttttttcccttgtggctgcttttagactttcctattttcgttggtttcttgcagttttattaygatgtagttaggtgtagatttctttttgtttatcttccttgcaatgtgtacaacttcgagaatctatggtttaggtatcatttccttttaaaatactgcttctgctatacatgtaactttccctctcctttcattattccaatttttgcgaaaacttttcgatgtatagtctatatcttttcctttcttctgtaatactgtatcttgaagcttcattccagatctctccttctaagccatcttccagtt

SEQ ID NO: 37 - DNA, 400 bp, Homo sapiens
ctcctttcaccaagtactcaaagtaggagtccacgccagccccgatgcctgcgtcctgggccacccacttgccagtgagcacatcaatgtggttgccgacctgagagagagaaagacacacggtcccaacgggaaggccgatggccaaagaaggatctactcacccccaaccctgactgcccagggagatgcagggcaggygccccagtgcttcttgggaaacatgcagaccctgagagggaagggcaatgctggatcatggccagccttcctgtacatctgcatagtagagatgcatctcatgcacatttatgaggacttaattatacacattgagcaaaaaatgaaaaagaaaaatgatttggagtgtttatgtcctgcctagagtgagtgtgagatg

SEQ ID NO: 38 - DNA, 400 bp, Homo sapiens
aaaaagaaaaatgatttggagtgtttatgtcctgcctagagtgagtgtgagatgggagatgagaatttgctgttgccgcaatctgtctgatttctcagcacccagcatgtgattccactatctgaagacacagacgtgctttacgtatttccataaattaactcaataagaacatccaccaagaagctgacagagtggttmtaaggagagaaaccgaatagctggagacaggggcaaaaggggacttcaccaatgtcactgagtacccttttttgtatcctttgacttttttttttttaattgttcagtctctgtagagactgtgaaaaattggcaatgccggccaggcgcggtggctcatgcctataatcccagcacgttgggaggctgaggggggcaa

SEQ ID NO: 39 - DNA, 400 bp, Homo sapiens
ctgccgtacaggtgacaggggtctctcctgggttcacgccatgaagtaagttcactgttccatatggcatgccagtgggggtctgaaaggctgaacaatcgacaaattatgatcccggacaggagcagggggatagggatagttctgatacacgcccaaagcctgggaccttagccagcacttccctctttctcctgggtrtcctgctagagtctgagccagagaaagataaatgtcataactggagggccctgagcagccacccagcccagatgctgtcaaacactgctctgcataaccttgggttcctgctcatcatgagggggcagggagcaggctgtgctccacacacactcgctttagctagagagctttacctatttttatttattttacacta

SEQ ID NO: 40 - DNA, 600 bp, Homo sapiens
ctgggggtttgggacagaacacacgcagcttcagtcagttggtaaacgggtccctttcctctggggcagaaacgctttggggtttgactcaaatcatggactccttgggggcctattcttcgggctaactctttgcatgttctgcagggagccaaacaagccgctcctacacttcgctggtcctgggcgtcctggtgggcrgtttcatcattgctggtgtggctgtaggcatcttcctgtgcacaggtggacggcgatgttaattactctccagccccctcagaaggggctggattgatggaggctggcaagggaaagtttcagctcactgtgaagccagactccccaactgaaacaccagaaggtttggagtgacagctcctttcttctcccacatctgcccactgaagatttgagggaggggagatggagaggagaggtggacaaagtacttggtttgctaagaacctaagaacgtgtatgctttgctgaattagtctgataagtgaatgtttatctatctttgtggaaaacagataatggagttggggcaggaagcctatggcccatcctccaaagacagacagaatcacctgaggc

SEQ ID NO: 41 - DNA, 450 bp, Homo sapiens
ctgatgtctcagctcaaatggaatattgtcgcaccccatgcactgtcagttgcaatattcctgtggtgtctggcaaaggtaactgattcataaacatatttttagagagttccagaagaactcacacaccaaaaataagagaacaacaacaacaacaaaaatgctaagtggattttcccaacagatcataatgacattacrgtacatcataaaaatatccttagccagttgtgttttggactggcctggtgcatttgctggttttgatgagcaggatggggcacaggtagtcccaggggtggctgatgtgtgcatctgcgtactggcttgaacagatggcagaaccacagatagatgtagaagtttctccattttgtgtgttctgggagctcatggatattccaggacacaaaaggtggagaagagctttgttcatcctcttagcagata

SEQ ID NO: 42 - DNA, 450 bp, Homo sapiens
ggagatttggataaaagcaactatcattattatcctcatcagacttgtaggtctaactttttaattttttaatttttaatttaaatttttttcttggtcttttatcattaattaattttttcgagacagggtctcactctgttgcccaggctggagtgtggtgacatgatcacggctcactgcagccttaacctcccaggygcaagtgatcctcctctcttagcctcccgagtagctgggactccaggcatgtgccaccatgcccagctaattttttgtagagagagggttttgccatattgcccaggctggtcttgaactgctgagctcaagtgatccacccggcttgggcatgagccacctcccctggtctggtccaactttttaaaagcattattctgcctgttgggtggagaatagactgtaggtgggcaaagaatgaaggaaact

SEQ ID NO: 43 - DNA, 422 bp, Homo sapiens
aaatacaaaattagctgggtatggtggtgcacacctgtagtcacagctacttgggaggctgaggcaccagaattgcctgaacctggaaggcagaggttgcagggaactgagattgtgccactgcactccagtctgagcaacagagtgagactctctctcaaaaaaaaaaagaggtggaattgggagttgaccacaggcctrtctctccgaagtgcaggctttctctaacaccccctatagaaaggaagccatctagactcccagcacctcttacagtagagaagtaaccccactgtgctccctagtacagtatggatttacctatttttgataattcatcaaaatatagaagcaaagtctgtgccctatcgccttggtagctcaggcccagcacagggaggtatttagtgagcatttatgca

SEQ ID NO: 44 - DNA, 401 bp, Homo sapiens
gtaatgggttaaaatgataaattgtaaaatcaatgacgtcttaggaataatgaaaaatagtttaatagtgaatgaagaactatgtaattttaactgttcacatttactcttgggtatgtttccagaggataactgaacggggatagattttaaaaagctttatttaactgggtacttccgcaatttagtgatcaacttctrtgtacaacaaggtactgtcctttgaggatgatgggagaatacagggaagaacgaaatcgcctctgatcgtactttctccacggatgtaagtgtccgggctctagtgggggaatgatactcttcgtgcgaaattcacttttaaaaaaggcttagaaaactgaccaccggctctcagctgcagcttatcaaccacagaactc

SEQ ID NO: 45 - DNA, 456 bp, Homo sapiens
tttgttggaagtttttttcttgcacattttacaggcgagaaaagtgatgtagagaaaagcccaggcagtcccttggcatgtttagcagagaatcagtaccagcagcccccggcccggccttgtgtccaggaggtgcgcagggtgcgagatatatggtgacaatagcagaggctccgcgtggtggcgggggaggggacatgragaggattttgttgttacctagaacccattccttctaagtgagttgaagagagagatccctccccaggatcgggctccctcgaacactgtgggatcccagtatttcttaacgagatttctgatccactgcaagaaggttgctcccctagaatattttccccactagtagtctatttttaagtatctggccacttgaccaaataaataaatttgattaatttatttggtcaaatattttctgtatccctttcccca

SEQ ID NO: 46 - DNA, 400 bp, Homo sapiens
taaaatcactaataaaatcaacccaagataggtttactttctattaccaccatgcattgacaattctaagcattgtctgtcataaggtagtggtgctgggactctgaggcatccaacagtgcctcccatccttgaaccgccaccgctgtgatagagtttattgtccaggattaccagctctgtgtgccaagaggggcggtraagccctcccagggctggccctgaccaccaagctgagccttcctccagcacttcccgactttttgttcccactcattttggcatttcctgccttgtcactttgtgtgtgtgtctcattctcccaactaagattataaagttttatttatccccatggtgactaaaacaaatgttcactcagcagatagttgttgagaaa

SEQ ID NO: 47 - DNA, 456 bp, Homo sapiens
ccttggcatgtttagcagagaatcagtaccagcagcccccggcccggccttgtgtccaggaggtgcgcagggtgcgagatatatggtgacaatagcagaggctccgcgtggtggcgggggaggggacatggagaggattttgttgttacctagaacccattccttctaagtgagttgaagagagagatccctccccaggaycgggctccctcgaacactgtgggatcccagtatttcttaacgagatttctgatccactgcaagaaggttgctcccctagaatattttccccactagtagtctatttttaagtatctggccacttgaccaaataaataaatttgattaatttatttggtcaaatattttctgtatccctttccccaagagcagcacagatgagttgtttttagcctgtaaaggcgctaattagaaagtgagaaaagtgtttttgaa

SEQ ID NO: 48 - DNA, 450 bp, Homo sapiens
catggcaggagtggtgcatgttaatatggacagtgctggtgtagacagaaaggcaggtggatgaacttggctagtttatcaacactggattctggaaccactttgggagggaaagaagaaaggagtatgatagaggaaaaggagcgcttgctaagtgccatattccatgtcaagccctgggccagaaggaattttcacttrgattgtctcatttcaccttgtcaaaacaccttgttaaggtgggtatttatccccttttgctgattctgcaactaagacccagagacagcggctaagcaagtggtggcgggtggggcagggaaggggcagtccacccaccctgggtgcaagcaattaggaataagtggggctttgtctttagaaaatttaaaatcactaataaaatcaacccaagataggtttactttctattaccaccatgcattgaca

SEQ ID NO: 49 - DNA, 400 bp, Homo sapiens
gctagtttatcaacactggattctggaaccactttgggagggaaagaagaaaggagtatgatagaggaaaaggagcgcttgctaagtgccatattccatgtcaagccctgggccagaaggaattttcacttggattgtctcatttcaccttgtcaaaacaccttgttaaggtgggtatttatccccttttgctgattctgmaactaagacccagagacagcggctaagcaagtggtggcgggtggggcagggaaggggcagtccacccaccctgggtgcaagcaattaggaataagtggggctttgtctttagaaaatttaaaatcactaataaaatcaacccaagataggtttactttctattaccaccatgcattgacaattctaagcattgtctgtc

SEQ ID NO: 50 - DNA, 550 bp, Homo sapiens
atgccctccccatatcccatacccgccacgttcatgtttaattaaaaacagctaccctctgtggagtactgactacagctgacatccttcttagggacgttacaatactatcttatttatttctcacaacagccctttgagtagatgtcatcctcattttactggttataaaacagagacccagaatggttaagtcacaakttgagaaagaggtggaattgggactgggtgcggtggctcatgcctgtaatcccagcactttgggaggccaaagcagggggatcacttgaggccaggagtttgagaccagcctgaccaacatggtgaaaccctgtctctactaaaaatacaaaattagctgggtatggtggtgcacacctgtagtcacagctacttgggaggctgaggcaccagaattgcctgaacctggaaggcagaggttgcagggaactgagattgtgccactgcactccagtctgagcaacagagtgagactctctctcaaaaaaaaaaagaggtggaattgggagttgaccacaggcctgtctct

SEQ ID NO: 51 - DNA, 400 bp, Homo sapiens
aagtgcaggctttctctaacaccccctatagaaaggaagccatctagactcccagcacctcttacagtagagaagtaaccccactgtgctccctagtacagtatggatttacctatttttgataattcatcaaaatatagaagcaaagtctgtgccctatcgccttggtagctcaggcccagcacagggaggtatttagtragcatttatgcacggactgtggtattctctcatttactttcgctaacagatgataaggcaggctctgaaaagatccctgctcatgaatacactaattaatcagatgttacaagagatattgctagtaaacctaaacagaaagacagaaaactgagcagtggttctaccgtaagcagaccagaaagctctataaagcctg

SEQ ID NO: 52 - DNA, 400 bp, Homo sapiens
gcacctgccaccacgcctggctaatttttgtatttttagtagagacgagattttatcacactgcccaggctgttctcaaactcctgggatcaagcgatccacctaccttggcctcccaaagtgctggaattctaggcgtgagccaccatgcccagtctttaactagttttcgtgagcacctaggctccccttccattgcgratactcacaaaaacatccttgttagaagagttattaggactcagggccttggtttatttttgactatgatactagtgttgaggactccatagttttaccattcataattttctgtttgtttccttttttgcgatttctttcattctgcctttttctttctcttgcttgtgcctaaaactgtcgtcataaatagctctgt

SEQ ID NO: 53 - DNA, 450 bp, Homo sapiens
ctaaaagaaagatatttaacaaaatggttgagtacagatccaagagtcaaatagctgtctggttcaaagtccagctgtgtgattttgagctagtcacccaatctcactttgtctcagtagccttatttgtaaaaacaaggcaaattacagagccatcccctgggttgctatgaggactcaaacatgcatcccaagtgctcrgtgttgctaggtatgatggctcacacctgtacattcagcactttgggaggccgaagcagaaggatcagcctgggcaacatagcaggaccccatctctacaaaacaatgtttaaaaaaaagcaaagtgctcagcacagtgactgcatcattaggattgattgtagggctcctgatgttagcacagaacaccacagccaggaagcagtctatcttgttgggtgcaaattgtaacattccatttatgtttct

SEQ ID NO: 54 - DNA, 450 bp, Homo sapiens
agatgacgaggatgaacacctttatgatgatccacttccactttatcaatagtaaatatattttctcttccttataattctttctcttccttccttctttcttttcttttcttttcttttttttctttctttccctttcttttttagacagagtctcgctctgtcacccaggccggagtgcagtggcgcaatctcagttcrctgcaacctcctccacctgggttgaagtgattctcctgcctcagcctcccaagtagctgggattacaggcacccacaaccacgcctggctaatttttgtatttttagtagagatggggtttcaccatgttggccaggctggtcttgaactcctgacctcaagtgatccacccgccttagcatcccaaagttctgagattacaggcacgagccaccatgcccagcctcttttccttataattttcttaat

SEQ ID NO: 55 - DNA, 450 bp, Homo sapiens
atgatgatccacttccactttatcaatagtaaatatattttctcttccttataattctttctcttccttccttctttcttttcttttcttttcttttttttctttctttccctttcttttttagacagagtctcgctctgtcacccaggccggagtgcagtggcgcaatctcagttcactgcaacctcctccacctgggtygaagtgattctcctgcctcagcctcccaagtagctgggattacaggcacccacaaccacgcctggctaatttttgtatttttagtagagatggggtttcaccatgttggccaggctggtcttgaactcctgacctcaagtgatccacccgccttagcatcccaaagttctgagattacaggcacgagccaccatgcccagcctcttttccttataattttcttaataacattttctttcctctagctta

SEQ ID NO: 56 - DNA, 500 bp, Homo sapiens
ttaataatattattaatagtggtcatgagagaatatatgtataacatgttattatgtagactcactatatagactctattctacatagaatatagaacattatataacaaacaactataataagtagactatagtaaacaacctcactttgtctcagttgcctcatcttgatggaaaactgctctttctctcctgttaccytgacagagagcgtctacattctaaaagaaagatatttaacaaaatggttgagtacagatccaagagtcaaatagctgtctggttcaaagtccagctgtgtgattttgagctagtcacccaatctcactttgtctcagtagccttatttgtaaaaacaaggcaaattacagagccatcccctgggttgctatgaggactcaaacatgcatcccaagtgctcggtgttgctaggtatgatggctcacacctgtacattcagcactttgggaggccgaagcagaaggatcagcctgggcaac

SEQ ID NO: 57 - DNA, 450 bp, Homo sapiens
tcagaacatttccatggaatgaatatcaccggtgacggtttgtgctaaggcttaagccaataacatttcccaaccaccactgaaaactgttagcaaaggtgaaaaatgcagttggagttccaagtaggggcttctgcacagcagtagtgtcctgcggctggagccaggctgcagtagtgagagcagtcgggagggaagagrggcagctgcttaagatgctaactgtagggagggaaaacaggcagagaggaaggccaactgaggagatgcagtgggcaagactttccttcttcctcccgctttggagcctcccatcagactgtggcagagccacctgagggatggtggtgtgtggatactgggtagactttggttccagacctgacatgggcactcaccatcagtgtagtcatgaataaatccctcacttctcagagcaacagtttcctc

SEQ ID NO: 58 - DNA, 711 bp, Homo sapiens
ttcagatttcaccaattgagaattagtaagtaatttctctgatacaggcctgaagtttaccttagtaaacactttacttccatatggtaaaaattagattttgggaggaatgcttacctcctaaatatattcaatctaatatttgaggacacatgggaatatatttatgattcatctgctttttaaacataagcctttgttaactgtaagttcttgaactttataaggctgctgttatttaaatgagcacagctcctgatctgcaaacagcagagcgcagggctacagcttgggggatgccagccgactcagggtggtcctgtggactgaacaatctcttgctgctgtactggagggcctgggagcttttccatcagcctcggcctgaggtgtgcactcttctcctgcccaccccaggaataaatgagattcctggttaaaaaggaccagagcagtcattttacagttgaggaaactgttgctctgagaagtgagggatttattcatgactacactgatggtgagtgcccatgtcaggtctggaaccaaagtctacccagtatccacacaccaccatccctcaggtggctctgccacagtctgatgggaggctccaaagcgggaggaagaaggaaagtcttgcccackgcatctcctcagttggccttcctctctgcctgttttccctccctacagttagcatcttaagca

SEQ ID NO: 59 - DNA, 482 bp, Homo sapiens
gaggtggggaggaatcccaatgtatacattgcccttaagcagtgtttgattcattcatctttggactccatgaatcgaaatctggtagaatacatgatcttagtggaggaggccaaatgcgtgactcactgagcctggcagagcagaaatactctgctgtctgcaccctctgggtctggtgtggctctgcttcttggtgcytcaactctgactggcagctgtccccaggaggcgataattcagcatgttcaatctaaaggttatgacttccttgatggttttcaccatattcttggcaagtttttggtttttgaaatgttctaggaggcttggtagagatcttatgaaatagagaatagctgctgtggaaattattttaatgctaattacataaaagtacaaaagtagcactagctaaaacaaaaggtattttgctgttctgttttgttttagcttgtgccaggccttttacagcattagga

SEQ ID NO: 60 - DNA, 455 bp, Homo sapiens
tgtttcctctctccttctttcccacgttttcccagggaagtcagtcttgcattttaatgcatactatatacatatctcgtttagcttactgaaccacttgttttaacagaataaaactgtgcaaaattttaattttcctcctttgcctgaactgaaatagcacatccaggtttagcccttgtagactttccttcctcgaarcagaaagttgcccttgatgatttcctctttgagctctctgccagctctgaaacccacaaaatttatgtttgcaaaactaagccatgcaatcctctttttatgcaggctctagcctgagtcattttccctaagagatcttcagctccacctgggatgtgattctttgctctctgggattgaaggtagctgaagagaaatagttacacttcaggtttgttacaagacccaagaaattgttgcaattccacttggag

SEQ ID NO: 61 - DNA, 561 bp, Homo sapiens
gccacagggttctcatccataaaagaatgtctgtgaggttctcccatcctctgacatcctaaaatccaatgagaaagggactggtcaagccagagagattattgttatagtttagtaactttttgaacttctcagagcctccaagatagatcatggaggagggaactgttaactgctaagcttgactttactgacaggagtaaaaaaaattgtgttaaggttagggaataattttaacagtcaatttgttcttgtgaacaaatttcaacagtgaaattttagatatgtactttttaatggtgccaagcagcagttattatagatcaactgctgtttggcaccattaaaaagtacacttcgcaccgtcaaaaagtagatctggccacaattagatcagtcagggaaaaacacttcgcaatgaaatattatttaccacgttttcttcctccctcttcttgaaaatagtaatgaytttagcatttttaaatcttgaagaatgtcattccgtactgactaaaaagcctgtgcaaacacccaacatcttctctttcctgtctattt

SEQ ID NO: 62 - DNA, 544 bp, Homo sapiens
acattctcttttgctcttaacggaatggaaatcttagaaatgttgatgggacaatgacatgaatcatgaaaagaaaggaatagtgggaatacraatattagaaagccaatgttttgtggatgtttgaaactctcatatacattcatagggcttaccccactggtatgggcaacaggtaactctggtagtttctaaagatgctcccagtgaattatgcctcctggtatgtggtccttatgcagttcccttctacactgaatcgagactggcctgtaatttgtgttcacaacgaatagaatgcagcaaaagcaaagccctgtctcctagagattctggtatgttgtctctttgttctcattggttttaaggaacttatttatttctgccttaatttcattatttacccagtagtcattcaggagcaggttgttcagtttccatgtagttgtgtggtttcgagtgagtttcttagtcctcagttctaatttgatttctctgtggtctgagagattgttatgaatttcgttgataaaattttatatat

SEQ ID NO: 63 - DNA, 1032 bp, Homo sapiens
atttctgccttaatttcattatttacccagtagtcattcaggagcaggttgttcagtttccatgtagttgtgtggtttcgagtgagtttcttagtcctcagttctaatttgatttctctgtggtctgagagattgttatgaatttcgttgataaaattttatatatttatggtatacataacattttgatatatgtacacrttacaggatgattaaatcaagctaagtaacaaatccataatctcacataaattttttttggttgctcttttaacactaagttttggggtggtttatttcagacaccccaaatgactgtctatctcatgtgattttaaggatgtctaaaggttccccagttgtgcaatatctacaggatcactgaatgccaagtccccagggaaaggaatgatgaaaggggaagttgctggaagaagagagaggaggaagttgaggccatagagaggaaggccctgaaagaaaactttaactgcttgccagtttggccagaggtctctttgagcaggaacaactgcatttagaccagcagttcccatgctctgttttacaggtctgagctttccagtaggtgaaattatgttttgaaactgtgtgccatgtagtaccagctagaataaagccaacattacacattcagttctaccatggttatttcagttctgttccatatctaatgaccaccaaccttgaatatcaatgtgtgcagtccttagggagaccaggacggattcacaatttcaatggggctactggaaagatgcttggctgtttttttactcatggaaagtcagaaaaatcattgttatatgggaaagacaggatattttaagtacttatttcatttgataatattgtttttctcttcactcaagaaaaaccattaaaaaatcatgtgtttgtgaaagttatccaggtctatcaattattatttaaagtaatatctgttttactagtgtgtaaaggatttaaaagagttataatgaagcattttaactacataaatattacttc

SEQ ID NO: 64 - DNA, 411 bp, Homo sapiens
  misc_feature (119)..(119): n is a, c, g, or t
ggggttgaaaaagttcttcacatgtcctttggtattagtatttccttcaaaaatctgaaagccaaataagagaaaatctttaatgacaacataaatggctaaggttttttgtttttgtntttttttagacagggtcttattctgttgcccaggcttgagtccagtggcacagtcatggctcactgcagccttaaactcctytcaagtaatcctcttgcctcagccttccaaagtgctggcattgcagacatgagccatcatgcccaacccagaaaattttttatcctattagctcaaaatgagcatatcaaagaacacatagaacactctcacagagatgatcctctatcagttatgaaatcacatgccagatagatttcatctcaccagtgatctgacttaaattagtga

SEQ ID NO: 65 - DNA, 450 bp, Homo sapiens
accttatatcctcaactaaagtgtataactttcccccttccatcatcagcaagttccccaaggtttacacacaccagaaataaaatcactctttttttttatgacaatgatatttatcctcctatgagggcaacctggtgtagtgagaaataaaacaaaccaaaacagacaaccaggagtttgtctgagaccaggcacctkaagaactaagatttagaagacttaaagaggtggtacatgtcactgcatatttgtcaaatgcaaaatactgttattctcattatagcacagtcttcagattgctttctctttgcccagatgccactctaccttgtccaccatggaggatttcagcctgtatggtttccattccactccctgctcactgtagtggatggtatagctctttacatacatttcagaggacagagacttgcagccctgtgttat

SEQ ID NO: 66 - DNA, 400 bp, Homo sapiens
tgaatgaataaatgagctttatttggaaaaataattaaactgctaaattatctgtgaatattattttcttaagcaaatttctttaaatactctggtaatattgctgcagggttatctgggaaataattggatagcattctctctctatttcacctgttttacaaaacacaacttctctttccccctgttaatgtcaaagakcagaaatagttcaatttcttctaatatttcaaataaatgtaacatttgagagacctgataaaaccataagtagagcttgatacacagtacagttattgtcctgtctctggcctaacaaaatgcctaatcacatggttggtgatatgtgtttggagaaagatcagaaggtctaaactgagttttagagtcatctaagata

SEQ ID NO: 67 - DNA, 698 bp, Homo sapiens
aatattgatatgtttcttcaacatcacccacacatacataagtgtatgtgtatatataggtgtatgtgtatatatgtgtatacacaaaaattcacatgctatatgtattttaaaagcaaattaactgaattagaatgattatatgagaaaacttttaagtctataacatcctctagtgacatctgggtggtatcttagtgwgtcttcatatcaaggttgtaccagtgatgcagagtataaccagctagaggttttcacaggcataaaagaggtagagacattttagttatggagagaacagcatctatgtgcatcactgcatatgtccagctttgattttcaacctctctaaagagaccatataaagacatttcatgtgaatggaagggggttaagtaaaacacatatttctgatcccgaattctaactttgaaatgacaccattaaaaattagcattttatctttgaggcccttgttatatcacaaagactaagagcaaattatagagagtgcaaatttttttaaaagattgtaaaatgaagtatggagtgacaaggttataccatgaacaagtctaaagagtcatctgaaaaagaattggataacatctctttaattattatttatattgctatgtaaattatataaatgtaatataacactatttttattattttaacttctctgggtgtcagct

SEQ ID NO: 68 - DNA, 565 bp, Homo sapiens
tccagggcctatccttaaattaggccactagaaaggaaaaaagaattgtggtgctggtggcgtaaatagaaaagattggattccacacagtcttgggaactgatatctgtgtcttgaaactcattctggcccaatatggaatcacagaatgttacagtagcaaggagcaaagcatctggtctagatttttccttttaattstaaatacacaggagattaaataattttagcttagtttggtagcagaatcaggactagaatcccattctcccagtaaacaggccatgctccttccaccatttgaagcagcccaaatacctcattttgcaattttgcagagggcaaagctgacacccagagaagttaaaataataaaaatagtgttatattacatttatataatttacatagcaatataaataataattaaagagatgttatccaattctttttcagatgactctttagacttgttcatggtataaccttgtcactccatacttcattttacaatcttttaaaaaaatttgcactctctataatttgctcttagtctttgtgatat

SEQ ID NO: 69 - DNA, 456 bp, Homo sapiens
tcagtgatctaagttaaacaaacaccttccagagagttatactgtccctgatattagcccactgagtaattcaggtgatttaatttgggggtaactcttaatatttgactcatttttattaattctttaaatgacctgagatatcagaatggcatgaataacttgatgatcccttcagccaactaaatccaaattccctamtttctatcctcatatctccctcccttaagatacctacactccaatttcctggctttctatagaattccagggcctatccttaaattaggccactagaaaggaaaaaagaattgtggtgctggtggggtaaatagaaaagattggattccacacagtcttgggaactgatatctgtgtcttgaaactcattctggcccaatatggaatcacagaatgttacagtagcaaggagcaaagcatctggtctagattttt

SEQ ID NO: 70 - DNA, 456 bp, Homo sapiens
tctgtgttttatttgggagatgttttagactactgctatctggacattggcaaaatacccatatccatccaagggtatactgtgccattatctgcttcaacaggaaactgattccaggtttcagctactttctccattgtgaatcatggtggcttctctccacccaaagggaagtactgcaactcctgacaggtgtgccayggcaggtttctagtgcacctatttattgatccctcttcccacctccagcccttcagcagccaagtggggcctggatcagccaagccttagatttattgcttcattcttttacctcagaatgccaggcagatattattttctttgtgtgctttgaagtaaaaaatattggaaagcactgctttaagagtccaagaagaacagttaagactcttaaacatccttgctatatatagtagaattttattatgaccattt

SEQ ID NO: 71 - DNA, 614 bp, Homo sapiens
caaaaacactcattgaacaccayatatatggcagtgatgttgccagatactgtcatgacattgaagtttgagtgacctgaggactttggaagagtcaggcctagtttgaatctcaggtaggtcttattgaaaatgggctgatggaggtaattccaaattagagcccttccttggagagttgtgatgtgtctatataatccaggcactttcttcacagagatgctgtcggcactctgattggcagaaccattcttggtctagatcacactgagagtttacctgagtagaacctctgtttcacaaaggttttcctaggagcctaagtcactgaaaagaactaaaaattctactcattctcctatacctcccaaatcttgattctttgagtggcagtgagaaaataatgcatctttgtaccttaccatttacctcacaaccttgcagttccaatcgaagggtaggtctgttataggctcgagttggagagatcctaatatatctagccacaataggtgggtcaaactgattctcttttattgtagaggcatctgaattgccattaaaatactagaagaaaagaggaaagtttagttatgtaacaatgatctataaag

SEQ ID NO: 72 - DNA, 614 bp, Homo sapiens
caaaaacactcattgaacaccatatatatggcagtgatgttgccagatactgtcatgacattgaagtttgagtgacctgaggactttggaagagtcaggcctagtttgaatctcaggtaggtcttattgaaaatgggctgatggaggtaattccaaattagagcccttccttggagrgttgtgatgtgtctatataatccaggcactttcttcacagagatgctgtcggcactctgattggcagaaccattcttggtctagatcacactgagagtttacctgagtagaacctctgtttcacaaaggttttcctaggagcctaagtcactgaaaagaactaaaaattctactcattctcctatacctcccaaatcttgattctttgagtggcagtgagaaaataatgcatctttgtaccttaccatttacctcacaaccttgcagttccaatcgaagggtaggtctgttataggctcgagttggagagatcctaatatatctagccacaataggtgggtcaaactgattctcttttattgtagaggcatctgaattgccattaaaatactagaagaaaagaggaaagtttagttatgtaacaatgatctataaag

SEQ ID NO: 73 - DNA, 565 bp, Homo sapiens
ctgtcatgacattgaagtttgagtgacctgaggactttggaagagtcaggcctagtttgaatctcaggtaggtcttattgaaaatgggctgatggaggtaattccaaattagagcccttccttggagagttgtgatgtgtctatataatccaggcactttcttcacagagatgctgtcggcactctgattggcagaaccaytcttggtctagatcacactgagagtttacctgagtagaacctctgtttcacaaaggttttcctaggagcctaagtcactgaaaagaactaaaaattctactcattctcctatacctcccaaatcttgattctttgagtggcagtgagaaaataatgcatctttgtaccttaccatttacctcacaaccttgcagttccaatcgaagggtaggtctgttataggctcgagttggagagatcctaatatatctagccacaataggtgggtcaaactgattctcttttattgtagaggcatctgaattgccattaaaatactagaagaaaagaggaaagtttagttatgtaacaatgatctataaag

SEQ ID NO: 74 - DNA, 456 bp, Homo sapiens
ttaccgatacctgctccaatcttctgttttaaaaagttggctttttctgacattgctctgtcaggaaaaggggtagggcacagcctgtttactgccaagtgggggtcaaagtccaggttccccactccattgccacctaagaagggattgttccttggtggctgggtgggaagggaagttccccatttggcctccactgayactgcaggggcaggagcttcattaggggctggagatgaaagccctaaatccctacatggccttttctgacacaaccccagtgagggtgtagggtgcctctttagcctcaggagcatagaagtctaggctccccattcagcctttgctgttgtgggttggggaggggcctcaggtttttctgtggtgtttggctaaaggagagaagtcagtgtccaccagttttctatcatatctcgctatgctgccctttactgg

SEQ ID NO: 75 - DNA, 399 bp, Homo sapiens
  misc_feature (201)..(201): n = absent or A
attgtcatctgcccctcctttcttttttgtgtgaatcttgctagagatttgtcgattttaaaaaaatcttcttggccgggcgcagtggctcacgtctgtaatcccagcactttgggaggccgaggtgggcagatcacgaggtcaagagatcaagaccatcctgaccaacatggtgaaaccccctctctactaaaaatattngcaggagtatcgcttgaatccggaaggcagaggttgcagtgagccgagatcacgccactgcactccagcctggtgacggagtgagactccgtctcaaaaaaaaaaaaaaaaaaaaaaaatcttttcacagaaccagccttgttttattgatttttctctattgtttttctgttttcagtattaccgatacctgctcca

SEQ ID NO: 76 - DNA, 456 bp, Homo sapiens
gtatatttcaattaactggtcaattccctctacgttgttaagtttatatgtgtagagttgtttgtatattcccttatcctttttcatgtttgtagtgattttgcctgtttcatctctgatattgtcatctgcccctcctttcttttttgtgtgaatcttgctagagatttgtcgattttaaaaaaatcttcttggccgggygcagtggctcacgtctgtaatcccagcactttgggaggccgaggtgggcagatcacgaggtcaagagatcaagaccatcctgaccaacatggtgaaaccccctctctactaaaaatattaaaaaaaaattaggcagggtggcgtgcacttgtaatcccagctacttgggaggctgaggcaggagtatcgcttgaatccggaaggcagaggttgcagtgagccgagatcacgccactgcactccagcctggtgacg

SEQ ID NO: 77 - DNA, 456 bp, Homo sapiens
ctgaggttggttctaataatatcttgttttatagattaaaaaacaaaaggcacagagatactacctaccttcccaaagtaacacacagctaattagtcattgagttgggattcaaatttaggtactctgtgcccaaatatgaattgcatgtagtcattgtatcagtttgctaagatcttatgtgctagctctttagttctraagaaagctgattgtataatgaatttaggcagtgtgtgacttgttgacaaggacagttctgtttactggctttcctatattgcaggtggacatgcaaaaggaagtcataatcacagggatccagacccaaggtgccaaacactacctgaagtcctgctataccacagagttctatgtagcttacagttccaaccagatcaactggcagatcttcaaagggaacagcacaaggaatgtgatggtttgtgtgcatat

SEQ ID NO: 78 - DNA, 613 bp, Homo sapiens
  misc_feature (408)..(408): n is a, c, g, or t
ttgtcatgtatggtttcataggctgcatgctgcacaactgtagggggtaccattcacagaccatggtgctacaacttacccagaaactctgaagccttgatctgtgaatcagatatgataccagtgcttagtcccattggcatcctacagtctatgaaaaacagaaaaaaaatgaataatttttgcttagaaaatatatayaataaagtaaaactccatggttagggattatgtttctgactcaataattagatatttttacctaccttgtgtggccctgacttaaatatatctgattataaaatggtaaatgcactaacaagactggtgctttaatagatatagtggcatcctagaagaaagcaactgaaatcctgaaatgtaatcattttttaaactgattttctnctgcttttatcttaaattatgctattttacaacattaggtaccatccattggccaggtgcagtggctcatgcctgtaatcctagcactctgggaggccgaggggggaggattacttgaggccaggagtttgatcttgggcaacctcttctctacaaaaaaaataaaaatacaaaatataaaaatatgaaaaaaagttctagtcct

SEQ ID NO: 79 - DNA, 456 bp, Homo sapiens
aaagcaaaaatgaaaagagggaaaggaaacatgtatgatatttttgaagcccaatgatgtcattcgaggtcactttgataattaggactgtgaagctgtattgtatgtgagttaatttgtaacattatatttagataagtgaaagtttcttctagtgaggtgtggtggtgatttaaaattttttttagttatctctgtgtrtgtgttgttattgttttgtttttatctgttatactttcagtcctacctaaagaaaaatggttaaattctatttgaaagcctcttgtgaagcaggaattttaggattcttagagaactatcaaccacaatatttacttgttaatttttgcaaatgtaatgttgttctttttattttaggttcatttaaaactcttgaaatgaaggcatcaaaacctggctggtggctcctaaacacagaggttggagaaaaccaga

SEQ ID NO: 80 - DNA, 456 bp, Homo sapiens
aactttatctttacagttttgtgttacatttttaaggtgatcaagtttctcaggaactttttaaaaaatccgcgtattttactgtggggagagtagataaagcctgagaaccctagttatctaatctgagaagtgggcagaggagttgtcataccaataagagaataaacatgacttgctatggttgctggatacaccaaygataaactcctaattttataaactcctagacttcctaatttgcctgaaactttctctgaatttagaaggccttaaggtgacatgctgcatatctccttgctgactctaatccctggagtttactttgtctgcccatatttgtatttatcttttgtagttaaggaaaattaagactgttaatggaaagtatacacgggtaaagcacggaactgtaaaagctgaggaaaagagtgataaactgcttggaaagggagt

SEQ ID NO: 81 - DNA, 701 bp, Homo sapiens
tcaccttaaaaatgtaacacaaaactgtaaagataaagtttagtgttaccaaacgtttaactgaaaccaaagtgaaggaagtgctttgtttcctccttcttaagttagaggaactgctgtgaaatttcagaaaccaggcaggtgaatttttggagagggacgggttgagatcatttgggcatccttcaaattatatcaggygtgagaacacctatctacatattcataacacaagctcgtacctttctcttctcagtttctgtttcagttactagcttacagggcaaattttccttgtgaatttccaaataggcacacttttcccagtgtgaccacccaaatgctctcttgccactattctcctattacctgggtacctagtttcttatctcattttgttctcatatgctttgggtgccacctggtagctgctgagagctaaaactgttagcgtcattcttattttagaaaagacacacaggtgatcatttcttttatataaacacttgtgcctttagagatccagaaacaagcttattaactttaaattgcagaacaatgttaattccttttcaatgtatgttaccagctgaagcacactggctttttgttgttgtggttttctgttggtttgttttgcagcagcttctctgtgctatctttcagactttctctttttgcctcacccacagtgtcacctg

SEQ ID NO: 82 - DNA, 701 bp, Homo sapiens
ccttatgtagtattcctttttggcagattaggaggggacctatcaagcctgagtgaatatctttttcctggaaaaacagagtaaattgtattgcctctttctcaggaatttccttgctcttctgataatcactcatcatatagaaaaggaaactttctgataggctctgaatttgaagatgaagattataaatgaatggcrtaataagcctggatatttatcacctaattctgttgtattcataatcctccttcctctgattgatctttatcccagtaacaatgatgataataataaattgataatataagggatactatttattgagaatctattactatattaaatgatttgcctgcaatctcccatttaatcctgacaactatcttatatggtaaatataaattcattcattgaacaaatatttaccaagtgcttaccatgcactatgtactgttatctcatttttaacttctgataccaggctaagagaggtcaaggaatttcctaggattatgcattgacagggtaaaaatttaaatctgagtctgtgctctttccactatgcctgaaatggaggagttgtttctctttttaattgacagataaaattgtatgcatatactgtgtatgacatattgttttgaagtatatatacattgcagaatgtcagataaaggagtcttgactttgcagttctt

SEQ ID NO: 83 - DNA, 657 bp, Homo sapiens
atgaatggcataataagcctggatatttatcacctaattctgttgtattcataatcctccttcctctgattgatctttatcccagtaacaatgatgataataataaattgataatataagggatactatttattgagaatctattactatattaaatgatttgcctgcaatctcccatttaatcctgacaactatcttatmtggtaaatataaattcattcattgaacaaatatttaccaagtgcttaccatgcactatgtactgttatctcatttttaacttctgataccaggctaagagaggtcaaggaatttcctaggattatgcattgacagggtaaaaatttaaatctgagtctgtgctctttccactatgcctgaaatggaggagttgtttctctttttaattgacagataaaattgtatgcatatactgtgtatgacatattgttttgaagtatatatacattgcagaatgtcagataaaggagtcttgactttgcagttcttttcataaagaaagagcagaacatagctaatacttgttcaagaaaatttcaaataaatgccatcttctgtaaatgtaggcattctaattcatggccaatcattcaagtaatctttcctcctctccactgaataaatgtttctctctct

SEQ ID NO: 84 - DNA, 519 bp, Homo sapiens
agggtaaaaatttaaatctgagtctgtgctctttccactatgcctgaaatggaggagttgtttctctttttaattgacagataaaattgtatgcatatactgtgtatgacatattgttttgaagtatatatacattgcagaatgwcagataaaggagtcttgactttgcagttcttttcataaagaaagagcagaacatagctaatacttgttcaagaaaatttcaaataaatgccatcttctgtaaatgtaggcattctaattcatggccaatcattcaagtaatctttcctcctctccactgaataaatgtttctctctctgtcatctgaagagctgcatggagagtccctggttatgataaatgcagactgttaaccacacccttatgcattcctcatgaaaagcaagacagacatttgacaagaaataaccccgactcttccatttggtggacttcagattacgaggttaggggaatgagaaaaactttcaatgaaagtacctactgggttcaca

SEQ ID NO: 85 - DNA, 456 bp, Homo sapiens
ttaaggaagataatgctgttcagccaaatagcagttatacctacgtatggcatgccactgagcgatcagggccagaaagtcctggctctgcctgtcgggcttgggcctactactcagctgtgaacccagtaggtactttcattgaaagtttttctcattcccctaacctcgtaatctgaagtccaccaaatggaagagtcrgggttatttcttgtcaaatgtctgtcttgcttttcatgaggaatgcataagggtgtggttaacagtctgcatttatcataaccagggactctccatgcagctcttcagatgacagagagagaaacatttattcagtggagaggaggaaagattacttgaatgattggccatgaattagaatgcctacatttacagaagatggcatttatttgaaattttcttgaacaagtattagctatgttctgctctttcttt

SEQ ID NO: 86 - DNA, 542 bp, Homo sapiens
  misc_feature (339)..(339): n is a, c, g, or t
tcttcacatctccactacctatcactctcatttcattagtagataatgtcagtactttagccttgagcctaagaacaaatatcttttggtatttctggagaaaactacttgggccatatctcacaggatggttatgaaaattaaatgaaataatatacataagttattttgtaccttatatcttagctccgattttataaycagccattttgacttataatgctgacatttttgtggtttagatttttgttaagcttaagtacatttgtggatcattccttttcctaggttcgttttaaaaatttagcatccagaccgtattctctacatgcccatggnctttcctatgaaaaatcatcagagggaaagacttatgaagatgactctcctgaatggtttaaggaagataatgctgttcagccaaatagcagttatacctacgtatggcatgccactgagcgatcagggccagaaagtcctggctctgcctgtcgggcttgggcctactactcagctgtgaacccagtaggtactttcattga

SEQ ID NO: 87 - DNA, 465 bp, Homo sapiens
tttaaaaaacttcagttgctttaggaatgcaagtggtttttggttacatggatgaattgtatagtggtgaagtctaggattttagtgtacctgtcacctgagtagtgtaccctgtacccaataggtagcttttcatccatcccccgttcccttttgagtctccaatgtccattataccaatctgtctgccttcgcataccygcagtttatctcccatttgtaagtgagaacatacagtgtctggtttttgattcctgagttacttagaataagttttttaaatattgtgttaaattatcgttcatcttgaaaaaggatcccctaatcataaaagagaatattgcctcccatagcttcatggaaaatttagaataattaagattcttatatccctatgtacttgtttacgtttttaaaaagagcaaatggtcactgaaaatgtagtgaatgcttacacaggtatag

SEQ ID NO: 88 - DNA, 571 bp, Homo sapiens
ataaatgagtaaatatataagtggataaaaacaaaagccagtaaatatctttcaattcctaactcaaatattattttataggtaagtcttcctatgaccttttagactaggtcaagtctctatatcaatttctcatatttctatagtattattatagtatatatagtaaattcctgtagtattcttataatatctctagartatatattgtagattatatataatttatttgtgagattatctattaatatctgtctctcactgggtataagtgccatgaggtcagccattgtgcctagtttgctcatagtactctctccagtggtaagcacattatctgacacacagcaggaacttaataaatttttgtcaaacatataaatgaatgtataaatataatcgtatgttaacacaccaaattttaagatcaaaggcagacaaagccatgtaatgggacagtgccagagcttggggctatcaggtgacaatggtcagattaattagaaggtcacacttatgaaagtcactggatgggtgaatgttttgtaccctaaaagtagccactcttctc

SEQ ID NO: 89 - DNA, 426 bp, Homo sapiens
tcacatttcccaagcctgtggaaacaagccaaacactcacccatcaaacccataatatttgctatatactgtgagtcatcaacagagaatctccttctgcctttcttctggtctacctcccctactaatcccatctttccagactctgagcataacatgcaaactcacagaacacaagggagtgggtaaagcaactccgamtgccataaaagtgggttgtgagccttgaatggaatacaagattttgaaggtggttccatccctattcactctggacaggccctgcatctcactccctcggggccttgcttagaaatactcaggtagctagttgttctcatgtggtattgagtgcaacatttaaataggaagtcataggaaaaggtgttttaaacagagttctaatgtggagatgtcaggcatcag

SEQ ID NO: 90 - DNA, 456 bp, Homo sapiens
ctcacagaacacaagggagtgggtaaagcaactccgaatgccataaaagtgggttgtgagccttgaatggaatacaagattttgaaggtggttccatccctattcactctggacaggccctgcatctcactccctcggggccttgcttagaaatactcaggtagctagttgttctcatgtggtattgagtgcaacatttawataggaagtcataggaaaaggtgttttaaacagagttctaatgtggagatgtcaggcatcagattaatgaactcatatgcataagtcacaccatacattgtgtttgcctactgtaattacactttggttttttaagtgattagtgtaacagctttagtaaaagctgtgggcatatctagacagctgatgcacagtcatcatgctatatccctgagaatttgtagttgactaagccttgctcctttcctcctctct

SEQ ID NO: 91 - DNA, 456 bp, Homo sapiens
aaagtcaagaacatgctaagcataagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggraggaccctcctagtgatctgttactcttaaaacaaagtaactcatctaagattttggttgggagatggcatttggcttctgagaaaggtagctatgaaataatccaagatactgatgaagacacagctgttaacaattggctgatcagcccccagaatgcctcacgtgcttggggagaaagcacccctcttgccaacaagcctggaaagcagagtggccacccaaagtttcctagagttagacataaatctctac

SEQ ID NO: 92 - DNA, 456 bp, Homo sapiens
ttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcaaaagtcaagaacatgctaagcrtaagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagattttggttgggagatggcatttggc

SEQ ID NO: 93 - DNA, 456 bp, Homo sapiens
tgcagaacctcagaaagccccttctcaccaacaagccaccacagctggttccccactgagacacctcattggcaagaactcagttctcaattcttccacagcagagcattccagcccatattctgaagaccctatagaggatcctctacagccagatgtcacagggatacgtctactttcacttggtgctggagaattcaraagtcaagaacatgctaagcataagggacccaaggtagaaagagatcaagcagcaaagcacaggttctcctggatgaaattactagcacataaagttgggagacacctaagccaagacactggttctccttccggaatgaggccctgggaggaccttcctagccaagacactggttctccttccagaatgaggccctggaaggaccctcctagtgatctgttactcttaaaacaaagtaactcatctaagatttt

SEQ ID NO: 94 - DNA, 400 bp, Homo sapiens
gtcctgatctgccaatcgattgctgtgtaaccttacacaagttacttggccacactgagccacaagtcatttatctggaaaacagtgtaatcacatctcacagagttactttgaccattaaaatagtaatatgcgccaagtgcctagcactcagtagacaccaacaatggtaactattggagactcaccaagaaatctttratgttccagcaatgcatgccatttcagagattcaaaattgtcctcgtgaattattacttagaaacatctaaatgtctcttatttgtggggatagagctcatcaccatccctttaattctaagacaagatgtgctgttaggatatttatgatattaaaagtccattttattcttgattcatccctttcttaatatatttt

SEQ ID NO: 95 - DNA, 445 bp, Homo sapiens
tcatcaggtacagtgccttaaaggctgcttcagcaacagctttggagtttgtcagactggaatgcaagtcctgatctgccaatcgattgctgtgtaaccttacacaagttacttggccacactgagccacaagtcatttatctggaaaacagtgtaatcacatctcacagagttactttgaccattaaaatagtaatatgygccaagtgcctagcactcagtagacaccaacaatggtaactattggagactcaccaagaaatctttgatgttccagcaatgcatgccatttcagagattcaaaattgtcctcgtgaattattacttagaaacatctaaatgtctcttatttgtggggatagagctcatcaccatccctttaattctaagacaagatgtgctgttaggatatttatgatattaaaagtccattttattcttgatt

SEQ ID NO: 96 - DNA, 456 bp, Homo sapiens
cacttcactgggcactcattcatctatggaaagaggcatgaggacaccttgaccctcttccccatgcgtggagaatctgtgacggtcacaatggataatgttggtgagtaagagtctggacactcacagaggaagcttgctttgaatttctggtctataaaggtctgctgcaactctccaggctaccagtgctcctctatktatctccctgaccccctgcaggcttttctttcaatgtttctcatgatttctctttgagaaattaatgacttaaatggatccagttctttagtgtgggttatatttttccttctctgggcaaagtaggaagtaaaaatatacaacagcagaaaaataaggcataactctgaggaagaagcataaatattttggccacaaaagagcatttcttttatcaaaatgccctattcggttttttgcaacagtcatcttcaa

SEQ ID NO: 97 - DNA, 400 bp, Homo sapiens
tgtagccagtggctaccatattgaacaatgcagttatagaccatttagaaaaacatctagaatgaggtataaactaataatacatcatgtaaattattaaagtcacttaagtgatttaagtaggtatttaaatatttgttaatgccagtcattatactggtactaaggctaagagtggtaatcgaggtagacatgagcccygcccttgtagaaatcatacctttggttttttactatgcttagtacataagtaaataaaaatattgcctacagcagtgtcccttttaacaataatgaaatgtatgaactggatactcaaatggaaactgtgaatactatgtagattataagacagcaataaaaactataaaatatgctaaatgggctttatttttaggga

SEQ ID NO: 98 - DNA, 400 bp, Homo sapiens
aagtgctcgataaccacttgtagccagtggctaccatattgaacaatgcagttatagaccatttagaaaaacatctagaatgaggtataaactaataatacatcatgtaaattattaaagtcacttaagtgatttaagtaggtatttaaatatttgttaatgccagtcattatactggtactaaggctaagagtggtaatsgaggtagacatgagccctgcccttgtagaaatcatacctttggttttttactatgcttagtacataagtaaataaaaatattgcctacagcagtgtcccttttaacaataatgaaatgtatgaactggatactcaaatggaaactgtgaatactatgtagattataagacagcaataaaaactataaaatatgctaaat

SEQ ID NO: 99 - DNA, 456 bp, Homo sapiens
attacctactagagaaggtgattaccatgactctagactctgaggatcagtagggggaccctggctgttgtggagaagtttgcttccagctggctcagtgacatttctctcagactgccatggcagtgcttttggccactgaactttagaactgcagcaaatgccggaaccctcattcagggaattccttttgttctgaamatcttactgatcacttgaaatgtcttcatgcatgcctttccaagactcttgggtccctatactcattttgctctactttgatgtgtcaatccatctttggattacttgccttctttctgtcctagccatatattcaccctgaactcagtctaggatactattgacatggactataacaccttccattagtcctactctctcacccttttcctcaccacgcagagtatgtctgtgtacacacacacacacacacac

SEQ ID NO: 100 - DNA, 456 bp, Homo sapiens
ttaccatgactctagactctgaggatcagtagggggaccctggctgttgtggagaagtttgcttccagctggctcagtgacatttctctcagactgccatggcagtgcttttggccactgaactttagaactgcagcaaatgccggaaccctcattcagggaattccttttgttctgaaaatcttactgatcacttgaaaygtcttcatgcatgcctttccaagactcttgggtccctatactcattttgctctactttgatgtgtcaatccatctttggattacttgccttctttctgtcctagccatatattcaccctgaactcagtctaggatactattgacatggactataacaccttccattagtcctactctctcacccttttcctcaccacgcagagtatgtctgtgtacacacacacacacacacacacacacacatgcttggaatag

SEQ ID NO: 101 - DNA, 456 bp, Homo sapiens
actgccatggcagtgcttttggccactgaactttagaactgcagcaaatgccggaaccctcattcagggaattccttttgttctgaaaatcttactgatcacttgaaatgtcttcatgcatgcctttccaagactcttgggtccctatactcattttgctctactttgatgtgtcaatccatctttggattacttgccttstttctgtcctagccatatattcaccctgaactcagtctaggatactattgacatggactataacaccttccattagtcctactctctcacccttttcctcaccacgcagagtatgtctgtgtacacacacacacacacacacacacacacatgcttggaatagaagatcaaacgcatttctaaggatgtgagcctttgacctcttgcttaaaaatgttgctatgatgtcacccacggatttcatcaccaagtctt

SEQ ID NO: 102 - DNA, 456 bp, Homo sapiens
agtgcttttggccactgaactttagaactgcagcaaatgccggaaccctcattcagggaattccttttgttctgaaaatcttactgatcacttgaaatgtcttcatgcatgcctttccaagactcttgggtccctatactcattttgctctactttgatgtgtcaatccatctttggattacttgccttctttctgtcctrgccatatattcaccctgaactcagtctaggatactattgacatggactataacaccttccattagtcctactctctcacccttttcctcaccacgcagagtatgtctgtgtacacacacacacacacacacacacacacatgcttggaatagaagatcaaacgcatttctaaggatgtgagcctttgacctcttgcttaaaaatgttgctatgatgtcacccacggatttcatcaccaagtctttggactggaag

SEQ ID NO: 103 - DNA, 497 bp, Homo sapiens
aaaatcttactgatcacttgaaatgtcttcatgcatgcctttccaagactcttgggtccctatactcattttgctctactttgatgtgtcaatccatctttggattacttgccttctttctgtcctagccatatattcaccctgaactcagtctaggatactattgacatggactataacaccttccattagtcctactcyctcacccttttcctcaccacgcagagtatgtctgtgtacacacacacacacacacacacacacacgcttggaatagaagatcaaacgcatttctaaggatgtgagcctttgacctcttgcttaaaaatgttgctatgatgtcacccacggatttcatcaccaagtctttggactggaagtgaggattggaggtgccccttagcgagtagattttaatccatgtctctgactctaggcacagtcatatttcaaccacaggaatgaaaaactgatgaacaaaaatagtactctgactt

SEQ ID NO: 104 - DNA, 452 bp, Homo sapiens
  misc_feature (201)..(201): n = absent or A or G
ttcattcctgtggttgaaatatgactgtgcctagagtcagagacatggattaaaatctactcgctaaggggcacctccaatcctcacttccagtccaaagacttggtgatgaaatccgtgggtgacatcatagcaacatttttaagcaagaggtcaaaggctcacatccttagaaatgcgtttgatcttctattccaagcnngtgtgtgtgtgtgtgtgtgtgtgtgtgtacacagacatactctgcgtggtgaggaaaagggtgagagagtaggactaatggaaggtgttatagtccatgtcaatagtatcctagactgagttcagggtgaatatatggctaggacagaaagaaggcaagtaatccaaagatggattgacacatcaaagtagagcaaaatgagtatagggacccaagagtcttggaaaggcatgcatgaagacatttcaag

SEQ ID NO: 105 - DNA, 597 bp, Homo sapiens
  misc_feature (391)..(391): n is a, c, g, or t
ggatactattgacatggactataacaccttccattagtcctactctctcacccttttcctcaccacgcagagtatgtctgtgtacacacacacacacacacacacacacacgcttggaatagaagatcaaacgcatttctaaggatgtgagcctttgacctcttgcttaaaaatgttgctatgatgtcacccacggatttyatcaccaagtctttggactggaagtgaggattggaggtgccccttagcgagtagattttaatccatgtctctgactctaggcacagtcatatttcaaccacaggaatgaaaaactgatgaacaaaaatagtactctgacttactgctcatgatgtttgattcataaaacttggggtcatcacgtttcacntcatcaggattttcacaaaacttgttgatgttgtcctcaaggtaccagcttttgttctcatcaaaccacagcaaacacagcctgctgttcgatgtctgctgccctctggaggacaaaacagtatagtactggtacaagaacagacgcatagaccaatggaacagaatagagaactcagaaataaggctgcgcacctacaactatct

SEQ ID NO: 106 - DNA, 544 bp, Homo sapiens
accaattaatattgcaaaaggaattcttttattttttatttgtttttaaattatactttaagttctagggtacatgtgcacaacgtgcaggtttgttacatatgtatacatgtgtcatgttggtgtgctgcacccattaacttgtcattaacattaggtatatctcctaatgctayccctccccccgccccccaccccccccgacaggccccagtgtgtgatgttccccatcctgtgtctaaatgttctcattgttcaattgaattctttaaatattctacttggaacctggataacatgtagccattagataatgctccactagaggccactatgacactaataaaagacaccatattttgttaccactaagagacaaaactcctgaagtgagaagggtttggctgtgatttttaggatactcctacatgtatactacctgactgcagtagtgacaccaccgggcaaggagaatagcagaaaaatgtggcagcctctcagaagttactagttggattcagtagaagtgaaagattcaaacctg

SEQ ID NO: 107 - DNA, 532 bp, Homo sapiens
tgcaaaaggaattcttttattttttatttgtttttaaattatactttaagttctagggtacatgtgcacaacgtgcaggtttgttacatatgtatacatgtgtcatgttggtgtgctgcacccattaacttgtcattaacattaggtatatctcctaatgctacccctccccccgccccccaccccccccgacaggccccrgtgtgtgatgttccccatcctgtgtctaaatgttctcattgttcaattgaattctttaaatattctacttggaacctggataacatgtagccattagataatgctccactagaggccactatgacactaataaaagacaccatattttgttaccactaagagacaaaactcctgaagtgagaagggtttggctgtgatttttaggatactcctacatgtatactacctgactgcagtagtgacaccaccgggcaaggagaatagcagaaaaatgtggcagcctctcagaagttactagttggattcagtagaagtgaaagattcaaacctg

SEQ ID NO: 108 - DNA, 624 bp, Homo sapiens
ctctccaagcaagtcctccttcccctgcccttctcttttcagcttgggccacatctcattttgaatctgcttctcatctctagaccatgatccccttcccctgcccggtagattttttaggacactgtctttgaagtcatcttctcagctaggttcagtggctcaaacctgtaaccccagcactttgggaggctgaagcasgtggatcacttgagctcaggagttcaagaccagcctgggcaacatggtgaaacctcatctctacaaaaaaatacaaaaattagccaggcgttggggcgtgtgcctgtagtcccagctacttgagaggctgaggtgcgagaatcgcctgagcccaggaagtggaggttgcagtgagccatgatcacaccactgcactccagcctgggtgacagagtaagacccttggtgggggggaaaagctacttgcttgagaggcacagacaaatagccagatcctgaacttttaataaggtatgtctacacttgctgagagcacataaaagggatcagaaatggaagaagggaaagaggcatggagaaaaaaaactgcttttgtcctttggctattttaaatgaacagaatagaccttgtcaggtgcataa

SEQ ID NO: 109 - DNA, 534 bp, Homo sapiens
tccccttcccctgcccggtagattttttaggacactgtctttgaagtcatcttctcagctaggttcagtggctcaaacctgtaaccccagcactttgggaggctgaagcaggtggatcacttgagctcaggagttcaagaccagcctgggcaacatggtgaaacctcatctctacaaaaaaatacaaaaattagccaggcrttggggcgtgtgcctgtagtcccagctacttgagaggctgaggtgcgagaatcgcctgagcccaggaagtggaggttgcagtgagccatgatcacaccactgcactccagcctgggtgacagagtaagacccttggtgggggggaaaagctacttgcttgagaggcacagacaaatagccagatcctgaacttttaataaggtatgtctacacttgctgagagcacataaaagggatcagaaatggaagaagggaaagaggcatggagaaaaaaaactgcttttgtcctttggctattttaaatgaacagaatagaccttgtcaggtgcataa

SEQ ID NO: 110 - DNA, 701 bp, Homo sapiens
aagtcatcttctcagctaggttcagtggctcaaacctgtaaccccagcactttgggaggctgaagcaggtggatcacttgagctcaggagttcaagaccagcctgggcaacatggtgaaacctcatctctacaaaaaaatacaaaaattagccaggcgttggggcgtgtgcctgtagtcccagctacttgagaggctgagrtgcgagaatcgcctgagcccaggaagtggaggttgcagtgagccatgatcacaccactgcactccagcctgggtgacagagtaagacccttggtgggggggaaaagctacttgcttgagaggcacagacaaatagccagatcctgaacttttaataaggtatgtctacacttgctgagagcacataaaagggatcagaaatggaagaagggaaagaggcatggagaaaaaaaactgcttttgtcctttggctattttaaatgaacagaatagaccttgtcaggtgcataaaacacacaggagtcctagttaggctctttaatctgcaaaagagaaccttaatcctatcttctatttggttgattgtcaaagcctttggatcatcctttgtctgtagattaactacactctaggattttgtcaaagattgcaacctttaattctctgctgtccaattcttatctatcttggtgaagtgacatgacatttaaggagaattgt

SEQ ID NO: 111 - DNA, 456 bp, Homo sapiens
tttttactcattttttaatgtagtctaggtatgcagctctctaatggttggactctgatgaaggtaaactccatgtatcaagagcacggagttttcctcagacaattctccttaaatgtcatgtcacttcaccaagatagataagaattggacagcagagaattaaaggttgcaatctttgacaaaatcctagagtgtagwtaatctacagacaaaggatgatccaaaggctttgacaatcaaccaaatagaagataggattaaggttctcttttgcagattaaagagcctaactaggactcctgtgtgttttatgcacctgacaaggtctattctgttcatttaaaatagccaaaggacaaaagcagttttttttctccatgcctctttcccttcttccatttctgatcccttttatgtgctctcagcaagtgtagacataccttattaaaagtt

SEQ ID NO: 112 - DNA, 456 bp, Homo sapiens
agccatatgactctctctccagaactcagtcagacaaacctttccccagccctcggtcagatgcccatttctccagacctcagccatacaaccctttctctagacttcagccagacaaacctctctccagaactcagtcaaacaaacctttccccagccctcggtcagatgcccctttctccagaccccagccatacaacyctttctctagacctcagccagacaaacctctctccagaactcagtcagacaaacctttccccagacctcagtgagatgcccctctttgcagatctcagtcaaattccccttaccccagacctcgaccagatgacactttctccagaccttggtgagacagatctttccccaaactttggtcagatgtccctttccccagacctcagccaggtgactctctctccagacatcagtgacaccacccttctcccggat

SEQ ID NO: 113 - DNA, 458 bp, Homo sapiens
  misc_feature (201)..(201): n = absent or A when 202 is A and when 203 is C
tgcccacagcttttactaaagctgttacactaatcacttaaaaaaccaaagtgtaattacagtaggcaaacacaatgtatggtgtgacttatgcatatgagttcattaatctgatgcctgacatctccacattagaactctgtttaaaacaccttttcctatgacttcctatttaaatgttgcactcaataccacatgagnnnaactagctacctgagtatttctaagcaaggccccgagggagtgagatgcagggcctgtccagagtgaatagggatggaaccaccttcaaaatcttgtattccattcaaggctcacaacccacttttatggcattcggagttgctttacccactcccttgtgttctgtgagtttgcatgttatgctcagagtctggaaagatgggattagtaggggaggtagaccagaagaaaggcagaaggagattctctgttga

SEQ ID NO: 114 - DNA, 701 bp, Homo sapiens
taaagttcagtggccaaaagcactgccatggcagtctgagagaaatgtcactgagccagctggaagcaaacttctccacaacagccagggtccccctactgatcctcagagtctagagtcatggtaatcaccttctctagtaggtaatcacacatgtgaggatgatttctgcatgttctgttcatatagcttcagatgacygacagctagggattatcagagctgacaggtgccaggtcaaataattcaaacagaaaattactctcagctttctttgcataacttccttttggcagtgaatctatcatagttctagaccagtgctgtccaatagaaactacgtatgagctacatgcgtaatttaaaattttctgatagcaacattaaaaagtaaagagaaataggtgacattaattttagtaatatgttttacttaactcagtatatcctgaatattatcatttcaacatgtaaccaatataaaaattaatgagacttactttattactaagtctttgaaattcaatgtatattttatactcacagcatatctcagtccagtctacccatatttcaagtgctcgataaccacttgtagccagtggctaccatattgaacaatgcagttatagaccatttagaaaaacatctagaatgaggtataaactaataatacatcatgtaaattattaaagtcactt

SEQ ID NO: 115 - DNA, 581 bp, Homo sapiens
cctggttgaactgctctgatcatggtgttgttcctgcctgaaagaaaatatattcaaaattgttttcatttgcaaagttatttcatgataataaataaataaataagctttcgctggaaccaattaatattgcaaaaggaattcttttatttttattttttttaaattatactttaagttctagggtacatgtgcacaacrtgcaggtttgttacatatgtatacatgtgtcatgttggtgtgctgcacccattaacttgtcattaacattaggtatatctcctaatgctatccctccccccgccccccaccccccccccccgacaggccccggtgtgtgatgttccccatcctgtgtctaaatgttctcattgttcaattgaattctttaaatattctacttggaacctggataacatgtagccattagataatgctccactagaggccactatgacactaataaaagacaccatattttgttaccactaagagacaaaactcctgaagtgagaagggtttggctgtgatttttaggatactcctacatgtatactacctgactgcagtagtgacaccac

SEQ ID NO: 116 - DNA, 456 bp, Homo sapiens
aacccttctcacttcaggagttttgtctcttagtggtaacaaaatatggtgtcttttattagtgtcatagtggcctctagtggagcattatctaatggctacatgttatccaggttccaagtagaatatttaaagaattcaattgaacaatgagaacatttagacacaggatggggaacatcacacaccggggcctgtcgsggggggggggtggggggcggggggagggatagcattaggagatatacctaatgttaatgacaagttaatgggtgcagcacaccaacatgacacatgtatacatatgtaacaaacctgcacgttgtgcacatgtaccctagaacttaaagtataatttaaaaaaaataaaaataaaagaattccttttgcaatattaattggttccagcgaaagcttatttatttatttattatcatgaaataactttgcaaatga

SEQ ID NO: 117 - DNA, 679 bp, Homo sapiens
tgtgttttatgcacctgacaaggtctattctgttcatttaaaatagccaaaggacaaaagcagttttttttctccatgcctctttcccttcttccatttctgatcccttttatgtgctctcagcaagtgtagacataccttattaaaagttcaggatctggctatttgtctgtgcctctcaagcaagtagcttttcccccycaccaagggtcttactctgtcacccaggctggagtgcagtggtgtgatcatggctcactgcaacctccacttcctgggctcaggcgattctcgcacctcagcctctcaagtagctgggactacaggcacacgccccaacgcctggctaatttttgtatttttttgtagagatgaggtttcaccatgttgcccaggctggtcttgaactcctgagctcaagtgatccacctgcttcagcctcccaaagtgctggggttacaggtttgagccactgaacctagctgagaagatgacttcaaagacagtgtcctaaaaaatctaccgggcaggggaaggggatcatggtctagagatgagaagcagattcaaaatgagatgtggcccaagctgaaaagagaagggcaggggaaggaggacttgcttggagagagtgatactgtgaggaaaacgtcacttctcctaacctatcctctaaagt

SEQ ID NO: 118 - DNA, 630 bp, Homo sapiens
ggatcttcacatcaggataaatggtgctttctttttgtagatgatgtaaacttcaccctgacatatttccttttttacactgactgccataaagcttaggacaaaatttgaagacagccttacagggtcacatggtatctacttatctgtggctttattttctttgtccscatattctatcccaattacatagactccttgttttatgcctttataacttgagaaactgtctcagatcctttgtattactgagtaagctgtaaataaatacaaatactaaataaaaactaaaagttgcatttgaatttaaaattatatgagcatctttttcttttaaaattaaaaaataaccaggtactccataatattttactatgtaatttctcccatgattctgtatttgtgttacttactttgagtgtgtctctgacctgggctctgataataggacccaaaatcccatcttctttcgtattgggattcactgtatgtttggtgaaggactcatcttcgtactgtgtgtacataactttcttataatgttttccaatttggtttgagaaattatccaaatgctgagacctgtattttcttaaagtgaagtaaaaaaaaattaaaccactttctcaa

SEQ ID NO: 119 - DNA, 601 bp, Homo sapiens
  misc_feature (201)..(201): n = absent or T
agttaaagtcctgaatcttgggaaacccctcagaccagaacagtcggttaccttcttggttcccttcttggcctaatcctttagcaatccctgtgtttttgagttcacatccaccatctctaagctctgttgttacctgtataatgccatttatcacaatgagttgtcactgcttgtttgctggtcactcagatgtctgcnttttgcagggcacaaactacaactggagtgtctgttccctcaaagctttatacaatgcctgacacacagcaggtgctcaatacacttggtgagtgaatgtttgatcacagagtacttgactgaatgcttattttggcctgtgtctctccctctttctcagatataacagtttgtgcccatgaccacatcagctggcatctgctgggaatgagctcggggccagaattattctccattcatttcaacggccaggtcctggagcagaaccatcataaggtctcagccatcacccttgtcagtgctacatccactaccgcaaatatgactgtgggcccagagggaaagtggatcatatcttctctcaccccaaaacatttgcaaggcaagaaactctcctgac

SEQ ID NO: 120 - DNA, 456 bp, Homo sapiens
tcttcctcttttcttctttcgaggaagttagagatctctttagcttttgcttaattaaaaaaaaaaaaaaaacctttgccaattccttcgctttctccatccccaaagagcaagttataaatctaagagcaaaatatctaagtttggttgttaggaactgaggaaagtttgtctgcggtgcaggtggcttgaaagggcaarggagaaagagggagttagtgcatgggaagaaaggattctgcattgagaagcaagactgtcaggagagtttcttgccttgcaaatgttttggggtgagagaagatatgatccactttccctctgggcccacagtcatatttgcggtagtggatgtagcactgacaagggtgatggctgagaccttatgatggttctgctccaggacctggccgttgaaatgaatggagaataattctggccccgagctcattccca

SEQ ID NO: 121 - DNA, 500 bp, Homo sapiens
ttatatctgagaaagagggagagacacaggccaaaataagcattcagtcaagtactctgtgatcaaacattcactcaccaagtgtattgagcacctgctgtgtgtcaggcattgtataaagctttgagggaacagacactccagttgtagtttgtgccctgcaaaagcagacatctgagtgaccagcaaacaagcagtgayaactcattgtgataaatggcattatacaggtaacaacagagcttagagatggtggatgtgaactcaaaaacacagggattgctaaaggattaggccaagaagggaaccaagaaggtaaccgactgttctggtctgaggggtttcccaagattcaggactttaactattagaactggtaaaaagtccacggcaaaatggaatgtttggtcaccccagtctgatctcagctggagggagtcaaatcaccagaaccgccaatagggctgagttcaagcacttaatcctctcagctctctagc

SEQ ID NO: 122 - DNA, 505 bp, Homo sapiens
cattcactcaccaagtgtattgagcacctgctgtgtgtcaggcattgtataaagctttgagggaacagacactccagttgtagtttgtgccctgcaaaagcagacatctgagtgaccagcaaacaagcagtgacaactcattgtgataaatggcattatacaggtaacaacagagcttagagatggtggatgtgaactcaraaacacagggattgctaaaggattaggccaagaagggaaccaagaaggtaaccgactgttctggtctgaggggtttcccaagattcaggactttaactattagaactggtaaaaagtccacggcaaaatggaatgtttggtcaccccagtctgatctcagctggagggagtcaaatcaccagaaccgccaatagggctgagttcaagcacttaatcctctcagctctctagctgtagtggtcgaagctctgcctaagggaagaagatgtgaagatgatatgaggattttcaattgttattttta

SEQ ID NO: 123 - DNA, 491 bp, Homo sapiens
gagggaacagacactccagttgtagtttgtgccctgcaaaagcagacatctgagtgaccagcaaacaagcagtgacaactcattgtgataaatggcattatacaggtaacaacagagcttagagatggtggatgtgaactcaaaaacacagggattgctaaaggattaggccaagaagggaaccaagaaggtaaccgactkttctggtctgaggggtttcccaagattcaggactttaactattagaactggtaaaaagtccacggcaaaatggaatgtttggtcaccccagtctgatctcagctggagggagtcaaatcaccagaaccgccaatagggctgagttcaagcacttaatcctctcagctctctagctgtagtggtcgaagctctgcctaagggaagaagatgtgaagatgatatgaggattttcaattgttatttttacttactttttgattgttttaatgacaagtcaacgaaatcacttt

SEQ ID NO: 124 - DNA, 645 bp, Homo sapiens
gctggagggagtcaaatcaccagaaccgccaatagggctgagttcaagcacttaatcctctcagctctctagctstagtggtcgaagctctgcctaagggaagaagatgtgaagatgatatgaggattttcaattgttatttttacttactttttgattgttttaatgacaagtcaacgaaatcactttggggttacacactctccttaaaatgcagtgtacaagtcctcattatgctgagccattgggagcttttcatggaaggagtagtgataactgaatataaaaacttagttttgctccttctttgctcctacagtcactgggaaaatgctcatttgctctgtggggagactccatccttggcttttaggtttctgttttacatctcagatacataatcactagatactagataatgggcctgagaatcagttccttacccacacaaaggcttgttttttttaggagaccattgcaacaaagccaggattccttgcatcactaggaggctttggtggaagcgtttatccatgccaaagaaatgcttatattgagttcagcaattaacatatctgtgttagcatttatcatatgtattcaatttattctgctttataaggcagagggtttttacaaatgttt

SEQ ID NO: 125 - DNA, 518 bp, Homo sapiens
ctggagggagtcaaatcaccagaaccgccaatagggctgagttcaagcacttaatcctctcagctctctagctgtagtggtcgaagctctgcctaagggaagaagatgtgaagatgatrtgaggattttcaattgttatttttacttactttttgattgttttaatgacaagtcaacgaaatcactttggggttacacactctccttaaaatgcagtgtacaagtcctcattatgctgagccattgggagcttttcatggaaggagtagtgataactgaatataaaaacttagttttgctccttctttgctcctacagtcactgggaaaatgctcatttgctctgtggggagactccatccttggcttttaggtttctgttttacatctcagatacataatcactagatactagataatgggcctgagaatcagttccttacccacacaaaggcttgttttttttaggagaccattgcaacaaagccaggattccttgcatcactaggaggctttggt

SEQ ID NO: 126 - DNA, 422 bp, Homo sapiens
agtcctcattatgctgagccattgggagcttttcatggaaggagtagtgataactgaatataaaaacttagttttgctccttctttgctcctacagtcactgggaaaatgctcatttgctctgtggggagactccatccttggcttttaggtttctgttttacatctcagatacataatcactagatactagataatgggsctgagaatcagttccttacccacacaaaggcttgttttttttaggagaccattgcaacaaagccaggattccttgcatcactaggaggctttggtggaagcgtttatccatgccaaagaaatgcttatattgagttcagcaattaacatatctgtgttagcatttatcatatgtattcaatttattctgctttataaggcagagggtttttacaaatgttt

SEQ ID NO: 127 - DNA, 283 bp, Homo sapiens
catcactaggaggctttggtggaagcgtttatccatgccaaagaaatgcttatattgagttcagcaattaacatatctgtgttagcatttatcatatgtattcaatttattctgctttataaggcagagggtttttacaaatgtttactattcttagtttctactgatggtctttccctaccttgtcccatttacccccarttattcttttgggaactataaattcgtaaactctaagctcagatcaatttatttttatttttgtgcatatggttttgctttt

SEQ ID NO: 128 - DNA, 456 bp, Homo sapiens
attttaattttttaatgtttttccctcaggctagtatcatataccctggatctttagttgtaggtcagttctaccagagagaaatagaagagatgtggtctaagtgggaattagttattttttctcttaacgattgctatgattttgccttctcataagtgcccaaatgcaaatggaaagagaaaatattctagatgccaytgaatattactcttctgtcaccttttgaaatgctggttttttttgtgtgttgaggattactcctaatctgttcatctttactgcaatcttcctgctgatccttcctgctagggcttgtccttgaagaagccatggtcatcatatagaggaactgcagaactcggagagagtggctgagctgataccttaaggtcttattcaaagtacaggccaaggtgaaacctactcaggtcactatacctaacaaggctgcac

SEQ ID NO: 129 - DNA, 456 bp, Homo sapiens
ataatgatttcaacttgaagaaattcttattttttaaaatagcatatttatcaagtaataatggttctagggttatttaaaatctggtgccacactgtcttctttgataccttcttgctttggggaacaggctttatttcatccagcttgggaagaaaacatttggtgactctcaagactctgtacaagtatccatttccyccagtctcaatagaagagtatatagatacgttagctcagttggtaaaagcatcatgccccaagtttaatgctgtcatcttatttgtatatgaaagggggcattagataattgggtggaagtatcagcattaatccattctcactatgagaaaaacatgctgctgatggtaaaatgcaagaagagtcttttgtaagttttaaggtttttttcagtaccatcacagtatcttatatataccaggaaatggaaaaaaa

SEQ ID NO: 130 - DNA, 457 bp, Homo sapiens
  misc_feature (201)..(201): n = absent or A or G or C when 202 is G
gttgaaaactttgggtggtaggtttcaaacacactcaagtgactttacaagaacttttattggctgggcttcttaggagcaagcttggattttcattttatcccatgagaatccagcccatctttcattaaaacaaacaaccacagtgagccacagcatgggaaataacgtgatggactcaccatctgcctatacatacannctcaggccatacctacatacatgcccaatgtatttactcattatacaaagactttttttccatttcctggtatatataagatactgtgatggtactgaaaaaaaccttaaaacttacaaaagactcttcttgcattttaccatcagcagcatgtttttctcatagtgagaatggattaatgctgatacttccacccaattatctaatgccccctttcatatacaaataagatgacagcattaaacttggggcatg

SEQ ID NO: 131 - DNA, 649 bp, Homo sapiens
ctgaacaattattatttaaatataatcagagaattgaatcgcctttgattagtaaaccatctcagaatataagaatgggtgatatcttctttgtggtstttaataagacatacttagcctattaacatgagatgggaggatagtcagagctgtttatgtattattttgttttgctatttcagatcttctttccaactgctgtgatcctcagctttgttttatctctttatttcatcttagccatctgattatctcaattttttcccccgagccacagcgtctaactcccctatgcaggctggttgtctgtatgaatatgaaatgtgtccatcccctgaaacttagatgtcttactttgtactttgtgcacccaaagacacatcattaacacctccatgtatattggactttccctactgataggagagggagaggatgaagaaattggtcttctttattgaagagaaggaactgagatactcaatattaaaagaagtgtagagggttgggtgaggttttcaataataaaatggagcaagtgaggtcaggaaggggagaggtgagtgccatagagagccaagtggaatgaacagtttcctctgcctgaggaattcctcatttaattaggtgaaagattccctatcatcag

SEQ ID NO: 132 - DNA, 601 bp, Homo sapiens
  misc_feature (201)..(201): n = absent or A or C
ataagaatgggtgatatcttctttgtggtgtttaataagacatacttagcctattaacatgagatgggaggatagtcagagctgtttatgtattattttgttttgctatttcagatcttctttccaactgctgtgatcctcagctttgttttatctctttatttcatcttagccatctgattatctcaattttttcccccngagccacagcgtctaactcccctatgcaggctggttgtctgtatgaatatgaaatgtgtccatcccctgaaacttagatgtcttactttgtactttgtgcacccaaagacacatcattaacacctccatgtatattggactttccctactgataggagagggagaggatgaagaaattggtcttctttattgaagagaaggaactgagatactcaatattaaaagaagtgtagagggttgggtgaggttttcaataataaaatggagcaagtgaggtcaggaaggggagaggtgagtgccatagagagccaagtggaatgaacagtttcctctgcctgaggaattcctcatttaattaggtgaaagattccctatcatcagctggtgacagaatttctct

SEQ ID NO: 133 - DNA, 681 bp, Homo sapiens
accctggattgagtggcttataaacaacggacacttcttcctcacagttctggaggctggaagtttaaaatcagggtgccagcatggttgggttctggtgcagggtctcttccaggttgtagactgccatcttctccttgtatcctcacatggtagaaagagggtgagtgagagggtcccttttataagggcactaatctyatcatgagggctccatcctcattacctaaaaatctcccaaaggaggggggagggatagcattgggagatatacctaatgctagatgacgagttagtgggtgcagcgcaccagcatggcacatgtatacatatgtaactaacctgcacaatgtgcacatgtaccctaaaacttaaagtataataaaaaaaaatctcccaaaggccccacctgctaatgccatcacactggaggttagatttcagtatgtgaatttttggaggacacaaacttccaatccattgtagtgatgtatctattccaaaggcgatgaaagtaaataagacttttttggtaaaagtactttttttttttttttttggtaataagtaagacaaagtacctgctcaaaattatcagcaaaatcaataattttaaagcaagggaaaaataatgcatagtccttactttctaatcagtcctcggcccctcagtttagttca

SEQ ID NO: 134 - DNA, 450 bp, Homo sapiens
gttctggaggctggaagtttaaaatcagggtgccagcatggttgggttctggtgcagggtctcttccaggttgtagactgccatcttctccttgtatcctcacatggtagaaagagggtgagtgagagggtcccttttataagggcactaatctcatcatgagggctccatcctcattacctaaaaatctcccaaaggagvggggagggatagcattgggagatatacctaatgctagatgacgagttagtgggtgcagcgcaccagcatggcacatgtatacatatgtaactaacctgcacaatgtgcacatgtaccctaaaacttaaagtataataaaaaaaaatctcccaaaggccccacctgctaatgccatcacactggaggttagatttcagtatgtgaatttttggaggacacaaacttccaatccattgtagtgatgtatct

SEQ ID NO: 135 - DNA, 687 bp, Homo sapiens
gtcctccaaaaattcacatactgaaatctaacctccagtgtgatggcattagcaggtggggcctttgggagatttttttttattatactttaagttttagggtacatgtgcacattgtgcaggttagttacatatgtatacatgtgccatgctggtgcgctgcacccactaactcgtcatctagcattaggtatatctccmaatgctatccctcccccctcctttgggagatttttaggtaatgaggatggagccctcatgatgagattagtgcccttataaaagggaccctctcactcaccctctttctaccatgtgaggatacaaggagaagatggcagtctacaacctggaagagaccctgcaccagaacccaaccatgctggcaccctgattttaaacttccagcctccagaactgtgaggaagaagtgtccgttgtttataagccactcaatccagggtactttgttaacagcaacccaaactaagaaaatcaccaatttctatcttactcacatgtgttaccagaactcaagcacacttaagatctatcttatctaaagtagaacataaaggaaagaggctgtattaattgcaacatgactgggaaagaaaatacttaaccaaaatgtgaacatgtattcccacaggctatcagagcatttgttaggagccagaaatttta

SEQ ID NO: 136 - DNA, 470 bp, Homo sapiens
ctccatcctcattacctaaaaatctcccaaaggaggggggagggatagcattgggagatatacctaatgctagatgacgagttagtgggtgcagcgcaccagcatggcacatgtatacatatgtaactaacctgcacaatgtgcacatgtaccctaaaacttaaagtataataaaaaaaaatctcccaaaggccccacctrctaatgccatcacactggaggttagatttcagtatgtgaatttttggaggacacaaacttccaatccattgtagtgatgtatctattccaaaggcgatgaaagtaaataagacttttttggtaaaagtactttttttttttttttttggtaataagtaagacaaagtacctgctcaaaattatcagcaaaatcaataattttaaagcaagggaaaaataatgcatagtccttactttctaatcagtcctcggcccctcagtttagttca

SEQ ID NO: 137 - DNA, 701 bp, Homo sapiens
gaagaaatctagaggtcaaacctatcacaattagggcaccaacatgtaccctagagttaactaggcagagcccctcaggggcccaggccctgtcttctttacatttgtactctggcatttagcacagagcctggaacctagtggatgatgatggtcatgatcatgatgatattaatcattactgaactaaactgaggggcygaggactgattagaaagtaaggactatgcattatttttcccttgctttaaaattattgattttgctgataattttgagcaggtactttgtcttacttattaccaaaaaaaaaaaaaaaaagtacttttaccaaaaaagtcttatttactttcatcgcctttggaatagatacatcactacaatggattggaagtttgtgtcctccaaaaattcacatactgaaatctaacctccagtgtgatggcattagcaggtggggcctttgggagatttttttttattatactttaagttttagggtacatgtgcacattgtgcaggttagttacatatgtatacatgtgccatgctggtgcgctgcacccactaactcgtcatctagcattaggtatatctcccaatgctatccctcccccctcctttgggagatttttaggtaatgaggatggagccctcatgatgagattagtgcccttataaaagggaccctctcactcacc

SEQ ID NO: 138 - DNA, 456 bp, Homo sapiens
tcaccctgctatacctagcacaagtgttatagcatcttgggcaatttttagatacttacattggttggttctcattccagattgacagtgtgttctaattgtagccctagaagaaatctagaggtcaaacctatcacaattagggcaccaacatgtaccctagagttaactaggcagagcccctcaggggcccaggccctktcttctttacatttgtactctggcatttagcacagagcctggaacctagtggatgatgatggtcatgatcatgatgatattaatcattactgaactaaactgaggggccgaggactgattagaaagtaaggactatgcattatttttcccttgctttaaaattattgattttgctgataattttgagcaggtactttgtcttacttattaccaaaaaaaaaaaaaaaaagtacttttaccaaaaaagtcttattt

SEQ ID NO: 139 - DNA, 456 bp, Homo sapiens
tactactaaaagttgaattatgagcatcttgaaacagtgaatgatatagacttgtacataggatatattctataattacactgagtgagatggctaatgagacaattggggtgtaattttatcaatgcttttattcttttcacttcaaattattttacctttagtctagaataaaacaggtttgttgtatctttgattttrcaacatacattaatataaagtataaaatacaaacagctattaagaggaagcatttgtgagatgcagttttggtgaatgtgattttgactttgtaatcaaaataaaaaaaattaagctctaaactgaaaagaagagaaatggacagggacaactattgtgctagagcacaagaagtccttgttcagctgcttgctggaataaaatctttacacaagggtagctcttcatttatatttactcagttctgttcatttc

SEQ ID NO: 140 - DNA, 456 bp, Homo sapiens
  misc_feature (201)..(201): n = absent or A
ataaatacaatacaaagctgactatcgactgagcaggaaatgaacagaactgagtaaatataaatgaagagctacccttgtgtaaagattttattccagcaagcagctgaacaaggacttcttgtgctctagcacaatagttgtccctgtccatttctcttcttttcagtttagagcttaattttttttattttgattacnaagtcaaaatcacattcaccaaaactgcatctcacaaatgcttcctcttaatagctgtttgtattttatactttatattaatgtatgttgcaaaatcaaagatacaacaaacctgttttattctagactaaaggtaaaataatttgaagtgaaaagaataaaagcattgataaaattacaccccaattgtctcattagccatctcactcagtgtaattatagaatatatcctatgtacaagtctatatcattcac

SEQ ID NO: 141 - DNA, 456 bp, Homo sapiens
tgtaactagcatgcatcaaattgacttcaatgctgcacctttgagcaaagtttgttgttcagtaaaattttcaaagtcgttttggtcaattacctttaaaaagcttgtttcatggagaatttaatattattcttttcttttagttatattctcatattttaaatatatgagttgcatatgaatgtgatgtcacaaaattasttctataatatatagccatctgctggtaagccagctctccaaataaaacacgttttctgccagacattgttcgttgtatctaagtgttgccaaattctctatacaaaaatttgctcttctagatttttccaaaacattccaatgcttgacttaatgtaggtgattacattttttcacttctcatctgaatattttggccttatgtagagacttccttgagtatatgataaacacctgaaacaaactataacggct

SEQ ID NO: 142 - DNA, 456 bp, Homo sapiens
gtagggggttaagatagtattacactggcttgagaaaccacaacagaacataagagaactctggataaccctttttgacagaatggacaaacggtgatttgtaaaaatgcttgtaaggacatttcctttaataatgtatttaataagactgtcttagatcagggaataaagtatctgcaactgtaactaatcacttaaaarctgtcactaaagggtttcaatttgaggttaaattttcagaaactctgtaactagcatgcatcaaattgacttcaatgctgcacctttgagcaaagtttgttgttcagtaaaattttcaaagtcgttttggtcaattacctttaaaaagcttgtttcatggagaatttaatattattcttttcttttagttatattctcatattttaaatatatgagttgcatatgaatgtgatgtcacaaaattagttctataat

SEQ ID NO: 143 - DNA, 456 bp, Homo sapiens
cctcttcctttactgtttatttgtccctacattccctacagccagcaattatttatgagcaattgcttaaaatgctcccacagctttcactggagacatgattattggcatataatattgcctctggtcctatgaatctaagaaaggtaaatacatattggtagggaaagtaaaattttaccttggaaatgagatgcaaayctagtaagcagatagtatcttttatggtaaaagacaagccttataagttgattttattatgccctctgcattaaaataaaacatctcctcctccatgtctgacactcaccctcacctatagcctccctctgtggtttgagctatttttttgggagtggggataaactaactgcatgctgagccagttacatgagaatccagattcctctatctcagaggtagatatttgagagataggtggtagggggttaagat

SEQ ID NO: 144 - DNA, 400 bp, Homo sapiens
aatggtaataataatcatgtctctgtcatacaggtttgttgtcgtgaaatcttagaataatgactggcatataacagataataaatattagccatgatagtaaagataattcttcaattaaaatgtgttcaaataatttagtatttatcttaaaacttgttagtttcagaaaaaatcagaaagtaatttttaaatttattrttttctatataaactgttgtcaaactcatacccactaaggtataagtgactgataataggtcaatgaactaccttccttaggagtacttgcattagcacttaatctttggcttgaggaaaaacaatgaagcattaactgtggcactgagatttcttccaaagtgaatttggcaagactctgggtgaggtagtgggaaca

SEQ ID NO: 145 - DNA, 456 bp, Homo sapiens
  misc_feature (201)..(201): n = absent or A
cctctgtgtgcctcatctttctcatctgtaaaatggtaataataatcatgtctctgtcatacaggtttgttgtcgtgaaatcttagaataatgactggcatataacagataataaatattagccatgatagtaaagataattcttcaattaaaatgtgttcaaataatttagtatttatcttaaaacttgttagtttcagnaaaaatcagaaagtaatttttaaatttattgttttctatataaactgttgtcaaactcatacccactaaggtataagtgactgataataggtcaatgaactaccttccttaggagtacttgcattagcacttaatctttggcttgaggaaaaacaatgaagcattaactgtggcactgagatttcttccaaagtgaatttggcaagactctgggtgaggtagtgggaacaggagtttctcctatgttcttaaaat

SEQ ID NO: 146 - DNA, 598 bp, Homo sapiens
caagttttaagataaatactaaattatttgaacacattttaattgaagaattatctttactatcatggctaatatttattatctgttatatgccagtcattattctaagatttcaygacaacaaacctgtatgacagagacatgattattattaccattttacagatgagaaagatgaggcacacagaggttaaataacttcataaaagtcactgagccaggatttgcacttattagtctagttctaaaacctgcacataaaccactctcctactcaattattctctcaaaggtatgatggctggaacatgtagaaggaaagatatttaaatgtgaaccatgaaaagtctgaaattattttaaaatgttctcataccaacaactattataatatggatacaattttttataccagtgcctgatggaactctactatgcttacaatgatctgaacatcagcataatgggataattagaaccatattaacatcaggtacttactattcagcggctgatacaataacttgcatgactattatctttatgattattgccatcattgtcatcattatttatagagagcttatccgatcccagg

SEQ ID NO: 147 - DNA, 603 bp, Homo sapiens
  misc_feature (201)..(201): n = absent or A when 202 is G and 203 is G
tcacgacaacaaacctgtatgacagagacatgattattattaccattttacagatgagaaagatgaggcacacagaggttaaataacttcataaaagtcactgagccaggatttgcacttattagtctagttctaaaacctgcacataaaccactctcctactcaattattctctcaaaggtatgatggctggaacatgtnnnagaaggaaagatatttaaatgtgaaccatgaaaagtctgaaattattttaaaatgttctcataccaacaactattataatatggatacaattttttataccagtgcctgatggaactctactatgcttacaatgatctgaacatcagcataatgggataattagaaccatattaacatcaggtacttactattcagcggctgatacaataacttgcatgactattatctttatgattattgccatcattgtcatcattatttatagagagcttatccgatcccaggaaccatgtttagtactctacctaagtgacttcatttaaatttcaggcaatcttatgggtggttataatcatttccattttatagatgagaaaactgaggctcagagatgctaaa

SEQ ID NO: 148 - DNA, 459 bp, Homo sapiens
  misc_feature (201)..(201): n = absent or T when 202 is T and  when 203 is T and when 204 is A
catgcaagttattgtatcagccgctgaatagtaagtacctgatgttaatatggttctaattatcccattatgctgatgttcagatcattgtaagcatagtagagttccatcaggcactggtataaaaaattgtatccatattataatagttgttggtatgagaacattttaaaataatttcagacttttcatggttcacannnnaatatctttccttctcctacatgttccagccatcatacctttgagagaataattgagtaggagagtggtttatgtgcaggttttagaactagactaataagtgcaaatcctggctcagtgacttttatgaagttatttaacctctgtgtgcctcatctttctcatctgtaaaatggtaataataatcatgtctctgtcatacaggtttgttgtcgtgaaatcttagaataatgactggcatataacagataataa

SEQ ID NO: 149 - DNA, 429 bp, Homo sapiens
gaaagatgaggcacacagaggttaaataacttcataaaagtcactgagccaggatttgcacttattagtctagttctaaaacctgcacataaaccactctcctactcaattattctctcaaaggtatgatggctggaacatgtagaaggaaagatatttaaatgtgaaccatgaaaagtctgaaattattttaaaatgttmtcataccaacaactattataatatggatacaattttttataccagtgcctgatggaactctactatgcttacaatgatctgaacatcagcataatgggataattagaaccatattaacatcaggtacttactattcagcggctgatacaataacttgcatgactattatctttatgattattgccatcattgtcatcattatttatagagagcttatccgatcccagg

SEQ ID NO: 150 - DNA, 341 bp, Homo sapiens
ataaaccactctcctactcaattattctctcaaaggtatgatggctggaacatgtagaaggaaagatatttaaatgtgaaccatgaaaagtctgaaattattttaaaatgttctcataccaacaactattataatatggatacaattttttataccagtgcctgatggaactctactatgcttacaatgatctgaacatcrgcataatgggataattagaaccatattaacatcaggtacttactattcagcggctgatacaataacttgcatgactattatctttatgattattgccatcattgtcatcattatttatagagagcttatccgatcccagg

SEQ ID NO: 151 - DNA, 456 bp, Homo sapiens
gtgagactagaagcattaatagtattagacttttggacatagtgaagtggttaagaaagtaagctttaaaggcagacaagttaaaattctagctttatcatttctgattctacgactttagcatctctgagcctcagttttctcatctataaaatggaaatgattataaccacccataagattgcctgaaatttaaatgargtcacttaggtagagtactaaacatggttcctgggatcggataagctctctataaataatgatgacaatgatggcaataatcataaagataatagtcatgcaagttattgtatcagccgctgaatagtaagtacctgatgttaatatggttctaattatcccattatgctgatgttcagatcattgtaagcatagtagagttccatcaggcactggtataaaaaattgtatccatattataatagttgttggtat

SEQ ID NO: 152 - DNA, 456 bp, Homo sapiens
ctttgcaggccataagatctctgttgcaagtactcaactctgccttttagtacaaaagcagccatagacgatacctatttttttggctttggttacagatgccagaactaattaactttgttaccgtattctttccctcaagtcatacaccagagtaagaccgcctctgacaatacaattttatttacaaaaacaggaaaktggttgggtttggcccatgagccactgacatagtgacatagaactagcctgatataggcttcctggcacatagatgacactcaataagtggtatttggtggtggtgagactagaagcattaatagtattagacttttggacatagtgaagtggttaagaaagtaagctttaaaggcagacaagttaaaattctagctttatcatttctgattctacgactttagcatctctgagcctcagttttctcatctataa

SEQ ID NO: 153 - DNA, 741 bp, Homo sapiens
gagggtagttactttagatttctctgacatgggtgtgctcagagacctagatgaattgaggaaccaaatcacacagctctctggctctgaggcaggagtagtcttgatgtatctaaggaacagtaagaaaaacagtacgggaagaaagaagagatgaggtggatggtaggtgtgtagaacaagggaggccctatgggctayggtaagaacttgaattttcttctaagtgaaatgggacatcactggagcatttttctttttttttttgagatggagtctcgctctgtctcctaggctggagtgcagtgatgcaatctcaggacccactgcaacctccgcctcctgggttcaagcaattctcctacctcaacctcccaagtagctgggattgcaggcatgcaccaccacacccagctaatttttgtatttttagtagagatgacttttcaccatgttggccaggctggtctcaaactcctgacctcaggtgatacacccgcctcagcctcccaaagtgctgggattacaggtgtgagccaccacgcctcgtctaagtgaaatggagcatttctaagtaaaatgaaaaaccactttgtacagcagagacatagtataacgtgtattaaaagttcatcctaaaacaaaaagttcatcatagctgctgtgtagagaatagtctgtgtagggacaagaatggaagcagagagattaggcagtataggaaagagactgcagtgtact

SEQ ID NO: 154 - DNA, 603 bp, Homo sapiens
  misc_feature (201)..(201): n = absent or G
aacatggtgaaaagtcatctctactaaaaatacaaaaattagctgggtgtggtggtgcatgcctgcaatcccagctacttgggaggttgaggtaggagaattgcttgaacccaggaggcggaggttgcagtgggtcctgagattgcatcactgcactccagcctaggagacagagcgagactccatctcaaaaaaaaaaannnaaatgctccagtgatgtcccatttcacttagaagaaaattcaagttcttaccgtagcccatagggcctcccttgttctacacacctaccatccacctcatctcttctttcttcccgtactgtttttcttactgttccttagatacatcaagactactcctgcctcagagccagagagctgtgtgatttggttcctcaattcatctaggtctctgagcacacccatgtcagagaaatctaaagtaactaccctctcgcatcaccctctgtaattgaaatgaacatctttcttaagcaaagaaagttattgctaaggaatgacagtgggtcaaagtgggcaagacttggccagaactatttctcctactctgttgacttcttttgggtccaggacaagtgtt

SEQ ID NO: 155 - DNA, 456 bp, Homo sapiens
tagaaagtaaaacctcattcttgatttctgcacccataacctcctgtctctgctgtgttctcccattgcagtaactaataacatcatttacccagttgttctgataaaacaccttgaggtcatccttgacttctgtctctcacaagccacatgcaatccatcagcaagtcttgtttgccttacctacaaaaatgtccagamtccaaccactactcatcacgttctgctacatgtgctggcccagtacactgcagtctctttcctatactgcctaatctctctgcttccattcttgtccctacacagactattctctacacagcagctatgatgaactttttgttttaggatgaacttttaatacacgttatactatgtctctgctgtacaaagtggtttttcattttacttagaaatgctccatttcacttagacgaggcgtggtggctcacacct

SEQ ID NO: 156 - DNA, 701 bp, Homo sapiens
tgttttttgaatatttttgatccaaaattggttgactccatggatgtgtaaccatgaatatgaaccatggtcatggagggctgactatatacaattcctcaaattctgactcagccatcaactttttccctaactccccagacagaatttgtgcttcatcctctcttcttctgtaatattttgcccatatctcagcagtcrctcctatcatacatctatctccaaactagcttgtgagttccttcagtacagggactgtatttgatccaggtagccagaaactttctaacacagtgcctggcatgtagaaagtaaaacctcattcttgatttctgcacccataacctcctgtctctgctgtgttctcccattgcagtaactaataacatcatttacccagttgttctgataaaacaccttgaggtcatccttgacttctgtctctcacaagccacatgcaatccatcagcaagtcttgtttgccttacctacaaaaatgtccagactccaaccactactcatcacgttctgctacatgtgctggcccagtacactgcagtctctttcctatactgcctaatctctctgcttccattcttgtccctacacagactattctctacacagcagctatgatgaactttttgttttaggatgaacttttaatacacgttatactatgtctctgctgtacaaagtgg

SEQ ID NO: 157 - DNA, 701 bp, Homo sapiens
ttggcagcatagtcagcaccctgttgggctcattggcagctggggtaggtgggaatagtcagtggaaacttccatcaggagccaagacagagggtagcagctggaagaggggggtcagtcagtggagtccgacatgtcaggagtcaaaagaggtgtagacatctggactattctataaagatgggggcctgagcctgcccygtggaaggaatagggagagctttggctcaggagtccagagatctgagttctgatcccagcaatgctaccagatcactgactgaccttgggcaagtcacctttgtcccccgattcctacctcccaggctttgttaactaaaataaggactgaactttgtgacctccacagtcctgtgtaaagctaacataaaaacaaacaaaaagcttcaggcacaagatcagaataaactccaggagtaggagttcaaggaaaggaataggacaaccgtgggtggcaagacaggggtagggaaagataggacctctgtcccagaaatcagagtttaccattgcttctgtaaccagttactgcaaacttagtggctaaaacaacacaaatttattcttttatgattatatatctgcagatgagaagtccaaaatgggttttattgggctaaaatcaaggtgttggcagagctgcattccttctggaggctctgggggagaattgtttcctt

SEQ ID NO: 158 - DNA, 701 bp, Homo sapiens
atgcggcacacaattggcagcatagtcagcaccctgttgggctcattggcagctggggtaggtgggaatagtcagtggaaacttccatcaggagccaagacagagggtagcagctggaagaggggggtcagtcagtggagtccgacatgtcaggagtcaaaagaggtgtagacatctggactattctataaagatgggggyctgagcctgccccgtggaaggaatagggagagctttggctcaggagtccagagatctgagttctgatcccagcaatgctaccagatcactgactgaccttgggcaagtcacctttgtcccccgattcctacctcccaggctttgttaactaaaataaggactgaactttgtgacctccacagtcctgtgtaaagctaacataaaaacaaacaaaaagcttcaggcacaagatcagaataaactccaggagtaggagttcaaggaaaggaataggacaaccgtgggtggcaagacaggggtagggaaagataggacctctgtcccagaaatcagagtttaccattgcttctgtaaccagttactgcaaacttagtggctaaaacaacacaaatttattcttttatgattatatatctgcagatgagaagtccaaaatgggttttattgggctaaaatcaaggtgttggcagagctgcattccttctggaggctctggggga

SEQ ID NO: 159 - DNA, 459 bp, Homo sapiens
  misc_feature (201)..(201): n = absent or G when 202 is T and when 203 is T and when 204 is T
tgggcattgaaataggcattgtggatgcaatagtggacaagacagtctcatatcctgggttcatggagattatggtctagtttaatgagcatagatgtgccttcgcaaaatgatattttgcttccatttggctccctagattcattcaaaggaactatgtatggctgactcttttgcatctctttcccgtctattcttctnnnnatttagtgtgatatatacaatagccataatttttaaagaaaatctcttattttttcctcatctcttcctttatcaagaagactgggttgaatagggttctaatatcttaactatgataccatattccgcagggatgatagagaccatcaagggagaacttcctccagttgaatccccttgtcagtaattcacctgtccctttccccaatcagatgaagacctgctgtctttcccagaatcatcctagtctgtcct

SEQ ID NO: 160 - DNA, 456 bp, Homo sapiens
gagttggctatgttgatattcacctagtgagtatcatagctctaggggcaggacaagttagaacaagttcttagtttaagcagccagatatatggattagatttttcataagaaaatgctagagagaaatgatatgatttagggttaaacaatatgacagtttgtctgggttgtgtttctatggttttgactcaacaattsctaccagaggaacgaatctccagaactttggaaacttacccacaggatgaggggacagaatgaccagtcatggttccctgtgtactcaccatgtgaatcagacctttgccctatctggttctgcctccggtattttgcttgttattttcaacgctaactacagacaagagaaaaaactcccaagcatgaacccagcatgctacaagaaagccaacaaccaagtctgcattctactcatgagcaggcaagattagt

SEQ ID NO: 161 - DNA, 299 bp, Homo sapiens
caacttgccttgagatgacaaccaaagttttcctggtgtcctcyacactcaagagtgactgtgaggcggaggggcccagcccttcttgcaggcgggaatgagtggatgggtggatcaacagaggctgccacaggagagagggaggcctggcctgggaacagagctgtgaccgtgcccttccccagggtaggggctgaaggaccctcccatcctagtgacagggccacagcatgtccaaggaggccccagaggaggtcccgggagtcctgggagagcctggttagcctccctgaagggag

SEQ ID NO: 162 - DNA, 441 bp, Homo sapiens
caacttgccttgagatgacaaccaaagttttcctggtgtcctccacactcaagagtgactgtgaggcggaggggcccagcccttcttgcaggcgggaatgagtggatgggtggatcaacagaggctgccacaggagagagggaggcctggcctgggaacagagctgtgaccgtgcccttccccagrgtaggggctgaaggaccctcccatcctagtgacagggccacagcatgtccaaggaggccccagaggaggtcccgggagtcctgggagagcctggttagcctccctgaagggaggaagtggggttttgtgagagggatggtgcagcagcccccacacctgctactccgtgtggccgggtccagccccaggcaaggttccaggcatgcccctgggacagacgtgggagggagaccagcaggcaggtccccctcaggg

SEQ ID NO: 163 - DNA, 458 bp, Homo sapiens
acttgaggtcaggagttcgaaaccagcctggtcaacacggtgaaaccccatctctgctaaaaaaaaaaaatatatatatataaattagccaggcatggtgacgtgcacctgtggtcccagctactcaggaggctgaggcacaagaatcacttgaacccgggaggtggaggttgcagtgagattgcaccagtgcactctccmgcctggcaacagagcaagactctgtctcaaacaaacaaaacaaaacaaacaaaaagacgtaagatgtggaccgctggagaatgggggtgctgcctgcagtcaaaacggagtgggggtgcccagctcagggccagaatgatcctattcccggcacttctcagtgaggctctgtggctcacctaagaaaccagcctcccttgcaggcaacggcctagctggcctggtctggaggctctcttcaaatatttacatccaca

SEQ ID NO: 164 - DNA, 418 bp, Homo sapiens
tgaaaccccatctctgctaaaaaaaaaaaatatatatatataaattagccaggcatggtgacgtgcacctgtggtcccagctactcaggaggctgaggcacaagaatcacttgaacccgggaggtggaggttgcagtgagattgcaccagtgcactctccagcctggcaacagagcaagactctgtctcaaacaaacaaarcaaaacaaacaaaaagacgtaagatgtggaccgctggagaatgggggtgctgcctgcagtcaaaacggagtgggggtgcccagctcagggccagaatgatcctattcccggcacttctcagtgaggctctgtggctcacctaagaaaccagcctcccttgcaggcaacggcctagctggcctggtctggaggctctcttcaaatatttacatccaca

SEQ ID NO: 165 - DNA, 372 bp, Homo sapiens
ggaccgctggagaatgggggtgctgcctgcagtcaaaacggagtgggggtgcccagctcagggccagaatgatcctattcccggcacttctcagtgaggctctgtggctcacctaagaaaccagcctcccttgcaggcaacggcctagctggcctggtctggaggctctcttcaaatatttacatccacacccaagatacdgtcttgagatttgactcgcatgattgctatgggacaagttttcatctgcagtttaaatctgtttcccaacttacattaggggtttggaattctagatcgtatttgaagtgttggtgccacacacaccttaacacctgcacgctggcaacaaaaccgtccgctctgcagcac

SEQ ID NO: 166 - DNA, 516 bp, Homo sapiens
gatggggtgtggaggatcgggggtggggatggcgtgtggggtgtgggggatgggccgtgggggggtggggcctgggaaacagcatgtggggcatggggtgtgggggtgaggtgtgggaaagtgtgtggggtgtgggggatggggcatggaaagggcgtgtggggtgcaggggatggggcatggaggtgtgggggatggggygtgtggggtgtcggggatggggcatgtggggtgtgggggatggggcatggaaagggcgtgtggggtgcagaggatggggcatgggggggtggggatggcgagtggggctggggcctgggaatggtgagtggggcatggggatggcgagtagggggtgtggcgtgaggatggctagtggggcgtggggatggcgtgtggggatggcgagtggggggtgggctgtgagggacagtgcctgggatgtggggctgcagccctagctcacagcatggccttatgaccccggccaccttcctgccccaggcggggtcgcta

SEQ ID NO: 167 - DNA, 600 bp, Homo sapiens
tattgctattaggaaaacacatatgcatgcatttcttctagattatcatctaagagtggcttctccagagagagacgactgaattaaaggttatcaacaagttccaattccagataagatgaagaaatcacattccacactgcctctcccactgagtgtagctccaaaacatggatagaatgcatgtagcagctatttgasgaccctaaaaagtaaatcgcagtgtattgcagaataagactacaattagatgtatgatatgatacaactggctgtgagtttatcattttttcctccagtcttccagacatcacttgacctgaatctaatggacatttataggattctcaacaatagcaaagtacactttccttccacatatggaaaattcctcaaggtagactatatcctgtgtcttaaagcatacctcaataaaaagattgaactcacataaagtatgttttctgaccataatggaattaaagtaaaaattactaacagaaaaataactggaaacttccctaagtactcggaaattaagtcacacatgtataaataatctgtgagtcaaagagaaaattttaaggggagtaagaaagt

SEQ ID NO: 168 - DNA, 456 bp, Homo sapiens
atttaaaaataaaatgttaacctaaaaaccaatagtcatggtctcggccagcgcctcgccgagttgcagtgagctgagatcgtgccctcccacgcccgcagcccgcgtcctgccttggcctccgtagtcgctgagagccacagcctagagcgccagcgcgcaggcgcacaactgacgccaggccacgaacccagtactgckcctgcacagcagaagcactagcactgaggccgggccgcgaacccggcactgcgcctgcgcagcaaaaggacacgcactgaggccaggccgcgaacccagcacggtgcctgcgcagcaggaagaccggcatccacaccggacgacgaacccagcatcgcgcctgcgcagtaggaggagagcaatgccaccaggccgcgattgcgcagccgcagcagccccgcgcggaagacgctaccctcctctcccccgaagagg

SEQ ID NO: 169 - DNA, 800 bp, Homo sapiens
aaaaacaaacaaacaaaaacaagaaaaaggacctatgttggaaatggaagagaggggacatcactacagaaactgtagatgttaaatgtataataagaaaatactttgaacaactctgcatatataaatttgcatgagatttgaacttggatgaaatgagcctattcttcaataccacaagccaccaaaacatacacaagrtgaaagagatacctgccaattcaattcttaatttaaaaccttctgaaaaagtaatgttcaggtacagatggtttcactggtagaattttaccaaacatttcaaaaagaacaccaattctatacaactcttccagaacatagaagagggaacacttcttagtttgtcttaggccagcattaccctgatgtcaaaaccagacaaatactgaaaacaaaaaccaccctacgtaacaatatctctcatgaatctagacataaaaatcctcaacaaaatattagcaaacggtgcagcaatatatttttaaaagagtaataatacaccatgaccaagtgagtttttctggggcacacatgactggctcaatatttaaaaataattatgtaatccaccatataaacaaaagagaacatccacataatcatgtcaattgatgcaacaaacaaatctggcaaaatttaacatccatttatgattttataaaaaacctatcagcagaatatgaataggagggaattttatgaacataataaagttcatctacaaagagtctacagttgatattatacttaaaggtgaaaactgaaggttttctccctga

SEQ ID NO: 170 - DNA, 550 bp, Homo sapiens
catggctgtaggagggagaagaatgagagccgagcaaaaggggaatcctcttaaaaaaaatcagatctcatgagaacatactcccacgagaacagcatggaggaaccaccctcacgattcagttacctcccacttggtccctctcactacacatggggattatgggaactacaattcaagatgagatttgggtggggacaragccaaaccatatcaatgctcctaaaatttgcaaatgagtgtaacaaggtcacagaatacaaggtcagcacatgtgttaatcacatttttatgtaatagcaatgcacagttatttgtaagccaaaaatttttaaatgccatttacaattgcttcaaagaaaattatatacttatatgtaaagctaataaaacatatacaggatctttatcccaaaatctacaaaattccaatgaaagtatttaaacagacctaaataaatagagacacatacagtgttcatggattgaaagactcaacatattaagatatcaattttcggccgggcgcggtggctcatgcctgtaatcc

SEQ ID NO: 171 - DNA, 450 bp, Homo sapiens
tgattaataaaagaaaaaagtcataaattggactttatcaaaattaaaaccttttgcacttcagaaataaacactgttaagaggatgaaaacacaagctacaaactaagagaaaatatttgcaaatcacatatccaacaaaggaatcatattcggaatatataaagaaatcttaacagatcagaagaagaaaataaacacycagttaaacaaaagaccttaacagccaactcgccaaagaggatatatggatagaaaataaacatgtgagaagatactcaacattattagctcttacagaaatgcagataaaaaccacaataagaacgactatatactcatagagtaaaaaacactgacacagaacagcgctggttaagacacggagaaagcagaactttgatacactgctcgtgggaatgcaaaatggcacggccactttgaaaaggaa

SEQ ID NO: 172 - DNA, 450 bp, Homo sapiens
actcttttacaataactattatggaaacaccctgagggaaaaagtcttaaatgagtaaaatgagaacaaaagcagaaaggaaattcaggaactattttcaaaattcaaatatcctgatgaacccctctcactgaaggttaaaacagaaaaaatactgacctttgtatttattccagaattcattctaaaaggaaaaaagaratttaaaaatatattagggaaaacagagacattttataccaataacaaccataattaagtgactcaagctgaataggatattttccccaatggaagtcacttttaggaatgaattgttctagaactattcaatctaaagaggaaagctattcagatcttctgcatctgtgaagatgtggcttcagtcatcttaaatgaaccatctttgttgaatattgaaaatattttcaagttaattacctagaaaca

SEQ ID NO: 173 - DNA, 450 bp, Homo sapiens
caggtaacagtgacaccaagaggacaggactgagccctgggctccgggcccaggtggttcaaacatgaagaccatgaggtttggaaacagacccattatttctgtaagccagatctgctgtttaacctcagcttccccatctgacaaatgggaccaacactattgcctgactgcttgggtgatccctggagcactttgcaygatgcctggcccaccgcaggccctcagtctgcattgggactgtggggggatccagtgcaagggctcaaagcaccagggcaggcaaagggcagagctggcccgaggaactggagctaaggtgcggggctgggataggagtcaggggacgctcaggctctgagctccttttaccaggaccagtgttcattgaacgtagtttttcttttccttgatgaatgtggacaacaggcggccagagggcagtgagca

SEQ ID NO: 174 - DNA, 450 bp, Homo sapiens
atgaggactgggatgaactggtggctgggtgtggggaaaatggaagtgaaggaaggccaaaagagacagagaaggcctggcgcggcgactcacgcctataatcccagcactttgggaggctgagaagggggattgcttgaggccagaagttgaataccagtctgggcagcatagcaagaccctgcctctacaaaaaaaaawttttttttaattagccaggcttggtgacatgcatctgtagtctactcaagaagctgaggtgaggccaggcacggtggctcacgcctgtattcccagcactttgggaggtcaaggcgggtggatgacctgaggtcaggagttcaagaccagcctggccaacatggtgaaaccccatctgtataaaaatacaaaaattagctgggcatgatagcaggtgcctgtaattccagctactcaggaggctgaggt

SEQ ID NO: 175 - DNA, 459 bp, Homo sapiens
  misc_feature (201)..(201): n = absent or C when 202 is A and  when 203 is A and when 204 is A
gtgaaaccccatctgtataaaaatacaaaaattagctgggcatgatagcaggtgcctgtaattccagctactcaggaggctgaggtgggagaatctattgaacccgggagggggaggttgcagtgagccgagatcatgccattgcactccagcctgggcgacagagtgagactccttctcaaaacaaacaaacaaacaaannnncaaaatacagaagctgaggcgggaggaacatttgaaccggattcggaggctgcagtgagctatgattgcaccactgcgctccagtctgtgtgacagtgagaccctgtctcttacacacacacacacacacacacacacacatgcacacacacagagagagagaaattagaagatactgaattggcagaagagaagggaaatagaaattaaaatactgaataggggagcagtgaacaggggatacccaaaagccaa

SEQ ID NO: 176 - DNA, 500 bp, Homo sapiens
tgatagcaggtgcctgtaattccagctactcaggaggctgaggtgggagaatctattgaacccgggagggggaggttgcagtgagccgagatcatgccattgcactccagcctgggcgacagagtgagactccttctcaaaacaaacaaacaaacaaacaaacaaaatacagaagctgaggcgggaggaacatttgaaccrgattcggaggctgcagtgagctatgattgcaccactgcgctccagtctgtgtgacagtgagaccctgtctcttacacacacacacacacacacacacatgcacacacacagagagagagaaattagaagatactgaattggcagaagagaagggaaatagaaattaaaatactgaataggggagcagtgaacaggggatacccaaaagccaagagcgagagagagcctggcttccagaaatagtggagaagccaggagaactaggtgaaaacccagtgctgggttgccatcagcgagag

SEQ ID NO: 177 - DNA, 595 bp, Homo sapiens
tctgcacctcctctccctccttcctctccccgtcatccctaaatcttgtcctcgagccactgccaccctgtgtaaaccctcatgcccagtcttgsgggtgccatcccttctctttgaagctgaatggaccaaacatacccattgagtgttgggtggggacatctctggaaagtcagcacctggaccagctccacccctctctgaggacaccttctttccctttcagaacaaagaacagccaccatgcagctcttcctcctcttgtgcctggtgcttctcagccctcagggggcctcccttcaccgccaccacccccgggagatgaagaagagagtcgaggacctccatgtaggtgccacggtggcccccagcagcagaagggactttacctttgacctctacagggccttggcttccgctgcccccagccagagcatcttcttctcccctgtgagcatctccatgagcctggccatgctctccctgggggctgggtccagcacaaagatgcagatcctggagggcctgggcctcaacctccagaaaagctcagagaaggagctgcacagaggctttcagcagctccttcaggaac

SEQ ID NO: 178 - DNA, 400 bp, Homo sapiens
tccatatcccatcctccaaaatgtgtcccttgatgtggatgggtagacaggattcctgccctggcagccagacccctgccttgggtctgcacctcctctccctccttcctctccccgtcatccctaaatcttgtcctcgagccactgccaccctgtgtaaaccctcatgtccagtcttgggggtgccatcccttctctttraagctgaatggaccaaacatacccattgagtgttgggtggggacatctctggaaagtcagcacctggaccagctccacccctctctgaggacaccttctttccctttcagaacaaagaacagccaccatgcagctcttcctcctcttgtgcctggtgcttctcagccctcagggggcctcccttcaccgccaccacccc

SEQ ID NO: 179 - DNA, 385 bp, Homo sapiens
aacagccaccatgcagctcttcctcctcttgtgcctggtgcttctcagccctcagggggcctcccttcaccgccaccacccccgggagatgaagaagagagtcgaggacctccatgtaggtgccacggtggcccccagcagcagaagggactttacctttgacctctacagggccttggcttccgctgcccccagccagarcatcttcttctcccctgtgagcatctccatgagcctggccatgctctccctgggggctgggtccagcacaaagatgcagatcctggagggcctgggcctcaacctccagaaaagctcagagaaggagctgcacagaggctttcagcagctccttcaggaactcaaccagcccagagatggcttc

SEQ ID NO: 180 - DNA, 400 bp, Homo sapiens
ctgagcctcggcaatgcccttttcaccgacctggtggtagacctgcaggacaccttcgtaagtgccatgaagacgctgtacctggcagacactttcccyaccaactttagggactctgcaggggccatgaagcagatcaatgattatgtggcaaagcaaacgaagggcaagattgtggacttgcttaagaacctcgatagcaatgcggtcgtgatcatggtgaattacatcttctttaaaggtaaggcccttgggcccaaacctgcactttctttggcttttctgctgcttttatctaaagaatacccaattccctcacatacataaaagacggggagtacgttaagttcttttgggtgcctgttgagaaaaattaagtaaacaagcagccagagaaggt

SEQ ID NO: 181 - DNA, 386 bp, Homo sapiens
ttcccaatgggaaaaaccattcatttccaggatccatactaacttctttctaaaatttaaatcaaaatattggaatgaaagtgcaaacagagaagttcacccagatatcaggtagcattcacagccagccacatttttcaccctcttcacttggagatttggtcttgagtaaaacgttagagaatcagagaacatcagggrtccagggcctctgaagatgtgaaaaccaacctccttgttttgcaaatgtggaaggaaaagtcccacgaaaagtccaagaatgtgcccaatgttataaagagacttgccttcatattcaagaggttcaacagtcactgctctggggctgccataaagatggtctccgctggctatctttactgtct

SEQ ID NO: 182 - DNA, 500 bp, Homo sapiens
ccttttccctttccaggcagctcgagctttaccttcccaaattctccattgagggctcctatcagctggagaaagtcctccccagtctggggatcagtaacgtcttcacctcccatgctgatctgtccggcatcagcaaccactcaaatatccaggtgtctgaggtgggttcagaagctcctatgcatctgcttcccaagrtctattctgttctattctttctattctactctaccccatttcattccattccattccactcaactccactccactccactccactccagttcactctattcaattccactccactccactccagttcactttattcaattccactccactccactccagttcactctattcagttccactccactccactccactccagttcactctattccattccactccattccactcctccactcctctcatccactccactctactcctccactccacatctccactccactcctccactccac

SEQ ID NO: 183 - DNA, 400 bp, Homo sapiens
attacaccttgctcaaagatgccatgagaattcaatgacagacacatgcgaagtcaccccccagcacagtgcctggggcagagtagctgctccattgttccatttcctacttgctccatggctcagttgaacagatacttagaggttgatgcccataggcagaagctttgccatttgctatgatgacttcacctgcccctrgtggcctggtgatgcctggtgtctcccctgcagatggtgcacaaagctgtggtggaggtggacgagtcgggaaccagagcagcggcagccacggggacaatattcactttcaggtcggcccgcctgaactctcagaggctagtgttcaacaggccctttctgatgttcattgtggataacaacatcctcttccttggca

SEQ ID NO: 184 - DNA, 400 bp, Homo sapiens
tttcctacttgctccatggctcagttgaacagatacttagaggttgatgcccataggcagaagctttgccatttgctatgatgacttcacctgcccctggtggcctggtgatgcctggtgtctcccctgcagatggtgcacaaagctgtggtggaggtggacgagtcgggaaccagagcagcggcagccacggggacaatmttcactttcaggtcggcccgcctgaactctcagaggctagtgttcaacaggccctttctgatgttcattgtggataacaacatcctcttccttggcaaagtgaaccgcccctgaggtggggcttctcctgaaatctacaggcctcagggtgggagatgaagggggctatgctatggcccatctgtatgctggtagctag

SEQ ID NO: 185 - DNA, 547 bp, Homo sapiens
gaaccagagcagcggcagccacggggacaatattcactttcaggtcggcccgcctgaactctcagaggctagtgttcaacaggccctttctgatgttcattgtggataacaacatcctcttccttggcaaagtgaaccgcccctgaggtggggcttctcctgaaatctacaggcctcagggtgggagatgaagggggctawgctatggcccatctgtatgctggtagctagtgatttacacaggtttagttgactaatgaggcattacaaataatattactctatgatgattgcttccacccacacgactgcaacatacaggtgccttggggaaatgtggagaacattcaatcttgccgtcactattcatcaatgaagattaacactgagatccagagaggctggatgacttgctcaagttcaccagcatggtagtggcaaagagaggtccagagtcctggcccttgatgcccagctcagtgccacaaagctcaataggagggatgttccagtggatgagggccaccaggaagcacaggtccaaggc

SEQ ID NO: 186 - DNA, 599 bp, Homo sapiens
gggagatgaagggggctaagctatggcccatctgtatgctggtagctagtgatttacacaggtttagttgactaatgaggcattacaaataatattactctatgatgattgcttccacccacacgactgcaacatacaggtgccttggggaaatgtggagaacattcaatcttgccgtcactattcatcaatgaagattarcactgagatccagagaggctggatgacttgctcaagttcaccagcatggtagtggcaaagagaggtccagagtcctggcccttgatgcccagctcagtgccacaaagctcaataggagggatgttccagtggatgagggccaccaggaagcacaggtccaaggctggtcccacacttatcagcagcaacaactgtcagttcatcctgcatgggaaaaatgttggaatgggagtctgaaatggggctactgtttcagtcctaatgtgctgtgtgacattgggacaacactttccctctctggacctcagtttccctctgtatacaaggatcagattcttgctgtgacccaagaactcctgaaatcatatagaaaggctggggtgggccctgtcattcgt

SEQ ID NO: 187 - DNA, 499 bp, Homo sapiens
ttccacccacacgactgcaacatacaggtgccttggggaaatgtggagaacattcaatcttgccgtcactattcatcaatgaagattaacactgagatccagagaggctggatgacttgctcaagttcaccagcatggtagtggcaaagagaggtccagagtcctggcccttgatgcccagctcagtgccacaaagctcartaggagggatgttccagtggatgagggccaccaggaagcacaggtccaaggctggtcccacacttatcagcagcaacaactgtcagttcatcctgcatgggaaaaatgttggaatgggagtctgaaatggggctactgtttcagtcctaatgtgctgtgtgacattgggacaacactttccctctctggacctcagtttccctctgtatacaaggatcagattcttgctgtgacccaagaactcctgaaatcatatagaaaggctggggtgggccctgtcattcgtggttgatttcaa

SEQ ID NO: 188 - DNA, 458 bp, Homo sapiens
aggtccagagtcctggcccttgatgcccagctcagtgccacaaagctcaataggagggatgttccagtggatgagggccaccaggaagcacaggtccaaggctggtcccacacttatcagcagcaacaactgtcagttcatcctgcatgggaaaaatgttggaatgggagtctgaaatggggctactgtttcagtcctaaygtgctgtgtgacattgggacaacactttccctctctggacctcagtttccctctgtatacaaggatcagattcttgctgtgacccaagaactcctgaaatcatatagaaaggctggggtgggccctgtcattcgtggttgatttcaatacactcaagtgccattcatcctttaagaaaaacatctggatatcaaggtggaaatggcccatttaatgattgattatatcattttgtggatatagttataatctgatgg

SEQ ID NO: 189 - DNA, 596 bp, Homo sapiens
tagaaaggctggggtgggccctgtcattcgtggttgatttcaatacactcaagtgccattcatcctttaagaaaaacatctggatatcaaggtggaaatggcccatttaatgattgattatatcattttgtggatatagttataatctgatgggcctggctgggagtggaagaagggaagccttttgcaaatagtagagtrtcagttgcaggtgccaatgactaactttttgaattctatgttggcattaacaataaagcattttgcaaacactggttataactgtctttatggaggcagctctgggaatggtgacattgatagcttaccatgctccaggccgggtgcctggcccttcacctggatggtcgcatttgcccctcataagactcccatgaagaaaggcaccactgttatcccatctgttattcacagatgggaaaggcaaggcttgaagtggttaggtggcttacccagtcacatatcttctaagtggtgcagccagaatttggcggggggagtgcgaccaagaaccctacactcagtcctgtgctctgtgctgtggaggagagatgaccaggagcagaaacttcatt

SEQ ID NO: 190 - DNA, 400 bp, Homo sapiens
aaggcaccactgttatcccatctgttattcacagatgggaaaggcaaggcttgaagtggttaggtggcttacccagtcacatatcttctaagtggtgcagccagaatttggcggggggagtgcgaccaagaaccctacactcagtcctgtgctctgtgctgtggaggagagatgaccaggagcagaaacttcattcagggrcatctcaggcaccagctcccccatgagccagctaagttccctccctcccttcaccaagcaccatgtgtttcctcatgtgccgaatgaagaggattagatactcaagaatggaatgagtgggtgagtgagtccttcgctgcacccaagtctgattttctgtgcgcctgctcaccccaccctgcatgttctaagcatgctt

SEQ ID NO: 191 - DNA, 400 bp, Homo sapiens
atcttctaagtggtgcagccagaatttggcggggggagtgcgaccaagaaccctacactcagtcctgtgctctgtgctgtggaggagagatgaccaggagcagaaacttcattcagggacatctcaggcaccagctcccccatgagccagctaagttccctccctcccttcaccaagcaccatgtgtttcctcatgtgccraatgaagaggattagatactcaagaatggaatgagtgggtgagtgagtccttcgctgcacccaagtctgattttctgtgcgcctgctcaccccaccctgcatgttctaagcatgcttccataaggctgtgccccaccctctgattctagagtctggactgtatcagaggtgagtgcctactagaggtaacaaggtcagg

SEQ ID NO: 192 - DNA, 102 bp, Homo sapiens
  misc_feature (51)..(51): n = absent or C when 52 is T
tttcattttcacaccaaagaatcccaccgcggcagaggaccaccgtctctnngtttagacaatcggtgaagaatggatgacctcactttccccaacaggcgg

SEQ ID NO: 193 - DNA, 399 bp, Homo sapiens
caaacctctggcacagagagcaaagtcctcactgggaggattcccaaggggtcacttgggagagggcagggcagcagccaacctcctctaagtgggctgaagcaggtgaagaaagtggcagaagccacgcggtggcaaaaaggagtcacacactccacctggagacgccttgaagtaactgcacgaaatttgaggrtggccaggcagttctacaacagccsctcacagggagagccagaacacagaagaactcagatgactggtagtattaccttcttcataatcccaggcttggggggctgcgatggagtcagaggaaactcagttcagaacatctttggtttttacaaatacaaattaactggaacgctaaattctagcctgttaatctggtcactg

SEQ ID NO: 194 - DNA, 400 bp, Homo sapiens
atctcagccctgagaaaggaggtgggtaggcttggcgatggggttgaagggcccggtgcgcatgcgttccccttgcccctgcgtgtggccgggggctgcctgcattaggaggtctttgctgggttctagagcactgtagatttgaggccaacggggccgactagactgacttctgtatttatcctttgctggtgtcaggargttcctttcctttctggaaaatgcagaatgggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagtt

SEQ ID NO: 195 - DNA, 550 bp, Homo sapiens
ggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagttgcagtactttttggttatgtaaactcaatggytaggattcctcaaagccattccagctaagattcatacctcagagcccaccaaagtggcaaatcataaataggttaaagcatctccccactttcaatgcaaggtattttggtcctgtttggtagaaagaaaagaacacaggaggggagattgggagcccacactcgaattctggttctgccaaaccagccttgtgatcttgggtaaattccctaccacctctggactccatcagtaaaattgggcgtggactaggtgatctcatagatccttcctgctggaacattctatggcttgaattatattctcctaattattgtcaaaattgctgttattaagtatctactgtgt

SEQ ID NO: 196 - DNA, 500 bp, Homo sapiens
tacttaatcctgagtctcagtttccttatctccaaaaaccttccttgcaaatttgttttgaagattagacacaatatttatttaaagtgcctggcacacagtagatacttaataacagcaattttgacaataattaggagaatataattcaagccatagaatgttccagcaggaaggatctatgagatcacctagtccacscccaattttactgatggagtccagaggtggtagggaatttacccaagatcacaaggctggtttggcagaaccagaattcgagtgtgggctcccaatctcccctcctgtgttcttttctttctaccaaacaggaccaaaataccttgcattgaaagtggggagatgctttaacctatttatgatttgccactttggtgggctctgaggtatgaatcttagctggaatggctttgaggaatcctagccattgagtttacataaccaaaaagtactgcaactgctctgagaagctgaacatg

SEQ ID NO: 197 - DNA, 550 bp, Homo sapiens
tcttgttacatgtctgggaaagaataccagaattgttatcacctaagtgtccctaaaacaaacaccactagagggccttttcattgttcaaccacagccaggaaagtctctaagaaaaatgaagctacaactcattggcatcctggcaagcaaattccagtggagtgggggcacacttgggttcagttccaagctcacctktgactttaggtgtgttacttaatcctgagtctcagtttccttatctccaaaaaccttccttgcaaatttgttttgaagattagacacaatatttatttaaagtgcctggcacacagtagatacttaataacagcaattttgacaataattaggagaatataattcaagccatagaatgttccagcaggaaggatctatgagatcacctagtccacgcccaattttactgatggagtccagaggtggtagggaatttacccaagatcacaaggctggtttggcagaaccagaattcgagtgtgggctcccaatctcccctcctgtgttcttttctttctaccaaacagga

SEQ ID NO: 198 - DNA, 391 bp, Homo sapiens
atccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactcccacaggtttaattagttcatcctgggaaaggtactckcagggccttttccctctctggctgcccctggcagggtccaggtctgccctccctccctgcccagctcattctccacagtgagataacctgcactgtcttctgattatttatcaaagggagtttccagctcagcatacacaaggcagagagtgcagacagaacatcaaggggacaattcagagaaggatcc

SEQ ID NO: 199 - DNA, 450 bp, Homo sapiens
ccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccrgatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtga

SEQ ID NO: 200 - DNA, 500 bp, Homo sapiens
gttctgcctccctcttctgggttcctaaagcactgcacctatctacctgtcaaagcatctaccacattgtaccacaccttaaaatcaatggtttttttcttctcagccagcatgtggatgcctcaataaagcagactcctttcatgacctaaaactaatttcaggggggaaaaaaagacgagctgggcgcagtggctcacrcctataatcccagcactttgggaggctgaggcgggaggatcacttgaggtcagaagaccagcctggccaacatggcaaaaccccgtctctactaaaaatacaaaaattagctgggcgtggtggcgcacctataatcccagctactcaggaagctgagacatgataatcgcttgagcctgggaggtagagcctggggctgcactccatcctgggcaacagagggagattctgtctcaaaaaataataataataatataaataaataaataatttttttaaaaaaagactctttcctatat

SEQ ID NO: 201 - DNA, 500 bp, Homo sapiens
tctcactcacctttggctcctgcccttagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtgtaagtagcagatcagttytttcccttgcagctgcccccaaaataccatctcctacagaccagcagggacactcacatccacagacacagcaaagacacagactggcagagctagctgtaaatgaggaaagactcctggagtcagatctcttgctcatttctctttgagcaggcgttgggggtggctgctaggcatttacatgtgaaatttgcaaacagctttcctgttatttgtgagtcatttgtgggttattaactactcccctctctcttcataaaaggagcccagagcttcagtcaggcctccactgcctcttt

SEQ ID NO: 202 - DNA, 540 bp, Homo sapiens
tctctaaatgaaagggcatcaaaaagaccgcatttcagttatttccccaaacctcaagttcattctccttttgttcttcctgcagcaaatgaaggatcagctggacaacttgttgttaaaggagtccttgctggaggactttaaggtgagagcaggggcggggtgctgggggagtgtgcagcatgattaagggaagggagrctctgcttcctgattgcagggaattgggtttgtttccttcgctttgaaaaggagaagtgggaagatgttaactcagcacatccagcagccagagggtttacaaagggctcagtccttcggggaggcttctggtgaaggaggatcgctagaaccaagctgtcctcttaagctagttgcagcagcccctcctcccagccacctccgccaatctctcactcacctttggctcctgcccttagggttacctgggttgccaagccttgtctgagatgatccagttttacctggaggaggtgatgccccaagctgagaaccaagacccagacatcaaggcgcatg

SEQ ID NO: 203 - DNA, 450 bp, Homo sapiens
tcctctgtttttaaaactccccttttgatttttttgggccagagccaatttkatttaaaaaaaaaaatctctaaatgaaagggcatcaaaaagaccgcatttcagttatttccccaaacctcaagttcattctccttttgttcttcctgcagcaaatgaaggatcagctggacaacttgttgttaaaggagtccttgctggaggactttaaggtgagagcaggggcggggtgctgggggagtgtgcagcatgattaagggaagggagactctgcttcctgattgcagggaattgggtttgtttccttcgctttgaaaaggagaagtgggaagatgttaactcagcacatccagcagccagagggtttacaaagggctcagtccttcggggaggcttctggtgaaggaggatcgctagaaccaagctgtcctcttaagctagttgcagcag

SEQ ID NO: 204 - DNA, 456 bp, Homo sapiens
ccaacacctattcccccaaacttaaattcttaagagaatcctagatcaagccatgggtttggtgagttaagctaagccagatgatacagtaaatgtgcaggaaacctgccttataaagtaaatgcgttctctctcgtgctgagaaacttataagatcctgctggcgctctatactttattggctaggagaagtaaagaaakgtctgattcgaggtgaagatgctccccatgccttgcagcagggaaatttaaattgcctctgcttagagcgtttccagacctgaaagaccagtggtttagggaagcactctacatgagggaaacctgcattagaaggagcttcttaatccctgggatctttccaagctaaactggatgtctacagtggggagaaagaaaagcagagaacaggacatgaggggggctcaaggccccgaagggttgacataggtgtcc

SEQ ID NO: 205 - DNA, 400 bp, Homo sapiens
gacttcttttccttgttatttcaacttcttccaccccatcttttaaactttagactccagccacagaagcttacaactaaaagaaactctaaggccaatttaatccaaggtttcattctatgtgctggagatggtgtacagtagggtgaggaaaccaaattctcagttggcactggtgtacccttgtacaggtgatgtaayatctctgtgcctcagtttgctcactataaaatagagacggtaggggtcatggtgagcactacctgactagcatataagaagctttcagcaagtgcagactactcttacccacttcccccaagcacagttggggtgggggacagctgaagaggtggaaacatgtgcctgagaatcctaatgaaatcggggtaaaggagcc

SEQ ID NO: 206 - DNA, 450 bp, Homo sapiens
tatcaagagacagcaaaaatatttgtaaagaaaggatgtccaacaatctgtgtggttgtttttctgtgttcctccaatggtagggcctctgttcaccagtgccgtctcttcttttagctgtaagaaaagcctggagtgcacgaagttgtgcctaccccagattgagaatgttaagggcactgaggactcaggtgaggagargtgacctggtgcccatgctcacctgccctctccctcttcttgcccccacccgtccatccatcccacccatccatctatccctgcggcccccctctgcccgctcctctgaccaacacctgctttgtctgcaggcaccacagtgctgttgcccctggtcattttctttggtctttgccttttatccctcctcttcattggtttaatgtatcgctaccaacggtggaagtccaagctctactccattggtga

SEQ ID NO: 207 - DNA, 450 bp, Homo sapiens
ggctcagcctccacctccagggctcaagccatcctcttgccttagcctcctgagtagctgggattagaggcacacaccactacacccagctaatgttttactttttgtagagacagggtcctactatattgcccaggctggcctcggactcctgggctcaagcgatcttccgcctcagcctcccaaagtgctaggattacsggcatgagccaccacgcctggcctgggccttagatttcttatatttaaagtaagcataatgacattcatttggtgaatttgtgagaaccaaaaacaaagaaacaaacaaaacctacaacacgtctgacacaaaactatttattttccattaatcttcttttttttttttttttttttttttgacacagagtcctgctctgtcgcccaggctggaatgcagtggcgcgatctcggctcactgcaacctct

SEQ ID NO: 208 - DNA, 450 bp, Homo sapiens
cgtgcacctctcctgtgagcgcagctctcctgaggccaagccctctccccaccccaggggttggccccttccccatgcggtggcacttcctttccttccccctcctgtattctgtgggtctgacaaccaactcctctctggccgcccccaccctgtccctcgtcacttcctctgtcctgtggggtgggggtgcaggcgctyctcctttagctgtgccgcacttctccctacaggccaggagaaacagaacaccgtgtgcacctgccatgcaggtttctttctaagagaaaacgagtgtgtctcctgtagtaagtgagtatctctgagagctgctgggcactggatggtggcatgggttgggacgggtgactggtgggaaccattagctgggcaacagatgccaggatgccccagagtgctcagggtcctactggctgagtaggagacact

SEQ ID NO: 209 - DNA, 450 bp, Homo sapiens
gggctctggaggctttcctgtattgccagtgggcttggggagggtctgtggagactcagaactggccttgtttcctaaggattgtctggggaccccagggaggcccccaaacccagcacaactggtcagaaccagccaggctgtgggaatgcggtgaacccagggtgggagggcagccttggcttgcttcctgctgggackggggagtgttgggggatggagtgagagctcacggaatgggtttagctgttggagacttgttgaactgggaggaggagctggggcggggcctcagctaaaggccgctgaggggctaggaggagccaagtggccctcagggaagggagggcacagacctgatgggcggaagccagggtcgagggagacttcccttcgggatggaatggggagagggaggcatttcccggaacatgtgggccaagtgggaca

SEQ ID NO: 210 - DNA, 550 bp, Homo sapiens
aagacattttttgatctctcatcttataaggttcgtggtcactttggggagatcatatctgtcacccaacataaccatattatgataagagccaaaagtagatagggtcagttcacgtgcttcgagttcacagggactatgggtctaaggagccggggtggaggaaacagacatygtcaatggtggcttcacgggagggagatgggatctcaactgggcccttggaggagaagctgccacgacctcccccaacaccttgacattaaatgaacagacacatgaatgagggggaaaggaagactaattgggtccctgcaaggtggctggatcggggtcagaccacaaggccgatctcagcgtcgcctccccactctgcagccccagcacaggaagtcacactttaaagcctcctctggcggaaattgtgggggagttggaggggtgttgggccaccccctcaactgtctctccacaggcaccccagcttcctgcccttctgctccaggctggagtctgggcctaaagagctcacctcctgtttctcctgttt

SEQ ID NO: 211 - DNA, 450 bp, Homo sapiens
ttgtgtgtggggaggtggggggatggtctgaaaactctcccccggagataaatatattcctaccaggggtgctgtctcctcacctccctctttgggaatcactggcttctactagagtggaagacagatgtatcattagatcgatcagttgatccatatttatctgctcccagtctggaggtctggttctgggagctgagrggacaccaggggaggataagacactttctgaccaagacatttttttgatctctcatcttataaggttcgtggtcactttggggagatcatatctgtcacccaacataaccatattatgataagagccaaaagtagatagggtcagttcacgtgcttcgagttcacagggactatgggtctaaggagccggggtggaggaaacagacatcgtcaatggtggcttcacgggagggagatgggatctcaact

SEQ ID NO: 212 - DNA, 450 bp, Homo sapiens
tgtgcttgtgtgtggggaggtggggggatggtctgaaaactctcccccggagataaatatattcctaccaggggtgctgtctcctcacctccctctttgggaatcactggcttctactagagtggaagacagatgtatcattagatcgatcagttgatccatatttatctgctcccagtctggaggtctggttctgggagytgagaggacaccaggggaggataagacactttctgaccaagacatttttttgatctctcatcttataaggttcgtggtcactttggggagatcatatctgtcacccaacataaccatattatgataagagccaaaagtagatagggtcagttcacgtgcttcgagttcacagggactatgggtctaaggagccggggtggaggaaacagacatcgtcaatggtggcttcacgggagggagatgggatct

SEQ ID NO: 213 - DNA, 450 bp, Homo sapiens
taggttgtagcaaatagaaagcactcaataaagtttttatattgctgtgactagtagtaattactgggtggctacctgtgttgggaaaacagagggtaaaggtagcctgaacaggtaaagggaagtgcctgcgtcctggggtgcttcagcccaggtgggattatgtctcctaagggacagaagcctggcctggagctggargaaagggaaaacaaagggaatgcaacatccttctgaatttctcaccattcagtgggcaatgcagagctcacagtgtgtgtgtgtgtgtgtgtgtgtgtgtgtgtgtgagagagagagagagagagagagagaagtggggtaggggagtagggaagaatgatacaggagagactgtggcaaagcaaacaggattttgctgctctcaaagagcttacagcctagtaaccaagatggcttacagtgaaaaat

SEQ ID NO: 214 - DNA, 448 bp, Homo sapiens
  misc_feature (343)..(343): n is a, c, g, or t
gcgcagcccctactccaaaaggcggatgaatggggaaccccacactggcagtggctgaggttaggacctgcaggcctgaggctggcgccaggaccaggcccgggcaggagaggctcggccccctcccggagagggcccacgccagccggaaggtgcctcgcccaccagcccactcttccctttgtccctggtctcaccagyggcagcagcaggtcaggcacggtggagaggcccatgccagacagctatggcctctcactcccccatttgggctcatggcagtgtggcagcggcagtgctggggcttcccgggactcggtctgtccaggacgtcccaagtgcncttgggtgacagttgagggttgagactcgggcatagagatcacggcctggtcccagtgatcttgaaccccaaaggccagaactggagcctcagtccagagaattc

SEQ ID NO: 215 - DNA, 500 bp, Homo sapiens
  misc_feature (52)..(52): n is a, c, g, or t
ggatcagtaaattcccaagaaagagggagactaggaggctagtgaagaactnggagtaaaggggaggattactaagggacatggagtacctatcatgtgtcggacgcttatytatatctctcccatctgaacaaatccttacaggaaccccaggagacaggttatctccactctgcaaattggaaaacagatccagacagkttcagttatgtgtctgagaagttcatttrtgtgtccaagacacattcttagctaaaaagctaagcattctgaattggaacccagagaatttgactcccagactctggatcttttcactgctgtgatccatctgggaaaggctagtgatgtgggcaaggggcttattgccccttggtgtttggttgggagtggtcggattggtgggttgggggcacaaggcagccagmtctgggactcctgtgcttgtgactggactacaaagagttaaagaacgttgggcctcctcctcccgcctcctg

SEQ ID NO: 216 - DNA, 400 bp, Homo sapiens
ggggtctgactcagtgacagaaaaagtggcagtgtgtctctcatagccaaaggggcccttggaccggcagtcgggagtctggggttctctgttggctctgcctcctggcacattgggtttctggacctcagttttctcctctataaaaccgggcagttgggtgggcacggtggctcacacctgtaatcctagcactttagsaggctgaggtgggcagatcatttgggcccaggagttcaagacctgcctgtgtaacatggtgagaccctgtctctacaaaaaatacaaaaattacccaggcgtggtggtatgcacctatagtcccagctgcttgggaggctgaggtgggaggattacttgaacctgggaggtcgaggctgcagtgagctgcgatggtacc

SEQ ID NO: 217 - DNA, 450 bp, Homo sapiens
tttctccacccccaaaggaatgcaaaccagggaagggaggggagatcccattaggctgagccctctgtgcctccagctcacacaggaagggtcacagttcccacaaatgggacatgtctatataggaaatgacactaaatgtccactctcccctgggagctaggggaaacaagggacacttcccccaacacctaggatccvtgaacactgtcttcctgctctgtgcgcacgactccttctccaaataaaattttactggaaagagcagaagaaaaaggcaacaagtcctacttctagcagagacctgaacagcggagagtcctcacgaaactgagggtgaacctcgtggtgcccagctctttctttcttgatccttatattcctgtgccccttccccttcctccccacagttctgaagaaaaaggaattaggccatccacccatcccctg

SEQ ID NO: 218 - DNA, 450 bp, Homo sapiens
aagtaggacttgttgcctttttcttctgctctttccagtaaaattttatttggagaaggagtcgtgcgcacagagcaggaagacagtgttcacggatcctaggtgttgggggaagtgtcccttgtttcccctagctcccaggggagagtggacatttagtgtcatttcctatatagacatgtcccatttgtgggaactgtracccttcctgtgtgagctggaggcacagagggctcagcctaatgggatctcccctcccttccctggtttgcattcctttgggggtggagaaaaccccacttgactatgttcgggtgctgtgaacttccctcccaggccagcagagggctggctgtagctcccaggcgccccgcccccctgcccaaccccgagtccgcctgccttttgttccgttgtggtttggatcctcccatttctctggggacaccc

SEQ ID NO: 219 - DNA, 450 bp, Homo sapiens
ccagggtgtccccagagaaatgggaggatccaaaccacaacggaacaaaaggcaggcggactcggggttgggcaggggggcggggcgcctgggagctacagccagccctctgctggcctgggagggaagttcacagcacccgaacatagtcaagtggggttttctccacccccaaaggaatgcaaaccagggaagggaggrgagatcccattaggctgagccctctgtgcctccagctcacacaggaagggtcacagttcccacaaatgggacatgtctatataggaaatgacactaaatgtccactctcccctgggagctaggggaaacaagggacacttcccccaacacctaggatccgtgaacactgtcttcctgctctgtgcgcacgactccttctccaaataaaattttactggaaagagcagaagaaaaaggcaacaagtccta

SEQ ID NO: 220 - DNA, 400 bp, Homo sapiens
cctccccagaggtggagagcacaggccacagtcagtggtggggagagccagggtgtccccagagaaatgggaggatccaaaccacaacggaacaaaaggcaggcggactcggggttgggcaggggggcggggcgcctgggagctacagccagccctctgctggcctgggagggaagttcacagcacccgaacatagtcaartggggttttctccacccccaaaggaatgcaaaccagggaagggaggggagatcccattaggctgagccctctgtgcctccagctcacacaggaagggtcacagttcccacaaatgggacatgtctatataggaaatgacactaaatgtccactctcccctgggagctaggggaaacaagggacacttcccccaacacct

SEQ ID NO: 221 - DNA, 451 bp, Homo sapiens
  misc_feature (201)..(201): n = absent or T
ctgacctaaatctggcgtggctgggtagtggccagcagtggtgatgcccagcctgttctgcctcctccttccccaccccaggagccctttccttggcctaggacctggcttctcagccactgaccggccccctgcttccagtgcgccacttaccccttccagcttcccagtggtctctggtctgggagaggcaggacaaanggtctttgtttgctggagaaaaggttgtctgcgataaataaggaaaaccacgaaagcctggttgttggagtgtacgtgtgtgctcccccaggcagtggaggccagccctccttggaggggcggctgcctgatgaaggatgcgggtgaggttccccgcctccacctcccatgggacttggggattcattccaaggggaagctttttgggggaattcctaccccaggtctttttaccctcagttaccaaccc

SEQ ID NO: 222 - DNA, 450 bp, Homo sapiens
acttactacatcctgagtactgtgtacagtagtccacagctatcatttcacacaagtttctccacatggtactattagacactattgagattccattttacagatggggaacaggagrctcaaagaggctaagtaagttgccccaaggccgcacagctagtaagtaaaggagtcagaattagctgacatcaaagtgttcccaagcctatattaggcaaaaacagaggaggcacctttcaggaggaggcacctttccccctgccagtcctcttccccagacatgagctgagaaggtggtgggcatcagcacaggggctgggccctcctggaacccacaggtggcagtgggcggacacgctgtgccagccctgccagccactgataaccccgcccaagagggcaaactgcttgcatcatggaaaaaacagtgctgccactgtagccacgaaa

SEQ ID NO: 223 - DNA, 450 bp, Homo sapiens
ggcccaggattcagttcagctgtcacagtgaggtggcgggatcagatgtggcaggccatgtcccttggaacttgagtacatcgtgtgatctctggaatgaaaacaggccttcaccagtgttgatggtggaaagcttagggaagtgcttcaaacacagtaggagggacttacgttagattttggaaggacttgcctgattcrgaagctccaaagagtggcattacagagctgggtggagagaggggctagccatcttttgtgtcgcccaccgggctcatgtgtcatcgcctctcatgcagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacga

SEQ ID NO: 224 - DNA, 450 bp, Homo sapiens
ctgaggagtccaacatcaccatgcaggtgggcatctttgggaagtggggcaagggggggatagggaggggggtaacactttgggaacaggtggtcccaggtcgtttcctggctagatttgccttgtctggctcctgcccctgagttgcacaggggaggtatggtggggtcttgccttctgtggagaagatgcttcattccyagcccaggttcccagcaagccccaaccatctccttctccctgatggttgcccatgggctcaggaggggacagatggatgcctgtgtcaggagcccctctctccctctcttggagagagtcctgagtgcccccccttcttgggggctttgtttgggaagctggatgagcctggtccatggagagtttaaaaagtcttttggtgttacctggtaatggggcacatctcagcccagatagggtgggagggag

SEQ ID NO: 225 - DNA, 450 bp, Homo sapiens
cttttggtggctgctgtgacggtgcagttggatgcgaggccggctggagggtggtttctcagtgcatgccctcctgtaggcggcaggcggcagacacacagccctcttggccagggagaaaaagttgaatgttggtcattttcagaggcttgtgagtgctccgtgttaaggggcaggtaggatggggtgggggacaaggtytggcggcagtaacccttcaagacagggtgggcggctggcatcagcaagagcttgcagggaaagagagactgagagagagcacctgtgccctgccctttcccccacaccatcttgtctgcctccagtgctgtgcggacattgaagcccccaccaggcctcaaccccttgcctcttccctcagctcccagcttccagagcgaggggatgcggaaaccttccttccaccctttggtgctttctcctaagggg

SEQ ID NO: 226 - DNA, 450 bp, Homo sapiens
gttgaatgttggtcattttcagaggcttgtgagtgctccgtgttaaggggcaggtaggatggggtgggggacaaggtttggcggcagtaacccttcaagacagggtgggcggctggcatcagcaagagcttgcagggaaagagagactgagagagagcacctgtgccctgccctttcccccacaccatcttgtctgcctcyagtgctgtgcggacattgaagcccccaccaggcctcaaccccttgcctcttccctcagctcccagcttccagagcgaggggatgcggaaaccttccttccaccctttggtgctttctcctaagggggacagacttgccctctctggtcccttctccccctcctttcttccctgtgacagacatcctgaggtgtgttctcttgggcttggcaggcatggagagctctggttctcttgaaggggacaggct

SEQ ID NO: 227 - DNA, 450 bp, Homo sapiens
ggcggcagtaacccttcaagacagggtgggcggctggcatcagcaagagcttgcagggaaagagagactgagagagagcacctgtgccctgccctttcccccacaccatcttgtctgcctccagtgctgtgcggacattgaagcccccaccaggcctcaaccccttgcctcttccctcagctcccagcttccagagcgagrggatgcggaaaccttccttccaccctttggtgctttctcctaagggggacagacttgccctctctggtcccttctccccctcctttcttccctgtgacagacatcctgaggtgtgttctcttgggcttggcaggcatggagagctctggttctcttgaaggggacaggctacagcctgccccccttcctgtttccccaaatgactgctctgccatggggagagtagggggctcgcctgggctcggaaga

SEQ ID NO: 228 - DNA, 450 bp, Homo sapiens
cgtgcagcgtcctcctccatgtagcctggctgcgtttttctctgacgttgtccggcgtgcatcgcatttccctctttacccccttgcttccttgaggagagaacagaatcccgattctgccttcttctatattttcctttttatgcattttaatcaaatttatatatgtatgaaactttaaaaatcagagttttacaactyttacatttcagcatgctgttccttggcatgggtccttttttcattcattttcattaaaaggtggacccttttaatgtggaaattcctatcttctgcctctagggacatttatcacttatttcttctacaatctcccctttacttcctctattttctctttctggacctcccattattcagacctctttcctctagttttattgtctcttctatttcccatctctttgactttgtgttttctttcaggga

SEQ ID NO: 229 - DNA, 400 bp, Homo sapiens
cagcaaccctggtacctggttaggaacgcagaccctctgcccccatcctcccaactctgaaaaacactggcttagggaaaggcgcgatgctcaggggtcccccaaagcccgcaggcagagggagtgatgggactggaaggaggccgagtgacttggtgagggattcgggtcccttgcatgccagaggctgctgtgggagcrgacagtcgcgagagcagcactgcagctgcatggggagagggtgttgctccagggacgtgggatggaggctgggcgcgggcgggtggcgctggagggcgggggaggggcagggagcaccagctcctagcagccaacgaccatcgggcgtcgatccctgtttgtctggaagccctcccctcccctgcccgctcacccgctg

SEQ ID NO: 230 - DNA, 336 bp, Homo sapiens
  misc_feature (308)..(308): n is a, c, g, or t
ttagctaatattctcagcccagtcatcagaccggcagaggcagccaccccactgtccccagggaggacacaaacatcctggcaccctctccactgcattctggagctgctttctaggcaggcagtgtgagctcagccccacgtagagcgggcagccgaggccttctgaggctatgtctctagcgaacaaggaccctcaatyccagcttccgccctgacggccagcacacagggacagccctttcattccgcttccacctgggggtgcaggcagagcagcagcgggggtaggcactgcccggagctcanaagtcctcctcagacaggtgccagtgcc

SEQ ID NO: 231 - DNA, 400 bp, Homo sapiens
gaggctgaggtgggaggattgcttgagcttgggagtttgagactagcctgggcaacacagtgagaccctgtctctatttttaaaaaaagtaaaaaaagatctaaaaatttaactttttattttgaaataattagatatttccaggaagctgcaaagaaatgcctggtgggcctgttggcctgtgggtttcctgcaaggccktgggaaggccctgtcattggcagaaccccagatcgtgagggctttccttttaggctgctttctaagaggactcctccaagctcttggaggatggaagacgctcacccatggtgttcggcccctcagagcagggtggggcaggggagctggtgcctgtgcaggctgtggacatttgcatgactccctgtggtcagctaag

SEQ ID NO: 232 - DNA, 400 bp, Homo sapiens
tattttagatttgacgaaatatggaatattacctgttgtgctgatcttgggcaaactataatatctctgggcaaaaatgtccccatctgaaaaacagggacaacgttcctccctcagccagccactatggggctaaaatgagaccacatctgtcaagggttttgccctcacctccctccctgctggacggcatccttggtrggcagaggtgggcttcgggcagaacaagccgtgctgagctaggaccaggagtgctagtgccactgtttgtctatggagagggaggcctcagtgctgagggccaagcaaatatttgtggttatggattaactcgaactccaggctgtcatggcggcaggacggcgaacttgcagtatctccacgacccgcccctgtgagt

SEQ ID NO: 233 - DNA, 400 bp, Homo sapiens
cctcacctccctccctgctggacggcatccttggtgggcagaggtgggcttcgggcagaacaagccgtgctgagctaggaccaggagtgctagtgccactgtttgtctatggagagggaggcctcagtgctgagggccaagcaaatatttgtggttatggattaactcgaactccaggctgtcatggcggcaggacggcgwacttgcagtatctccacgacccgcccctgtgagtccccctccaggcaggtctatgaggggtgtggagggagggctgcccccgggagaagagagctaggtggtgatgagggctgaatcctccagccagggtgctcaacaagcctgagcttggggtgaaaggacacaaggccctccacaggccaggcctggcagccacagt

SEQ ID NO: 234 - DNA, 400 bp, Homo sapiens
ccaccacagcccagcatggtgtggtgcctcagcaggaggcatctggttacaatcaacacaagctgttccagccaatttaaagaaacttcaggaggaatagggttttaggagggcatggggaccctcctgcacccgaagccaggatgtgccaccaatcataaggaggcaggggcctccttccgctgctccctgggactctcyaggtgtccgtggcctcagtccccctctgcacacctgcatcttccttctcatcagcttcctctgctttaagcgtaaacatggatgcccaggacctggcctcaatcttccgagtctggtacttatggtgtactgacagtgtgagaccctactcctctgatcaatcccctgggttggtgacttccctgtgcaatcaatggaa

SEQ ID NO: 235 - DNA, 101 bp, Homo sapiens
ggcctccttccgctgctccctgggactctccaggtgtccgtggcctcagcscccctctgcacacctgcatcttccttctcatcagcttcctctgctttaag

SEQ ID NO: 236 - DNA, 400 bp, Homo sapiens
cccctttcctggtctccacagccaacgggaggaggccatgattcttggggaggtccgcaggacacatgggcccctaaagccacaccaggctgttggtttcatttgtgcctttatagagctgtttatctgcttgggacctgcacctccaccctttcccaaggtgccctcagctcaggcataccctcctctaggatgcctttycccccatcccttcttgctcacacccccaacttgatctctccctcctaactgtgccctgcacccaagacagacacttcacagagcccaggagacacctggggacccttcctgggtgataggtctgtctatcctccaggtgtccctgcccaaggggagaagcatggggaatacttggttgggggaggagaggaagactggg

SEQ ID NO: 237 - DNA, 400 bp, Homo sapiens
ggcccctaaagccacaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagasagacacttcacagagcccaggagacacctggggacccttcctgggtgataggtctgtctatcctccaggtgtccctgcccaaggggagaagcatggggaatacttggttgggggaggagaggaagactggggggatgtgtcaagatggggctgcacgtggtgtactggcagaagagtgagaggatttaacttggcagcc

SEQ ID NO: 238 - DNA, 400 bp, Homo sapiens
acaccaggctgttggtttcatttgtgcctttatagagctgtttatctgcttgggacctgcacctccaccctttcccaaggtgccctcagctcaggcataccctcctctaggatgccttttcccccatcccttcttgctcacacccccaacttgatctctccctcctaactgtgccctgcacccaagacagacacttcacaragcccaggagacacctggggacccttcctgggtgataggtctgtctatcctccaggtgtccctgcccaaggggagaagcatggggaatacttggttgggggaggagaggaagactggggggatgtgtcaagatggggctgcacgtggtgtactggcagaagagtgagaggatttaacttggcagcctttacagcagcag

SEQ ID NO: 239 - DNA, 101 bp, Homo sapiens
  misc_feature (51)..(51): n = absent or C
tttccctgcttcctttcttcctggcgtccccgccttcctccgggcgccccntgcgcacctggggccacctcctggagcgcaagcccagtggtggctccgct

SEQ ID NO: 240 - DNA, 400 bp, Homo sapiens
ctgaaacgagacacagaagaccaagaagaccaagtagatccgcggctcattgatgggaagatgaccaggcggggagacagcccctggcaggtgggaggcgaggcagcaccggctgctcacgtgctgggtccgggatcactgagtccatcctggcagctatgctcagggtgcagaaaccgagagggaagcgctgccattgcktttgggggatgatgaaggtgggggatgcttcagggaaagatggacgcaacctgaggggagaggagcagccagggtgggtgaggggaggggcatgggggcatggaggggtctgcaggagggagggttacagtttctaaaaagagctggaaagacactgctctgctggcgggattttaggcagaagccctgctgatgggag

SEQ ID NO: 241 - DNA, 400 bp, Homo sapiens
ctccctggcagtgccgtgttctgggggtcctcctctctgggtctcactgcccctggggtctctccagctacctttgctccacgttcctttgtggctctggtctgtgtctggggtttccaggggtctcgggcttccctgctgcccattccttctctggtctcacggctccgtgactcctgaaaaccaaccagcatcctaccyctttgggattgacacctgttggccactccttctggcaggaaaagtcaccgttgatagggttccacggcatagacaggtggctccgcgccagtgcctgggacgtgtgggtgcacagtctccgggtgaaccttcttcaggccctctgcccaggcctgcaggggcacagcagtgggtgggcctcaggaaagtgccactgggg

SEQ ID NO: 242 - DNA, 101 bp, Homo sapiens
tgtccactaataaattatgacctcagtttcaaaaagattgctttaggtaascaatcatcttctgagatttatacagattgctcataattctctcctatttt

SEQ ID NO: 243 - DNA, 400 bp, Homo sapiens
cctggtgccaaaaacgttgaggaccactgctccacagaatctatcggtcactcttcctcccctcacccccttgccctaaaagcacaccctgcaaacctgccatgaattgacactctgtttctatcccttttccccttgtgtctgtgtctggaggaagaggataaaggacaagctgccccaagtcctagcgggcagctcgargaagtgaaacttacacgttggtctcctgtttccttaccaagcttttaccatggtaacccctggtcccgttcagccaccaccaccccacccagcacacctccaacctcagccagacaaggttgttgacacaagagagccctcaggggcacagagagagtctggacacgtggggagtcagccgtgtatcatcggaggcggc

(19) United States
(12) Patent Application Publication
(10) Pub. No.: US 2011/0027184 A1
(43) Pub. Date: Feb. 3, 2011

(54) PROTEIN C PATHWAY ASSOCIATED POLYMORPHISMS AS RESPONSE PREDICTORS TO ACTIVATED PROTEIN C OR PROTEIN C-LIKE COMPOUND ADMINISTRATION

(76) Inventors: James A. Russell, Vancouver (CA); Keith R. Walley, North Vancouver (CA)

Correspondence Address: Browdy and Neimark, PLLC, 1625 K Street, N.W., Suite 1100, Washington, DC, 20006, US

(21) Appl. No.: 12/160,889

(22) PCT Filed: Jan. 12, 2007
(86) PCT No.: PCT/CA07/00054

§ 371 Date: Aug. 30, 2010

Related U.S. Application Data
(60) Provisional application No. 60/758,193, filed on Jan. 12, 2006.
(60) Provisional application No. 60/783,021, filed on Mar. 17, 2006.

(51) Int. Cl. A61K 49/00; C12Q 1/68; G01N 33/50; C40B 40/08

(52) U.S. Cl. 424/9.2; 435/6; 436/94; 506/17

(57) ABSTRACT

The invention provides methods, nucleic acids, compositions and kits for predicting a subject's response to treatment with activated protein C or protein C like compound to identify subjects having a greater benefit from treatment with activated protein C. The method generally comprises determining a protein C pathway associated gene polymorphism genotype(s) of a subject for one or more polymorphisms in the these genes, comparing the determined genotype with known genotypes for the polymorphism that correspond with an improved response polymorphism to identify potential subjects having an inflammatory condition who are more likely to benefit from treatment with activated protein C or protein C like compound and subsequent to treatment recover from the inflammatory condition. The invention also provides for methods of treating such subjects with an anti-inflammatory agent or anti-coagulant agent based on the subject's genotype.

### FIELD OF THE INVENTION

The field of the invention relates to the assessment and/or treatment of subjects with an inflammatory condition.

### BACKGROUND OF THE INVENTION

The septic inflammatory response involves counter-regulation between pro- and anti-inflammatory cytokines, pro-coagulant and fibrinolytic factors, pro-apoptotic and anti-apoptotic activity, and further counter-regulatory activity in related pathways. Altered balance of these counter-regulatory pathways leads to altered clinical outcome in subjects having an inflammatory condition, for example severe sepsis. Genetic variation between individuals is one factor that can alter the balance of these pathways and may lead to altered clinical outcome. Indeed, genotype has been shown to play a role in the prediction of subject outcome in inflammatory and infectious diseases (MCGUIRE W. et al. Nature (1994) 371(6497):508-10; MIRA J. P. et al. JAMA (1999) 282(6):561-8; NADEL S. et al. Journal of Infectious Diseases (1996) 174(4):878-80; MAJETSCHAK M. et al. Ann Surg (1999) 230(2):207-14; STUBER F. et al. Crit. Care Med (1996) 24(3):381-4; STUBER F. et al. Journal of Inflammation (1996) 46(1):42-50; and WEITKAMP J H. et al. Infection (2000) 28(2):92-6).

New therapies for severe sepsis often aim to beneficially alter this counter-regulatory balance using strategies targeting one or more of these specific pathways. In particular, XIGRIS™ (drotrecogin alpha activated, activated protein C, APC) which has anti-inflammatory, anti-coagulant, pro-fibrinolytic and anti-apoptotic activity, improved 28-day mortality in patients having severe sepsis in the Phase III PROWESS trial (BERNARD G R. et al. New England Journal of Medicine (2001) 344(10):699-709).

Protein C, when activated to form activated protein C or protein C like compound (APC), plays a major role in regulating the inflammatory, coagulation, fibrinolysis and apoptosis pathways (“protein C associated pathways”) triggered by septic or non-septic stimuli such as major surgery. APC inactivates coagulation factor Va (WALKER F J. et al. Biochim Biophys Acta (1979) 571(2):333-42) and coagulation factor VIIIa (FULCHER C A. et al. Blood (1984) 63(2):486-9) and decreases synthesis of plasminogen activator inhibitor type1 (SERPINE1) (VAN HINSBERGH V W. et al. Blood (1985) 65(2):444-51). APC bound to the endothelial protein C receptor activates the protease-activated receptor 1 (RIEWALD M. et al. Science (2002) 296(5574):1880-2) to decrease downstream NFκB and subsequent TNFα, IL1β, and IL6 expression (MURAKAMI K. et al. American Journal of Physiology (1997) 272(2 Pt 1):L197-202; HANCOCK W W. et al. Transplantation (1995) 60(12):1525-32; and GREY S T. et al. Journal of Immunology (1994) 153(8):3664-72). Activated protein C or protein C like compound also decreases adhesion and activation of neutrophils to endothelial cells, decreases apoptosis of endothelial cells and neurons, and decreases neutrophil chemotaxis (JOYCE D E. et al. J Biol Chem (2001) 276(14):11199-203; GRINNELL B W. et al. Glycobiology (1994) 4(2):221-5; LIU D. et al. Nat Med (2004) 10(12):1379-83; and STURN D H. et al. Blood (2003) 102(4):1499-505). Accordingly, protein C has been implicated as having a central role in the pathophysiology of the systemic inflammatory response syndrome.

Infection and inflammation impact protein C regulation. Protein C is produced in its inactive form by the liver. Acute inflammatory states due to infection, major surgery, or shock decrease levels of protein C (BLAMEY S L. et al. Thromb Haemost (1985) 54(3):622-5; FIJNVANDRAAT K. et al. Thrombosis & Haemostasis (1995) 73(1):15-20; GRIFFIN J H. et al. Blood (1982) 60(1):261-4; HESSELVIK J F. et al. Thromb Haemost (1991) 65(2):126-9; and TAYLOR F B. et al. Journal of Clinical Investigation (1987) 79(3):918-25) which is related to poor prognosis (LORENTE J A. et al. Chest (1993) 103(5):1536-42; FISHER C J. Jr. and YAN S B. Crit. Care Med (2000) 28(9 Suppl):S49-56; VERVLOET M G. et al. Semin Thromb Hemost (1998) 24(1):33-44; and YAN S B. and DHAINAUT J F. Crit. Care Med (2001) 29(7 Suppl):S69-74). Endothelial pathways required for protein C activation, including thrombomodulin and endothelial cell protein C receptor (EPCR) expression on endothelial cells, are impaired by pro-inflammatory cytokines (STEARNS-KUROSAWA D J. et al. Proceedings of the National Academy of Sciences of the United States of America (1996) 93(19):10212-6) and in severe menigococcal sepsis (FAUST S N. et al. N Engl J Med (2001) 345(6):408-16).

Genotype can alter response to therapeutic interventions. Genentech's HERCEPTIN® was not effective in its overall Phase III trial but was shown to be effective in a genetic subset of patients with human epidermal growth factor receptor 2 (HER2)-positive metastatic breast cancer. Similarly, Novartis' GLEEVEC® is only indicated for the subset of chronic myeloid leukemia patients who carry a reciprocal translocation between chromosomes 9 and 22.

Numerous genes are known within the coagulation, fibrinolysis and inflammatory pathways and reported to have an association with activated protein C or protein C like compound action, for example, fibrinogen B beta polypeptide (FGB), coagulation factor II (F2), coagulation factor II receptor (F2R), coagulation factor 111 (F3), coagulation factor V (F5), coagulation factor VII (F7), coagulation factor X (F10), serine (or cysteine) proteinase inhibitor, Glade E type 1 (SERPINE1 or PAI-1), protein C inhibitor (SERPINA5), interleukin 6 (IL6), interleukin 10 (IL10), interleukin 12A (IL12A), tumor necrosis factor alpha receptor-1 (TNFRSF1A), vascular endothelial growth factor (VEGF), protein C (PROC) and protein C receptor (PROCR).

Human fibrinogen B beta polypeptide (FGB) or fibrinogen-beta polypeptide chain is encoded by the beta component of fibrinogen and maps to chromosome 4q28. Representative Homo sapiens FGB gene sequences are listed in GenBank under accession numbers AF388026.1 (GI:14423574) and M64983.1 (GI:182597). FGB is a blood-borne glycoprotein comprised of three pairs of nonidentical polypeptide chains. Fibrinogen is cleaved by thrombin to form fibrin for blood clot formation following vascular injury. Furthermore, cleavage products of fibrinogen and fibrin have been reported to regulate cell adhesion and spreading, display vasoconstrictor and chemotactic activities, and as mitogens for several cell types. Mutations in this gene have been associated with afibrinogenemia, dysfibrinogenemia, hypodysfibrinogenemia and thrombotic tendency.

Human coagulation factor II (F2) maps to chromosome 11p1-q12. Representative Homo sapiens F2 gene sequences are listed in GenBank under accession numbers AF478696.1 (GI:18653447) and BC051332.1 (GI:30802114). F2 is proteolytically cleaved to form thrombin in the first step of the coagulation cascade and is involved in maintenance of vascular integrity. Mutations in this gene have been associated with thrombosis and dysprothrombinemia.

Human coagulation factor II receptor (F2R or CF2R), thrombin receptor (TR), or protease-activated receptor 1 (PAR1) maps to chromosome 5q13. Representative Homo sapiens F2R gene sequences are listed in GenBank under accession numbers AF391809.2 (GI:14971463) and M62424.1 (GI:339676). F2R is a 7-transmembrane receptor involved in the regulation of thrombotic response. F2R is a G-protein coupled receptor family member and proteolytic cleavage of the receptor leads to activation.

Human coagulation factor 111 (F3) or tissue factor (TF) or tissue thromboplastin maps to chromosome 1p22-p21. Representative Homo sapiens F3 gene sequences are listed in GenBank under accession numbers AF540377.1 (GI:22536175) and J02846.1 (GI:339505). The F3 gene encodes a cell surface glycoprotein, which is involved in the initiation of the blood coagulation cascades, and acts as a high-affinity receptor for coagulation factor VII. The F3-F7 complex catalyses the initiation of the coagulation protease cascades. To date F3 has not been associated with a congenital deficiency.

Human coagulation factor V (F5) or protein c cofactor maps to chromosome 1q23. Representative Homo sapiens F5 gene sequences are listed in GenBank under accession numbers AY364535.1 (GI:33867366) and M16967.1 (GI:182411). The F5 gene is essential in the blood coagulation cascade and circulates in blood plasma. F5 is converted to the active form by the release of the activation peptide by thrombin during coagulation. Active F5 is a cofactor with activated coagulation factor X, which activates prothrombin to thrombin. Mutations in this gene have been associated with an autosomal recessive hemorrhagic diathesis or an autosomal dominant form of thrombophilia, which is known as activated protein C or protein C like compound resistance.

Human coagulation factor VII (F7) maps to chromosome 13q34. Representative Homo sapiens F7 gene sequences are listed in GenBank under accession numbers AY212252.1 (GI:37781362) and AF466933.2 (GI:38112686). F7 is a vitamin K-dependent factor essential for hemostasis, circulates in the blood in an inactive form, and is converted to an active form by either factor IXa, factor Xa, factor XIIa, or thrombin following minor proteolysis. Active F7 and F3, when in the presence of calcium ions activate the coagulation cascade by converting factor IX to factor IXa and/or factor X to factor Xa. Mutations in this gene have been associated with coagulopathy.

Human coagulation factor X (F10) maps to chromosome 13q34. Representative Homo sapiens F10 gene sequences are listed in GenBank under accession numbers AF503510.1 (GI:20336662) and NM_{—}000504.2 (GI:9961350). F10 encodes a vitamin K-dependent coagulation factor X precursor involved in the blood coagulation cascade and is converted to a mature two-chain form by the excision of the tripeptide RKR. Mature F10 is activated by the cleavage of the activation peptide by factor IXa (in the intrinsic pathway), or by factor VIIa (in the extrinsic pathway). Activated F10 can convert prothrombin to thrombin in the presence of factor Va, Ca+2, and phospholipid during blood clotting. Mutations of this gene have been associated with factor X deficiency, a hemorrhagic condition of variable severity.

The human SERPINE1 (plasminogen activator inhibitor type 1 (PAI-1)) gene maps to chromosome 7q21-q22. A representative Homo sapiens SERPINE1 gene sequence is listed in GenBank under accession number AF386492.2 (GI:14488407) DAWSON et al. (Journal of Biological Chemistry (1993) 268(15):10739-45) identified an insertion/deletion polymorphism (4G/5G) at position −675 of the SERPINE1 promoter sequence, which corresponds to position 201 of SEQ ID NO:14. This polymorphism also has an A allele associated with it, but the frequency of this allele is generally low in the populations tested. The 4G (or “del” or “−”) allele is a single base pair deletion promoter polymorphism of the SERPINE1 gene and is associated with increased protein levels of SERPINE1 (DAWSON S J et al. (1993); DAWSON S J et al. Arteriosclerosis & Thrombosis (1991) 11(1):183-90). The 4G allele of this single nucleotide polymorphism (SNP) is associated with increased risk of deep venous thrombosis (SEGUI R et al. British Journal of Haematology (2000) 111(1):122-8), stroke (HINDORFF L A et al. Journal of Cardiovascular Risk (2002) 9(2):131-7), acute myocardial infarction (BOEKHOLDT S M et al. Circulation (2001) 104(25):3063-8; ERIKSSON P et al. PNAS (1995) 92(6):1851-5.), late lumen loss after coronary artery stent placement (ORTLEPPG J R et al. Clinical Cardiology (2001) 24(9):585-91), and sudden cardiac death (ANVARI A et al. Thrombosis Research (2001) 103(2):103-7; MIKKELSSON J et al. Thrombosis & Haemostasis (2000) 84(1):78-82). In the critically ill, the 4G allele is also associated with decreased survival in patients who have had severe trauma (MENGES T et al. Lancet (2001) 357(9262):1096-7) and patients who had meningococcemia (HERMANS P W et al. Lancet. (1999) 354(9178):556-60) as well as increased risk of shock in patients who had meningococcemia (WESTENDORP R G et al. Lancet (1999) 354(9178):561-3). The SERPINE1 4G genotype has also been associated with adverse patient outcomes ((MENGES et al. (2001); HERMANS et al. (1999); WESTENDORP R G et al. (1999); ENDLER G et al. British Journal of Haematology (2000) 110(2):469-71; GARDEMANN A et al. Thrombosis & Haemostasis (1999) 82(3):1121-6; HOOPER W C et al. Thrombosis Research (2000) 99(3):223-30; JONES K et al. European Journal of Vascular & Endovascular Surgery (2002) 23(5):421-5; HARALAMBOUS E. et al. Crit. Care Med (2003) 31(12):2788-93; and ROEST M et al. Circulation (2000) 101(1):67-70). The 4G/4G (−/−) genotype of SERPINE1 was associated with SERPINE1 levels in patients suffering from acute lung injury (RUSSELL J A Crit. Care Med. (2003) 31(4):S243-S247).

Human serine (or cysteine) proteinase inhibitor, Glade A (alpha-1 antiproteinase, antitrypsin), member 5 (SERPINA5), protein C inhibitor, or plasminogen activator inhibitor-3 (PAI-3) maps to chromosome 14q32.1. Representative Homo sapiens SERPINA5 gene sequences are listed in GenBank under accession numbers AF361796.1 (GI:13448931) and NM_{—}000624.3 (GI:34147643).

Human interleukin 6 (IL6) or interferon beta 2 (IFNB2), BSF2, HGF or HSF maps to chromosome 7p21. Representative Homo sapiens IL6 gene sequences are listed in GenBank under accession numbers AF372214.2 (GI:14278708) and M54894.1 (GI:186351).

Human interleukin 10 (IL10) maps to chromosome 1q31-q32. Representative Homo sapiens IL10 gene sequences are listed in GenBank under accession numbers NM_{—}000572, M57627 and AF418271.

Human interleukin 12A (IL12A) maps to chromosome 3 p12-q13.2 and the cDNA extends over about 1.4 kb. Representative Homo sapiens IL12A gene sequences are listed in GenBank under accession numbers NM_{—}000882 and AF404773. The IL12A gene encodes a subunit of the IL12 cytokine. IL-12 is a heterodimer composed of the 35-10 subunit encoded by the IL12A gene, and a 40-kD subunit (IL-12B). Il-12 is required for the T-cell-independent induction of interferon (IFN)-gamma, and is important for the differentiation of both Th1 and Th2 cells. The responses of lymphocytes to IL-12 are mediated by the activator of transcription protein STAT4. Nitric oxide synthase 2A (NOS2A/NOS2) is found to be required for the signaling process of this cytokine in innate immunity.

Human tumor necrosis factor alpha receptor-1 (TNFRSF1A) maps to chromosome 12 p13.2 and the cDNA extends over about 2.2 kb. Representative Homo sapiens TNFRSF1A gene sequences are listed in GenBank under accession numbers NM_{—}001065 and AY131997. The TNFRSF1A gene is a member of the TNF-receptor superfamily and is one of the major receptors for the tumor necrosis factor-alpha. TNFRSF1A is known to activate NF-kappaB, mediate apoptosis, and regulate inflammation. Antiapoptotic protein BCL2-associated athanogene 4 (BAG4/SODD) and adaptor proteins TRADD and TRAF2 have been shown to interact with TNFRSF1A, and likely have roles in the signal transduction mediated by TNFRSF1A. Germline mutations of the extracellular domains of this receptor have been associated with autosomal dominant periodic fever syndrome, whereby the associated impaired receptor clearance is thought to be a mechanism of the disease.

Human vascular endothelial growth factor (VEGF) maps to chromosome 6 p12. Representative Homo sapiens VEGF gene sequences are listed in GenBank under accession numbers AF022375, AF437895, AL136131, NM_{—}001025366, NM_{—}003376, NM_{—}001025367, NM_{—}001025368, NM_{—}001025369, NM_{—}001025370 and NM_{—}001033756. The VEGF gene is a member of the PDGF/VEGF growth factor family and encodes a protein that is a glycosylated mitogen that specifically acts on endothelial cells and has various effects, including mediating increased vascular permeability, inducing angiogenesis, vasculogenesis and endothelial cell growth, promoting cell migration, and inhibiting apoptosis. Elevated levels of this protein have been associated with POEMS syndrome. VEGF gene mutations have been associated with proliferative and nonproliferative diabetic retinopathy.

Human protein C (PROC) maps to chromosome 2q13-q14 and extends over 11 kb. A representative Homo sapiens protein C gene sequence is listed in GenBank under accession number AF378903. Three single nucleotide polymorphisms (SNPs) have been identified in the 5′ untranslated promoter region of the protein C gene and are characterized as −1654 C/T, −1641 A/G and −1476 VT (according to the numbering scheme of FOSTER D C. et al. Proc Natl Acad Sci USA (1985) 82(14):4673-4677), or as −153C/T, −140A/G and +26A/T respectively by (MILLAR D S. et al. Hum. Genet. (2000) 106:646-653 at 651).

The genotype homozygous for −1654 C/−1641 G/−1476 T has been associated with reduced rates of transcription of the protein C gene as compared to the −1654 T/−1641 A/−1476 A homozygous genotype (SCOPES D. et al. Blood Coagul. Fibrinolysis (1995) 6(4):317-321). Patients homozygous for the −1654 C/−1641 G/−1476 T genotype show a decrease of 22% in plasma protein C levels and protein C activity levels as compared to patients homozygous for the −1654 T/−1641 A/−1476 A genotype (SPEK C A. et al. Arteriosclerosis, Thrombosis, and Vascular Biology (1995) 15:214-218). The −1654 C/−1641 G haplotype has been associated with lower protein C concentrations in both homozygotes and heterozygotes as compared to −1654 T/−1641 A (AIACH M. et al. Arterioscler Thromb Vasc Biol. (1999) 19(6):1573-1576).

Human endothelial protein C receptor (PROCR) is located on chromosome 20 and maps to chromosome 20q11.2. A representative human PROCR gene sequence with promoter is listed in GenBank under accession number AF106202 (8167 bp). A number of polymorphisms have been observed in the gene (BIGUZZI E. et al. Thromb Haemost (2002) 87:1085-6 and FRANCHI F. et al. Br J Haematol (2001) 114:641-6). Furthermore, polymorphisms of PROCR are also described in (BIGUZZI E. et al. Thromb Haemost (2001) 86:945-8; GALLIGAN L. et al. Thromb Haemost (2002) 88:163-5; ZECCHINA G. et al. Br J Haematol (2002) 119:881-2; FRENCH J K. et al. Am Heart J (2003) 145:118-24; and VON DEPKA M. et al. Thromb Haemost (2001) 86:1360-2; and SAPOSNIK B. et al. Blood (2004 Feb. 15) 103(4):1311-8.).

### SUMMARY OF THE INVENTION

This invention is based in part on the surprising discovery that protein C pathway associated SNPs selected from fibrinogen B beta polypeptide (FGB), coagulation factor II (F2), coagulation factor II receptor (F2R), coagulation factor 111 (F3), coagulation factor V (F5), coagulation factor VII (F7), coagulation factor X (F10), serine (or cysteine) proteinase inhibitor, Glade E type 1 (SERPINE1), protein C inhibitor (SERPINA5), interleukin 6 (IL6), interleukin 10 (IL10), interleukin 12A (IL12A), tumor necrosis factor alpha receptor-1 (TNFRSF1A), vascular endothelial growth factor (VEGF), protein C (PROC) and protein C receptor (PROCR) genes are predictive of subject response to treatment with activated protein C or protein C like compound.

This invention is also based in part on the surprising discovery that protein C pathway associated SNPs selected from fibrinogen B beta polypeptide (FGB), coagulation factor II (F2), coagulation factor II receptor (F2R), coagulation factor 111 (F3), coagulation factor V (F5), coagulation factor VII (F7), coagulation factor X (F10), serine (or cysteine) proteinase inhibitor, Glade E type 1 (SERPINE1), protein C inhibitor (SERPINA5), interleukin 6 (IL6), interleukin 10 (11.10), interleukin 12A (IL12A), tumor necrosis factor alpha receptor-1 (TNFRSF1A), vascular endothelial growth factor (VEGF), protein C (PROC) and protein C receptor (PROCR) alone or in combination are useful in predicting the response a subject with an inflammatory condition will have to treatment with activated protein C. Whereby the subjects having an improved response polymorphism are more likely to benefit from and have an improved response to activated protein C or protein C like compound treatment or treatment with a similar agent.

In accordance with one aspect of the invention, methods are provided for identifying a subject having an improved response polymorphism in a protein C pathway associated gene, the method including determining a genotype of the subject at one or more polymorphic sites in the subject's protein C pathway associated gene sequences or a combination thereof, wherein said genotype is indicative of the subject's response to activated protein C or protein C like compound administration. The method may further include comparing the genotype determined with known genotypes, which are known to be indicative of the subject's response, to activated protein C or protein C like compound administration. The method may further include obtaining protein C pathway associated gene sequence information for the subject. The method may further include obtaining the nucleic acid sample from the subject. The method may further include selecting a subject having one or more improved response polymorphism(s) in their protein C pathway associated gene sequences for administration of activated protein C or a protein C like compound. The method may further include excluding a subject not having one or more improved response polymorphism(s) in their protein C pathway associated gene sequences from administration of activated protein C or a protein C like compound.

In accordance with another aspect of the invention, there is provided a method of identifying a polymorphism in a protein C pathway associated gene sequence that correlates with an improved response to activated protein C or protein C like compound administration, the method including: obtaining protein C pathway associated gene sequence information from a group of subjects having an inflammatory condition; identifying at least one polymorphic nucleotide position in the protein C pathway associated gene sequence in the subjects; determining a genotypes at the polymorphic site for individual subjects in the group; determining response to activated protein C or protein C like compound administration; and correlating the genotypes determined in step (c) with the response to activated protein C or protein C like compound administration in step (d) thereby identifying said protein C pathway associated gene sequence polymorphisms that correlate with response to activated protein C or protein C like compound administration.

In accordance with another aspect of the invention, there is provided a kit for determining a genotype at a defined nucleotide position within a polymorphic site in a protein C pathway associated gene sequence in a subject to predict a subject's response to activated protein C or protein C like compound administration, the kit including: a restriction enzyme capable of distinguishing alternate nucleotides at the polymorphic site; or a labeled oligonucleotide having sufficient complementary to the polymorphic site so as to be capable of hybridizing distinctively to said alternate. The kit may further include an oligonucleotide or a set of oligonucleotides operable to amplify a region including the polymorphic site. The kit may further include a polymerization agent. The kit may further include instructions for using the kit to determine genotype.

In accordance with another aspect of the invention, there is provided a method for selecting a group of subjects for determining the efficacy of a candidate drug known or suspected of being useful for the treatment of an inflammatory condition, the method including determining a genotype at one or more polymorphic sites in a protein C pathway associated gene sequence for each subject, wherein said genotype is indicative of the subject's response to the candidate drug and sorting subjects based on their genotype. The method may further include, administering the candidate drug to the subjects or a subset of subjects and determining each subject's ability to recover from the inflammatory condition. The method may further include comparing subject response to the candidate drug based on genotype of the subject.

In accordance with another aspect of the invention, there is provided a method of treating an inflammatory condition in a subject in need thereof, the method including administering to the subject activated protein C or protein C like compound, wherein said subject has an improved response polymorphism in their protein C pathway associated gene sequence.

In accordance with another aspect of the invention, there is provided a method of treating an inflammatory condition in a subject in need thereof, the method including: selecting a subject having an improved response polymorphism in their protein C pathway associated gene sequence; and administering to said subject activated protein C or protein C like compound.

In accordance with another aspect of the invention, there is provided a method of treating a subject with an inflammatory condition by administering activated protein C, the method including administering the activated protein C or protein C like compound to subjects that have an improved response polymorphism in their protein C pathway associated gene sequence, wherein the improved response polymorphism is predictive of increased responsiveness to the treatment of the inflammatory condition with activated protein C or protein C like compound.

In accordance with another aspect of the invention, there is provided a method of identifying a subject with increased responsiveness to treatment of an inflammatory condition with activated protein C or protein C like compound, including the step of screening a population of subjects to identify those subjects that have an improved response polymorphism in their protein C pathway associated gene sequence, wherein the identification of a subject with an improved response polymorphism in their protein C pathway associated gene sequence is predictive of increased responsiveness to the treatment of the inflammatory condition with the activated protein C or protein C like compound.

In accordance with another aspect of the invention, there is provided a method of selecting a subject for the treatment of an inflammatory condition with an activated protein C or protein C like compound, including the step of identifying a subject having an improved response polymorphism in their protein C pathway associated gene sequence, wherein the identification of a subject with the improved response polymorphism is predictive of increased responsiveness to the treatment of the inflammatory condition with the activated protein C or protein C like compound.

In accordance with another aspect of the invention, there is provided a method of treating an inflammatory condition in a subject, the method including administering an activated protein C or protein C like compound to the subject, wherein said subject has an improved response polymorphism in their protein C pathway associated gene sequence.

In accordance with another aspect of the invention, there is provided a method of treating an inflammatory condition in a subject, the method including: identifying a subject having an improved response polymorphism in their protein C pathway associated gene sequence; and administering activated protein C or protein C like compound to the subject.

In accordance with another aspect of the invention, there is provided a use of an activated protein C or protein C like compound in the manufacture of a medicament for the treatment of an inflammatory condition, wherein the subjects treated have an improved response polymorphism in their protein C pathway associated gene sequence.

In accordance with another aspect of the invention, there is provided a use of an activated protein C or protein C like compound in the manufacture of a medicament for the treatment of an inflammatory condition in a subset of subjects, wherein the subset of subjects have an improved response polymorphism in their protein C pathway associated gene sequence.

In accordance with another aspect of the invention, there is provided a commercial package containing, as active pharmaceutical ingredient, use of an activated protein C or protein C like compound, or a pharmaceutically acceptable salt thereof, together with instructions for its use for the curative or prophylactic treatment of an inflammatory condition in a subject, wherein the subject treated has an improved response polymorphism in their protein C pathway associated gene sequence.

In accordance with another aspect of the invention, there are provided two or more oligonucleotides or peptide nucleic acids of about 10 to about 400 nucleotides that hybridize specifically to a sequence contained in a human target sequence consisting of a subject's protein C pathway associated gene sequence, a complementary sequence of the target sequence or RNA equivalent of the target sequence and wherein the oligonucleotides or peptide nucleic acids are operable in determining the presence or absence of two or more improved response polymorphism(s) in their protein C pathway associated gene sequence selected from of the following polymorphic sites: rs1800791; rs3136516; rs253073; rs2227750; rs1361600; rs9332575; rs4656687; rs9332630; rs9332546; rs2774030; rs2026160; rs3211719; rs3093261; rs1799889; rs1050813; rs2069972; rs2069840; rs1800795; rs1800872; rs2243154; rs4149577; rs1413711; rs2069895; rs2069898; rs2069904; rs1799808; rs2069910; rs2069915; rs2069916; rs2069918; rs2069919; rs2069920; rs2069924; rs5937; rs2069931; rs777556; rs1033797; rs1033799; rs2295888; and rs867186 or one or more polymorphic sites in linkage disequilibrium thereto.

In accordance with another aspect of the invention, oligonucleotides or peptide nucleic acids are provided that may be used in the identification of protein C pathway associated gene sequence polymorphisms in accordance with the methods described herein, the oligonucleotides or peptide nucleic acids are characterized in that the oligonucleotides or peptide nucleic acids hybridize under normal hybridization conditions with a region of one of sequences identified by SEQ ID NO:1-243 or their complements to determine the presence or absence of one or more protein C pathway associated gene sequence polymorphisms within a target sequence.

In accordance with another aspect of the invention, an oligonucleotide primer is provided including a portion of SEQ ID NO:1-243 or their complements, wherein said primer is 12 to 54 nucleotides in length and wherein the primer specifically hybridizes to a region of SEQ ID NO:1-243 or their complements and is capable of identifying protein C pathway associated gene sequence polymorphisms described herein. Alternatively, the primers may be between sixteen to twenty-four nucleotides in length.

In accordance with another aspect of the invention, oligonucleotide or peptide nucleic acids are provided of about 10 to about 400 nucleotides that hybridize specifically to a sequence contained in a human target sequence including SEQ ID NO:1-243, a complementary sequence of the target sequence or RNA equivalent of the target sequence and wherein the oligonucleotide or peptide nucleic acid is operable in determining the allele or genotype at a polymorphism at one or more of positions of the protein C pathway associated gene sequence polymorphisms as described herein.

In accordance with another aspect of the invention, two or more oligonucleotides or peptide nucleic acids are provided selected from: an oligonucleotide or peptide nucleic acid capable of hybridizing under high stringency conditions to an oligonucleotide or peptide nucleic acid molecule including a first allele for a given polymorphism selected from the polymorphisms listed in TABLE 1C but not capable of hybridizing under high stringency conditions to an oligonucleotide or peptide nucleic acid molecule comprising a second allele for the given polymorphism selected from the polymorphisms listed in TABLE 1C; and an oligonucleotide or peptide nucleic acid capable of hybridizing under high stringency conditions to an oligonucleotide or peptide nucleic acid molecule comprising the second allele for a given polymorphism selected from the polymorphisms listed in TABLE 1C but not capable of hybridizing under high stringency conditions to an oligonucleotide or peptide nucleic acid molecule comprising the first allele for the given polymorphism selected from the polymorphisms listed in TABLE 1C.

In accordance with another aspect of the invention, two or more oligonucleotides or peptide nucleic acids are provided selected from: an oligonucleotide or peptide nucleic acid capable of hybridizing under high stringency conditions to an oligonucleotide or peptide nucleic acid molecule including a first allele for a given polymorphism selected from the polymorphisms listed in TABLE 1D but not capable of hybridizing under high stringency conditions to an oligonucleotide or peptide nucleic acid molecule comprising a second allele for the given polymorphism selected from the polymorphisms listed in TABLE 1D; and an oligonucleotide or peptide nucleic acid capable of hybridizing under high stringency conditions to an oligonucleotide or peptide nucleic acid molecule comprising the second allele for a given polymorphism selected from the polymorphisms listed in TABLE 1D but not capable of hybridizing under high stringency conditions to an oligonucleotide or peptide nucleic acid molecule comprising the first allele for the given polymorphism selected from the polymorphisms listed in TABLE 1D.

In accordance with another aspect of the invention, there is provided an array of oligonucleotides or peptide nucleic acids attached to a solid support, the array including two or more of the oligonucleotides or peptide nucleic acids set out herein.

In accordance with another aspect of the invention, there is provided a composition including an addressable collection of two or more oligonucleotides or peptide nucleic acids, the two or more oligonucleotides or peptide nucleic acids selected from the oligonucleotides or peptide nucleic acids set out herein.

In accordance with another aspect of the invention, there is provided a composition comprising an addressable collection of two or more oligonucleotides or peptide nucleic acids, the two or more oligonucleotides or peptide nucleic acids consisting essentially of two or more nucleic acid molecules set out in SEQ ID NO:1-243 or compliments, fragments, variants, or analogs thereof.

In accordance with another aspect of the invention, there is provided a composition comprising an addressable collection of two or more oligonucleotides or peptide nucleic acids, the two or more oligonucleotides or peptide nucleic acids consisting essentially of two or more nucleic acid molecules set out in TABLES 1C and 1D or compliments, fragments, variants, or analogs thereof.

In accordance with another aspect of the invention, there is provided a computer readable medium comprising a plurality of encoded genotype correlations selected from the protein C pathway associated gene SNP correlations in TABLE 1E, wherein each correlation of the plurality has a value representing an indication of responsiveness to treatment with activated protein C. The encoded genotype correlations may be digitally encoded.

The genotype may be determined using a nucleic acid sample from the subject. Genotype may be determined using one or more of the following techniques: restriction fragment length analysis; sequencing; micro-sequencing assay; hybridization; invader assay; gene chip hybridization assays; oligonucleotide ligation assay; ligation rolling circle amplification; 5′ nuclease assay; polymerase proofreading methods; allele specific PCR; matrix assisted laser desorption ionization time of flight (MALDI-TOF) mass spectroscopy; ligase chain reaction assay; enzyme-amplified electronic transduction; single base pair extension assay; and reading sequence data.

The polymorphic site may be selected from one or more of the following: rs1800791; rs3136516; rs253073; rs2227750; rs1361600; rs9332575; rs4656687; rs9332630; rs9332546; rs2774030; rs2026160; rs3211719; rs3093261; rs1799889; rs1050813; rs2069972; rs2069840; rs1800795; rs1800872; rs2243154; rs4149577; rs1413711; rs2069895; rs2069898; rs2069904; rs1799808; rs2069910; rs2069915; rs2069916; rs2069918; rs2069919; rs2069920; rs2069924; rs5937; rs2069931; rs777556; rs1033797; rs1033799; rs2295888; and rs867186; or one or more polymorphic sites in linkage disequilibrium thereto. The improved response polymorphism may be selected from one or more of the following: rs1800791A; rs3136516G; rs3136516GG; rs253073G; rs253073GG; rs2227750GG; rs1361600GG; rs9332575G; rs4656687T; rs9332630A; rs9332546A; rs2774030AG; rs2026160C; rs3211719G; rs3093261T; rs1799889G; rs1050813A; rs1050813AG; rs2069972TT; rs2069840C; rs1800795G; rs1800872A; rs2243154A; rs2243154AG; rs4149577CT; rs1413711AA; rs2069895AG; rs2069898CT; rs2069904AG; rs1799808CT; rs2069910C; rs2069910CT; rs2069915AG; rs2069916CT; rs2069918A; rs2069918AA; rs2069919AG; rs2069920CT; rs2069924CT; rs5937CT; rs2069931 CT; rs777556C; rs1033797C; rs1033799A; rs2295888G; rs867186AG; and rs867186G; or one or more polymorphic sites in linkage disequilibrium thereto. The one or more polymorphic sites in linkage disequilibrium thereto may be selected from one or more of the polymorphic sites listed in TABLE 1B.

The genotype of the subject may be indicative of the subject's response to activated protein C or protein C like compound administration. The subject may be critically ill with an inflammatory condition. The inflammatory condition may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects, subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis. The inflammatory condition may be SIRS or sepsis.

The activated protein C or protein C like compound may be drotecogin alfa activated. The activated protein C or protein C like compound may have one or more of the following activities: serine protease activity; anticoagulant; anti-inflammatory; pro-fibrinolytic; and anti-apoptotic activities.

The method or use may further include determining the subject's APACHE II score as an assessment of subject risk. Subject risk may be used as a further indicator that activated protein C or protein C like compound administration is appropriate. The method or use may further include determining the number of organ system failures for the subject as an assessment of subject risk. The subject's APACHE II score may be indicative of an increased risk when ≧25. Similarly, 2 or more organ system failures may be indicative of increased subject risk.

The oligonucleotides or peptide nucleic acids may further include one or more of the following: a detectable label; a quencher; a mobility modifier; a contiguous non-target sequence situated 5′ or 3′ to the target sequence or 5′ and 3′ to the target sequence. The oligonucleotides or peptide nucleic acids may alternatively be of about 10 to about 400 nucleotides, about 15 to about 300 nucleotides. The oligonucleotides or peptide nucleic acids may alternatively be of about 20 to about 200 nucleotides, about 25 to about 100 nucleotides. The oligonucleotides or peptide nucleic acids may alternatively be of about 20 to about 80 nucleotides, about 25 to about 50 nucleotides.

### DETAILED DESCRIPTION OF THE INVENTION

### 1. Definitions

In the description that follows, a number of terms are used extensively, the following definitions are provided to facilitate understanding of the invention.

“Activated protein C” or “protein C like compound” as used herein includes any protein C molecule, protein C derivative, protein C variant, protein C analog and any prodrug thereof, metabolite thereof, isomer thereof, combination of isomers thereof, or pharmaceutical composition of any of the preceding. Activated protein C or protein C like compound or protein C like compounds may be synthesized or purified. For example, Drotrecogin alfa (activated) is sold as XIGRIS™ by Eli Lilly and Company and has the same amino acid sequence as human plasma-derived Activated Protein C. Examples of derivatives, variants, analogs, or compositions etc. may be found in US patent applications: 20050176083; 20050143283; 20050095668; 20050059132; 20040028670; 20030207435; 20030027299; 20030022354; and 20030018175 and issued U.S. Pat. Nos. 6,933,367; 6,841,371; 6,815,533; 6,630,138; 6,630,137; 6,436,397; 6,395,270; 6,162,629; 6,159,468; 5,837,843; 5,453,373; 5,330,907; 5,766,921; 5,753,224; 5,516,650; and 5,358,932.

“Genetic material” includes any nucleic acid and can be a deoxyribonucleotide or ribonucleotide polymer in either single or double-stranded form.

A “purine” is a heterocyclic organic compound containing fused pyrimidine and imidazole rings, and acts as the parent compound for purine bases, adenine (A) and guanine (G).

“Nucleotides” are generally a purine (R) or pyrimidine (Y) base covalently linked to a pentose, usually ribose or deoxyribose, where the sugar carries one or more phosphate groups.

Nucleic acids are generally a polymer of nucleotides joined by 3′-5′ phosphodiester linkages. As used herein “purine” is used to refer to the purine bases, A and G, and more broadly to include the nucleotide monomers, deoxyadenosine-5′-phosphate and deoxyguanosine-5′-phosphate, as components of a polynucleotide chain.

A “pyrimidine” is a single-ringed, organic base that forms nucleotide bases, cytosine (C), thymine (T) and uracil (U). As used herein “pyrimidine” is used to refer to the pyrimidine bases, C, T and U, and more broadly to include the pyrimidine nucleotide monomers that along with purine nucleotides are the components of a polynucleotide chain.

A nucleotide represented by the symbol M may be either an A or C, a nucleotide represented by the symbol W may be either an T/U or A, a nucleotide represented by the symbol Y may be either an C or T/U, a nucleotide represented by the symbol S may be either an G or C, while a nucleotide represented by the symbol R may be either an G or A, and a nucleotide represented by the symbol K may be either an G or T/U. Similarly, a nucleotide represented by the symbol V may be either A or G or C, while a nucleotide represented by the symbol D may be either A or G or T, while a nucleotide represented by the symbol B may be either G or C or T, and a nucleotide represented by the symbol H may be either A or C or T. Furthermore, a deletion or an insertion may be represented by either a “−” or “del” and “+” or “ins” or “I” respectively. Alternatively, polymorphisms may be represented as follows A/- (SEQ ID NO:75), -/A/AT/G (SEQ ID NO:104), -/AAC (SEQ ID NO:113), -/T (SEQ ID NO:119), -/A/CG/G (SEQ ID NO:130), -/A/C (SEQ ID NO:132, A/- (SEQ ID NO:140), -/A (SEQ ID NO:145), -/AGG (SEQ ID NO:147), -/TTTA (SEQ ID NO:148), -/G/GGA (SEQ ID NO:154), -/GTTT (SEQ ID NO:159), -/CAAA (SEQ ID NO:175, -/CT (SEQ ID NO:192), -/T (SEQ ID NO:221), and -/A/G (SEQ ID NO:14), wherein the allele options at a polymorphic site are separated by a forward slash (“/”). For example, “-/AGG” may be either a deletion or AGG.

A “polymorphic site” or “polymorphism site” or “polymorphism” or “single nucleotide polymorphism site” (SNP site) or single nucleotide polymorphism” (SNP) as used herein is the locus or position with in a given sequence at which divergence occurs. A “Polymorphism” is the occurrence of two or more forms of a gene or position within a gene (allele), in a population, in such frequencies that the presence of the rarest of the forms cannot be explained by mutation alone. The implication is that polymorphic alleles confer some selective advantage on the host. Preferred polymorphic sites have at least two alleles, each occurring at frequency of greater than 1%, and more preferably greater than 10% or 20% of a selected population. Polymorphic sites may be at known positions within a nucleic acid sequence or may be determined to exist using the methods described herein. Polymorphisms may occur in both the coding regions and the noncoding regions (for example, promoters, enhancers and introns) of genes. Polymorphisms may occur at a single nucleotide site (SNPs) or may involve an insertion or deletion as described herein.

An “improved response polymorphism” as used herein refers to an allelic variant or genotype at one or more polymorphic sites within the protein C pathway associated polymorphisms selected from fibrinogen B beta polypeptide (FGB), coagulation factor II (F2), coagulation factor II receptor (F2R), coagulation factor III (F3), coagulation factor V (F5), coagulation factor VII (F7), coagulation factor X (F10), serine (or cysteine) proteinase inhibitor, Glade E type I (SERPINE1), protein C inhibitor (SERPINA5), interleukin 6 (IL6), interleukin 10 (IL10), interleukin 12A (IL12A), tumor necrosis factor alpha receptor-1 (TNFRSF1A), vascular endothelial growth factor (VEGF), protein C (PROC) and protein C receptor (PROCR) as described herein as being predictive of a subject's response to activated protein C or protein C like compound or protein C like compound treatment (for example rs1800791A, rs3136516G, rs3136516GG, rs253073G, rs253073GG, rs2227750GG, rs1361600GG, rs9332575G, rs4656687T, rs9332630A, rs9332546A, rs2774030AG, rs2026160C, rs3211719G, rs3093261T, rs1799889G, rs1050813A, rs1050813AG, rs2069972TT, rs2069840C, rs1800795G, rs1800872A, rs2243154A, rs2243154AG, rs4149577CT, rs1413711AA, rs2069895AG; rs2069898CT; rs2069904AG; rs1799808CT; rs2069910C; rs2069910CT; rs2069915AG; rs2069916CT; rs2069918A; rs2069918AA; rs2069919AG; rs2069920CT; rs2069924CT; rs5937CT; rs2069931CT; rs777556C; rs1033797C; rs1033799A; rs2295888G; rs867186AG; and rs867186G).

As used herein “haplotype” is a set of alleles of closely linked loci on a chromosome that tend to be inherited together. Such allele sets occur in patterns, which are called haplotypes. Accordingly, a specific SNP or other polymorphism allele at one SNP site is often associated with a specific SNP or other polymorphism allele at a nearby second SNP site or other polymorphism site. When this occurs, the two SNPs or other polymorphisms are said to be in linkage disequilibrium because the two SNPs or other polymorphisms are not just randomly associated (in linkage equilibrium).

In general, the detection of nucleic acids in a sample depends on the technique of specific nucleic acid hybridization in which the oligonucleotide is annealed under conditions of “high stringency” to nucleic acids in the sample, and the successfully annealed oligonucleotides are subsequently detected (see for example Spiegelman, S., Scientific American, Vol. 210, p. 48 (1964)). Hybridization under high stringency conditions primarily depends on the method used for hybridization, the oligonucleotide length, base composition and position of mismatches (if any). High stringency hybridization is relied upon for the success of numerous techniques routinely performed by molecular biologists, such as high stringency PCR, DNA sequencing, single strand conformational polymorphism analysis, and in situ hybridization. In contrast to Northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998.

“Oligonucleotides” as used herein are variable length nucleic acids, which may be useful as probes, primers and in the manufacture of microarrays (arrays) for the detection and/or amplification of specific nucleic acids. Such DNA or RNA strands may be synthesized by the sequential addition (5′-3′ or 3′-5′) of activated monomers to a growing chain, which may be linked to an insoluble support. Numerous methods are known in the art for synthesizing oligonucleotides for subsequent individual use or as a part of the insoluble support, for example in arrays (BERNFIELD M R. and ROTTMAN F M. J. Biol. Chem. (1967) 242(18):4134-43; SULSTON J. et al. PNAS (1968) 60(2):409-415; GILLAM S. et al. Nucleic Acid Res. (1975) 2(5):613-624; BONORA G M. et al. Nucleic Acid Res. (1990) 18(11):3155-9; LASHKARI D A. et al. PNAS (1995) 92(17):7912-5; MCGALL G. et al. PNAS (1996) 93(24):13555-60; ALBERT T J. et al. Nucleic Acid Res. (2003) 31(7):e35; GAO X. et al. Biopolymers (2004) 73(5):579-96; and MOORCROFT M J. et al. Nucleic Acid Res. (2005) 33(8):e75). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

“Peptide nucleic acids” (PNA) as used herein refer to modified nucleic acids in which the sugar phosphate skeleton of a nucleic acid has been converted to an N-(2-aminoethyl)-glycine skeleton. Although the sugar-phosphate skeletons of DNA/RNA are subjected to a negative charge under neutral conditions resulting in electrostatic repulsion between complementary chains, the backbone structure of PNA does not inherently have a charge. Therefore, there is no electrostatic repulsion. Consequently, PNA has a higher ability to form double strands as compared with conventional nucleic acids, and has a high ability to recognize base sequences. Furthermore, PNAs are generally more robust than nucleic acids. PNAs may also be used in arrays and in other hybridization or other reactions as described above and herein for oligonucleotides.

An “addressable collection” as used herein is a combination of nucleic acid molecules or peptide nucleic acids capable of being detected by, for example, the use of hybridization techniques or by any other means of detection known to those of ordinary skill in the art. An DNA microarray would be considered an example of an “addressable collection”.

In general the term “linkage”, as used in population genetics, refers to the co-inheritance of two or more nonallelic genes or sequences due to the close proximity of the loci on the same chromosome, whereby after meiosis they remain associated more often than the 50% expected for unlinked genes. However, during meiosis, a physical crossing between individual chromatids may result in recombination. “Recombination” generally occurs between large segments of DNA, whereby contiguous stretches of DNA and genes are likely to be moved together in the recombination event (crossover). Conversely, regions of the DNA that are far apart on a given chromosome are more likely to become separated during the process of crossing-over than regions of the DNA that are close together. Polymorphic molecular markers, like single nucleotide polymorphisms (SNPs), are often useful in tracking meiotic recombination events as positional markers on chromosomes.

The pattern of a set of markers along a chromosome is referred to as a “Haplotype”. Accordingly, groups of alleles on the same small chromosomal segment tend to be transmitted together. Haplotypes along a given segment of a chromosome are generally transmitted to progeny together unless there has been a recombination event. Absent a recombination event, haplotypes can be treated as alleles at a single highly polymorphic locus for mapping.

Furthermore, the preferential occurrence of a disease gene in association with specific alleles of linked markers, such as SNPs or other polymorphisms, is called “Linkage Disequilibrium” (LD). This sort of disequilibrium generally implies that most of the disease chromosomes carry the same mutation and the markers being tested are relatively close to the disease gene(s).

For example, in SNP-based association analysis and linkage disequilibrium mapping, SNPs can be useful in association studies for identifying polymorphisms, associated with a pathological condition, such as sepsis. Unlike linkage studies, association studies may be conducted within the general population and are not limited to studies performed on related individuals in affected families. In a SNP association study the frequency of a given allele (i.e. SNP allele) is determined in numerous subjects having the condition of interest and in an appropriate control group. Significant associations between particular SNPs or SNP haplotypes and phenotypic characteristics may then be determined by numerous statistical methods known in the art.

Association analysis can either be direct or LD based. In direct association analysis, potentially causative SNPs may be tested as candidates for the pathogenic sequence. In LD based SNP association analysis, SNPs may be chosen at random over a large genomic region or even genome wide, to be tested for SNPs in LD with a pathogenic sequence or pathogenic SNP. Alternatively, candidate sequences associated with a condition of interest may be targeted for SNP identification and association analysis. Such candidate sequences usually are implicated in the pathogenesis of the condition of interest. In identifying SNPs associated with inflammatory conditions, candidate sequences may be selected from those already implicated in the pathway of the condition or disease of interest. Once identified, SNPs found in or associated with such sequences, may then be tested for statistical association with an individual's prognosis or susceptibility to the condition.

For an LD based association analysis, high density SNP maps are useful in positioning random SNPs relative to an unknown pathogenic locus. Furthermore, SNPs tend to occur with great frequency and are often spaced uniformly throughout the genome. Accordingly, SNPs as compared with other types of polymorphisms are more likely to be found in close proximity to a genetic locus of interest. SNPs are also mutationally more stable than variable number tandem repeats (VNTRs).

In population genetics linkage disequilibrium refers to the “preferential association of a particular allele, for example, a mutant allele for a disease with a specific allele at a nearby locus more frequently than expected by chance” and implies that alleles at separate loci are inherited as a single unit (Gelehrter, T. D., Collins, F. S. (1990). Principles of Medical Genetics. Baltimore: Williams & Wilkens). Accordingly, the alleles at these loci and the haplotypes constructed from their various combinations serve as useful markers of phenotypic variation due to their ability to mark clinically relevant variability at a particular position, such as position 86 of SEQ ID NO:1 (see Akey, J. et al. (2001). Haplotypes vs single marker linkage disequilibrium tests: what do we gain? European Journal of Human Genetics. 9:291-300; and Zhang, K. et al. (2002). Haplotype block structure and its applications to association studies: power and study designs. American Journal of Human Genetics. 71:1386-1394). This viewpoint is further substantiated by Khoury et al. ((1993). Fundamentals of Genetic Epidemiology. New York: Oxford University Press at p. 160) who state, “[w]henever the marker allele is closely linked to the true susceptibility allele and is in [linkage] disequilibrium with it, one can consider that the marker allele can serve as a proxy for the underlying susceptibility allele.”

As used herein “linkage disequilibrium” (LD) is the occurrence in a population of certain combinations of linked alleles in greater proportion than expected from the allele frequencies at the loci. For example, the preferential occurrence of a disease gene in association with specific alleles of linked markers, such as SNPs, or between specific alleles of linked markers, are considered to be in LD. This sort of disequilibrium generally implies that most of the disease chromosomes carry the same mutation and that the markers being tested are relatively close to the disease gene(s). Accordingly, if the genotype of a first locus is in LD with a second locus (or third locus etc.), the determination of the allele at only one locus would necessarily provide the identity of the allele at the other locus. When evaluating loci for LD those sites within a given population having a high degree of linkage disequilibrium (i.e. an absolute value for D′ of ≧0.5 or r²≧0.5) are potentially useful in predicting the identity of an allele of interest (i.e. associated with the condition of interest). A high degree of linkage disequilibrium may be represented by an absolute value for D′ of ≧0.6 or r²≧0.6. Alternatively, a high degree of linkage disequilibrium may be represented by an absolute value for D′ of ≧0.7 or r²≧0.7 or by an absolute value for D′ of ≧0.8 or r²≧0.8. Additionally, a high degree of linkage disequilibrium may be represented by an absolute value for D′ of ≧0.85 or r²≧0.85 or by an absolute value for D′ of ≧0.9 or r²≧0.9. Accordingly, two SNPs that have a high degree of LD may be equally useful in determining the identity of the allele of interest or disease allele. Therefore, we may assume that knowing the identity of the allele at one SNP may be representative of the allele identity at another SNP in LD. Accordingly, the determination of the genotype of a single locus can provide the identity of the genotype of any locus in LD therewith and the higher the degree of linkage disequilibrium the more likely that two SNPs may be used interchangeably. For example, in the population from which the tagged SNPs were identified from the SNP identified by rs2069972 is in “linkage disequilibrium” with the SNP identified by rs2069973, whereby when the genotype of rs2069972 is T the genotype of rs2069973 is G. Similarly, when the genotype of rs2069972 is C the genotype of rs2069973 is C. Accordingly, the determination of the genotype at rs2069972 will provide the identity of the genotype at rs2069973 or any other locus in “linkage disequilibrium” therewith. Particularly, where such a locus has a high degree of linkage disequilibrium thereto.

Linkage disequilibrium is useful for genotype-phenotype association studies. For example, if a specific allele at one SNP site (e.g. “A”) is the cause of a specific clinical outcome (e.g. call this clinical outcome “B”) in a genetic association study then, by mathematical inference, any SNP (e.g. “C”) which is in significant linkage disequilibrium with the first SNP, will show some degree of association with the clinical outcome. That is, if A is associated (˜) with B, i.e. A˜B and C˜A then it follows that C˜B. Of course, the SNP that will be most closely associated with the specific clinical outcome, B, is the causal SNP—the genetic variation that is mechanistically responsible for the clinical outcome. Thus, the degree of association between any SNP, C, and clinical outcome will depend on linkage disequilibrium between A and C.

Until the mechanism underlying the genetic contribution to a specific clinical outcome is fully understood, linkage disequilibrium helps identify potential candidate causal SNPs and also helps identify a range of SNPs that may be clinically useful for prognosis of clinical outcome or of treatment effect. If one SNP within a gene is found to be associated with a specific clinical outcome, then other SNPs in linkage disequilibrium will also have some degree of association and therefore some degree of prognostic usefulness. By way of prophetic example, if multiple polymorphisms were tested for individual association with an improved response to activated protein C or protein C like compound or protein C like compound administration in our SIRS/sepsis cohort of ICU patients, wherein the multiple polymorphisms had a range of linkage disequilibrium with SERPINA5 polymorphism rs2069972 and it was assumed that rs2069972 was the causal polymorphism, and we were to order the polymorphisms by the degree of linkage disequilibrium with rs2069972, we would expect to find that polymorphisms with high degrees of linkage disequilibrium with rs2069972 would also have a high degree of association with this specific clinical outcome. As linkage disequilibrium decreased, we would expect the degree of association of the polymorphism with an improved response to activated protein C or protein C like compound or protein C like compound administration to also decrease. Accordingly, logic dictates that if A˜B and C˜A, then C˜B. That is, any polymorphism, whether already discovered or as yet undiscovered, that is in linkage disequilibrium with one of the improved response polymorphisms described herein will likely be a predictor of the same clinical outcomes that rs2069972 is a predictor of. The similarity in prediction between this known or unknown polymorphism and rs2069972 would depend on the degree of linkage disequilibrium between such a polymorphism and rs2069972.

Numerous sites have been identified as polymorphic sites in the protein C pathway associated genes (see TABLE 1A). Furthermore, the polymorphisms in TABLE 1A are linked to (in linkage disequilibrium with) numerous polymorphisms as set out in TABLE 1B below and may also therefore be indicative of subject prognosis.

TABLE 1A
Polymorphisms in the protein C pathway associated genes (coagulation, fibrinolysis
and inflammation pathways) genotyped in a cohort of critically ill patients who had severe sepsis
and no XIGRIS ™ contraindications. Minor allele frequency is given for the entire patient cohort
(XIGRIS ™-treated patients and matched controls).

| | | | | Minor allele | |
|---|---|---|---|---|---|
| Polymorphism Name | | | May 2004 | frequency in | |
| (HUGO name. chromosomal | | | Chromosomal | the patient | SEATTLE |
| position. major allele/minor allele | | | position | population | SNPS |
| according to public databases) | Official Gene Name | rs# | (Build 35) | (minor allele) | IDENTIFYER |
| FGB.155840914.G/A | fibrinogen, B beta | 1800791 | 155840914 | 0.15 (A) | 1038 |
| | polypeptide | | | | |
| F2.46717332.G/A | coagulation factor II | 3136516 | 46717332 | 0.47 (A) | 21239 |
| | (thrombin) | | | | |
| F2R.76059983.A/G | coagulation factor II | 253073± | 76059983 | 0.41 (G) | 14244 |
| | (thrombin) receptor | | | | |
| F2R.76049220.G/C | coagulation factor II | 2227750 | 76049220 | 0.22 (C) | 3481 |
| | (thrombin) receptor | | | | |
| F3.94719939.A/G | coagulation factor III | 1361600 | 94719939 | 0.44 (G) | 1826 |
| | (thromboplastin, tissue | | | | |
| | factor) | | | | |
| F5.166258759.A/G | coagulation factor V | 9332575 | 166258759 | 0.11 (G) | 30539 |
| | (proaccelerin, labile factor) | | | | |
| F5.166236816.T/C* | coagulation factor V | 4656687 | 166236816 | 0.41 (C) | 52485 |
| | (proaccelerin, labile factor) | | | | |
| F5.166227911.A/G | coagulation factor V | 9332630 | 166227911 | 0.46 (A) | 61390 |
| | (proaccelerin, labile factor) | | | | |
| F5.166269905.G/A | coagulation factor V | 9332546 | 166269905 | 0.32 (A) | 19390 |
| | (proaccelerin, labile factor) | | | | |
| F7.112808416.A/G | coagulation factor VII | 2774030 | 112808416 | 0.40 (G) | 2643 |
| | (serum prothrombin | | | | |
| | conversion accelerator) | | | | |
| F10.112840894.A/C | coagulation factor X | 2026160 | 112840894 | 0.26 (C) | 17396 |
| F10.112825510.A/G | coagulation factor X | 3211719 | 112825510 | 0.24 (G) | 2011 |
| F10.112824083.T/C | coagulation factor X | 3093261 | 112824083 | 0.35 (T) | 577 |
| SERPINE1.100363146.4G/5G | serine (or cysteine) | 1799889 | 100363146 | 0.49 (5G) | 837 |
| (or —/G) | proteinase inhibitor, clade | | | | |
| | E (nexin, plasminogen | | | | |
| | activator inhibitor type 1), | | | | |
| | member 1 | | | | |
| SERPINE1.100375050.G/A | serine (or cysteine) | 1050813 | 100375050 | 0.18 (A) | 12750 |
| | proteinase inhibitor, clade | | | | |
| | E (nexin, plasminogen | | | | |
| | activator inhibitor type 1), | | | | |
| | member 1 | | | | |
| SERPINA5.94123294.C/T | serine (or cysteine) | 2069972 | 94123294 | 0.47 (T) | 1328 |
| | proteinase inhibitor, clade | | | | |
| | A (alpha-1 antiproteinase, | | | | |
| | antitrypsin), member 5 | | | | |
| IL6.22541812.C/G | interleukin 6 (interferon, | 2069840 | 22541812 | 0.28 (G) | 3437 |
| | beta 2) | | | | |
| IL6.22539885.G/C | interleukin 6 (interferon, | 1800795 | 22539885 | 0.29 (C) | 1510 |
| | beta 2) | | | | |
| IL10.203334802.C/A | interleukin 10 | 1800872 | 203334802 | 0.30 (A) | 472 |
| IL12A.161198944.G/A | interleukin 12A (natural | 2243154 | 161198944 | 0.08 (A) | 11494 |
| | killer cell stimulatory factor | | | | |
| | 1, cytotoxic lymphocyte | | | | |
| | maturation factor 2, p40) | | | | |
| TNFRSF1A.6317783.T/C | tumor necrosis factor | 4149577 | 6317783 | 0.48 (T) | 5664 |
| | receptor superfamily, | | | | |
| | member 1A | | | | |
| VEGF.43848656.G/A* | vascular endothelial growth | 1413711 | 43848656 | 0.45 (A) | 674 |
| | factor | | | | |
| PROC.127890298.A/G | protein C (inactivator of | 2069895 | 127890298 | 0.3 (G) | 611 |
| | coagulation factors Va and | | | | |
| | VIIIa) | | | | |
| PROC.127890457.T/C | protein C (inactivator of | 2069898 | 127890457 | 0.3 (C) | 770 |
| | coagulation factors Va and | | | | |
| | VIIIa) | | | | |
| PROC.127892009.G/A | protein C (inactivator of | 2069904 | 127892009 | 0.3 (A) | 2322 |
| | coagulation factors Va and | | | | |
| | VIIIa) | | | | |
| PROC.127892092.C/T | protein C (inactivator of | 1799808 | 127892092 | 0.4 (T) | 2405 |
| | coagulation factors Va and | | | | |
| | VIIIa) | | | | |
| PROC.127894204.T/C | protein C (inactivator of | 2069910 | 127894204 | 0.49 (T) | 4515 |
| | coagulation factors Va and | | | | |
| | VIIIa) | | | | |
| PROC.127894608.G/A | protein C (inactivator of | 2069915 | 127894608 | 0.44 (A) | 4919 |
| | coagulation factors Va and | | | | |
| | VIIIa) | | | | |
| PROC.127894645.C/T | protein C (inactivator of | 2069916 | 127894645 | 0.4 (T) | 4956 |
| | coagulation factors Va and | | | | |
| | VIIIa) | | | | |
| PROC.127895556.G/A | protein C (inactivator of | 2069918 | 127895556 | 0.21 (A) | 5867 |
| | coagulation factors Va and | | | | |
| | VIIIa) | | | | |
| PROC.127895783.G/A | protein C (inactivator of | 2069919 | 127895783 | 0.3 (A) | 6094 |
| | coagulation factors Va and | | | | |
| | VIIIa) | | | | |
| PROC.127895876.T/C | protein C (inactivator of | 2069920 | 127895876 | 0.44 (C) | 6187 |
| | coagulation factors Va and | | | | |
| | VIIIa) | | | | |
| PROC.127899224.C/T | protein C (inactivator of | 2069924 | 127899224 | 0.4 (T) | 9534 |
| | coagulation factors Va and | | | | |
| | VIIIa) | | | | |
| PROC.127901000.T/C | protein C (inactivator of | 5937 | 127901000 | 0.29 (C) | 11310 |
| | coagulation factors Va and | | | | |
| | VIIIa) | | | | |
| PROC.127901799.C/T | protein C (inactivator of | 2069931 | 127901799 | 0.4 (T) | 12109 |
| | coagulation factors Va and | | | | |
| | VIIIa) | | | | |
| PROC.127975205.T/C | protein C (inactivator of | 777556 | 127975205 | 0.31 (C) | |
| | coagulation factors Va and | | | | |
| | VIIIa) | | | | |
| PROCR.33183348.T/C | protein C receptor, | 1033797 | 33183348 | 0.11 (C) | |
| | endothelial (EPCR) | | | | |
| PROCR.33183694.C/A | protein C receptor, | 1033799 | 33183694 | 0.11 (A) | |
| | endothelial (EPCR) | | | | |
| PROCR.33186524.A/G | protein C receptor, | 2295888 | 33186524 | 0.08 (G) | |
| | endothelial (EPCR) | | | | |
| PROCR.33228215.A/G | protein C receptor, | 867186 | 33228215 | 0.1 (G) | 6118 |
| | endothelial (EPCR) | | | | |

*Note:
SNPs marked with * were genotyped on the complementary strand. SNP nomenclature is consistent with that of Goldenpath.
±Amended from rs10307480 to rs253073 as a result of a consolidation of rs number redundancies, whereby rs10307480, rs10393898, rs2227785 and rs253073 all represented the same polymorphism. The current rs identifier for this polymorphism site is rs253073.

TABLE 1B
Polymorphisms in linkage disequilibrium with those listed in TABLE 1A above, as
identified using the LD-select algorithm (CARLSON CS. et al. Am. J. Hum. Genet. (2004)
74: 106-120), r² ≧ 0.5/minor allele frequency (MAF) = 0.05. The gene is identified, along with
the alleles, rs designation and the chromosomal positions (May 2004 Build 35).

| | Tag | Alleles | Polymorphism | | LD | rsIDs of |
|---|---|---|---|---|---|---|
| Gene | Polymorphisms | (IRP allele) | rsID | Polymorphisms in LD | Alleles | Polymorphisms in LD |
| FGB | 155840914 | G/A (G) | rs1800791 | n/a | n/a | n/a |
| | | | | 155846700 | G | rs2227412 |
| F2 | 46717332 | G/A (G) | rs3136516 | 46716696 | G | rs3136512 |
| F2R | 76059983 | A/G (G) | rs253073 | 76051211 | A | rs37245 |
| | | | | 76046105 | A | rs2227744 |
| | | | | 76048599 | A | rs27135 |
| | | | | 76049220 | G | rs2227750 |
| | | | | 76050075 | A | rs37243 |
| F2R | 76049220 | G/C (GG) | rs2227750 | 76051211 | AA | rs37245 |
| | | | | 76046105 | AA | rs2227744 |
| | | | | 76048599 | AA | rs27135 |
| | | | | 76050075 | AA | rs37243 |
| | | | | 76059983 | DD | rs253073 |
| | | | | 76046105 & 76048669 | G & T | rs2227744 & rs27593 |
| | | | | 76046105 & 76049687 | G & A | rs2227744 & rs37242 |
| | | | | 76046105 & 76049756 | G & A | rs2227744 & rs253061 |
| | | | | 76046105 & 76050867 | G & T | rs2227744 & rs37244 |
| | | | | 76046105 & 76051420 | G & A | rs2227744 & rs37246 |
| | | | | 76048599 & 76048669 | G & T | rs27135 & rs27593 |
| | | | | 76048599 & 76049687 | G & A | rs27135 & rs37242 |
| | | | | 76048599 & 76049756 | G & A | rs27135 & rs253061 |
| | | | | 76048599 & 76050867 | G & T | rs27135 & rs37244 |
| | | | | 76048599 & 76051420 | G & A | rs27135 & rs37246 |
| | | | | 76050075 & 76048669 | G & T | rs37243 & rs27593 |
| | | | | 76050075 & 76049687 | G & A | rs37243 & rs37242 |
| | | | | 76050075 & 76049756 | G & A | rs37243 & rs253061 |
| | | | | 76050075 & 76050867 | G & T | rs37243 & rs37244 |
| | | | | 76050075 & 76051420 | G & A | rs37243 & rs37246 |
| | | | | 76051211 & 76048669 | G & T | rs37245 & rs27593 |
| | | | | 76051211 & 76049687 | G & A | rs37245 & rs37242 |
| | | | | 76051211 & 76049756 | G & A | rs37245 & rs253061 |
| | | | | 76051211 & 76050867 | G & T | rs37245 & rs37244 |
| | | | | 76051211 & 76051420 | G & A | rs37245 & rs37246 |
| | | | | 76052731 & 76048669 | G & T | rs37249 & rs27593 |
| | | | | 76052731 & 76049687 | G & A | rs37249 & rs37242 |
| | | | | 76052731 & 76049756 | G & A | rs37249 & rs253061 |
| | | | | 76052731 & 76050867 | G & T | rs37249 & rs37244 |
| | | | | 76052731 & 76051420 | G & A | rs37249 & rs37246 |
| F3 | 94719939 | A/G (G) | rs1361600 | 94714011 | A | rs3917615 |
| | | | | 94711518 | T | rs841695 |
| | | | | 94711541 | G | rs2794470 |
| | | | | 94714232 | T | rs1144300 |
| | | | | 94716035 | C | rs841697 |
| | | | | 94716105 | G | rs762485 |
| | | | | 94717241 | C | rs696619 |
| | | | | 94720676 | G | rs3761955 |
| | | | | 94721166 | T | rs958587 |
| F5 | 166258759 | A/G (G) | rs9332575 | n/a | n/a | n/a |
| F5 | 166236816 | T/C (A) | rs4656687 | 166213608 | C | rs2187952 |
| | | | | 166214094 | T | rs2040444 |
| | | | | 166215502 | G | rs4656685 |
| | | | | 166216210 | A | rs3820060 |
| | | | | 166217058 | A | rs6670407 |
| | | | | 166217517 | T | rs2420369 |
| | | | | 166218159 | C | rs9332667 |
| | | | | 166218425 | A | rs9332665 |
| | | | | 166220585 | A | rs3766103 |
| | | | | 166221016 | A | rs2227244 |
| | | | | 166221170 | T | rs2213866 |
| | | | | 166221243 | A | rs2213867 |
| | | | | 166222250 | T | rs9332655 |
| | | | | 166222687 | D | rs9332652 |
| | | | | 166222807 | C | rs9332651 |
| | | | | 166224334 | G | rs9332643 |
| | | | | 166225854 | G | rs2301515 |
| | | | | 166227091 | A | rs9332635 |
| | | | | 166229478 | C | rs9332627 |
| | | | | 166229839 | G | rs2420373 |
| | | | | 166230848 | T | rs2157581 |
| | | | | 166231039 | G | rs2187953 |
| | | | | 166231317 | A | rs916438 |
| | | | | 166231609 | G | rs9332620 |
| | | | | 166232006 | C | rs9332619 |
| | | | | 166236487 | G | rs4656187 |
| | | | | 166237899 | T | rs7535409 |
| | | | | 166240234 | T | rs1557572 |
| | | | | 166240397 | T | rs3766109 |
| | | | | 166243213 | A | rs6032 |
| | | | | 166243392 | A | rs4525 |
| | | | | 166243413 | A | rs4524 |
| | | | | 166244571 | G | rs9332600 |
| | | | | 166244638 | C | rs9332599 |
| | | | | 166245094 | G | rs9287092 |
| | | | | 166245995 | T | rs9332596 |
| | | | | 166246013 | C | rs9332595 |
| | | | | 166246841 | T | rs3766110 |
| | | | | 166246862 | A | rs3766111 |
| | | | | 166246954 | G | rs3766112 |
| | | | | 166246965 | T | rs3766113 |
| | | | | 166247039 | A | rs1894694 |
| | | | | 166247104 | D | rs9332589 |
| | | | | 166247194 | G | rs6672595 |
| | | | | 166251166 | A | rs1988607 |
| | | | | 166251207 | C | rs1988608 |
| | | | | 166252117 | C | rs2420375 |
| | | | | 166252207 | C | rs2420376 |
| | | | | 166252250 | C | rs2420377 |
| | | | | 166252651 | T | rs2298909 |
| F5 | 166227911 | A/G (A) | rs9332630 | 166241891 | T | rs9332607 |
| | | | | 166240367 | D | rs9332611 |
| | | | | 166246588 | C | rs9332590 |
| | | | | 166251075 | T | rs7537742 |
| | | | | 166251195 | C | rs9332587 |
| | | | | 166252346 | T | rs9332586 |
| | | | | 166253209 | C | rs721161 |
| F5 | 166269905 | G/A (A) | rs9332546 | 166257923 | T | rs9332577 |
| | | | | 166257466 | T | rs2239854 |
| | | | | 166257958 | A | rs4656688 |
| | | | | 166258025 | C | rs4656689 |
| | | | | 166258083 | A | rs4656188 |
| | | | | 166258259 | G | rs1894697 |
| | | | | 166258304 | C | rs1894698 |
| | | | | 166258608 | C | rs1894699 |
| | | | | 166258884 | C | rs1981491 |
| | | | | 166259603 | A | rs7548857 |
| | | | | 166260488 | A | rs6427202 |
| | | | | 166260796 | A | rs9287093 |
| | | | | 166262019 | G | rs1894700 |
| | | | | 166262188 | G | rs5778621 |
| | | | | 166268097 | A | rs7542281 |
| | | | | 166268143 | G | rs2187954 |
| | | | | 166268160 | A | rs9332556 |
| | | | | 166268308 | T | rs2187955 |
| | | | | 166268559 | T | rs9332554 |
| | | | | 166268668 | T | rs9332553 |
| | | | | 166269336 | C | rs6670678 |
| | | | | 166269427 | D | rs9332548 |
| | | | | 166270254 | C | rs2298907 |
| | | | | 166270500 | A | rs2298905 |
| | | | | 166270941 | T | rs9332542 |
| | | | | 166271581 | A | rs9332538 |
| | | | | 166271612 | A | rs9332537 |
| | | | | 166271738 | A | rs2227245 |
| | | | | 166271935 | I | rs5778622 |
| | | | | 166271950 | D | rs9332534 |
| | | | | 166271992 | T | rs2213870 |
| | | | | 166272080 | C | rs2213871 |
| | | | | 166272250 | G | rs9332533 |
| | | | | 166272554 | G | rs9332531 |
| | | | | 166273793 | A | rs6691048 |
| | | | | 166273848 | D | rs9332520 |
| | | | | 166274375 | A | rs9332516 |
| | | | | 166274680 | A | rs9332513 |
| | | | | 166277480 | T | rs9332511 |
| | | | | 166277493 | T | rs9332510 |
| | | | | 166282732 | D | rs9332500 |
| | | | | 166285716 | C | rs3753305 |
| F7 | 112808416 | A/G (AG) | rs2774030 | 112805827 | C | rs3093229 |
| | | | | 112805969 | G | rs3093230 |
| | | | | 112807487 | A | rs762635 |
| | | | | 112807527 | A | rs762636 |
| | | | | 112807755 | G | rs510317 |
| | | | | 112808856 | G | rs3093237 |
| F10 | 112840894 | A/C (C) | rs2026160 | 112834948 | C | rs483743 |
| | | | | 112832408 | T | rs483949 |
| | | | | 112835822 | G | rs3211753 |
| | | | | 112836955 | G | rs473950 |
| | | | | 112838379 | T | rs3211758 |
| | | | | 112840755 | T | rs2251102 |
| | | | | 112843672 | T | rs776897 |
| F10 | 112825510 | A/G (G) | rs3211719 | n/a | n/a | n/a |
| F10 | 112824083 | T/C (T) | rs3093261 | n/a | n/a | n/a |
| SERPINE1 | 100363146 | del4G/ins5 | rs1799889 | 100362973 | G | rs2227631 |
| | | G (insG) | | | | |
| SERPINE1 | 100375050 | G/A (A) | rs1050813 | 100369665 | T | rs2227676 |
| | | | | 100370029 | I | rs2227681 |
| | | | | 100370071 | A | rs2227683 |
| SERPINA5 | 94123294 | C/T (TT) | rs2069972 | 94123304 | G | rs2069973 |
| | | | | 94123325 | A | rs2069974 |
| | | | | 94123643 | A | rs6115 |
| | | | | 94123929 | C | rs6112 |
| | | | | 94125866 | A | rs2066969 |
| | | | | 94127023 | A | rs6107 |
| | | | | 94128113 | G | rs6109 |
| | | | | 94128215 | C | rs6116 |
| | | | | 94128384 | T | rs6108 |
| | | | | 94128566 | G | rs938 |
| | | | | 94128678 | G | rs1050013 |
| | | | | 94128829 | C | rs9113 |
| | | | | 94129134 | G | rs7070 |
| | | | | 94129535 | G | rs2069995 |
| | | | | 94129617 | A | rs2069996 |
| IL6 | 22541812 | C/G (C) | rs2069840 | 22538581 | D | rs2069825 |
| | | | | 22539461 | A | rs1800797 |
| | | | | 22539885 | C | rs1800795 |
| | | | | 22540673 | A | rs2069832 |
| | | | | 22540904 | C | rs2069833 |
| | | | | 22541148 | C | rs1474348 |
| | | | | 22541364 | C | rs1474347 |
| | | | | 22541947 | T | rs1554606 |
| | | | | 22543389 | G | rs2069845 |
| | | | | 22545967 | G | rs1818879 |
| IL6 | 22539885 | G/C (G) | rs1800795 | 22538581 | I | rs2069825 |
| | | | | 22539461 | G | rs1800797 |
| | | | | 22540673 | G | rs2069832 |
| | | | | 22540904 | T | rs2069833 |
| | | | | 22541148 | G | rs1474348 |
| | | | | 22541364 | A | rs1474347 |
| | | | | 22541812 | G | rs2069840 |
| | | | | 22541947 | G | rs1554606 |
| | | | | 22543389 | A | rs2069845 |
| | | | | 22545967 | A | rs1818879 |
| IL10 | 203334802 | C/A (A) | rs1800872 | 203332628 | T | rs1554286 |
| | | | | 203333040 | A | rs1518111 |
| | | | | 203333256 | T | rs1518110 |
| | | | | 203333706 | T | rs3024490 |
| | | | | 203335029 | T | rs1800871 |
| IL12A | 161198944 | G/A (A) | rs2243154 | n/a | n/a | n/a |
| TNFRSF1A | 6317783 | T/C (CT) | rs4149577 | 6310270 | A | rs1800693 |
| | | | | 6311609 | G | rs4149587 |
| | | | | 6312607 | T | rs1800692 |
| | | | | 6316243 | T | rs887477 |
| | | | | 6317038 | C | rs1860545 |
| | | | | 6317246 | A | rs4149581 |
| | | | | 6317251 | C | rs4149580 |
| | | | | 6319376 | G | rs4149576 |
| | | | | 6321206 | A | rs767455 |
| | | | | 6321851 | T | rs4149570 |
| | | | | 6322729 | G | rs4149569 |
| VEGF | 43848656 | G/A (AA) | rs1413711 | 43850397 | GG | rs865577 |
| | | | | 43850505 | GG | rs833068 |
| | | | | 43850557 | TT | rs833069 |
| | | | | 43850604 | TT | rs833070 |
| | | | | 43851038 | DD | rs3024991 |
| | | | | 43852599 | CC | rs735286 |
| | | | | 43853085 | GG | rs3024997 |
| | | | | 43853555 | CC | rs3024998 |
| | | | | 43855226 | CC | rs3025006 |
| | | | | 43855349 | TT | rs3025007 |
| | | | | 43855428 | AA | rs3025009 |
| PROC | 127890298 | A/G (AG) | rs2069895 | 127890457 | C | rs2069898 |
| | | | | 127891073 | C | rs2069901 |
| | | | | 127891093 | G | rs2069902 |
| | | | | 127892009 | A | rs2069904 |
| | | | | 127892105 | G | rs1799809 |
| | | | | 127892270 | T | rs1799810 |
| | | | | 127893607 | C | rs1158867 |
| | | | | 127895783 | A | rs2069919 |
| | | | | 127901000 | C | rs5937 |
| PROC | 127890457 | C/T (CT) | rs2069898 | 127890298 | G | rs2069895 |
| | | | | 127891073 | C | rs2069901 |
| | | | | 127891093 | G | rs2069902 |
| | | | | 127892009 | A | rs2069904 |
| | | | | 127892105 | G | rs1799809 |
| | | | | 127892270 | T | rs1799810 |
| | | | | 127893607 | C | rs1158867 |
| | | | | 127895783 | A | rs2069919 |
| | | | | 127901000 | C | rs5937 |
| PROC | 127892009 | A/G (AG) | rs2069904 | 127890298 | G | rs2069895 |
| | | | | 127890457 | C | rs2069898 |
| | | | | 127891073 | C | rs2069901 |
| | | | | 127891093 | G | rs2069902 |
| | | | | 127892105 | G | rs1799809 |
| | | | | 127892270 | T | rs1799810 |
| | | | | 127893607 | C | rs1158867 |
| | | | | 127895783 | A | rs2069919 |
| | | | | 127901000 | C | rs5937 |
| PROC | 127892092 | C/T (CT) | rs1799808 | 127894608 | A | rs2069915 |
| | | | | 127894645 | T | rs2069916 |
| | | | | 127895876 | C | rs2069920 |
| | | | | 127899224 | T | rs2069924 |
| | | | | 127894204 | T | rs2069910 |
| PROC | 127894204 | C/T (C) | rs2069910 | 127892092 | C | rs1799808 |
| | | | | 127894608 | G | rs2069915 |
| | | | | 127894645 | C | ys2069916 |
| | | | | 127895876 | T | rs2069920 |
| | | | | 127899224 | C | rs2069924 |
| PROC | 127894608 | A/G (AG) | rs2069915 | 127892092 | 4 | rs1799808 |
| | | | | 127894645 | T | rs2069916 |
| | | | | 127895876 | C | rs2069920 |
| | | | | 127899224 | T | rs2069924 |
| | | | | 127894204 | T | rs2069910 |
| PROC | 127894645 | C/T (CT) | rs2069916 | 127892092 | T | rs1799808 |
| | | | | 127894608 | A | rs2069915 |
| | | | | 127895876 | C | rs2069920 |
| | | | | 127899224 | T | rs2069924 |
| | | | | 127894204 | T | rs2069910 |
| PROC | 127895556 | G/A (A) | rs2069918 | 127894421 | C | rs2069912 |
| | | | | 127894489 | G | rs2069913 |
| | | | | 127894502 | A | rs2069914 |
| | | | | 127896068 | G | rs2069915 |
| | | | | 127897748 | T | rs971207 |
| | | | | 127896451 | A | rs973760 |
| | | | | 127897469 | C | rs2069922 |
| | | | | 127898605 | T | rs1518759 |
| | | | | 127900144 | T | rs2069928 |
| | | | | 127901918 | T | rs2069933 |
| PROC | 127895783 | A/G (AG) | rs2069919 | 127890298 | G | rs2069895 |
| | | | | 127890457 | C | rs2069898 |
| | | | | 127891073 | C | rs2069901 |
| | | | | 127891093 | G | rs2069902 |
| | | | | 127892009 | A | rs2069904 |
| | | | | 127892105 | G | rs1799809 |
| | | | | 127892270 | T | rs1799810 |
| | | | | 127893607 | C | rs1158867 |
| | | | | 127901000 | C | rs5937 |
| PROC | 127895876 | C/T (CT) | rs2069920 | 127892092 | T | rs1799808 |
| | | | | 127894608 | A | rs2069915 |
| | | | | 127894645 | T | rs2069916 |
| | | | | 127899224 | T | rs2069924 |
| | | | | 127894204 | T | rs2069910 |
| PROC | 127899224 | C/T (CT) | rs2069924 | 127892092 | T | rs1799808 |
| | | | | 127894608 | A | rs2069915 |
| | | | | 127894645 | T | rs2069916 |
| | | | | 127895876 | C | rs2069920 |
| | | | | 127894204 | T | rs2069910 |
| PROC | 127901000 | C/T (CT) | rs5937 | 127890298 | G | rs2069895 |
| | | | | 127890457 | C | rs2069898 |
| | | | | 127891073 | C | rs2069901 |
| | | | | 127891093 | G | rs2069902 |
| | | | | 127892009 | A | rs2069904 |
| | | | | 127892105 | G | rs1799809 |
| | | | | 127892270 | T | rs1799810 |
| | | | | 127893607 | C | rs1158867 |
| | | | | 127895783 | A | rs2069919 |
| PROC | 127901799 | C/T (CT) | rs2069931 | NA | | |
| PROC | 127975205 | C/T (C) | rs777556 | NA | | |
| PROCR | 33183348 | C/T (C) | rs1033797 | NA | | |
| PROCR | 33183694 | A/C (A) | rs1033799 | NA | | |
| PROCR | 33186524 | A/G (G) | rs2295888 | NA | | |
| PROCR | 33228215 | A/G (G) | rs867186 | 33222933 | G | rs2069940 |

It will be appreciated by a person of skill in the art that further linked polymorphic sites and combined polymorphic sites may be determined. The haplotype of protein C pathway associated genes can be created by assessing polymorphisms in protein C pathway associated genes in normal subjects using a program that has an expectation maximization algorithm (i.e. PHASE). A constructed haplotype of protein C pathway associated genes may be used to find combinations of SNP's that are in linkage disequilibrium (LD) with the haplotype tagged SNPs (htSNPs) identified herein. Accordingly, the haplotype of an individual could be determined by genotyping other SNPs or other polymorphisms that are in LD with the htSNPs identified herein. Single polymorphic sites or combined polymorphic sites in LD may also be genotyped for assessing subject response to activated protein C or protein C like compound or protein C like compound treatment.

It will be appreciated by a person of skill in the art, that the numerical designations of the positions of polymorphisms within a sequence are relative to the specific sequence. Also the same positions may be assigned different numerical designations depending on the way in which the sequence is numbered and the sequence chosen, as illustrated by the alternative numbering of the equivalent polymorphism (rs1799889), whereby the same polymorphism identified as an insertion/deletion polymorphism (4G/5G) at position −675 of the SERPINE1 promoter sequence (by DAWSON et al. Journal of Biological Chemistry (1993) 268(15):10739-45), which corresponds to position 201 of SEQ ID NO:14 and to position 201 of SEQ ID NO:14. Furthermore, sequence variations within the population, such as insertions or deletions, may change the relative position and subsequently the numerical designations of particular nucleotides at and around a polymorphic site.

Polymorphic sites in SEQ ID NO:1-40 and SEQ ID NO:41-243 are identified by their variant designation (i.e. M, W, Y, S, R, K, V, B, D, H or by “−” for a deletion, a “+” or “G” etc. for an insertion).

An “rs” prefix designates a SNP in the database is found at the NCBI SNP database (http://www.ncbi.nlm.nih.gov/entrez/query.fcgi?db.Snp). The “rs” numbers are the NCBI|rsSNP ID form.

TABLE 1C below shows the flanking sequences for a selection of protein C pathway associated gene SNPs providing their rs designations, alleles and corresponding SEQ ID NO designations. Each polymorphism is at position 201 within the flanking sequence, unless otherwise indicated, and identified in bold and underlined.

TABLE 1C

| | | | |
|---|---|---|---|
| | | SEQ ID | |
| GENE | SNP | NO: | FLANKING SEQUENCE |

| | | | |
|---|---|---|---|
| FGB | rs1800791 | 1 | TTCCTATTGATTCTTGTAGGAGTTATTAATCCTGATTGCAACACACAAGTGAA |
| | (position | | CAGACAAGAGAGATAAATTTTGTGGCTTGTGGRAAATGAAGGAAAATGGGCCT |
| | 86) | | CATTTAGTCTGTGAGCATACTAATTGAAATAGATGTATGAAGACTTCACCAGT |
| | | | GTTAAAATAACATTGTTTTTATAAATCATATGATATAAACTATATAACAATAA |
| | | | AATAGAATGTTRAACATGTATTTAATCATCATCATAATTTTGATTCAGAAATC |
| | | | TATAATTTATTAGTTATCTTAATAATGTTTAGAATTTGTTGAACATTTTACCT |
| | | | TATGTGAATTAAGGACAAAATATTAAAGCTATTCAGCACAAAAAAAGGGTCTT |
| | | | TCTGATGTGTATTTTTCATAGAATAGGGTATGAATTTGTTATTTTGTTATTTT |
| | | | GATTAATGTCTAAAACAAAAGATAAACACATTATGATATAACATTACTATTGA |
| | | | TTTTAATRGCCCCTTTTGAAATAGAATTATGTCATTGTCAGAAAACATAAGCA |
| | | | TTTATGGTATATCATTAATGAGT |
| F2 | rs3136516 | 2 | ATGAGCTATGCTCCTGAGCACAGACGGCTGTTCTCTTTCAAGGTTACAAGCCT |
| | | | GATGAAGGGAAACGAGGGGATGCCTGTGAAGGTGACAGTGGGGGACCCTTTGT |
| | | | CATGAAGGTAAGCTTCTCTAAAGCCCAGGGCCTGGTGAACACATCTTCTGGGG |
| | | | GTGGGGAGAAACTCTAGTATCTAGAAACAGTTGCCTGGCAGRGGAATACTGAT |
| | | | GTGACCTTGAACTTGACTCTATTGGAAACCTCATCTTTCTTCTTCAGAGCCCC |
| | | | TTTAACAACCGCTGGTATCAAATGGGCATCGTCTCATGGGGTGAAGGCTGTGA |
| | | | CCGGGATGGGAAATATGGCTTCTACACACATGTGTTCCGCCTGAAGAAGTGGA |
| | | | TACAGAAGGTCATTGATCAGTTTGGAGAGTAGGGGGCCACTCATATTCTGGGC |
| | | | TCCTGGAACCAATCCCGTGAAAGAATTATTTT |
| F2R | rs253073 | 3 | CAAACTTATAACATGTATTACCTCAAATACTTATCATTTTTTGTGGTGAGACC |
| | | | ACTTAAAGTCTACTCTCTTAGCAATTGTCAAGTATACAACACATTGTTATTAA |
| | | | CTATAGTCACCGTATTATACAAGAGATTTTTCTAACTAATTCCTCCATCCAAC |
| | | | CCCAGCCTCTACTAACCACCATTCTCTTCTCTGCTTCCATGRGTTCAACTGTT |
| | | | TTCGATTCCACATATAAGTGAAATCATGCTATATTTGTCTTTCGGTGCCTGCC |
| | | | ATATTTCACTTAACATTATGCCCTCTCGGCTCATCCATGTTGTTGCAAATGAC |
| | | | AGAATTTCCTTCTTTTTTAAGGCTGAATAGTATTCCACTGTGTATATACATCA |
| | | | TATTTTTTATTCATTCATTCATTCATCAGTGGATACCTAGTTTGATTCCATAA |
| | | | CTGGGCTATTATAAATAATTCTGCAATGAACA |
| F2R | rs2227750 | 4 | GCACTCTATATTGCTCCCACACTCAAAAAAAAGTGTAGACACATCAAGATTAA |
| | | | GAGGTGACAAAGACATAGCATGTTCTCGCCTCTCTGTCTTTGTTCAGGGTGAG |
| | | | TTTTGAGATGCTTTTGGGAAAACTAAGAGCTCCAGACTGGGGCCCAGTGTTTA |
| | | | GCAGTAACTAGCCTGCCTGCAGATAAGTGAGCATTGTTGCCSAAAGTGTTTGA |
| | | | GAGAACACCGAGAACTCCTGAAAAATTGTTTGCGATGAGATATGATTTCACAT |
| | | | ACCATTATGTAATTTGCACAATGTAGTTTGAGGACACGCTCTTGAGAATCCAG |
| | | | TGTTTTGTTTGAGATTTGGATCATGGGGTGGAGAACAGAGCTTATAGAAATGC |
| | | | TGCACCCTTTCTCACAGTGGCCTCCCAGCAAGGTGTGTAGCCTCATTAGGGAG |
| | | | TGAAGTCAAAGCGTACTGGTTTCTGCCAAGCT |
| F3 | rs1361600 | 5 | CGCTGGAATTCTCCCAGAGGCAAACTGCCAGATGTGAGGCTGCTCTTCCTCAG |
| | | | TCACTATCTCTGGTCGTACCGGGCGATGCCTGAGCCAACTGACCCTCAGACCT |
| | | | GTGAGCCGAGCCGGTCACACCGTGGCTGACACCGGCATTCCCACCGCCTTTCT |
| | | | CCTGTGCGACCCGCTAAGGGCCCCGCGAGGTGGGCAGGCCARGTATTCTTGAC |
| | | | CTTCGTGGGGTAGAAGAAGCCACCGTGGCTGGGAGAGGGCCCTGCTCACAGCC |
| | | | ACACGTTTACTTCGCTGCAGGTCCCGAGCTTCTGCCCCAGGTGGGCAAAGCAT |
| | | | CCGGGAAATGCCCTCCGCTGCCCGAGGGGAGCCCAGAGCCCGTGCTTTCTATT |
| | | | AAATGTTGTAAATGCCGCCTCTCCCACTTTATCACCAAATGGAAGGGAAGAAT |
| | | | TCTTCCAAGGCGCCCTCCCTTTCCTG |
| F5 | rs9332575 | 6 | AAACCATATGCACAAAAATAAAAATAAATTGATCTGAGCTTAGAGTTTACGAA |
| | | | TTTATAGTTCCCAAAAGAATAACTGGGGGTAAATGGGACAAGGTAGGGAAAGA |
| | | | CCATCAGTAGAAACTAAGAATAGTAAACATTTGTAAAAACCCTCTGCCTTATA |
| | | | AAGCAGAATAAATTGAATACATATGATAAATGCTAACACAGRTATGTTAATTG |
| | | | CTGAACTCAATATAAGCATTTCTTTGGCATGGATAAACGCTTCCACCAAAGCC |
| | | | TCCTAGTGATGCAAGGAATCCTGGCTTTGTTGCAATGGTCTCCTAAAAAAAAC |
| | | | AAGCCTTTGTGTGGGTAAGGAACTGATTCTCAGCCCCATTATCTAGTATCTAG |
| | | | TGATTATGTATCTGAGATGTAAAACAGAAACCTAAAAGCCAAGGATGGAGTCT |
| | | | CCCCACAGAGCAAATGAGCATTTTCCCAGTGA |
| F5 | rs4656687 | 7 | AAAAGAGAATATTGCCTCCCATAGCTTCATGGAAAATTTAGAATAATTAAGAT |
| | | | TCTTATATCCCTATGTACTTGTTTACGTTTTTAAAAAGAGCAAATGGTCACTG |
| | | | AAAATGTAGTGAATGCTTACACAGGTATAGTAGATTATATTTTAAAATCTAGA |
| | | | ATAGCTTACTTTAGAATCAGGGTTCTTTCTGGGTTTTTGAAYGTGAGCGGTTA |
| | | | GCAAAAATGGCGGAAGTAAACTTTGTTATAAAAGCAAGTTATAATCGTGGGTC |
| | | | TGGACACAAGATTTTGAAAGAAATTACCAGAACTAGGAAGACACTGAAGAAAC |
| | | | TTGCTCACTTTTGGAGGGCATGGAGACATCTTTACTTTCCTTACTCATTTTAT |
| | | | TTAACTTCATTTTATTTAATTTTTAGGAAAACACCTGAAGATATTTCTAAATT |
| | | | ACTATTACTACTAGCACTGCTACTGCCACCAC |
| F5 | rs9332630 | 8 | AAAGTAGATTTTGGGCAGAATTCCAAGGAGTCTGTATTTTTAACAAGCAGCCT |
| | | | TCCTTCTATTTTGCCTTCTATTAAAAGTAATGGCAAAAACCGCAATTATAGTT |
| | | | TGCACCAACCTAACACATGCTGCCTGAGGAGTTAGTGAAGGCAGCCCCTCGAC |
| | | | AGCACTTTGGGTGACGTTGTGTGAATCTGCCTCAGATGCAGRCACAGAAGTCC |
| | | | AAATGGACTGGTTTGATTAAGAGCAGGGAAAAAAAGAGGGTTCTTATTGGTTT |
| | | | TTCACATGCCAGTAACTCACTAATACATCTAGAGAGTATTAATTGTATTATAT |
| | | | TAATATCATATTAATTAATTAATATAATAATTAATAGATAATTTATTGTATTA |
| | | | AAATCAGAGACAGAAGAGATTCAGTCAAATTTACTCATCTTTTCATCAAGTAT |
| | | | TAGAAGATCAGTCATCCTTCCTATCAGCCTGCAGACAGACTGAGAAGCTAG |
| F5 | rs9332546 | 9 | ACTTAATGTAGGTGATTACATTTTTTCACTTCTCATCTGAATATTTTGGCCTT |
| | | | ATGTAGAGACTTCCTTGAGTATATGATAAACACCTGAAACAAACTATAACGGC |
| | | | TATAGTTTGTATATTTAATAAATCACAAAAATGTGGATCCCTTTTCCAAAGAG |
| | | | ACTTGCAAGTCCTGTGCAATGAAATCAGCTCTTTATAAAACRTCAGCATTGAT |
| | | | TCGCCCATTGCCTACATTATAGCTTCTAAAAGAATATTCTGAATCTTTGAATG |
| | | | TAGTGAAGCATTCACTAAGTTTATTAACTTAGGAGAGGAAAAGAGTTGTTCAC |
| | | | AAAAATAAGTAACAAGGGAAGAGTTACTGAGTGAGCACCCTAATGAGTAACCA |
| | | | AAAAGGCTCTTTATGTAACTTCACTTTCCCAAAGCTTGTATGCACCATTTTTC |
| | | | ATTTTTAAAAAATACTGAGAGCCTTTGGCAAG |
| F7 | rs2774030 | 10 | TGCAGGTGCGTCCGGGGAGGTTTTCTCCATAAACTTGGTGGAAGGGCAGTGGG |
| | | | CAAATCCAGGAGCCAGCCCGGGCTTCCCAAACCCCGCCCTTGCTCCGGACACC |
| | | | CCCATCCACCAGGAGGGTTTTCTGGCGGCTCCTGTTCAATTTCTTTCCTTCTA |
| | | | GAAACCAGCATCCAGGCACAGGAGGGGAGGCCCTTCTTGGTRGCCCAGGCTTT |
| | | | GGCGGGATTATTTTTCAAAGAACTTTAGGAGTGGGTGGTGCTTTCCTGGCCCC |
| | | | CATGGGCCCCTGCCTGTGAGGTCGGACAAGCGCAGGGAGTCTGGGGCCTCTCA |
| | | | GAGTGCAGGAAGTGCGCACAGGGTGCTCCCAGGCTGGGGAGCACAGGTAGGGG |
| | | | ACGGTGCGTGGGGGATGGCGCCTGGGGCATGGGGGATGGGGTGTGGGAAACGG |
| | | | CATGTGGGGCGTAAGGGATGGGGTGTGGAGGA |
| F10 | rs2026160 | 11 | CACTTAATTATGGTTGTTATTGGTATAAAATGTCTCTGTTTTCCCTAATATAT |
| | | | TTTTAAATCTCTTTTTTCCTTTTAGAATGAATTCTGGAATAAATACAAAGGTC |
| | | | AGTATTTTTTCTGTTTTAACCTTCAGTGAGAGGGGTTCATCAGGATATTTGAA |
| | | | TTTTGAAAATAGTTCCTGAATTTCCTTTCTGCTTTTGTTCTMATTTTACTCAT |
| | | | TTAAGACTTTTTCCCTCAGGGTGTTTCCATAATAGTTATTGTAAAAGAGTTTT |
| | | | TAGAGTAATTTTATACTAATCCTAGTTTTGTTATTGAGTTAGAGATATATATT |
| | | | TAAATCAGTTCATTCTCATTTGAGGATACCAAATTCCATGATAACTTTTCTTA |
| | | | AATAAAAGTGTATTC |
| F10 | rs3211719 | 12 | CCCTCTCATCTCTGCAGCCTGGACGGTGGGTGCCTTGAGTGCTGCCAGAGGCT |
| | | | GGGCTCGGATGGCTGGGCTTGGCCTTTCCAGCCAACGGCATCCTCAAGGCCAG |
| | | | CTGTGGCTCCCTGGGGCTGAGAGTCAGACGGGCGGATCAGAGGTCACAGAGAC |
| | | | AAAAACACAAGGACAGAGTCAGAGAGAGAAAGGGAGAGGGARGGAGAAACGGA |
| | | | GACACAGTGAGATGGGAGGCCAAGAGGCAGAGACAGAGGTAGAAAGACGGAGA |
| | | | CAGAGAGAGAGGGAGGGGTTGGGGCAGGCAGAGACAGGACAGTTAGCCATCTG |
| | | | CCACCACAGGGAGGCACAGGACGAGGGGCACAGCAGAGGAGCTCCCAGGGAGG |
| | | | AGGAGGCTGAGCCGAGCCAGTGCCACCACTCTCGGACTGGCTCCGTCGGGGAA |
| | | | GGAGCTGCCTAATGCACAGCTGGACAGGTGGG |
| F10 | rs3093261 | 13 | CTGCTGTTGGTGCACACACCGCATTGGTCTCTCCATACAAACATGCCTAGAGG |
| | | | CGATGTCAGAGGGTGGAGACCAGGAGAGGCAGGAGTCAGACATCTGGTGCCAC |
| | | | CAGGAAGGCCCTTCTCAGAGGACCAGGCTGTGCGTGGTGCCCGCCGTGGGAGG |
| | | | CCAGCCTGGCGTTGGCATCCAGCATCATCAGTTTGTGCAGTYGGGTGGGGCTC |
| | | | AGTGAGTGCCTCCTGTGTGCCAGGCACAATGACGCACAATGTGTGCACACCAG |
| | | | GCTCATGTGCAGGTGGCTGCGAGACAGGGCGACCCATCAAGGCAGATGCACCA |
| | | | TGAGGCAGTGGCCAGTGCTGTGGGTGTTAGGGGCATTGCTCCCCGGCCACTAC |
| | | | GGCATAGCAGGCAGTGATCGCCACACTGGCCAAGCTTTAGACCATTTATTCCA |
| | | | GAGACCCCAGAGGCAAAAAGCCCGGCTGCACC |
| SERPINE1 | rs1799889 | 14 | TGCCCCAAGTCCTAGCGGGCAGCTCGAGGAAGTGAAACTTACACGTTGGTCTC |
| | | | CTGTTTCCTTACCAAGCTTTTACCATGGTAACCCCTGGTCCCGTTCAGCCACC |
| | | | ACCACCCCACCCAGCACACCTCCAACCTCAGCCAGACAAGGTTGTTGACACAA |
| | | | GAGAGCCCTCAGGGGCACAGAGAGAGTCTGGACACGTGGGG-/A/G |
| | | | AGTCAGCCGTGTATCATCGGAGGCGGCCGGGCACATGGCAGGGATGAGGGAAA |
| | | | GACCAAGAGTCCTCTGTTGGGCCCAAGTCCTAGACAGACAAAACCTAGACAAT |
| | | | CACGTGGCTGGCTGCATGCCCTGTGGCTGTTGGGCTGGGCCCAGGAGGAGGGA |
| | | | GGGGCGCTCTTTCCTGGAGGTGGTCCAGAGCACCGGGTGGACAGCCCTGGGGG |
| | | | AAAACTTCCACGTTTTGATGGAGGTTATCTTTGATAAC |
| SERPINE1 | rs1050813 | 15 | CTTTTATTTTTATAGGAATAGAGGAAGAAATGTCAGATGCGTGCCCAGCTCTT |
| | | | CACCCCCCAATCTCTTGGTGGGGAGGGGTGTACCTAAATATTTATCATATCCT |
| | | | TGCCCTTGAGTGCTTGTTAGAGAGAAAGAGAACTACTAAGGAAAATAATATTA |
| | | | TTTAAACTCGCTCCTAGTGTTTCTTTGTGGTCTGTGTCACCRTATCTCAGGAA |
| | | | GTCCAGCCACTTGACTGGCACACACCCCTCCGGACATCCAGCGTGACGGAGCC |
| | | | CACACTGCCACCTTGTGGCCGCCTGAGACCCTCGCGCCCCCCGCGCCCCCCGC |
| | | | GCCCCTCTTTTTCCCCTTGATGGAAATTGACCATACAATTTCATCCTCCTTCA |
| | | | GGGGATCAAAAGGACGGAGTGGGGGGACAGAGACTCAGATGAGGACAGAGTGG |
| | | | TTTCCAATGTGTTCAATAGATTTAGGAGCAGA |
| SERPINA5 | rs2069972 | 16 | AGACAGAGCAGAGCAGAGGGAACCCTCTCCCTCCATATCCCATCCTCCAAAAT |
| | | | GTGTCCCTTGATGTGGATGGGTAGACAGGATTCCTGCCCTGGCAGCCAGACCC |
| | | | CTGCCTTGGGTCTGCACCTCCTCTCCCTCCTTCCTCTCCCCGTCATCCCTAAA |
| | | | TCTTGTCCTCGAGCCACTGCCACCCTGTGTAAACCCTCATGYCCAGTCTTGGG |
| | | | GGTGCCATCCCTTCTCTTTAAAGCTGAATGGACCAAACATACCCATTGAGTGT |
| | | | TGGGTGGGGACATCTCTGGAAAGTCAGCACCTGGACCAGCTCCACCCCTCTCT |
| | | | GAGGACACCTTCTTTCCCTTTCAGAACAAAGAACAGCCACCATGCAGCTCTTC |
| | | | CTCCTCTTGTGCCTGGTGCTTCTCAGCCCT |
| IL6 | rs2069840 | 17 | AACCTTCCAAAGATGGCTGAAAAAGATGGATGCTTCCAATCTGGATTCAATGA |
| | | | GGTACCAACTTGTCGCACTCACTTTTCACTATTCCTTAGGCAAAACTTCTCCC |
| | | | TCTTGCATGCAGTGCCTGTATACATATAGATCCAGGCAGCAACAAAAAGTGGG |
| | | | TAAATGTAAAGAATGTTATGTAAATTTCATGAGGAGGCCAASTTCAAGCTTTT |
| | | | TTAAAGGCAGTTTATTCTTGGACAGGTATGGCCAGAGATGGTGCCACTGTGGT |
| | | | GAGATTTTAACAACTGTCAAATGTTTAAAACTCCCACAGGTTTAATTAGTTCA |
| | | | TCCTGGGAAAGGTACTCTCAGGGCCTTTTCCCTCTCTGGCTGCCCCTGGCAGG |
| | | | GTCCAGGTCTGCCCTCCCTCCCTGCCCAGC |
| IL6 | rs1800795 | 18 | CAAAAAACATAGCTTTAGCTTATTTTTTTTCTCTTTGTAAAACTTCGTGCATG |
| | | | ACTTCAGCTTTACTCTTTGTCAAGACATGCCAAAGTGCTGAGTCACTAATAAA |
| | | | AGAAAAAAAGAAAGTAAAGGAAGAGTGGTTCTGCTTCTTAGCGCTAGCCTCAA |
| | | | TGACGACCTAAGCTGCACTTTTCCCCCTAGTTGTGTCTTGCSATGCTAAAGGA |
| | | | CGTCACATTGCACAATCTTAATAAGGTTTCCAATCAGCCCCACCCGCTCTGGC |
| | | | CCCACCCTCACCCTCCAACAAAGATTTATCAAATGTGGGATTTTCCCATGAGT |
| | | | CTCAATATTAGAGTCTCAACCCCCAATAAATATAGGACTGGAGATGTCTGAGG |
| | | | CTCATTCTGCCCTCGAGCCCACCGGGAACG |
| IL10 | rs1800872 | 19 | TAAAATAGAGACGGTAGGGGTCATGGTGAGCACTACCTGACTAGCATATAAGA |
| | | | AGCTTTCAGCAAGTGCAGACTACTCTTACCCACTTCCCCCAAGCACAGTTGGG |
| | | | GTGGGGGACAGCTGAAGAGGTGGAAACATGTGCCTGAGAATCCTAATGAAATC |
| | | | GGGGTAAAGGAGCCTGGAACACATCCTGTGACCCCGCCTGTMCTGTAGGAAGC |
| | | | CAGTCTCTGGAAAGTAAAATGGAAGGGCTGCTTGGGAACTTTGAGGATATTTA |
| | | | GCCCACCCCCTCATTTTTACTTGGGGAAACTAAGGCCCAGAGACCTAAGGTGA |
| | | | CTGCCTAAGTTAGCAAGGAGAAGTCTTGGGTATTCATCCCAGGTTGGGGGGAC |
| | | | CCAATTATTTCTCAATCCCATTGTATTCTGGAATGGGCAATTTGTCCACGTCA |
| | | | CTGTGACCTAGGAACACGCGAATGAGAACCCACAGCTGAGGGCCTCTGCGCAC |
| | | | AGAACAGCTGTTCTCCCCAGGAAAT |
| IL12A | rs2243154 | 20 | AATCATTCCAATGCTCCCCATTGGTCTCCTCTTCTGAAAAAGGAAGGTAATAC |
| | | | TAGAATCTACCTAAAAGGATCAGAGAAAGGGTAAAATGGAACAACTCGTGCAA |
| | | | AGGGCTAGCGTTGCACCTGGCACATAGTAAGTGCACAATAAATGTAAGCACAT |
| | | | TTTGAAATGTATTATTAGTCTTTGGGCTAAGCACCTGCACCRAATTTGTTACC |
| | | | TCCTCTTTGCTGCTATTTCCTCATTGATGAAATTCAGAAAACGGTGGGACCTA |
| | | | ATTAACTGTGTTATTGTGAAGATTAAATGACACAATACAGTGCCAGCACCTAG |
| | | | TTATTACTCAACATAAATTTGTCACAGTTCTCACAAGACATCAGAACACCCGC |
| | | | TGATGTGCTGTCCCCCATGGCACTCAGCATATTAAGTGTGGTCGGCACAAGCG |
| | | | GCTGCCTGGTGTGAAGTATGAGGGCAAAAGGC |
| TNFRSF1A | rs4149577 | 21 | TTCAGATCATTTCCATGACCATGGAAATGCTGTTTGGAGCCAGGCCCTGGAGA |
| | | | TGGAGAGGAAGGTTCACACACTTGTGCGTGCAAGTTAAAGCCTGAATGAAGAT |
| | | | TTAAAAAGTGTGTAGGACGGATGGGAGCAGGAGAGAGGCTAGAAGACACTTGC |
| | | | AATAACCCAGGTGTGAGGCAACCCAGGAATGCGGAGAGGACYGAGAGATCACA |
| | | | GGGGGAGGCCTCGCAAGATGAACTGACACATGGGATGGCGGCAGGGATAGGGA |
| | | | TGGGGCCCTGGGGAGAGAGCGTGGCAAGTTCTCAGCATTCGTCCGGGAAGTCG |
| | | | ATGGTGTGTCATTTGTCTAGGTGAGGAGATGGATGAATTCCGTCTGGGGCATG |
| | | | TTAAGGGTCAGGGAAATGGTCATGTGGAAGGGTGCGCCTACCAAGCTGGAGGA |
| | | | GAGGTGCTGCAACTTCTTTCTGCCTTTGTATC |
| VEGF | rs1413711 | 22 | ACAGTTACCAGGCTTCCAGCTGGACAGCTTACCACTGCGGCTCCTGCAGGGAC |
| | | | CCCCTGGATTCTGCACCTCAGCCCCCTCACCCATTCCCATGACACCCCCTGCC |
| | | | TTCCCCCTGACAATATTCTCCCGGGACCCTCCACTCCTCCTGGGCCCCAAGGA |
| | | | GGAAAGGGGACGGAAATTTCATACCCCTTCCAAGGCCAGGGRGCACAGGAGGG |
| | | | GCGGTTCTAGGCAGGCAGGGGCCAGGTGTCCTTCTCTGGGGGCCTCTGAAGGT |
| | | | CACACTGTGGCCAGGCAGCCACTCCTCCCCCTCCTCCCTACTTGGAGGCCTGT |
| | | | AGCCAAGGCCTTTGTGCCAGGGTCTGAGGAACTTGCGGTGTTAGCAGCGACCC |
| | | | CTGTCCATGGCTTTCCTCTTGCCTC |
| PROC | rs2069895 | 23 | GCTGCCTGTGCTGGGGTGGGGAGGAGTAGAGGGCGAGAAGTTGGTGGGGARGG |
| | (at position | | GAAGCGGCGCCAAAAGAATACCCACAACATCTTGCACCTGGAAGGCAA |
| | 51) | | |
| PROC | rs2069898 | 24 | ATACAAGCTGGTGCCTTCTGTGGTTGTGCATGGGGTCTTCATGCTTCCTGYCT |
| | (at position | | GAGTTCCCAGAAGCTTGTCTCTGCTTTTCTAGGCAGCTGCCACAGCCT |
| | 51) | | |
| PROC | rs2069904 | 25 | ACTATAATATCTCTGGGCAAAAATGTCCCCATCTGAAAAACAGGGACAACRTT |
| | (at position | | CCTCCCTCAGCCAGCCACTATGGGGCTAAAATGAGACCACATCTGTCA |
| | 51) | | |
| PROC | rs1799808 | 26 | TTATAATTAATGGTATTTTAGATTTGACGAAATATGGAATATTACCTGTTGTG |
| | | | CTGATCTTGGGCAAACTATAATATCTCTGGGCAAAAATGTCCCCATCTGAAAA |
| | | | ACAGGGACAACGTTCCTCCCTCAGCCAGCCACTATGGGGCTAAAATGAGACCA |
| | | | CATCTGTCAAGGGTTTTGCCCTCACCTCCCTCCCTGCTGGAYGGCATCCTTGG |
| | | | TGGGCAGAGGTGGGCTTCGGGCAGAACAAGCCGTGCTGAGCTAGGACCAGGAG |
| | | | TGCTAGTGCCACTGTTTGTCTATGGAGAGGGAGGCCTCAGTGCTGAGGGCCAA |
| | | | GCAAATATTTGTGGTTATGGATTAACTCGAACTCCAGGCTGTCATGGCGGCAG |
| | | | GACGGCGAACTTGCAGTATCTCCACGACC |
| PROC | rs2069910 | 27 | CTGCCAGGGCAGGCATGCGTGATGGCAGGGAGCCCCGCGATGACCTCCTAAAG |
| | | | CTCCCTCCTCCACACGGGGATGGTCACAGAGTCCCCTGGGCCTTCCCTCTCCA |
| | | | CCCACTCACTCCCTCAACTGTGAAGACCCCAGGCCCAGGCTACCGTCCACACT |
| | | | ATCCAGCACAGCCTCCCCTACTCAAATGCACACTGGCCTCAYGGCTGCCCTGC |
| | | | CCCAACCCCTTTCCTGGTCTCCACAGCCAACGGGAGGAGGCCATGATTCTTGG |
| | | | GGAGGTCCGCAGGACACATGGGCCCCTAAAGCCACACCAGGCTGTTGGTTTCA |
| | | | TTTGTGCCTTTATAGAGCTGTTTATCTGCTTGGGACCTGCACCTCCACCCTTT |
| | | | CCCAAGGTGCCCTCAGCTCAGGCATACCC |
| PROC | rs2069915 | 28 | TCTAGGATGCCTTTTCCCCCATCCCTTCTTGCTCACACCCCCAACTTGATCTC |
| | | | TCCCTCCTAACTGTGCCCTGCACCAAGACAGACACTTCACAGAGCCCAGGACA |
| | | | CACCTGGGGACCCTTCCTGGGTGATAGGTCTGTCTATCCTCCAGGTGTCCCTG |
| | | | CCCAAGGGGAGAAGCATGGGGAATACTTGGTTGGGGGAGGARAGGAAGACTGG |
| | | | GGGGATGTGTCAAGATGGGGCTGCATGTGGTGTACTGGCAGAAGAGTGAGAGG |
| | | | ATTTAACTTGGCAGCCTTTACAGCAGCAGCCAGGGCTTGAGTACTTATCTCTG |
| | | | GGCCAGGCTGTATTGGATGTTTTACATGACGGTCTCATCCCCATGTTTTTGGA |
| | | | TGAGTAAATTGAACCTTAGAAAGGTAAAG |
| PROC | rs2069916 | 29 | CCCCAACTTGATCTCTCCCTCCTAACTGTGCCCTGCACCCAAGACAGACACTT |
| | | | CACAGAGCCCAGGAGACACCTGGGGACCCTTCCTGGGTGATAGGTCTGTCTAT |
| | | | CCTCCAGGTGTCCCTGCCCAAGGGGAGAAGCATGGGGAATACTTGGTTGGGGG |
| | | | AGGAGAGGAAGACTGGGGGGATGTGTCAAGATGGGGCTGCAYGTGGTGTACTG |
| | | | GCAGAAGAGTGAGAGGATTTAACTTGGCAGCCTTTACAGCAGCAGCCAGGGCT |
| | | | TGAGTACTTATCTCTGGGCCAGGGACTGTATTGGATGTTTTACATGACGGTCT |
| | | | CATCCCCATGTTTTTGGATGAGTAAATTGAACCTTAGAAAGGTAAAGACACTG |
| | | | GCTCAAGGTCACACAGAGATCGGGGTGGGGTTCACAGGGAGGCCTGTCCATCT |
| | | | CAGAGCAAGGCTTCGTCCTCCAACTG |
| PROC | rs2069918 | 30 | GGAGTTGTGGGGGTGGCTGAGTGGAGCGATTAGGATGCTGGCCCTATGATGTC |
| | | | GGCCAGGCACATGTGACTGCAAGAAACAGAATTCAGGAAGAAGCTCCAGGAAA |
| | | | GAGTGTGGGGTGACCCTAGGTGGGGACTCCCACCAGCCACAGTGTAGGTGGTT |
| | | | CAGTCCACCCTCCAGCCACTGCTGAGCACCACTGCCTCCCCRTCCCACCTCAC |
| | | | AAAGAGGGGACCTAAAGACCACCCTGCTTCCACCCATGCCTCTGCTGATCAGG |
| | | | GTGTGTGTGTGACCGAAACTCACTTCTGTCCACATAAAATCGCTCACTCTGTG |
| | | | CCTCACATCAAAGGGAGAAAATCTGATTGTTCAGGGGGTCGGAAGACAGGGTC |
| | | | TGTGTCCTATTTGTCTAAGGGTCAGAGTC |
| PROC | rs2069919 | 31 | AGACCACCCTGCTTCCACCCATGCCTCTGCTGATCAGGGTGTGTGTGTGACCG |
| | | | AAACTCACTTCTGTCCACATAAAATCGCTCACTCTGTGCCTCACATCAAAGGG |
| | | | AGAAAATCTGATTGTTCAGGGGGTCGGAAGACAGGGTCTGTGTCCTATTTGTC |
| | | | TAAGGGTCAGAGTCCTTTGGAGCCCCCAGAGTCCTGTGGACRTGGCCCTAGGT |
| | | | AGTAGGGTGAGCTTGGTAACGGGGCTGGCTTCCTGAGACAAGGCTCAGACCCG |
| | | | CTCTGTCCCTGGGGATCGCTTCAGCCACTAGGACCTGAAAATTGTGCACGGCC |
| | | | TGGGCCCCCTTCCAAGGCATCCAGGGATGCTTTCCAGTGGAGGCTTTCAGGGC |
| | | | AGGAGACCCTCTGGCCTGCACCCTCTCTT |
| PROC | rs2069920 | 32 | TCACATCAAAGGGAGAAAATCTGATTGTTCAGGGGGTCGGAAGACAGGGTCTG |
| | | | TGTCCTATTTGTCTAAGGGTCAGAGTCCTTTGGAGCCCCCAGAGTCCTGTGGA |
| | | | CGTGGCCCTAGGTAGTAGGGTGAGCTTGGTAACGGGGCTGGCTTCCTGAGACA |
| | | | AGGCTCAGACCCGCTCTGTCCCTGGGGATCGCTTCAGCCACYAGGACCTGAAA |
| | | | ATTGTGCACGCCTGGGCCCCCTTCCAAGGCATCCAGGGATGCTTTCCAGTGGA |
| | | | GGCTTTCAGGGCAGGAGACCCTCTGGCCTGCACCCTCTCTTGCCCTCAGCCTC |
| | | | CACCTCCTTGACTGGACCCCCATCTGGACCTCCATCCCCACCACCTCTTTCCC |
| | | | CAGTGGCCTCCCTGGCAGACACCACAGTG |
| PROC | rs2069924 | 33 | CCCCTCAGAGCAGGGTGGGGCAGGGGAGCTGGTGCCTGTGCAGGCTGTGGACA |
| | (at position | | TTTGCATGACTCCCTGTGGTCAGCTAAGAGCACCACTCCTTCCTGAAGCGGGG |
| | 501) | | CCTGAAGTCCCTAGTCAGAGCCTCTGGTTCACCTTCTGCAGGCAGGGAGAGGG |
| | | | GAGTCAAGTCAGTGAGGAGGGCTTTCGCAGTTTCTCTTACAAACTCTCAACAT |
| | | | GCCCTCCCACCTGCACTGCCTTCCTGGAAGCCCCACAGCCTCCTATGGTTCCG |
| | | | TGGTCCAGTCCTTCAGCTTCTGGGCGCCCCCATCACGGGCTGAGATTTTTGCT |
| | | | TTCCAGTCTGCCAAGTCAGTTACTGTGTCCATCCATCTGCTGTCAGCTTCTGG |
| | | | AATTGTTGCTGTTGTGCCCTTTCCATTCTTTTGTTATGATGCAGCTCCCCTGC |
| | | | TGACGACGTCCCATTGCTCTTTTAAGTCTAGATATCTGGACTGGGCATTCAAG |
| | | | GCCCATTTTGAGCAGAGTCGGGCYGACCTTTCAGCCCTCAGTTCTCCATGGAG |
| | | | TATGCGCTCTCTTCTTGGCAGGGAGGCCTCACAAACATGCCAT |
| PROC | rs5937 | 34 | CTATGCCCATATGACCAGGGAACCCAGGAAAGTGCATATGAAACCCAGGTGCC |
| | | | CTGGACTGGAGGCTGTCAGGAGGCAGCCCTGTGATGTCATCATCCCACCCCAT |
| | | | TCCAGGTGGTCCTGCTGGACTCAAAGAAGAAGCTGGCCTGCGGGGCAGTGCTC |
| | | | ATCCACCCCTCCTGGGTGCTGACAGCGGCCCACTGCATGGAYGAGTCCAAGAA |
| | | | GCTCCTTGTCAGGCTTGGTATGGGCTGGAGCCAGGCAGAAGGGGGCTGCCAGA |
| | | | GGCCTGGGTAGGGGGACCAGGCAGGCTGTTCAGGTTTGGGGGACCCCGCTCCC |
| | | | CAGGTGCTTAAGCAAGAGGCTTCTTGAGCTCCACAGAAGGTGTTTGGGGGGAA |
| | | | GAGGCCTATGTGCCCCCACCCTGCCCACC |
| PROC | rs2069931 | 35 | AGCATAATCTATGGCCAGTGCCCCCGTGGGCTTGGCTTAGAATTCCCAGGTGC |
| | | | TCTTCCCAGGGAACCATCAGTCTGGACTGAGAGGACCTTCTCTCTCAGGTGGG |
| | | | ACCCGGCCCTGTCCTCCCTGGCAGTGCCGTGTTCTGGGGGTCCTCCTCTCTGG |
| | | | GTCTCACTGCCCCTGGGGTCTCTCCAGCTACCTTTGCTCCAYGTTCCTTTGTG |
| | | | GCTCTGGTCTGTGTCTGGGGTTTCCAGGGGTCTCGGGCTTCCCTGCTGCCCAT |
| | | | TCCTTCTCTGGTCTCACGGCTCCGTGACTCCTGAAAACCAACCAGCATCCTAC |
| | | | CTCTTTGGGATTGACACCTGTTGGCCACTCCTTCTGGCAGGAAAAGTCACCGT |
| | | | TGATAGGGTTCCACGGCATAGACAGGTGG |
| PROC | rs777556 | 36 | TTCCCTCCTTATTTCATCTTCATTCCTGGAAAGTATTTTTGCTAAATTTAACA |
| | | | AAATTCTAGGTTTGCAGTTAGTAGATTCTATTGTTTCTGTTGTGAAGTCAGCT |
| | | | GTTAGTCTAATCATTACTTTTCTGAACGTATTTTTTTTCCCTTGTGGCTGCTT |
| | | | TTAGACTTTCCTATTTTCGTTGGTTTCTTGCAGTTTTATTAYGATGTAGTTAG |
| | | | GTGTAGATTTCTTTTTGTTTATCTTCCTTGCAATGTGTACAACTTCGAGAATC |
| | | | TATGGTTTAGGTATCATTTCCTTTTAAAATACTGCTTCTGCTATACATGTAAC |
| | | | TTTCCCTCTCCTTTCATTATTCCAATTTTTGCGAAAACTTTTCGATGTATAGT |
| | | | CTATATCTTTTCCTTTCTTCTGTAATACTGTATCTTGAAGCTTCATTCCAGAT |
| | | | CTCTCCTTCTAAGCCATCTTCCAGTT |
| PROCR | rs1033797 | 37 | CTCCTTTCACCAAGTACTCAAAGTAGGAGTCCACGCCAGCCCCGATGCCTGCG |
| | | | TCCTGGGCCACCCACTTGCCAGTGAGCACATCAATGTGGTTGCCGACCTGAGA |
| | | | GAGAGAAAGACACACGGTCCCAACGGGAAGGCCGATGGCCAAAGAAGGATCTA |
| | | | CTCACCCCCAACCCTGACTGCCCAGGGAGATGCAGGGCAGGYGCCCCAGTGCT |
| | | | TCTTGGGAAACATGCAGACCCTGAGAGGGAAGGGCAATGCTGGATCATGGCCA |
| | | | GCCTTCCTGTACATCTGCATAGTAGAGATGCATCTCATGCACATTTATGAGGA |
| | | | CTTAATTATACACATTGAGCAAAAAATGAAAAAGAAAAATGATTTGGAGTGTT |
| | | | TATGTCCTGCCTAGAGTGAGTGTGAGATG |
| PROCR | rs1033799 | 38 | AAAAAGAAAAATGATTTGGAGTGTTTATGTCCTGCCTAGAGTGAGTGTGAGAT |
| | | | GGGAGATGAGAATTTGCTGTTGCCGCAATCTGTCTGATTTCTCAGCACCCAGC |
| | | | ATGTGATTCCACTATCTGAAGACACAGACGTGCTTTACGTATTTCCATAAATT |
| | | | AACTCAATAAGAACATCCACCAAGAAGCTGACAGAGTGGTTMTAAGGAGAGAA |
| | | | ACCGAATAGCTGGAGACAGGGGCAAAAGGGGACTTCACCAATGTCACTGAGTA |
| | | | CCCTTTTTTGTATCCTTTGACTTTTTTTTTTTTAATTGTTCAGTCTCTGTAGA |
| | | | GACTGTGAAAAATTGGCAATGCCGGCCAGGCGCGGTGGCTCATGCCTATAATC |
| | | | CCAGCACGTTGGGAGGCTGAGGGGGGCAA |
| PROCR | rs2295888 | 39 | CTGCCGTACAGGTGACAGGGGTCTCTCCTGGGTTCACGCCATGAAGTAAGTTC |
| | | | ACTGTTCCATATGGCATGCCAGTGGGGGTCTGAAAGGCTGAACAATCGACAAA |
| | | | TTATGATCCCGGACAGGAGCAGGGGGATAGGGATAGTTCTGATACACGCCCAA |
| | | | AGCCTGGGACCTTAGCCAGCACTTCCCTCTTTCTCCTGGGTRTCCTGCTAGAG |
| | | | TCTGAGCCAGAGAAAGATAAATGTCATAACTGGAGGGCCCTGAGCAGCCACCC |
| | | | AGCCCAGATGCTGTCAAACACTGCTCTGCATAACCTTGGGTTCCTGCTCATCA |
| | | | TGAGGGGGCAGGGAGCAGGCTGTGCTCCACACACACTCGCTTTAGCTAGAGAG |
| | | | CTTTACCTATTTTTATTTATTTTACACTA |
| PROCR | rs867186 | 40 | CTGGGGGTTTGGGACAGAACACACGCAGCTTCAGTCAGTTGGTAAACGGGTCC |
| | | | CTTTCCTCTGGGGCAGAAACGCTTTGGGGTTTGACTCAAATCATGGACTCCTT |
| | | | GGGGGCCTATTCTTCGGGCTAACTCTTTGCATGTTCTGCAGGGAGCCAAACAA |
| | | | GCCGCTCCTACACTTCGCTGGTCCTGGGCGTCCTGGTGGGCRGTTTCATCATT |
| | | | GCTGGTGTGGCTGTAGGCATCTTCCTGTGCACAGGTGGACGGCGATGTTAATT |
| | | | ACTCTCCAGCCCCCTCAGAAGGGGCTGGATTGATGGAGGCTGGCAAGGGAAAG |
| | | | TTTCAGCTCACTGTGAAGCCAGACTCCCCAACTGAAACACCAGAAGGTTTGGA |
| | | | GTGACAGCTCCTTTCTTCTCCCACATCTGCCCACTGAAGATTTGAGGGAGGGG |
| | | | AGATGGAGAGGAGAGGTGGACAAAGTACTTGGTTTGCTAAGAACCTAAGAACG |
| | | | TGTATGCTTTGCTGAATTAGTCTGATAAGTGAATGTTTATCTATCTTTGTGGA |
| | | | AAACAGATAATGGAGTTGGGGCAGGAAGCCTATGGCCCATCCTCCAAAGACAG |
| | | | ACAGAATCACCTGAGGC |

The Sequences given in TABLE 1C (SEQ ID NO:1-40) above and in TABLE 1D (SEQ ID NO:41-243) would be useful to a person of skill in the art in the design of primers and probes or other oligonucleotides or peptide nucleic acids for the identification of protein C pathway associated gene SNP alleles and or genotypes as described herein.

TABLE 1D below shows the flanking sequences for a selection of protein C pathway associated gene SNPs in LD with the tagged SNPs in TABLE 1C (unless the LD SNP is already in TABLE 1C), providing their rs designations, alleles and corresponding SEQ ID NO designations. Each SNP is at position 201 of the flanking sequence, unless otherwise indicated, and identified in bold and underlined.

TABLE 1D

| | | | |
|---|---|---|---|
| | | SEQ | |
| | | ID | |
| GENE | SNP | NO: | FLANKING SEQUENCE |

| | | | |
|---|---|---|---|
| FGB | rs2227412 | 41 | CTGATGTCTCAGCTCAAATGGAATATTGTCGCACCCCATGCACTGTCAGT |
| | | | TGCAATATTCCTGTGGTGTCTGGCAAAGGTAACTGATTCATAAACATATT |
| | | | TTTAGAGAGTTCCAGAAGAACTCACACACCAAAAATAAGAGAACAACAAC |
| | | | AACAACAAAAATGCTAAGTGGATTTTCCCAACAGATCATAATGACATTAC |
| | | | RGTACATCATAAAAATATCCTTAGCCAGTTGTGTTTTGGACTGGCCTGGT |
| | | | GCATTTGCTGGTTTTGATGAGCAGGATGGGGCACAGGTAGTCCCAGGGGT |
| | | | GGCTGATGTGTGCATCTGCGTACTGGCTTGAACAGATGGCAGAACCACAG |
| | | | ATAGATGTAGAAGTTTCTCCATTTTGTGTGTTCTGGGAGCTCATGGATAT |
| | | | TCCAGGACACAAAAGGTGGAGAAGAGCTTTGTTCATCCTCTTAGCAGATA |
| F2 | rs3136512 | 42 | GGAGATTTGGATAAAAGCAACTATCATTATTATCCTCATCAGACTTGTAG |
| | | | GTCTAACTTTTTAATTTTTTAATTTTTAATTTAAATTTTTTTCTTGGTCT |
| | | | TTTATCATTAATTAATTTTTTCGAGACAGGGTCTCACTCTGTTGCCCAGG |
| | | | CTGGAGTGTGGTGACATGATCACGGCTCACTGCAGCCTTAACCTCCCAGG |
| | | | YGCAAGTGATCCTCCTCTCTTAGCCTCCCGAGTAGCTGGGACTCCAGGCA |
| | | | TGTGCCACCATGCCCAGCTAATTTTTTGTAGAGAGAGGGTTTTGCCATAT |
| | | | TGCCCAGGCTGGTCTTGAACTGCTGAGCTCAAGTGATCCACCCGGCTTGG |
| | | | GCATGAGCCACCTCCCCTGGTCTGGTCCAACTTTTTAAAAGCATTATTCT |
| | | | GCCTGTTGGGTGGAGAATAGACTGTAGGTGGGCAAAGAATGAAGGAAACT |
| F2R | rs37245 | 43 | AAATACAAAATTAGCTGGGTATGGTGGTGCACACCTGTAGTCACAGCTAC |
| | | | TTGGGAGGCTGAGGCACCAGAATTGCCTGAACCTGGAAGGCAGAGGTTGC |
| | | | AGGGAACTGAGATTGTGCCACTGCACTCCAGTCTGAGCAACAGAGTGAGA |
| | | | CTCTCTCTCAAAAAAAAAAAGAGGTGGAATTGGGAGTTGACCACAGGCCT |
| | | | RTCTCTCCGAAGTGCAGGCTTTCTCTAACACCCCCTATAGAAAGGAAGCC |
| | | | ATCTAGACTCCCAGCACCTCTTACAGTAGAGAAGTAACCCCACTGTGCTC |
| | | | CCTAGTACAGTATGGATTTACCTATTTTTGATAATTCATCAAAATATAGA |
| | | | AGCAAAGTCTGTGCCCTATCGCCTTGGTAGCTCAGGCCCAGCACAGGGAG |
| | | | GTATTTAGTGAGCATTTATGCA |
| F2R | rs2227744 | 44 | GTAATGGGTTAAAATGATAAATTGTAAAATCAATGACGTCTTAGGAATAA |
| | | | TGAAAAATAGTTTAATAGTGAATGAAGAACTATGTAATTTTAACTGTTCA |
| | | | CATTTACTCTTGGGTATGTTTCCAGAGGATAACTGAACGGGGATAGATTT |
| | | | TAAAAAGCTTTATTTAACTGGGTACTTCCGCAATTTAGTGATCAACTTCT |
| | | | RTGTACAACAAGGTACTGTCCTTTGAGGATGATGGGAGAATACAGGGAAG |
| | | | AACGAAATCGCCTCTGATCGTACTTTCTCCACGGATGTAAGTGTCCGGGC |
| | | | TCTAGTGGGGGAATGATACTCTTCGTGCGAAATTCACTTTTAAAAAAGGC |
| | | | TTAGAAAACTGACCACCGGCTCTCAGCTGCAGCTTATCAACCACAGAACT |
| | | | C |
| F2R | rs27135 | 45 | TTTGTTGGAAGTTTTTTTCTTGCACATTTTACAGGCGAGAAAAGTGATGT |
| | | | AGAGAAAAGCCCAGGCAGTCCCTTGGCATGTTTAGCAGAGAATCAGTACC |
| | | | AGCAGCCCCCGGCCCGGCCTTGTGTCCAGGAGGTGCGCAGGGTGCGAGAT |
| | | | ATATGGTGACAATAGCAGAGGCTCCGCGTGGTGGCGGGGGAGGGGACATG |
| | | | RAGAGGATTTTGTTGTTACCTAGAACCCATTCCTTCTAAGTGAGTTGAAG |
| | | | AGAGAGATCCCTCCCCAGGATCGGGCTCCCTCGAACACTGTGGGATCCCA |
| | | | GTATTTCTTAACGAGATTTCTGATCCACTGCAAGAAGGTTGCTCCCCTAG |
| | | | AATATTTTCCCCACTAGTAGTCTATTTTTAAGTATCTGGCCACTTGACCA |
| | | | AATAAATAAATTTGATTAATTTATTTGGTCAAATATTTTCTGTATCCCTT |
| | | | TCCCCA |
| F2R | rs37243 | 46 | TAAAATCACTAATAAAATCAACCCAAGATAGGTTTACTTTCTATTACCAC |
| | | | CATGCATTGACAATTCTAAGCATTGTCTGTCATAAGGTAGTGGTGCTGGG |
| | | | ACTCTGAGGCATCCAACAGTGCCTCCCATCCTTGAACCGCCACCGCTGTG |
| | | | ATAGAGTTTATTGTCCAGGATTACCAGCTCTGTGTGCCAAGAGGGGCGGT |
| | | | RAAGCCCTCCCAGGGCTGGCCCTGACCACCAAGCTGAGCCTTCCTCCAGC |
| | | | ACTTCCCGACTTTTTGTTCCCACTCATTTTGGCATTTCCTGCCTTGTCAC |
| | | | TTTGTGTGTGTGTCTCATTCTCCCAACTAAGATTATAAAGTTTTATTTAT |
| | | | CCCCATGGTGACTAAAACAAATGTTCACTCAGCAGATAGTTGTTGAGAAA |
| F2R | rs27593 | 47 | CCTTGGCATGTTTAGCAGAGAATCAGTACCAGCAGCCCCCGGCCCGGCCT |
| | | | TGTGTCCAGGAGGTGCGCAGGGTGCGAGATATATGGTGACAATAGCAGAG |
| | | | GCTCCGCGTGGTGGCGGGGGAGGGGACATGGAGAGGATTTTGTTGTTACC |
| | | | TAGAACCCATTCCTTCTAAGTGAGTTGAAGAGAGAGATCCCTCCCCAGGA |
| | | | YCGGGCTCCCTCGAACACTGTGGGATCCCAGTATTTCTTAACGAGATTTC |
| | | | TGATCCACTGCAAGAAGGTTGCTCCCCTAGAATATTTTCCCCACTAGTAG |
| | | | TCTATTTTTAAGTATCTGGCCACTTGACCAAATAAATAAATTTGATTAAT |
| | | | TTATTTGGTCAAATATTTTCTGTATCCCTTTCCCCAAGAGCAGCACAGAT |
| | | | GAGTTGTTTTTAGCCTGTAAAGGCGCTAATTAGAAAGTGAGAAAAGTGTT |
| | | | TTTGAA |
| F2R | rs37242 | 48 | CATGGCAGGAGTGGTGCATGTTAATATGGACAGTGCTGGTGTAGACAGAA |
| | | | AGGCAGGTGGATGAACTTGGCTAGTTTATCAACACTGGATTCTGGAACCA |
| | | | CTTTGGGAGGGAAAGAAGAAAGGAGTATGATAGAGGAAAAGGAGCGCTTG |
| | | | CTAAGTGCCATATTCCATGTCAAGCCCTGGGCCAGAAGGAATTTTCACTT |
| | | | RGATTGTCTCATTTCACCTTGTCAAAACACCTTGTTAAGGTGGGTATTTA |
| | | | TCCCCTTTTGCTGATTCTGCAACTAAGACCCAGAGACAGCGGCTAAGCAA |
| | | | GTGGTGGCGGGTGGGGCAGGGAAGGGGCAGTCCACCCACCCTGGGTGCAA |
| | | | GCAATTAGGAATAAGTGGGGCTTTGTCTTTAGAAAATTTAAAATCACTAA |
| | | | TAAAATCAACCCAAGATAGGTTTACTTTCTATTACCACCATGCATTGACA |
| F2R | rs253061 | 49 | GCTAGTTTATCAACACTGGATTCTGGAACCACTTTGGGAGGGAAAGAAGA |
| | | | AAGGAGTATGATAGAGGAAAAGGAGCGCTTGCTAAGTGCCATATTCCATG |
| | | | TCAAGCCCTGGGCCAGAAGGAATTTTCACTTGGATTGTCTCATTTCACCT |
| | | | TGTCAAAACACCTTGTTAAGGTGGGTATTTATCCCCTTTTGCTGATTCTG |
| | | | MAACTAAGACCCAGAGACAGCGGCTAAGCAAGTGGTGGCGGGTGGGGCAG |
| | | | GGAAGGGGCAGTCCACCCACCCTGGGTGCAAGCAATTAGGAATAAGTGGG |
| | | | GCTTTGTCTTTAGAAAATTTAAAATCACTAATAAAATCAACCCAAGATAG |
| | | | GTTTACTTTCTATTACCACCATGCATTGACAATTCTAAGCATTGTCTGTC |
| F2R | rs37244 | 50 | ATGCCCTCCCCATATCCCATACCCGCCACGTTCATGTTTAATTAAAAACA |
| | | | GCTACCCTCTGTGGAGTACTGACTACAGCTGACATCCTTCTTAGGGACGT |
| | | | TACAATACTATCTTATTTATTTCTCACAACAGCCCTTTGAGTAGATGTCA |
| | | | TCCTCATTTTACTGGTTATAAAACAGAGACCCAGAATGGTTAAGTCACAA |
| | | | KTTGAGAAAGAGGTGGAATTGGGACTGGGTGCGGTGGCTCATGCCTGTAA |
| | | | TCCCAGCACTTTGGGAGGCCAAAGCAGGGGGATCACTTGAGGCCAGGAGT |
| | | | TTGAGACCAGCCTGACCAACATGGTGAAACCCTGTCTCTACTAAAAATAC |
| | | | AAAATTAGCTGGGTATGGTGGTGCACACCTGTAGTCACAGCTACTTGGGA |
| | | | GGCTGAGGCACCAGAATTGCCTGAACCTGGAAGGCAGAGGTTGCAGGGAA |
| | | | CTGAGATTGTGCCACTGCACTCCAGTCTGAGCAACAGAGTGAGACTCTCT |
| | | | CTCAAAAAAAAAAAGAGGTGGAATTGGGAGTTGACCACAGGCCTGTCTCT |
| F2R | rs37246 | 51 | AAGTGCAGGCTTTCTCTAACACCCCCTATAGAAAGGAAGCCATCTAGACT |
| | | | CCCAGCACCTCTTACAGTAGAGAAGTAACCCCACTGTGCTCCCTAGTACA |
| | | | GTATGGATTTACCTATTTTTGATAATTCATCAAAATATAGAAGCAAAGTC |
| | | | TGTGCCCTATCGCCTTGGTAGCTCAGGCCCAGCACAGGGAGGTATTTAGT |
| | | | RAGCATTTATGCACGGACTGTGGTATTCTCTCATTTACTTTCGCTAACAG |
| | | | ATGATAAGGCAGGCTCTGAAAAGATCCCTGCTCATGAATACACTAATTAA |
| | | | TCAGATGTTACAAGAGATATTGCTAGTAAACCTAAACAGAAAGACAGAAA |
| | | | ACTGAGCAGTGGTTCTACCGTAAGCAGACCAGAAAGCTCTATAAAGCCTG |
| F2R | rs37249 | 52 | GCACCTGCCACCACGCCTGGCTAATTTTTGTATTTTTAGTAGAGACGAGA |
| | | | TTTTATCACACTGCCCAGGCTGTTCTCAAACTCCTGGGATCAAGCGATCC |
| | | | ACCTACCTTGGCCTCCCAAAGTGCTGGAATTCTAGGCGTGAGCCACCATG |
| | | | CCCAGTCTTTAACTAGTTTTCGTGAGCACCTAGGCTCCCCTTCCATTGCG |
| | | | RATACTCACAAAAACATCCTTGTTAGAAGAGTTATTAGGACTCAGGGCCT |
| | | | TGGTTTATTTTTGACTATGATACTAGTGTTGAGGACTCCATAGTTTTACC |
| | | | ATTCATAATTTTCTGTTTGTTTCCTTTTTTGCGATTTCTTTCATTCTGCC |
| | | | TTTTTCTTTCTCTTGCTTGTGCCTAAAACTGTCGTCATAAATAGCTCTGT |
| F3 | rs3917615 | 53 | CTAAAAGAAAGATATTTAACAAAATGGTTGAGTACAGATCCAAGAGTCAA |
| | | | ATAGCTGTCTGGTTCAAAGTCCAGCTGTGTGATTTTGAGCTAGTCACCCA |
| | | | ATCTCACTTTGTCTCAGTAGCCTTATTTGTAAAAACAAGGCAAATTACAG |
| | | | AGCCATCCCCTGGGTTGCTATGAGGACTCAAACATGCATCCCAAGTGCTC |
| | | | RGTGTTGCTAGGTATGATGGCTCACACCTGTACATTCAGCACTTTGGGAG |
| | | | GCCGAAGCAGAAGGATCAGCCTGGGCAACATAGCAGGACCCCATCTCTAC |
| | | | AAAACAATGTTTAAAAAAAAGCAAAGTGCTCAGCACAGTGACTGCATCAT |
| | | | TAGGATTGATTGTAGGGCTCCTGATGTTAGCACAGAACACCACAGCCAGG |
| | | | AAGCAGTCTATCTTGTTGGGTGCAAATTGTAACATTCCATTTATGTTTCT |
| F3 | rs841695 | 54 | AGATGACGAGGATGAACACCTTTATGATGATCCACTTCCACTTTATCAAT |
| | | | AGTAAATATATTTTCTCTTCCTTATAATTCTTTCTCTTCCTTCCTTCTTT |
| | | | CTTTTCTTTTCTTTTCTTTTTTTTCTTTCTTTCCCTTTCTTTTTTAGACA |
| | | | GAGTCTCGCTCTGTCACCCAGGCCGGAGTGCAGTGGCGCAATCTCAGTTC |
| | | | RCTGCAACCTCCTCCACCTGGGTTGAAGTGATTCTCCTGCCTCAGCCTCC |
| | | | CAAGTAGCTGGGATTACAGGCACCCACAACCACGCCTGGCTAATTTTTGT |
| | | | ATTTTTAGTAGAGATGGGGTTTCACCATGTTGGCCAGGCTGGTCTTGAAC |
| | | | TCCTGACCTCAAGTGATCCACCCGCCTTAGCATCCCAAAGTTCTGAGATT |
| | | | ACAGGCACGAGCCACCATGCCCAGCCTCTTTTCCTTATAATTTTCTTAAT |
| F3 | rs2794470 | 55 | ATGATGATCCACTTCCACTTTATCAATAGTAAATATATTTTCTCTTCCTT |
| | | | ATAATTCTTTCTCTTCCTTCCTTCTTTCTTTTCTTTTCTTTTCTTTTTTT |
| | | | TCTTTCTTTCCCTTTCTTTTTTAGACAGAGTCTCGCTCTGTCACCCAGGC |
| | | | CGGAGTGCAGTGGCGCAATCTCAGTTCACTGCAACCTCCTCCACCTGGGT |
| | | | YGAAGTGATTCTCCTGCCTCAGCCTCCCAAGTAGCTGGGATTACAGGCAC |
| | | | CCACAACCACGCCTGGCTAATTTTTGTATTTTTAGTAGAGATGGGGTTTC |
| | | | ACCATGTTGGCCAGGCTGGTCTTGAACTCCTGACCTCAAGTGATCCACCC |
| | | | GCCTTAGCATCCCAAAGTTCTGAGATTACAGGCACGAGCCACCATGCCCA |
| | | | GCCTCTTTTCCTTATAATTTTCTTAATAACATTTTCTTTCCTCTAGCTTA |
| F3 | rs1144300 | 56 | TTAATAATATTATTAATAGTGGTCATGAGAGAATATATGTATAACATGTT |
| | | | ATTATGTAGACTCACTATATAGACTCTATTCTACATAGAATATAGAACAT |
| | | | TATATAACAAACAACTATAATAAGTAGACTATAGTAAACAACCTCACTTT |
| | | | GTCTCAGTTGCCTCATCTTGATGGAAAACTGCTCTTTCTCTCCTGTTACC |
| | | | YTGACAGAGAGCGTCTACATTCTAAAAGAAAGATATTTAACAAAATGGTT |
| | | | GAGTACAGATCCAAGAGTCAAATAGCTGTCTGGTTCAAAGTCCAGCTGTG |
| | | | TGATTTTGAGCTAGTCACCCAATCTCACTTTGTCTCAGTAGCCTTATTTG |
| | | | TAAAAACAAGGCAAATTACAGAGCCATCCCCTGGGTTGCTATGAGGACTC |
| | | | AAACATGCATCCCAAGTGCTCGGTGTTGCTAGGTATGATGGCTCACACCT |
| | | | GTACATTCAGCACTTTGGGAGGCCGAAGCAGAAGGATCAGCCTGGGCAAC |
| F3 | rs841697 | 57 | TCAGAACATTTCCATGGAATGAATATCACCGGTGACGGTTTGTGCTAAGG |
| | | | CTTAAGCCAATAACATTTCCCAACCACCACTGAAAACTGTTAGCAAAGGT |
| | | | GAAAAATGCAGTTGGAGTTCCAAGTAGGGGCTTCTGCACAGCAGTAGTGT |
| | | | CCTGCGGCTGGAGCCAGGCTGCAGTAGTGAGAGCAGTCGGGAGGGAAGAG |
| | | | RGGCAGCTGCTTAAGATGCTAACTGTAGGGAGGGAAAACAGGCAGAGAGG |
| | | | AAGGCCAACTGAGGAGATGCAGTGGGCAAGACTTTCCTTCTTCCTCCCGC |
| | | | TTTGGAGCCTCCCATCAGACTGTGGCAGAGCCACCTGAGGGATGGTGGTG |
| | | | TGTGGATACTGGGTAGACTTTGGTTCCAGACCTGACATGGGCACTCACCA |
| | | | TCAGTGTAGTCATGAATAAATCCCTCACTTCTCAGAGCAACAGTTTCCTC |
| F3 | rs762485 | 58 | TTCAGATTTCACCAATTGAGAATTAGTAAGTAATTTCTCTGATACAGGCC |
| | (at position | | TGAAGTTTACCTTAGTAAACACTTTACTTCCATATGGTAAAAATTAGATT |
| | 648) | | TTGGGAGGAATGCTTACCTCCTAAATATATTCAATCTAATATTTGAGGAC |
| | | | ACATGGGAATATATTTATGATTCATCTGCTTTTTAAACATAAGCCTTTGT |
| | | | TAACTGTAAGTTCTTGAACTTTATAAGGCTGCTGTTATTTAAATGAGCAC |
| | | | AGCTCCTGATCTGCAAACAGCAGAGCGCAGGGCTACAGCTTGGGGGATGC |
| | | | CAGCCGACTCAGGGTGGTCCTGTGGACTGAACAATCTCTTGCTGCTGTAC |
| | | | TGGAGGGCCTGGGAGCTTTTCCATCAGCCTCGGCCTGAGGTGTGCACTCT |
| | | | TCTCCTGCCCACCCCAGGAATAAATGAGATTCCTGGTTAAAAAGGACCAG |
| | | | AGCAGTCATTTTACAGTTGAGGAAACTGTTGCTCTGAGAAGTGAGGGATT |
| | | | TATTCATGACTACACTGATGGTGAGTGCCCATGTCAGGTCTGGAACCAAA |
| | | | GTCTACCCAGTATCCACACACCACCATCCCTCAGGTGGCTCTGCCACAGT |
| | | | CTGATGGGAGGCTCCAAAGCGGGAGGAAGAAGGAAAGTCTTGCCCACKGC |
| | | | ATCTCCTCAGTTGGCCTTCCTCTCTGCCTGTTTTCCCTCCCTACAGTTAG |
| | | | CATCTTAAGCA |
| F3 | rs696619 | 59 | GAGGTGGGGAGGAATCCCAATGTATACATTGCCCTTAAGCAGTGTTTGAT |
| | | | TCATTCATCTTTGGACTCCATGAATCGAAATCTGGTAGAATACATGATCT |
| | | | TAGTGGAGGAGGCCAAATGCGTGACTCACTGAGCCTGGCAGAGCAGAAAT |
| | | | ACTCTGCTGTCTGCACCCTCTGGGTCTGGTGTGGCTCTGCTTCTTGGTGC |
| | | | YTCAACTCTGACTGGCAGCTGTCCCCAGGAGGCGATAATTCAGCATGTTC |
| | | | AATCTAAAGGTTATGACTTCCTTGATGGTTTTCACCATATTCTTGGCAAG |
| | | | TTTTTGGTTTTTGAAATGTTCTAGGAGGCTTGGTAGAGATCTTATGAAAT |
| | | | AGAGAATAGCTGCTGTGGAAATTATTTTAATGCTAATTACATAAAAGTAC |
| | | | AAAAGTAGCACTAGCTAAAACAAAAGGTATTTTGCTGTTCTGTTTTGTTT |
| | | | TAGCTTGTGCCAGGCCTTTTACAGCATTAGGA |
| F3 | rs3761955 | 60 | TGTTTCCTCTCTCCTTCTTTCCCACGTTTTCCCAGGGAAGTCAGTCTTGC |
| | | | ATTTTAATGCATACTATATACATATCTCGTTTAGCTTACTGAACCACTTG |
| | | | TTTTAACAGAATAAAACTGTGCAAAATTTTAATTTTCCTCCTTTGCCTGA |
| | | | ACTGAAATAGCACATCCAGGTTTAGCCCTTGTAGACTTTCCTTCCTCGAA |
| | | | RCAGAAAGTTGCCCTTGATGATTTCCTCTTTGAGCTCTCTGCCAGCTCTG |
| | | | AAACCCACAAAATTTATGTTTGCAAAACTAAGCCATGCAATCCTCTTTTT |
| | | | ATGCAGGCTCTAGCCTGAGTCATTTTCCCTAAGAGATCTTCAGCTCCACC |
| | | | TGGGATGTGATTCTTTGCTCTCTGGGATTGAAGGTAGCTGAAGAGAAATA |
| | | | GTTACACTTCAGGTTTGTTACAAGACCCAAGAAATTGTTGCAATTCCACT |
| | | | TGGAG |
| F3 | rs958587 | 61 | GCCACAGGGTTCTCATCCATAAAAGAATGTCTGTGAGGTTCTCCCATCCT |
| | (at position | | CTGACATCCTAAAATCCAATGAGAAAGGGACTGGTCAAGCCAGAGAGATT |
| | 472) | | ATTGTTATAGTTTAGTAACTTTTTGAACTTCTCAGAGCCTCCAAGATAGA |
| | | | TCATGGAGGAGGGAACTGTTAACTGCTAAGCTTGACTTTACTGACAGGAG |
| | | | TAAAAAAAATTGTGTTAAGGTTAGGGAATAATTTTAACAGTCAATTTGTT |
| | | | CTTGTGAACAAATTTCAACAGTGAAATTTTAGATATGTACTTTTTAATGG |
| | | | TGCCAAGCAGCAGTTATTATAGATCAACTGCTGTTTGGCACCATTAAAAA |
| | | | GTACACTTCGCACCGTCAAAAAGTAGATCTGGCCACAATTAGATCAGTCA |
| | | | GGGAAAAACACTTCGCAATGAAATATTATTTACCACGTTTTCTTCCTCCC |
| | | | TCTTCTTGAAAATAGTAATGAYTTTAGCATTTTTAAATCTTGAAGAATGT |
| | | | CATTCCGTACTGACTAAAAAGCCTGTGCAAACACCCAACATCTTCTCTTT |
| | | | CCTGTCTATTT |
| F5 | rs2187952 | 62 | ACATTCTCTTTTGCTCTTAACGGAATGGAAATCTTAGAAATGTTGATGGG |
| | (at position | | ACAATGACATGAATCATGAAAAGAAAGGAATAGTGGGAATACRAATATTA |
| | 93) | | GAAAGCCAATGTTTTGTGGATGTTTGAAACTCTCATATACATTCATAGGG |
| | | | CTTACCCCACTGGTATGGGCAACAGGTAACTCTGGTAGTTTCTAAAGATG |
| | | | CTCCCAGTGAATTATGCCTCCTGGTATGTGGTCCTTATGCAGTTCCCTTC |
| | | | TACACTGAATCGAGACTGGCCTGTAATTTGTGTTCACAACGAATAGAATG |
| | | | CAGCAAAAGCAAAGCCCTGTCTCCTAGAGATTCTGGTATGTTGTCTCTTT |
| | | | GTTCTCATTGGTTTTAAGGAACTTATTTATTTCTGCCTTAATTTCATTAT |
| | | | TTACCCAGTAGTCATTCAGGAGCAGGTTGTTCAGTTTCCATGTAGTTGTG |
| | | | TGGTTTCGAGTGAGTTTCTTAGTCCTCAGTTCTAATTTGATTTCTCTGTG |
| | | | GTCTGAGAGATTGTTATGAATTTCGTTGATAAAATTTTATATAT |
| F5 | rs2040444 | 63 | ATTTCTGCCTTAATTTCATTATTTACCCAGTAGTCATTCAGGAGCAGGTT |
| | | | GTTCAGTTTCCATGTAGTTGTGTGGTTTCGAGTGAGTTTCTTAGTCCTCA |
| | | | GTTCTAATTTGATTTCTCTGTGGTCTGAGAGATTGTTATGAATTTCGTTG |
| | | | ATAAAATTTTATATATTTATGGTATACATAACATTTTGATATATGTACAC |
| | | | RTTACAGGATGATTAAATCAAGCTAAGTAACAAATCCATAATCTCACATA |
| | | | AATTTTTTTTGGTTGCTCTTTTAACACTAAGTTTTGGGGTGGTTTATTTC |
| | | | AGACACCCCAAATGACTGTCTATCTCATGTGATTTTAAGGATGTCTAAAG |
| | | | GTTCCCCAGTTGTGCAATATCTACAGGATCACTGAATGCCAAGTCCCCAG |
| | | | GGAAAGGAATGATGAAAGGGGAAGTTGCTGGAAGAAGAGAGAGGAGGAAG |
| | | | TTGAGGCCATAGAGAGGAAGGCCCTGAAAGAAAACTTTAACTGCTTGCCA |
| | | | GTTTGGCCAGAGGTCTCTTTGAGCAGGAACAACTGCATTTAGACCAGCAG |
| | | | TTCCCATGCTCTGTTTTACAGGTCTGAGCTTTCCAGTAGGTGAAATTATG |
| | | | TTTTGAAACTGTGTGCCATGTAGTACCAGCTAGAATAAAGCCAACATTAC |
| | | | ACATTCAGTTCTACCATGGTTATTTCAGTTCTGTTCCATATCTAATGACC |
| | | | ACCAACCTTGAATATCAATGTGTGCAGTCCTTAGGGAGACCAGGACGGAT |
| | | | TCACAATTTCAATGGGGCTACTGGAAAGATGCTTGGCTGTTTTTTTACTC |
| | | | ATGGAAAGTCAGAAAAATCATTGTTATATGGGAAAGACAGGATATTTTAA |
| | | | GTACTTATTTCATTTGATAATATTGTTTTTCTCTTCACTCAAGAAAAACC |
| | | | ATTAAAAAATCATGTGTTTGTGAAAGTTATCCAGGTCTATCAATTATTAT |
| | | | TTAAAGTAATATCTGTTTTACTAGTGTGTAAAGGATTTAAAAGAGTTATA |
| | | | ATGAAGCATTTTAACTACATAAATATTACTTC |
| F5 | rs4656685 | 64 | GGGGTTGAAAAAGTTCTTCACATGTCCTTTGGTATTAGTATTTCCTTCAA |
| | | | AAATCTGAAAGCCAAATAAGAGAAAATCTTTAATGACAACATAAATGGCT |
| | | | AAGGTTTTTTGTTTTTGTNTTTTTTTAGACAGGGTCTTATTCTGTTGCCC |
| | | | AGGCTTGAGTCCAGTGGCACAGTCATGGCTCACTGCAGCCTTAAACTCCT |
| | | | YTCAAGTAATCCTCTTGCCTCAGCCTTCCAAAGTGCTGGCATTGCAGACA |
| | | | TGAGCCATCATGCCCAACCCAGAAAATTTTTTATCCTATTAGCTCAAAAT |
| | | | GAGCATATCAAAGAACACATAGAACACTCTCACAGAGATGATCCTCTATC |
| | | | AGTTATGAAATCACATGCCAGATAGATTTCATCTCACCAGTGATCTGACT |
| | | | TAAATTAGTGA |
| F5 | rs3820060 | 65 | ACCTTATATCCTCAACTAAAGTGTATAACTTTCCCCCTTCCATCATCAGC |
| | | | AAGTTCCCCAAGGTTTACACACACCAGAAATAAAATCACTCTTTTTTTTT |
| | | | ATGACAATGATATTTATCCTCCTATGAGGGCAACCTGGTGTAGTGAGAAA |
| | | | TAAAACAAACCAAAACAGACAACCAGGAGTTTGTCTGAGACCAGGCACCT |
| | | | KAAGAACTAAGATTTAGAAGACTTAAAGAGGTGGTACATGTCACTGCATA |
| | | | TTTGTCAAATGCAAAATACTGTTATTCTCATTATAGCACAGTCTTCAGAT |
| | | | TGCTTTCTCTTTGCCCAGATGCCACTCTACCTTGTCCACCATGGAGGATT |
| | | | TCAGCCTGTATGGTTTCCATTCCACTCCCTGCTCACTGTAGTGGATGGTA |
| | | | TAGCTCTTTACATACATTTCAGAGGACAGAGACTTGCAGCCCTGTGTTAT |
| F5 | rs6670407 | 66 | TGAATGAATAAATGAGCTTTATTTGGAAAAATAATTAAACTGCTAAATTA |
| | | | TCTGTGAATATTATTTTCTTAAGCAAATTTCTTTAAATACTCTGGTAATA |
| | | | TTGCTGCAGGGTTATCTGGGAAATAATTGGATAGCATTCTCTCTCTATTT |
| | | | CACCTGTTTTACAAAACACAACTTCTCTTTCCCCCTGTTAATGTCAAAGA |
| | | | KCAGAAATAGTTCAATTTCTTCTAATATTTCAAATAAATGTAACATTTGA |
| | | | GAGACCTGATAAAACCATAAGTAGAGCTTGATACACAGTACAGTTATTGT |
| | | | CCTGTCTCTGGCCTAACAAAATGCCTAATCACATGGTTGGTGATATGTGT |
| | | | TTGGAGAAAGATCAGAAGGTCTAAACTGAGTTTTAGAGTCATCTAAGATA |
| F5 | rs2420369 | 67 | AATATTGATATGTTTCTTCAACATCACCCACACATACATAAGTGTATGTG |
| | | | TATATATAGGTGTATGTGTATATATGTGTATACACAAAAATTCACATGCT |
| | | | ATATGTATTTTAAAAGCAAATTAACTGAATTAGAATGATTATATGAGAAA |
| | | | ACTTTTAAGTCTATAACATCCTCTAGTGACATCTGGGTGGTATCTTAGTG |
| | | | WGTCTTCATATCAAGGTTGTACCAGTGATGCAGAGTATAACCAGCTAGAG |
| | | | GTTTTCACAGGCATAAAAGAGGTAGAGACATTTTAGTTATGGAGAGAACA |
| | | | GCATCTATGTGCATCACTGCATATGTCCAGCTTTGATTTTCAACCTCTCT |
| | | | AAAGAGACCATATAAAGACATTTCATGTGAATGGAAGGGGGTTAAGTAAA |
| | | | ACACATATTTCTGATCCCGAATTCTAACTTTGAAATGACACCATTAAAAA |
| | | | TTAGCATTTTATCTTTGAGGCCCTTGTTATATCACAAAGACTAAGAGCAA |
| | | | ATTATAGAGAGTGCAAATTTTTTTAAAAGATTGTAAAATGAAGTATGGAG |
| | | | TGACAAGGTTATACCATGAACAAGTCTAAAGAGTCATCTGAAAAAGAATT |
| | | | GGATAACATCTCTTTAATTATTATTTATATTGCTATGTAAATTATATAAA |
| | | | TGTAATATAACACTATTTTTATTATTTTAACTTCTCTGGGTGTCAGCT |
| F5 | rs9332667 | 68 | TCCAGGGCCTATCCTTAAATTAGGCCACTAGAAAGGAAAAAAGAATTGTG |
| | | | GTGCTGGTGGCGTAAATAGAAAAGATTGGATTCCACACAGTCTTGGGAAC |
| | | | TGATATCTGTGTCTTGAAACTCATTCTGGCCCAATATGGAATCACAGAAT |
| | | | GTTACAGTAGCAAGGAGCAAAGCATCTGGTCTAGATTTTTCCTTTTAATT |
| | | | STAAATACACAGGAGATTAAATAATTTTAGCTTAGTTTGGTAGCAGAATC |
| | | | AGGACTAGAATCCCATTCTCCCAGTAAACAGGCCATGCTCCTTCCACCAT |
| | | | TTGAAGCAGCCCAAATACCTCATTTTGCAATTTTGCAGAGGGCAAAGCTG |
| | | | ACACCCAGAGAAGTTAAAATAATAAAAATAGTGTTATATTACATTTATAT |
| | | | AATTTACATAGCAATATAAATAATAATTAAAGAGATGTTATCCAATTCTT |
| | | | TTTCAGATGACTCTTTAGACTTGTTCATGGTATAACCTTGTCACTCCATA |
| | | | CTTCATTTTACAATCTTTTAAAAAAATTTGCACTCTCTATAATTTGCTCT |
| | | | TAGTCTTTGTGATAT |
| F5 | rs9332665 | 69 | TCAGTGATCTAAGTTAAACAAACACCTTCCAGAGAGTTATACTGTCCCTG |
| | | | ATATTAGCCCACTGAGTAATTCAGGTGATTTAATTTGGGGGTAACTCTTA |
| | | | ATATTTGACTCATTTTTATTAATTCTTTAAATGACCTGAGATATCAGAAT |
| | | | GGCATGAATAACTTGATGATCCCTTCAGCCAACTAAATCCAAATTCCCTA |
| | | | MTTTCTATCCTCATATCTCCCTCCCTTAAGATACCTACACTCCAATTTCC |
| | | | TGGCTTTCTATAGAATTCCAGGGCCTATCCTTAAATTAGGCCACTAGAAA |
| | | | GGAAAAAAGAATTGTGGTGCTGGTGGGGTAAATAGAAAAGATTGGATTCC |
| | | | ACACAGTCTTGGGAACTGATATCTGTGTCTTGAAACTCATTCTGGCCCAA |
| | | | TATGGAATCACAGAATGTTACAGTAGCAAGGAGCAAAGCATCTGGTCTAG |
| | | | ATTTTT |
| F5 | rs3766103 | 70 | TCTGTGTTTTATTTGGGAGATGTTTTAGACTACTGCTATCTGGACATTGG |
| | | | CAAAATACCCATATCCATCCAAGGGTATACTGTGCCATTATCTGCTTCAA |
| | | | CAGGAAACTGATTCCAGGTTTCAGCTACTTTCTCCATTGTGAATCATGGT |
| | | | GGCTTCTCTCCACCCAAAGGGAAGTACTGCAACTCCTGACAGGTGTGCCA |
| | | | YGGCAGGTTTCTAGTGCACCTATTTATTGATCCCTCTTCCCACCTCCAGC |
| | | | CCTTCAGCAGCCAAGTGGGGCCTGGATCAGCCAAGCCTTAGATTTATTGC |
| | | | TTCATTCTTTTACCTCAGAATGCCAGGCAGATATTATTTTCTTTGTGTGC |
| | | | TTTGAAGTAAAAAATATTGGAAAGCACTGCTTTAAGAGTCCAAGAAGAAC |
| | | | AGTTAAGACTCTTAAACATCCTTGCTATATATAGTAGAATTTTATTATGA |
| | | | CCATTT |
| F5 | rs2227244 | 71 | CAAAAACACTCATTGAACACCAYATATATGGCAGTGATGTTGCCAGATAC |
| | (at position | | TGTCATGACATTGAAGTTTGAGTGACCTGAGGACTTTGGAAGAGTCAGGC |
| | 23) | | CTAGTTTGAATCTCAGGTAGGTCTTATTGAAAATGGGCTGATGGAGGTAA |
| | | | TTCCAAATTAGAGCCCTTCCTTGGAGAGTTGTGATGTGTCTATATAATCC |
| | | | AGGCACTTTCTTCACAGAGATGCTGTCGGCACTCTGATTGGCAGAACCAT |
| | | | TCTTGGTCTAGATCACACTGAGAGTTTACCTGAGTAGAACCTCTGTTTCA |
| | | | CAAAGGTTTTCCTAGGAGCCTAAGTCACTGAAAAGAACTAAAAATTCTAC |
| | | | TCATTCTCCTATACCTCCCAAATCTTGATTCTTTGAGTGGCAGTGAGAAA |
| | | | ATAATGCATCTTTGTACCTTACCATTTACCTCACAACCTTGCAGTTCCAA |
| | | | TCGAAGGGTAGGTCTGTTATAGGCTCGAGTTGGAGAGATCCTAATATATC |
| | | | TAGCCACAATAGGTGGGTCAAACTGATTCTCTTTTATTGTAGAGGCATCT |
| | | | GAATTGCCATTAAAATACTAGAAGAAAAGAGGAAAGTTTAGTTATGTAAC |
| | | | AATGATCTATAAAG |
| F5 | rs2213866 | 72 | CAAAAACACTCATTGAACACCATATATATGGCAGTGATGTTGCCAGATAC |
| | (at position | | TGTCATGACATTGAAGTTTGAGTGACCTGAGGACTTTGGAAGAGTCAGGC |
| | 177) | | CTAGTTTGAATCTCAGGTAGGTCTTATTGAAAATGGGCTGATGGAGGTAA |
| | | | TTCCAAATTAGAGCCCTTCCTTGGAGRGTTGTGATGTGTCTATATAATCC |
| | | | AGGCACTTTCTTCACAGAGATGCTGTCGGCACTCTGATTGGCAGAACCAT |
| | | | TCTTGGTCTAGATCACACTGAGAGTTTACCTGAGTAGAACCTCTGTTTCA |
| | | | CAAAGGTTTTCCTAGGAGCCTAAGTCACTGAAAAGAACTAAAAATTCTAC |
| | | | TCATTCTCCTATACCTCCCAAATCTTGATTCTTTGAGTGGCAGTGAGAAA |
| | | | ATAATGCATCTTTGTACCTTACCATTTACCTCACAACCTTGCAGTTCCAA |
| | | | TCGAAGGGTAGGTCTGTTATAGGCTCGAGTTGGAGAGATCCTAATATATC |
| | | | TAGCCACAATAGGTGGGTCAAACTGATTCTCTTTTATTGTAGAGGCATCT |
| | | | GAATTGCCATTAAAATACTAGAAGAAAAGAGGAAAGTTTAGTTATGTAAC |
| | | | AATGATCTATAAAG |
| F5 | rs2213867 | 73 | CTGTCATGACATTGAAGTTTGAGTGACCTGAGGACTTTGGAAGAGTCAGG |
| | | | CCTAGTTTGAATCTCAGGTAGGTCTTATTGAAAATGGGCTGATGGAGGTA |
| | | | ATTCCAAATTAGAGCCCTTCCTTGGAGAGTTGTGATGTGTCTATATAATC |
| | | | CAGGCACTTTCTTCACAGAGATGCTGTCGGCACTCTGATTGGCAGAACCA |
| | | | YTCTTGGTCTAGATCACACTGAGAGTTTACCTGAGTAGAACCTCTGTTTC |
| | | | ACAAAGGTTTTCCTAGGAGCCTAAGTCACTGAAAAGAACTAAAAATTCTA |
| | | | CTCATTCTCCTATACCTCCCAAATCTTGATTCTTTGAGTGGCAGTGAGAA |
| | | | AATAATGCATCTTTGTACCTTACCATTTACCTCACAACCTTGCAGTTCCA |
| | | | ATCGAAGGGTAGGTCTGTTATAGGCTCGAGTTGGAGAGATCCTAATATAT |
| | | | CTAGCCACAATAGGTGGGTCAAACTGATTCTCTTTTATTGTAGAGGCATC |
| | | | TGAATTGCCATTAAAATACTAGAAGAAAAGAGGAAAGTTTAGTTATGTAA |
| | | | CAATGATCTATAAAG |
| F5 | rs9332655 | 74 | TTACCGATACCTGCTCCAATCTTCTGTTTTAAAAAGTTGGCTTTTTCTGA |
| | | | CATTGCTCTGTCAGGAAAAGGGGTAGGGCACAGCCTGTTTACTGCCAAGT |
| | | | GGGGGTCAAAGTCCAGGTTCCCCACTCCATTGCCACCTAAGAAGGGATTG |
| | | | TTCCTTGGTGGCTGGGTGGGAAGGGAAGTTCCCCATTTGGCCTCCACTGA |
| | | | YACTGCAGGGGCAGGAGCTTCATTAGGGGCTGGAGATGAAAGCCCTAAAT |
| | | | CCCTACATGGCCTTTTCTGACACAACCCCAGTGAGGGTGTAGGGTGCCTC |
| | | | TTTAGCCTCAGGAGCATAGAAGTCTAGGCTCCCCATTCAGCCTTTGCTGT |
| | | | TGTGGGTTGGGGAGGGGCCTCAGGTTTTTCTGTGGTGTTTGGCTAAAGGA |
| | | | GAGAAGTCAGTGTCCACCAGTTTTCTATCATATCTCGCTATGCTGCCCTT |
| | | | TACTGG |
| F5 | rs9332652 | 75 | ATTGTCATCTGCCCCTCCTTTCTTTTTTGTGTGAATCTTGCTAGAGATTT |
| | | | GTCGATTTTAAAAAAATCTTCTTGGCCGGGCGCAGTGGCTCACGTCTGTA |
| | | | ATCCCAGCACTTTGGGAGGCCGAGGTGGGCAGATCACGAGGTCAAGAGAT |
| | | | CAAGACCATCCTGACCAACATGGTGAAACCCCCTCTCTACTAAAAATATT |
| | | | A/- |
| | | | GCAGGAGTATCGCTTGAATCCGGAAGGCAGAGGTTGCAGTGAGCCGAGAT |
| | | | CACGCCACTGCACTCCAGCCTGGTGACGGAGTGAGACTCCGTCTCAAAAA |
| | | | AAAAAAAAAAAAAAAAAAATCTTTTCACAGAACCAGCCTTGTTTTATTGA |
| | | | TTTTTCTCTATTGTTTTTCTGTTTTCAGTATTACCGATACCTGCTCCA |
| F5 | rs9332651 | 76 | GTATATTTCAATTAACTGGTCAATTCCCTCTACGTTGTTAAGTTTATATG |
| | | | TGTAGAGTTGTTTGTATATTCCCTTATCCTTTTTCATGTTTGTAGTGATT |
| | | | TTGCCTGTTTCATCTCTGATATTGTCATCTGCCCCTCCTTTCTTTTTTGT |
| | | | GTGAATCTTGCTAGAGATTTGTCGATTTTAAAAAAATCTTCTTGGCCGGG |
| | | | YGCAGTGGCTCACGTCTGTAATCCCAGCACTTTGGGAGGCCGAGGTGGGC |
| | | | AGATCACGAGGTCAAGAGATCAAGACCATCCTGACCAACATGGTGAAACC |
| | | | CCCTCTCTACTAAAAATATTAAAAAAAAATTAGGCAGGGTGGCGTGCACT |
| | | | TGTAATCCCAGCTACTTGGGAGGCTGAGGCAGGAGTATCGCTTGAATCCG |
| | | | GAAGGCAGAGGTTGCAGTGAGCCGAGATCACGCCACTGCACTCCAGCCTG |
| | | | GTGACG |
| F5 | rs9332643 | 77 | CTGAGGTTGGTTCTAATAATATCTTGTTTTATAGATTAAAAAACAAAAGG |
| | | | CACAGAGATACTACCTACCTTCCCAAAGTAACACACAGCTAATTAGTCAT |
| | | | TGAGTTGGGATTCAAATTTAGGTACTCTGTGCCCAAATATGAATTGCATG |
| | | | TAGTCATTGTATCAGTTTGCTAAGATCTTATGTGCTAGCTCTTTAGTTCT |
| | | | RAAGAAAGCTGATTGTATAATGAATTTAGGCAGTGTGTGACTTGTTGACA |
| | | | AGGACAGTTCTGTTTACTGGCTTTCCTATATTGCAGGTGGACATGCAAAA |
| | | | GGAAGTCATAATCACAGGGATCCAGACCCAAGGTGCCAAACACTACCTGA |
| | | | AGTCCTGCTATACCACAGAGTTCTATGTAGCTTACAGTTCCAACCAGATC |
| | | | AACTGGCAGATCTTCAAAGGGAACAGCACAAGGAATGTGATGGTTTGTGT |
| | | | GCATAT |
| F5 | rs2301515 | 78 | TTGTCATGTATGGTTTCATAGGCTGCATGCTGCACAACTGTAGGGGGTAC |
| | | | CATTCACAGACCATGGTGCTACAACTTACCCAGAAACTCTGAAGCCTTGA |
| | | | TCTGTGAATCAGATATGATACCAGTGCTTAGTCCCATTGGCATCCTACAG |
| | | | TCTATGAAAAACAGAAAAAAAATGAATAATTTTTGCTTAGAAAATATATA |
| | | | YAATAAAGTAAAACTCCATGGTTAGGGATTATGTTTCTGACTCAATAATT |
| | | | AGATATTTTTACCTACCTTGTGTGGCCCTGACTTAAATATATCTGATTAT |
| | | | AAAATGGTAAATGCACTAACAAGACTGGTGCTTTAATAGATATAGTGGCA |
| | | | TCCTAGAAGAAAGCAACTGAAATCCTGAAATGTAATCATTTTTTAAACTG |
| | | | ATTTTCTNCTGCTTTTATCTTAAATTATGCTATTTTACAACATTAGGTAC |
| | | | CATCCATTGGCCAGGTGCAGTGGCTCATGCCTGTAATCCTAGCACTCTGG |
| | | | GAGGCCGAGGGGGGAGGATTACTTGAGGCCAGGAGTTTGATCTTGGGCAA |
| | | | CCTCTTCTCTACAAAAAAAATAAAAATACAAAATATAAAAATATGAAAAA |
| | | | AAGTTCTAGTCCT |
| F5 | rs9332635 | 79 | AAAGCAAAAATGAAAAGAGGGAAAGGAAACATGTATGATATTTTTGAAGC |
| | | | CCAATGATGTCATTCGAGGTCACTTTGATAATTAGGACTGTGAAGCTGTA |
| | | | TTGTATGTGAGTTAATTTGTAACATTATATTTAGATAAGTGAAAGTTTCT |
| | | | TCTAGTGAGGTGTGGTGGTGATTTAAAATTTTTTTTAGTTATCTCTGTGT |
| | | | RTGTGTTGTTATTGTTTTGTTTTTATCTGTTATACTTTCAGTCCTACCTA |
| | | | AAGAAAAATGGTTAAATTCTATTTGAAAGCCTCTTGTGAAGCAGGAATTT |
| | | | TAGGATTCTTAGAGAACTATCAACCACAATATTTACTTGTTAATTTTTGC |
| | | | AAATGTAATGTTGTTCTTTTTATTTTAGGTTCATTTAAAACTCTTGAAAT |
| | | | GAAGGCATCAAAACCTGGCTGGTGGCTCCTAAACACAGAGGTTGGAGAAA |
| | | | ACCAGA |
| F5 | rs9332627 | 80 | AACTTTATCTTTACAGTTTTGTGTTACATTTTTAAGGTGATCAAGTTTCT |
| | | | CAGGAACTTTTTAAAAAATCCGCGTATTTTACTGTGGGGAGAGTAGATAA |
| | | | AGCCTGAGAACCCTAGTTATCTAATCTGAGAAGTGGGCAGAGGAGTTGTC |
| | | | ATACCAATAAGAGAATAAACATGACTTGCTATGGTTGCTGGATACACCAA |
| | | | YGATAAACTCCTAATTTTATAAACTCCTAGACTTCCTAATTTGCCTGAAA |
| | | | CTTTCTCTGAATTTAGAAGGCCTTAAGGTGACATGCTGCATATCTCCTTG |
| | | | CTGACTCTAATCCCTGGAGTTTACTTTGTCTGCCCATATTTGTATTTATC |
| | | | TTTTGTAGTTAAGGAAAATTAAGACTGTTAATGGAAAGTATACACGGGTA |
| | | | AAGCACGGAACTGTAAAAGCTGAGGAAAAGAGTGATAAACTGCTTGGAAA |
| | | | GGGAGT |
| F5 | rs2420373 | 81 | TCACCTTAAAAATGTAACACAAAACTGTAAAGATAAAGTTTAGTGTTACC |
| | | | AAACGTTTAACTGAAACCAAAGTGAAGGAAGTGCTTTGTTTCCTCCTTCT |
| | | | TAAGTTAGAGGAACTGCTGTGAAATTTCAGAAACCAGGCAGGTGAATTTT |
| | | | TGGAGAGGGACGGGTTGAGATCATTTGGGCATCCTTCAAATTATATCAGG |
| | | | YGTGAGAACACCTATCTACATATTCATAACACAAGCTCGTACCTTTCTCT |
| | | | TCTCAGTTTCTGTTTCAGTTACTAGCTTACAGGGCAAATTTTCCTTGTGA |
| | | | ATTTCCAAATAGGCACACTTTTCCCAGTGTGACCACCCAAATGCTCTCTT |
| | | | GCCACTATTCTCCTATTACCTGGGTACCTAGTTTCTTATCTCATTTTGTT |
| | | | CTCATATGCTTTGGGTGCCACCTGGTAGCTGCTGAGAGCTAAAACTGTTA |
| | | | GCGTCATTCTTATTTTAGAAAAGACACACAGGTGATCATTTCTTTTATAT |
| | | | AAACACTTGTGCCTTTAGAGATCCAGAAACAAGCTTATTAACTTTAAATT |
| | | | GCAGAACAATGTTAATTCCTTTTCAATGTATGTTACCAGCTGAAGCACAC |
| | | | TGGCTTTTTGTTGTTGTGGTTTTCTGTTGGTTTGTTTTGCAGCAGCTTCT |
| | | | CTGTGCTATCTTTCAGACTTTCTCTTTTTGCCTCACCCACAGTGTCACCT |
| | | | G |
| F5 | rs2157581 | 82 | CCTTATGTAGTATTCCTTTTTGGCAGATTAGGAGGGGACCTATCAAGCCT |
| | | | GAGTGAATATCTTTTTCCTGGAAAAACAGAGTAAATTGTATTGCCTCTTT |
| | | | CTCAGGAATTTCCTTGCTCTTCTGATAATCACTCATCATATAGAAAAGGA |
| | | | AACTTTCTGATAGGCTCTGAATTTGAAGATGAAGATTATAAATGAATGGC |
| | | | RTAATAAGCCTGGATATTTATCACCTAATTCTGTTGTATTCATAATCCTC |
| | | | CTTCCTCTGATTGATCTTTATCCCAGTAACAATGATGATAATAATAAATT |
| | | | GATAATATAAGGGATACTATTTATTGAGAATCTATTACTATATTAAATGA |
| | | | TTTGCCTGCAATCTCCCATTTAATCCTGACAACTATCTTATATGGTAAAT |
| | | | ATAAATTCATTCATTGAACAAATATTTACCAAGTGCTTACCATGCACTAT |
| | | | GTACTGTTATCTCATTTTTAACTTCTGATACCAGGCTAAGAGAGGTCAAG |
| | | | GAATTTCCTAGGATTATGCATTGACAGGGTAAAAATTTAAATCTGAGTCT |
| | | | GTGCTCTTTCCACTATGCCTGAAATGGAGGAGTTGTTTCTCTTTTTAATT |
| | | | GACAGATAAAATTGTATGCATATACTGTGTATGACATATTGTTTTGAAGT |
| | | | ATATATACATTGCAGAATGTCAGATAAAGGAGTCTTGACTTTGCAGTTCT |
| | | | T |
| F5 | rs2187953 | 83 | ATGAATGGCATAATAAGCCTGGATATTTATCACCTAATTCTGTTGTATTC |
| | | | ATAATCCTCCTTCCTCTGATTGATCTTTATCCCAGTAACAATGATGATAA |
| | | | TAATAAATTGATAATATAAGGGATACTATTTATTGAGAATCTATTACTAT |
| | | | ATTAAATGATTTGCCTGCAATCTCCCATTTAATCCTGACAACTATCTTAT |
| | | | MTGGTAAATATAAATTCATTCATTGAACAAATATTTACCAAGTGCTTACC |
| | | | ATGCACTATGTACTGTTATCTCATTTTTAACTTCTGATACCAGGCTAAGA |
| | | | GAGGTCAAGGAATTTCCTAGGATTATGCATTGACAGGGTAAAAATTTAAA |
| | | | TCTGAGTCTGTGCTCTTTCCACTATGCCTGAAATGGAGGAGTTGTTTCTC |
| | | | TTTTTAATTGACAGATAAAATTGTATGCATATACTGTGTATGACATATTG |
| | | | TTTTGAAGTATATATACATTGCAGAATGTCAGATAAAGGAGTCTTGACTT |
| | | | TGCAGTTCTTTTCATAAAGAAAGAGCAGAACATAGCTAATACTTGTTCAA |
| | | | GAAAATTTCAAATAAATGCCATCTTCTGTAAATGTAGGCATTCTAATTCA |
| | | | TGGCCAATCATTCAAGTAATCTTTCCTCCTCTCCACTGAATAAATGTTTC |
| | | | TCTCTCT |
| F5 | rs916438 | 84 | AGGGTAAAAATTTAAATCTGAGTCTGTGCTCTTTCCACTATGCCTGAAAT |
| | (at position | | GGAGGAGTTGTTTCTCTTTTTAATTGACAGATAAAATTGTATGCATATAC |
| | 145) | | TGTGTATGACATATTGTTTTGAAGTATATATACATTGCAGAATGWCAGAT |
| | | | AAAGGAGTCTTGACTTTGCAGTTCTTTTCATAAAGAAAGAGCAGAACATA |
| | | | GCTAATACTTGTTCAAGAAAATTTCAAATAAATGCCATCTTCTGTAAATG |
| | | | TAGGCATTCTAATTCATGGCCAATCATTCAAGTAATCTTTCCTCCTCTCC |
| | | | ACTGAATAAATGTTTCTCTCTCTGTCATCTGAAGAGCTGCATGGAGAGTC |
| | | | CCTGGTTATGATAAATGCAGACTGTTAACCACACCCTTATGCATTCCTCA |
| | | | TGAAAAGCAAGACAGACATTTGACAAGAAATAACCCCGACTCTTCCATTT |
| | | | GGTGGACTTCAGATTACGAGGTTAGGGGAATGAGAAAAACTTTCAATGAA |
| | | | AGTACCTACTGGGTTCACA |
| F5 | rs9332620 | 85 | TTAAGGAAGATAATGCTGTTCAGCCAAATAGCAGTTATACCTACGTATGG |
| | | | CATGCCACTGAGCGATCAGGGCCAGAAAGTCCTGGCTCTGCCTGTCGGGC |
| | | | TTGGGCCTACTACTCAGCTGTGAACCCAGTAGGTACTTTCATTGAAAGTT |
| | | | TTTCTCATTCCCCTAACCTCGTAATCTGAAGTCCACCAAATGGAAGAGTC |
| | | | RGGGTTATTTCTTGTCAAATGTCTGTCTTGCTTTTCATGAGGAATGCATA |
| | | | AGGGTGTGGTTAACAGTCTGCATTTATCATAACCAGGGACTCTCCATGCA |
| | | | GCTCTTCAGATGACAGAGAGAGAAACATTTATTCAGTGGAGAGGAGGAAA |
| | | | GATTACTTGAATGATTGGCCATGAATTAGAATGCCTACATTTACAGAAGA |
| | | | TGGCATTTATTTGAAATTTTCTTGAACAAGTATTAGCTATGTTCTGCTCT |
| | | | TTCTTT |
| F5 | rs9332619 | 86 | TCTTCACATCTCCACTACCTATCACTCTCATTTCATTAGTAGATAATGTC |
| | | | AGTACTTTAGCCTTGAGCCTAAGAACAAATATCTTTTGGTATTTCTGGAG |
| | | | AAAACTACTTGGGCCATATCTCACAGGATGGTTATGAAAATTAAATGAAA |
| | | | TAATATACATAAGTTATTTTGTACCTTATATCTTAGCTCCGATTTTATAA |
| | | | YCAGCCATTTTGACTTATAATGCTGACATTTTTGTGGTTTAGATTTTTGT |
| | | | TAAGCTTAAGTACATTTGTGGATCATTCCTTTTCCTAGGTTCGTTTTAAA |
| | | | AATTTAGCATCCAGACCGTATTCTCTACATGCCCATGGNCTTTCCTATGA |
| | | | AAAATCATCAGAGGGAAAGACTTATGAAGATGACTCTCCTGAATGGTTTA |
| | | | AGGAAGATAATGCTGTTCAGCCAAATAGCAGTTATACCTACGTATGGCAT |
| | | | GCCACTGAGCGATCAGGGCCAGAAAGTCCTGGCTCTGCCTGTCGGGCTTG |
| | | | GGCCTACTACTCAGCTGTGAACCCAGTAGGTACTTTCATTGA |
| F5 | rs4656187 | 87 | TTTAAAAAACTTCAGTTGCTTTAGGAATGCAAGTGGTTTTTGGTTACATG |
| | | | GATGAATTGTATAGTGGTGAAGTCTAGGATTTTAGTGTACCTGTCACCTG |
| | | | AGTAGTGTACCCTGTACCCAATAGGTAGCTTTTCATCCATCCCCCGTTCC |
| | | | CTTTTGAGTCTCCAATGTCCATTATACCAATCTGTCTGCCTTCGCATACC |
| | | | YGCAGTTTATCTCCCATTTGTAAGTGAGAACATACAGTGTCTGGTTTTTG |
| | | | ATTCCTGAGTTACTTAGAATAAGTTTTTTAAATATTGTGTTAAATTATCG |
| | | | TTCATCTTGAAAAAGGATCCCCTAATCATAAAAGAGAATATTGCCTCCCA |
| | | | TAGCTTCATGGAAAATTTAGAATAATTAAGATTCTTATATCCCTATGTAC |
| | | | TTGTTTACGTTTTTAAAAAGAGCAAATGGTCACTGAAAATGTAGTGAATG |
| | | | CTTACACAGGTATAG |
| F5 | rs7535409 | 88 | ATAAATGAGTAAATATATAAGTGGATAAAAACAAAAGCCAGTAAATATCT |
| | | | TTCAATTCCTAACTCAAATATTATTTTATAGGTAAGTCTTCCTATGACCT |
| | | | TTTAGACTAGGTCAAGTCTCTATATCAATTTCTCATATTTCTATAGTATT |
| | | | ATTATAGTATATATAGTAAATTCCTGTAGTATTCTTATAATATCTCTAGA |
| | | | RTATATATTGTAGATTATATATAATTTATTTGTGAGATTATCTATTAATA |
| | | | TCTGTCTCTCACTGGGTATAAGTGCCATGAGGTCAGCCATTGTGCCTAGT |
| | | | TTGCTCATAGTACTCTCTCCAGTGGTAAGCACATTATCTGACACACAGCA |
| | | | GGAACTTAATAAATTTTTGTCAAACATATAAATGAATGTATAAATATAAT |
| | | | CGTATGTTAACACACCAAATTTTAAGATCAAAGGCAGACAAAGCCATGTA |
| | | | ATGGGACAGTGCCAGAGCTTGGGGCTATCAGGTGACAATGGTCAGATTAA |
| | | | TTAGAAGGTCACACTTATGAAAGTCACTGGATGGGTGAATGTTTTGTACC |
| | | | CTAAAAGTAGCCACTCTTCTC |
| F5 | rs1557572 | 89 | TCACATTTCCCAAGCCTGTGGAAACAAGCCAAACACTCACCCATCAAACC |
| | | | CATAATATTTGCTATATACTGTGAGTCATCAACAGAGAATCTCCTTCTGC |
| | | | CTTTCTTCTGGTCTACCTCCCCTACTAATCCCATCTTTCCAGACTCTGAG |
| | | | CATAACATGCAAACTCACAGAACACAAGGGAGTGGGTAAAGCAACTCCGA |
| | | | MTGCCATAAAAGTGGGTTGTGAGCCTTGAATGGAATACAAGATTTTGAAG |
| | | | GTGGTTCCATCCCTATTCACTCTGGACAGGCCCTGCATCTCACTCCCTCG |
| | | | GGGCCTTGCTTAGAAATACTCAGGTAGCTAGTTGTTCTCATGTGGTATTG |
| | | | AGTGCAACATTTAAATAGGAAGTCATAGGAAAAGGTGTTTTAAACAGAGT |
| | | | TCTAATGTGGAGATGTCAGGCATCAG |
| F5 | rs3766109 | 90 | CTCACAGAACACAAGGGAGTGGGTAAAGCAACTCCGAATGCCATAAAAGT |
| | | | GGGTTGTGAGCCTTGAATGGAATACAAGATTTTGAAGGTGGTTCCATCCC |
| | | | TATTCACTCTGGACAGGCCCTGCATCTCACTCCCTCGGGGCCTTGCTTAG |
| | | | AAATACTCAGGTAGCTAGTTGTTCTCATGTGGTATTGAGTGCAACATTTA |
| | | | WATAGGAAGTCATAGGAAAAGGTGTTTTAAACAGAGTTCTAATGTGGAGA |
| | | | TGTCAGGCATCAGATTAATGAACTCATATGCATAAGTCACACCATACATT |
| | | | GTGTTTGCCTACTGTAATTACACTTTGGTTTTTTAAGTGATTAGTGTAAC |
| | | | AGCTTTAGTAAAAGCTGTGGGCATATCTAGACAGCTGATGCACAGTCATC |
| | | | ATGCTATATCCCTGAGAATTTGTAGTTGACTAAGCCTTGCTCCTTTCCTC |
| | | | CTCTCT |
| F5 | rs6032 | 91 | AAAGTCAAGAACATGCTAAGCATAAGGGACCCAAGGTAGAAAGAGATCAA |
| | | | GCAGCAAAGCACAGGTTCTCCTGGATGAAATTACTAGCACATAAAGTTGG |
| | | | GAGACACCTAAGCCAAGACACTGGTTCTCCTTCCGGAATGAGGCCCTGGG |
| | | | AGGACCTTCCTAGCCAAGACACTGGTTCTCCTTCCAGAATGAGGCCCTGG |
| | | | RAGGACCCTCCTAGTGATCTGTTACTCTTAAAACAAAGTAACTCATCTAA |
| | | | GATTTTGGTTGGGAGATGGCATTTGGCTTCTGAGAAAGGTAGCTATGAAA |
| | | | TAATCCAAGATACTGATGAAGACACAGCTGTTAACAATTGGCTGATCAGC |
| | | | CCCCAGAATGCCTCACGTGCTTGGGGAGAAAGCACCCCTCTTGCCAACAA |
| | | | GCCTGGAAAGCAGAGTGGCCACCCAAAGTTTCCTAGAGTTAGACATAAAT |
| | | | CTCTAC |
| F5 | rs4525 | 92 | TTCTCACCAACAAGCCACCACAGCTGGTTCCCCACTGAGACACCTCATTG |
| | | | GCAAGAACTCAGTTCTCAATTCTTCCACAGCAGAGCATTCCAGCCCATAT |
| | | | TCTGAAGACCCTATAGAGGATCCTCTACAGCCAGATGTCACAGGGATACG |
| | | | TCTACTTTCACTTGGTGCTGGAGAATTCAAAAGTCAAGAACATGCTAAGC |
| | | | RTAAGGGACCCAAGGTAGAAAGAGATCAAGCAGCAAAGCACAGGTTCTCC |
| | | | TGGATGAAATTACTAGCACATAAAGTTGGGAGACACCTAAGCCAAGACAC |
| | | | TGGTTCTCCTTCCGGAATGAGGCCCTGGGAGGACCTTCCTAGCCAAGACA |
| | | | CTGGTTCTCCTTCCAGAATGAGGCCCTGGAAGGACCCTCCTAGTGATCTG |
| | | | TTACTCTTAAAACAAAGTAACTCATCTAAGATTTTGGTTGGGAGATGGCA |
| | | | TTTGGC |
| F5 | rs4524 | 93 | TGCAGAACCTCAGAAAGCCCCTTCTCACCAACAAGCCACCACAGCTGGTT |
| | | | CCCCACTGAGACACCTCATTGGCAAGAACTCAGTTCTCAATTCTTCCACA |
| | | | GCAGAGCATTCCAGCCCATATTCTGAAGACCCTATAGAGGATCCTCTACA |
| | | | GCCAGATGTCACAGGGATACGTCTACTTTCACTTGGTGCTGGAGAATTCA |
| | | | RAAGTCAAGAACATGCTAAGCATAAGGGACCCAAGGTAGAAAGAGATCAA |
| | | | GCAGCAAAGCACAGGTTCTCCTGGATGAAATTACTAGCACATAAAGTTGG |
| | | | GAGACACCTAAGCCAAGACACTGGTTCTCCTTCCGGAATGAGGCCCTGGG |
| | | | AGGACCTTCCTAGCCAAGACACTGGTTCTCCTTCCAGAATGAGGCCCTGG |
| | | | AAGGACCCTCCTAGTGATCTGTTACTCTTAAAACAAAGTAACTCATCTAA |
| | | | GATTTT |
| F5 | rs9332600 | 94 | GTCCTGATCTGCCAATCGATTGCTGTGTAACCTTACACAAGTTACTTGGC |
| | | | CACACTGAGCCACAAGTCATTTATCTGGAAAACAGTGTAATCACATCTCA |
| | | | CAGAGTTACTTTGACCATTAAAATAGTAATATGCGCCAAGTGCCTAGCAC |
| | | | TCAGTAGACACCAACAATGGTAACTATTGGAGACTCACCAAGAAATCTTT |
| | | | RATGTTCCAGCAATGCATGCCATTTCAGAGATTCAAAATTGTCCTCGTGA |
| | | | ATTATTACTTAGAAACATCTAAATGTCTCTTATTTGTGGGGATAGAGCTC |
| | | | ATCACCATCCCTTTAATTCTAAGACAAGATGTGCTGTTAGGATATTTATG |
| | | | ATATTAAAAGTCCATTTTATTCTTGATTCATCCCTTTCTTAATATATTTT |
| F5 | rs9332599 | 95 | TCATCAGGTACAGTGCCTTAAAGGCTGCTTCAGCAACAGCTTTGGAGTTT |
| | | | GTCAGACTGGAATGCAAGTCCTGATCTGCCAATCGATTGCTGTGTAACCT |
| | | | TACACAAGTTACTTGGCCACACTGAGCCACAAGTCATTTATCTGGAAAAC |
| | | | AGTGTAATCACATCTCACAGAGTTACTTTGACCATTAAAATAGTAATATG |
| | | | YGCCAAGTGCCTAGCACTCAGTAGACACCAACAATGGTAACTATTGGAGA |
| | | | CTCACCAAGAAATCTTTGATGTTCCAGCAATGCATGCCATTTCAGAGATT |
| | | | CAAAATTGTCCTCGTGAATTATTACTTAGAAACATCTAAATGTCTCTTAT |
| | | | TTGTGGGGATAGAGCTCATCACCATCCCTTTAATTCTAAGACAAGATGTG |
| | | | CTGTTAGGATATTTATGATATTAAAAGTCCATTTTATTCTTGATT |
| F5 | rs9287092 | 96 | CACTTCACTGGGCACTCATTCATCTATGGAAAGAGGCATGAGGACACCTT |
| | | | GACCCTCTTCCCCATGCGTGGAGAATCTGTGACGGTCACAATGGATAATG |
| | | | TTGGTGAGTAAGAGTCTGGACACTCACAGAGGAAGCTTGCTTTGAATTTC |
| | | | TGGTCTATAAAGGTCTGCTGCAACTCTCCAGGCTACCAGTGCTCCTCTAT |
| | | | KTATCTCCCTGACCCCCTGCAGGCTTTTCTTTCAATGTTTCTCATGATTT |
| | | | CTCTTTGAGAAATTAATGACTTAAATGGATCCAGTTCTTTAGTGTGGGTT |
| | | | ATATTTTTCCTTCTCTGGGCAAAGTAGGAAGTAAAAATATACAACAGCAG |
| | | | AAAAATAAGGCATAACTCTGAGGAAGAAGCATAAATATTTTGGCCACAAA |
| | | | AGAGCATTTCTTTTATCAAAATGCCCTATTCGGTTTTTTGCAACAGTCAT |
| | | | CTTCAA |
| F5 | rs9332596 | 97 | TGTAGCCAGTGGCTACCATATTGAACAATGCAGTTATAGACCATTTAGAA |
| | | | AAACATCTAGAATGAGGTATAAACTAATAATACATCATGTAAATTATTAA |
| | | | AGTCACTTAAGTGATTTAAGTAGGTATTTAAATATTTGTTAATGCCAGTC |
| | | | ATTATACTGGTACTAAGGCTAAGAGTGGTAATCGAGGTAGACATGAGCCC |
| | | | YGCCCTTGTAGAAATCATACCTTTGGTTTTTTACTATGCTTAGTACATAA |
| | | | GTAAATAAAAATATTGCCTACAGCAGTGTCCCTTTTAACAATAATGAAAT |
| | | | GTATGAACTGGATACTCAAATGGAAACTGTGAATACTATGTAGATTATAA |
| | | | GACAGCAATAAAAACTATAAAATATGCTAAATGGGCTTTATTTTTAGGGA |
| F5 | rs9332595 | 98 | AAGTGCTCGATAACCACTTGTAGCCAGTGGCTACCATATTGAACAATGCA |
| | | | GTTATAGACCATTTAGAAAAACATCTAGAATGAGGTATAAACTAATAATA |
| | | | CATCATGTAAATTATTAAAGTCACTTAAGTGATTTAAGTAGGTATTTAAA |
| | | | TATTTGTTAATGCCAGTCATTATACTGGTACTAAGGCTAAGAGTGGTAAT |
| | | | SGAGGTAGACATGAGCCCTGCCCTTGTAGAAATCATACCTTTGGTTTTTT |
| | | | ACTATGCTTAGTACATAAGTAAATAAAAATATTGCCTACAGCAGTGTCCC |
| | | | TTTTAACAATAATGAAATGTATGAACTGGATACTCAAATGGAAACTGTGA |
| | | | ATACTATGTAGATTATAAGACAGCAATAAAAACTATAAAATATGCTAAAT |
| F5 | rs3766110 | 99 | ATTACCTACTAGAGAAGGTGATTACCATGACTCTAGACTCTGAGGATCAG |
| | | | TAGGGGGACCCTGGCTGTTGTGGAGAAGTTTGCTTCCAGCTGGCTCAGTG |
| | | | ACATTTCTCTCAGACTGCCATGGCAGTGCTTTTGGCCACTGAACTTTAGA |
| | | | ACTGCAGCAAATGCCGGAACCCTCATTCAGGGAATTCCTTTTGTTCTGAA |
| | | | MATCTTACTGATCACTTGAAATGTCTTCATGCATGCCTTTCCAAGACTCT |
| | | | TGGGTCCCTATACTCATTTTGCTCTACTTTGATGTGTCAATCCATCTTTG |
| | | | GATTACTTGCCTTCTTTCTGTCCTAGCCATATATTCACCCTGAACTCAGT |
| | | | CTAGGATACTATTGACATGGACTATAACACCTTCCATTAGTCCTACTCTC |
| | | | TCACCCTTTTCCTCACCACGCAGAGTATGTCTGTGTACACACACACACAC |
| | | | ACACAC |
| F5 | rs3766111 | 100 | TTACCATGACTCTAGACTCTGAGGATCAGTAGGGGGACCCTGGCTGTTGT |
| | | | GGAGAAGTTTGCTTCCAGCTGGCTCAGTGACATTTCTCTCAGACTGCCAT |
| | | | GGCAGTGCTTTTGGCCACTGAACTTTAGAACTGCAGCAAATGCCGGAACC |
| | | | CTCATTCAGGGAATTCCTTTTGTTCTGAAAATCTTACTGATCACTTGAAA |
| | | | YGTCTTCATGCATGCCTTTCCAAGACTCTTGGGTCCCTATACTCATTTTG |
| | | | CTCTACTTTGATGTGTCAATCCATCTTTGGATTACTTGCCTTCTTTCTGT |
| | | | CCTAGCCATATATTCACCCTGAACTCAGTCTAGGATACTATTGACATGGA |
| | | | CTATAACACCTTCCATTAGTCCTACTCTCTCACCCTTTTCCTCACCACGC |
| | | | AGAGTATGTCTGTGTACACACACACACACACACACACACACACATGCTTG |
| | | | GAATAG |
| F5 | rs3766112 | 101 | ACTGCCATGGCAGTGCTTTTGGCCACTGAACTTTAGAACTGCAGCAAATG |
| | | | CCGGAACCCTCATTCAGGGAATTCCTTTTGTTCTGAAAATCTTACTGATC |
| | | | ACTTGAAATGTCTTCATGCATGCCTTTCCAAGACTCTTGGGTCCCTATAC |
| | | | TCATTTTGCTCTACTTTGATGTGTCAATCCATCTTTGGATTACTTGCCTT |
| | | | STTTCTGTCCTAGCCATATATTCACCCTGAACTCAGTCTAGGATACTATT |
| | | | GACATGGACTATAACACCTTCCATTAGTCCTACTCTCTCACCCTTTTCCT |
| | | | CACCACGCAGAGTATGTCTGTGTACACACACACACACACACACACACACA |
| | | | CATGCTTGGAATAGAAGATCAAACGCATTTCTAAGGATGTGAGCCTTTGA |
| | | | CCTCTTGCTTAAAAATGTTGCTATGATGTCACCCACGGATTTCATCACCA |
| | | | AGTCTT |
| F5 | rs3766113 | 102 | AGTGCTTTTGGCCACTGAACTTTAGAACTGCAGCAAATGCCGGAACCCTC |
| | | | ATTCAGGGAATTCCTTTTGTTCTGAAAATCTTACTGATCACTTGAAATGT |
| | | | CTTCATGCATGCCTTTCCAAGACTCTTGGGTCCCTATACTCATTTTGCTC |
| | | | TACTTTGATGTGTCAATCCATCTTTGGATTACTTGCCTTCTTTCTGTCCT |
| | | | RGCCATATATTCACCCTGAACTCAGTCTAGGATACTATTGACATGGACTA |
| | | | TAACACCTTCCATTAGTCCTACTCTCTCACCCTTTTCCTCACCACGCAGA |
| | | | GTATGTCTGTGTACACACACACACACACACACACACACACATGCTTGGAA |
| | | | TAGAAGATCAAACGCATTTCTAAGGATGTGAGCCTTTGACCTCTTGCTTA |
| | | | AAAATGTTGCTATGATGTCACCCACGGATTTCATCACCAAGTCTTTGGAC |
| | | | TGGAAG |
| F5 | rs1894694 | 103 | AAAATCTTACTGATCACTTGAAATGTCTTCATGCATGCCTTTCCAAGACT |
| | | | CTTGGGTCCCTATACTCATTTTGCTCTACTTTGATGTGTCAATCCATCTT |
| | | | TGGATTACTTGCCTTCTTTCTGTCCTAGCCATATATTCACCCTGAACTCA |
| | | | GTCTAGGATACTATTGACATGGACTATAACACCTTCCATTAGTCCTACTC |
| | | | YCTCACCCTTTTCCTCACCACGCAGAGTATGTCTGTGTACACACACACAC |
| | | | ACACACACACACACACGCTTGGAATAGAAGATCAAACGCATTTCTAAGGA |
| | | | TGTGAGCCTTTGACCTCTTGCTTAAAAATGTTGCTATGATGTCACCCACG |
| | | | GATTTCATCACCAAGTCTTTGGACTGGAAGTGAGGATTGGAGGTGCCCCT |
| | | | TAGCGAGTAGATTTTAATCCATGTCTCTGACTCTAGGCACAGTCATATTT |
| | | | CAACCACAGGAATGAAAAACTGATGAACAAAAATAGTACTCTGACTT |
| F5 | rs9332589 | 104 | TTCATTCCTGTGGTTGAAATATGACTGTGCCTAGAGTCAGAGACATGGAT |
| | | | TAAAATCTACTCGCTAAGGGGCACCTCCAATCCTCACTTCCAGTCCAAAG |
| | | | ACTTGGTGATGAAATCCGTGGGTGACATCATAGCAACATTTTTAAGCAAG |
| | | | AGGTCAAAGGCTCACATCCTTAGAAATGCGTTTGATCTTCTATTCCAAGC |
| | | | -/A/AT/G |
| | | | GTGTGTGTGTGTGTGTGTGTGTGTGTGTACACAGACATACTCTGCGTGGT |
| | | | GAGGAAAAGGGTGAGAGAGTAGGACTAATGGAAGGTGTTATAGTCCATGT |
| | | | CAATAGTATCCTAGACTGAGTTCAGGGTGAATATATGGCTAGGACAGAAA |
| | | | GAAGGCAAGTAATCCAAAGATGGATTGACACATCAAAGTAGAGCAAAATG |
| | | | AGTATAGGGACCCAAGAGTCTTGGAAAGGCATGCATGAAGACATTTCAAG |
| F5 | rs6672595 | 105 | GGATACTATTGACATGGACTATAACACCTTCCATTAGTCCTACTCTCTCA |
| | | | CCCTTTTCCTCACCACGCAGAGTATGTCTGTGTACACACACACACACACA |
| | | | CACACACACACGCTTGGAATAGAAGATCAAACGCATTTCTAAGGATGTGA |
| | | | GCCTTTGACCTCTTGCTTAAAAATGTTGCTATGATGTCACCCACGGATTT |
| | | | YATCACCAAGTCTTTGGACTGGAAGTGAGGATTGGAGGTGCCCCTTAGCG |
| | | | AGTAGATTTTAATCCATGTCTCTGACTCTAGGCACAGTCATATTTCAACC |
| | | | ACAGGAATGAAAAACTGATGAACAAAAATAGTACTCTGACTTACTGCTCA |
| | | | TGATGTTTGATTCATAAAACTTGGGGTCATCACGTTTCACNTCATCAGGA |
| | | | TTTTCACAAAACTTGTTGATGTTGTCCTCAAGGTACCAGCTTTTGTTCTC |
| | | | ATCAAACCACAGCAAACACAGCCTGCTGTTCGATGTCTGCTGCCCTCTGG |
| | | | AGGACAAAACAGTATAGTACTGGTACAAGAACAGACGCATAGACCAATGG |
| | | | AACAGAATAGAGAACTCAGAAATAAGGCTGCGCACCTACAACTATCT |
| F5 | rs1988607 | 106 | ACCAATTAATATTGCAAAAGGAATTCTTTTATTTTTTATTTGTTTTTAAA |
| | (at position | | TTATACTTTAAGTTCTAGGGTACATGTGCACAACGTGCAGGTTTGTTACA |
| | 176) | | TATGTATACATGTGTCATGTTGGTGTGCTGCACCCATTAACTTGTCATTA |
| | | | ACATTAGGTATATCTCCTAATGCTAYCCCTCCCCCCGCCCCCCACCCCCC |
| | | | CCGACAGGCCCCAGTGTGTGATGTTCCCCATCCTGTGTCTAAATGTTCTC |
| | | | ATTGTTCAATTGAATTCTTTAAATATTCTACTTGGAACCTGGATAACATG |
| | | | TAGCCATTAGATAATGCTCCACTAGAGGCCACTATGACACTAATAAAAGA |
| | | | CACCATATTTTGTTACCACTAAGAGACAAAACTCCTGAAGTGAGAAGGGT |
| | | | TTGGCTGTGATTTTTAGGATACTCCTACATGTATACTACCTGACTGCAGT |
| | | | AGTGACACCACCGGGCAAGGAGAATAGCAGAAAAATGTGGCAGCCTCTCA |
| | | | GAAGTTACTAGTTGGATTCAGTAGAAGTGAAAGATTCAAACCTG |
| F5 | rs1988608 | 107 | TGCAAAAGGAATTCTTTTATTTTTTATTTGTTTTTAAATTATACTTTAAG |
| | | | TTCTAGGGTACATGTGCACAACGTGCAGGTTTGTTACATATGTATACATG |
| | | | TGTCATGTTGGTGTGCTGCACCCATTAACTTGTCATTAACATTAGGTATA |
| | | | TCTCCTAATGCTACCCCTCCCCCCGCCCCCCACCCCCCCCGACAGGCCCC |
| | | | RGTGTGTGATGTTCCCCATCCTGTGTCTAAATGTTCTCATTGTTCAATTG |
| | | | AATTCTTTAAATATTCTACTTGGAACCTGGATAACATGTAGCCATTAGAT |
| | | | AATGCTCCACTAGAGGCCACTATGACACTAATAAAAGACACCATATTTTG |
| | | | TTACCACTAAGAGACAAAACTCCTGAAGTGAGAAGGGTTTGGCTGTGATT |
| | | | TTTAGGATACTCCTACATGTATACTACCTGACTGCAGTAGTGACACCACC |
| | | | GGGCAAGGAGAATAGCAGAAAAATGTGGCAGCCTCTCAGAAGTTACTAGT |
| | | | TGGATTCAGTAGAAGTGAAAGATTCAAACCTG |
| F5 | rs2420375 | 108 | CTCTCCAAGCAAGTCCTCCTTCCCCTGCCCTTCTCTTTTCAGCTTGGGCC |
| | | | ACATCTCATTTTGAATCTGCTTCTCATCTCTAGACCATGATCCCCTTCCC |
| | | | CTGCCCGGTAGATTTTTTAGGACACTGTCTTTGAAGTCATCTTCTCAGCT |
| | | | AGGTTCAGTGGCTCAAACCTGTAACCCCAGCACTTTGGGAGGCTGAAGCA |
| | | | SGTGGATCACTTGAGCTCAGGAGTTCAAGACCAGCCTGGGCAACATGGTG |
| | | | AAACCTCATCTCTACAAAAAAATACAAAAATTAGCCAGGCGTTGGGGCGT |
| | | | GTGCCTGTAGTCCCAGCTACTTGAGAGGCTGAGGTGCGAGAATCGCCTGA |
| | | | GCCCAGGAAGTGGAGGTTGCAGTGAGCCATGATCACACCACTGCACTCCA |
| | | | GCCTGGGTGACAGAGTAAGACCCTTGGTGGGGGGGAAAAGCTACTTGCTT |
| | | | GAGAGGCACAGACAAATAGCCAGATCCTGAACTTTTAATAAGGTATGTCT |
| | | | ACACTTGCTGAGAGCACATAAAAGGGATCAGAAATGGAAGAAGGGAAAGA |
| | | | GGCATGGAGAAAAAAAACTGCTTTTGTCCTTTGGCTATTTTAAATGAACA |
| | | | GAATAGACCTTGTCAGGTGCATAA |
| F5 | rs2420376 | 109 | TCCCCTTCCCCTGCCCGGTAGATTTTTTAGGACACTGTCTTTGAAGTCAT |
| | | | CTTCTCAGCTAGGTTCAGTGGCTCAAACCTGTAACCCCAGCACTTTGGGA |
| | | | GGCTGAAGCAGGTGGATCACTTGAGCTCAGGAGTTCAAGACCAGCCTGGG |
| | | | CAACATGGTGAAACCTCATCTCTACAAAAAAATACAAAAATTAGCCAGGC |
| | | | RTTGGGGCGTGTGCCTGTAGTCCCAGCTACTTGAGAGGCTGAGGTGCGAG |
| | | | AATCGCCTGAGCCCAGGAAGTGGAGGTTGCAGTGAGCCATGATCACACCA |
| | | | CTGCACTCCAGCCTGGGTGACAGAGTAAGACCCTTGGTGGGGGGGAAAAG |
| | | | CTACTTGCTTGAGAGGCACAGACAAATAGCCAGATCCTGAACTTTTAATA |
| | | | AGGTATGTCTACACTTGCTGAGAGCACATAAAAGGGATCAGAAATGGAAG |
| | | | AAGGGAAAGAGGCATGGAGAAAAAAAACTGCTTTTGTCCTTTGGCTATTT |
| | | | TAAATGAACAGAATAGACCTTGTCAGGTGCATAA |
| F5 | rs2420377 | 110 | AAGTCATCTTCTCAGCTAGGTTCAGTGGCTCAAACCTGTAACCCCAGCAC |
| | | | TTTGGGAGGCTGAAGCAGGTGGATCACTTGAGCTCAGGAGTTCAAGACCA |
| | | | GCCTGGGCAACATGGTGAAACCTCATCTCTACAAAAAAATACAAAAATTA |
| | | | GCCAGGCGTTGGGGCGTGTGCCTGTAGTCCCAGCTACTTGAGAGGCTGAG |
| | | | RTGCGAGAATCGCCTGAGCCCAGGAAGTGGAGGTTGCAGTGAGCCATGAT |
| | | | CACACCACTGCACTCCAGCCTGGGTGACAGAGTAAGACCCTTGGTGGGGG |
| | | | GGAAAAGCTACTTGCTTGAGAGGCACAGACAAATAGCCAGATCCTGAACT |
| | | | TTTAATAAGGTATGTCTACACTTGCTGAGAGCACATAAAAGGGATCAGAA |
| | | | ATGGAAGAAGGGAAAGAGGCATGGAGAAAAAAAACTGCTTTTGTCCTTTG |
| | | | GCTATTTTAAATGAACAGAATAGACCTTGTCAGGTGCATAAAACACACAG |
| | | | GAGTCCTAGTTAGGCTCTTTAATCTGCAAAAGAGAACCTTAATCCTATCT |
| | | | TCTATTTGGTTGATTGTCAAAGCCTTTGGATCATCCTTTGTCTGTAGATT |
| | | | AACTACACTCTAGGATTTTGTCAAAGATTGCAACCTTTAATTCTCTGCTG |
| | | | TCCAATTCTTATCTATCTTGGTGAAGTGACATGACATTTAAGGAGAATTG |
| | | | T |
| F5 | rs2298909 | 111 | TTTTTACTCATTTTTTAATGTAGTCTAGGTATGCAGCTCTCTAATGGTTG |
| | | | GACTCTGATGAAGGTAAACTCCATGTATCAAGAGCACGGAGTTTTCCTCA |
| | | | GACAATTCTCCTTAAATGTCATGTCACTTCACCAAGATAGATAAGAATTG |
| | | | GACAGCAGAGAATTAAAGGTTGCAATCTTTGACAAAATCCTAGAGTGTAG |
| | | | WTAATCTACAGACAAAGGATGATCCAAAGGCTTTGACAATCAACCAAATA |
| | | | GAAGATAGGATTAAGGTTCTCTTTTGCAGATTAAAGAGCCTAACTAGGAC |
| | | | TCCTGTGTGTTTTATGCACCTGACAAGGTCTATTCTGTTCATTTAAAATA |
| | | | GCCAAAGGACAAAAGCAGTTTTTTTTCTCCATGCCTCTTTCCCTTCTTCC |
| | | | ATTTCTGATCCCTTTTATGTGCTCTCAGCAAGTGTAGACATACCTTATTA |
| | | | AAAGTT |
| F5 | rs9332607 | 112 | AGCCATATGACTCTCTCTCCAGAACTCAGTCAGACAAACCTTTCCCCAGC |
| | | | CCTCGGTCAGATGCCCATTTCTCCAGACCTCAGCCATACAACCCTTTCTC |
| | | | TAGACTTCAGCCAGACAAACCTCTCTCCAGAACTCAGTCAAACAAACCTT |
| | | | TCCCCAGCCCTCGGTCAGATGCCCCTTTCTCCAGACCCCAGCCATACAAC |
| | | | YCTTTCTCTAGACCTCAGCCAGACAAACCTCTCTCCAGAACTCAGTCAGA |
| | | | CAAACCTTTCCCCAGACCTCAGTGAGATGCCCCTCTTTGCAGATCTCAGT |
| | | | CAAATTCCCCTTACCCCAGACCTCGACCAGATGACACTTTCTCCAGACCT |
| | | | TGGTGAGACAGATCTTTCCCCAAACTTTGGTCAGATGTCCCTTTCCCCAG |
| | | | ACCTCAGCCAGGTGACTCTCTCTCCAGACATCAGTGACACCACCCTTCTC |
| | | | CCGGAT |
| F5 | rs9332611 | 113 | TGCCCACAGCTTTTACTAAAGCTGTTACACTAATCACTTAAAAAACCAAA |
| | | | GTGTAATTACAGTAGGCAAACACAATGTATGGTGTGACTTATGCATATGA |
| | | | GTTCATTAATCTGATGCCTGACATCTCCACATTAGAACTCTGTTTAAAAC |
| | | | ACCTTTTCCTATGACTTCCTATTTAAATGTTGCACTCAATACCACATGAG |
| | | | -/AAC |
| | | | AACTAGCTACCTGAGTATTTCTAAGCAAGGCCCCGAGGGAGTGAGATGCA |
| | | | GGGCCTGTCCAGAGTGAATAGGGATGGAACCACCTTCAAAATCTTGTATT |
| | | | CCATTCAAGGCTCACAACCCACTTTTATGGCATTCGGAGTTGCTTTACCC |
| | | | ACTCCCTTGTGTTCTGTGAGTTTGCATGTTATGCTCAGAGTCTGGAAAGA |
| | | | TGGGATTAGTAGGGGAGGTAGACCAGAAGAAAGGCAGAAGGAGATTCTCT |
| | | | GTTGA |
| F5 | rs9332590 | 114 | TAAAGTTCAGTGGCCAAAAGCACTGCCATGGCAGTCTGAGAGAAATGTCA |
| | | | CTGAGCCAGCTGGAAGCAAACTTCTCCACAACAGCCAGGGTCCCCCTACT |
| | | | GATCCTCAGAGTCTAGAGTCATGGTAATCACCTTCTCTAGTAGGTAATCA |
| | | | CACATGTGAGGATGATTTCTGCATGTTCTGTTCATATAGCTTCAGATGAC |
| | | | YGACAGCTAGGGATTATCAGAGCTGACAGGTGCCAGGTCAAATAATTCAA |
| | | | ACAGAAAATTACTCTCAGCTTTCTTTGCATAACTTCCTTTTGGCAGTGAA |
| | | | TCTATCATAGTTCTAGACCAGTGCTGTCCAATAGAAACTACGTATGAGCT |
| | | | ACATGCGTAATTTAAAATTTTCTGATAGCAACATTAAAAAGTAAAGAGAA |
| | | | ATAGGTGACATTAATTTTAGTAATATGTTTTACTTAACTCAGTATATCCT |
| | | | GAATATTATCATTTCAACATGTAACCAATATAAAAATTAATGAGACTTAC |
| | | | TTTATTACTAAGTCTTTGAAATTCAATGTATATTTTATACTCACAGCATA |
| | | | TCTCAGTCCAGTCTACCCATATTTCAAGTGCTCGATAACCACTTGTAGCC |
| | | | AGTGGCTACCATATTGAACAATGCAGTTATAGACCATTTAGAAAAACATC |
| | | | TAGAATGAGGTATAAACTAATAATACATCATGTAAATTATTAAAGTCACT |
| | | | T |
| F5 | rs7537742 | 115 | CCTGGTTGAACTGCTCTGATCATGGTGTTGTTCCTGCCTGAAAGAAAATA |
| | | | TATTCAAAATTGTTTTCATTTGCAAAGTTATTTCATGATAATAAATAAAT |
| | | | AAATAAGCTTTCGCTGGAACCAATTAATATTGCAAAAGGAATTCTTTTAT |
| | | | TTTTATTTTTTTTAAATTATACTTTAAGTTCTAGGGTACATGTGCACAAC |
| | | | RTGCAGGTTTGTTACATATGTATACATGTGTCATGTTGGTGTGCTGCACC |
| | | | CATTAACTTGTCATTAACATTAGGTATATCTCCTAATGCTATCCCTCCCC |
| | | | CCGCCCCCCACCCCCCCCCCCCGACAGGCCCCGGTGTGTGATGTTCCCCA |
| | | | TCCTGTGTCTAAATGTTCTCATTGTTCAATTGAATTCTTTAAATATTCTA |
| | | | CTTGGAACCTGGATAACATGTAGCCATTAGATAATGCTCCACTAGAGGCC |
| | | | ACTATGACACTAATAAAAGACACCATATTTTGTTACCACTAAGAGACAAA |
| | | | ACTCCTGAAGTGAGAAGGGTTTGGCTGTGATTTTTAGGATACTCCTACAT |
| | | | GTATACTACCTGACTGCAGTAGTGACACCAC |
| F5 | rs9332587 | 116 | AACCCTTCTCACTTCAGGAGTTTTGTCTCTTAGTGGTAACAAAATATGGT |
| | | | GTCTTTTATTAGTGTCATAGTGGCCTCTAGTGGAGCATTATCTAATGGCT |
| | | | ACATGTTATCCAGGTTCCAAGTAGAATATTTAAAGAATTCAATTGAACAA |
| | | | TGAGAACATTTAGACACAGGATGGGGAACATCACACACCGGGGCCTGTCG |
| | | | SGGGGGGGGGGTGGGGGGCGGGGGGAGGGATAGCATTAGGAGATATACCT |
| | | | AATGTTAATGACAAGTTAATGGGTGCAGCACACCAACATGACACATGTAT |
| | | | ACATATGTAACAAACCTGCACGTTGTGCACATGTACCCTAGAACTTAAAG |
| | | | TATAATTTAAAAAAAATAAAAATAAAAGAATTCCTTTTGCAATATTAATT |
| | | | GGTTCCAGCGAAAGCTTATTTATTTATTTATTATCATGAAATAACTTTGC |
| | | | AAATGA |
| F5 | rs9332586 | 117 | TGTGTTTTATGCACCTGACAAGGTCTATTCTGTTCATTTAAAATAGCCAA |
| | | | AGGACAAAAGCAGTTTTTTTTCTCCATGCCTCTTTCCCTTCTTCCATTTC |
| | | | TGATCCCTTTTATGTGCTCTCAGCAAGTGTAGACATACCTTATTAAAAGT |
| | | | TCAGGATCTGGCTATTTGTCTGTGCCTCTCAAGCAAGTAGCTTTTCCCCC |
| | | | YCACCAAGGGTCTTACTCTGTCACCCAGGCTGGAGTGCAGTGGTGTGATC |
| | | | ATGGCTCACTGCAACCTCCACTTCCTGGGCTCAGGCGATTCTCGCACCTC |
| | | | AGCCTCTCAAGTAGCTGGGACTACAGGCACACGCCCCAACGCCTGGCTAA |
| | | | TTTTTGTATTTTTTTGTAGAGATGAGGTTTCACCATGTTGCCCAGGCTGG |
| | | | TCTTGAACTCCTGAGCTCAAGTGATCCACCTGCTTCAGCCTCCCAAAGTG |
| | | | CTGGGGTTACAGGTTTGAGCCACTGAACCTAGCTGAGAAGATGACTTCAA |
| | | | AGACAGTGTCCTAAAAAATCTACCGGGCAGGGGAAGGGGATCATGGTCTA |
| | | | GAGATGAGAAGCAGATTCAAAATGAGATGTGGCCCAAGCTGAAAAGAGAA |
| | | | GGGCAGGGGAAGGAGGACTTGCTTGGAGAGAGTGATACTGTGAGGAAAAC |
| | | | GTCACTTCTCCTAACCTATCCTCTAAAGT |
| F5 | rs721161 | 118 | GGATCTTCACATCAGGATAAATGGTGCTTTCTTTTTGTAGATGATGTAAA |
| | (at position | | CTTCACCCTGACATATTTCCTTTTTTACACTGACTGCCATAAAGCTTAGG |
| | 170) | | ACAAAATTTGAAGACAGCCTTACAGGGTCACATGGTATCTACTTATCTGT |
| | | | GGCTTTATTTTCTTTGTCCSCATATTCTATCCCAATTACATAGACTCCTT |
| | | | GTTTTATGCCTTTATAACTTGAGAAACTGTCTCAGATCCTTTGTATTACT |
| | | | GAGTAAGCTGTAAATAAATACAAATACTAAATAAAAACTAAAAGTTGCAT |
| | | | TTGAATTTAAAATTATATGAGCATCTTTTTCTTTTAAAATTAAAAAATAA |
| | | | CCAGGTACTCCATAATATTTTACTATGTAATTTCTCCCATGATTCTGTAT |
| | | | TTGTGTTACTTACTTTGAGTGTGTCTCTGACCTGGGCTCTGATAATAGGA |
| | | | CCCAAAATCCCATCTTCTTTCGTATTGGGATTCACTGTATGTTTGGTGAA |
| | | | GGACTCATCTTCGTACTGTGTGTACATAACTTTCTTATAATGTTTTCCAA |
| | | | TTTGGTTTGAGAAATTATCCAAATGCTGAGACCTGTATTTTCTTAAAGTG |
| | | | AAGTAAAAAAAAATTAAACCACTTTCTCAA |
| F5 | rs9332577 | 119 | AGTTAAAGTCCTGAATCTTGGGAAACCCCTCAGACCAGAACAGTCGGTTA |
| | | | CCTTCTTGGTTCCCTTCTTGGCCTAATCCTTTAGCAATCCCTGTGTTTTT |
| | | | GAGTTCACATCCACCATCTCTAAGCTCTGTTGTTACCTGTATAATGCCAT |
| | | | TTATCACAATGAGTTGTCACTGCTTGTTTGCTGGTCACTCAGATGTCTGC |
| | | | -/T |
| | | | TTTTGCAGGGCACAAACTACAACTGGAGTGTCTGTTCCCTCAAAGCTTTA |
| | | | TACAATGCCTGACACACAGCAGGTGCTCAATACACTTGGTGAGTGAATGT |
| | | | TTGATCACAGAGTACTTGACTGAATGCTTATTTTGGCCTGTGTCTCTCCC |
| | | | TCTTTCTCAGATATAACAGTTTGTGCCCATGACCACATCAGCTGGCATCT |
| | | | GCTGGGAATGAGCTCGGGGCCAGAATTATTCTCCATTCATTTCAACGGCC |
| | | | AGGTCCTGGAGCAGAACCATCATAAGGTCTCAGCCATCACCCTTGTCAGT |
| | | | GCTACATCCACTACCGCAAATATGACTGTGGGCCCAGAGGGAAAGTGGAT |
| | | | CATATCTTCTCTCACCCCAAAACATTTGCAAGGCAAGAAACTCTCCTGAC |
| F5 | rs2239854 | 120 | TCTTCCTCTTTTCTTCTTTCGAGGAAGTTAGAGATCTCTTTAGCTTTTGC |
| | | | TTAATTAAAAAAAAAAAAAAAACCTTTGCCAATTCCTTCGCTTTCTCCAT |
| | | | CCCCAAAGAGCAAGTTATAAATCTAAGAGCAAAATATCTAAGTTTGGTTG |
| | | | TTAGGAACTGAGGAAAGTTTGTCTGCGGTGCAGGTGGCTTGAAAGGGCAA |
| | | | RGGAGAAAGAGGGAGTTAGTGCATGGGAAGAAAGGATTCTGCATTGAGAA |
| | | | GCAAGACTGTCAGGAGAGTTTCTTGCCTTGCAAATGTTTTGGGGTGAGAG |
| | | | AAGATATGATCCACTTTCCCTCTGGGCCCACAGTCATATTTGCGGTAGTG |
| | | | GATGTAGCACTGACAAGGGTGATGGCTGAGACCTTATGATGGTTCTGCTC |
| | | | CAGGACCTGGCCGTTGAAATGAATGGAGAATAATTCTGGCCCCGAGCTCA |
| | | | TTCCCA |
| F5 | rs4656688 | 121 | TTATATCTGAGAAAGAGGGAGAGACACAGGCCAAAATAAGCATTCAGTCA |
| | | | AGTACTCTGTGATCAAACATTCACTCACCAAGTGTATTGAGCACCTGCTG |
| | | | TGTGTCAGGCATTGTATAAAGCTTTGAGGGAACAGACACTCCAGTTGTAG |
| | | | TTTGTGCCCTGCAAAAGCAGACATCTGAGTGACCAGCAAACAAGCAGTGA |
| | | | YAACTCATTGTGATAAATGGCATTATACAGGTAACAACAGAGCTTAGAGA |
| | | | TGGTGGATGTGAACTCAAAAACACAGGGATTGCTAAAGGATTAGGCCAAG |
| | | | AAGGGAACCAAGAAGGTAACCGACTGTTCTGGTCTGAGGGGTTTCCCAAG |
| | | | ATTCAGGACTTTAACTATTAGAACTGGTAAAAAGTCCACGGCAAAATGGA |
| | | | ATGTTTGGTCACCCCAGTCTGATCTCAGCTGGAGGGAGTCAAATCACCAG |
| | | | AACCGCCAATAGGGCTGAGTTCAAGCACTTAATCCTCTCAGCTCTCTAGC |
| F5 | rs4656689 | 122 | CATTCACTCACCAAGTGTATTGAGCACCTGCTGTGTGTCAGGCATTGTAT |
| | | | AAAGCTTTGAGGGAACAGACACTCCAGTTGTAGTTTGTGCCCTGCAAAAG |
| | | | CAGACATCTGAGTGACCAGCAAACAAGCAGTGACAACTCATTGTGATAAA |
| | | | TGGCATTATACAGGTAACAACAGAGCTTAGAGATGGTGGATGTGAACTCA |
| | | | RAAACACAGGGATTGCTAAAGGATTAGGCCAAGAAGGGAACCAAGAAGGT |
| | | | AACCGACTGTTCTGGTCTGAGGGGTTTCCCAAGATTCAGGACTTTAACTA |
| | | | TTAGAACTGGTAAAAAGTCCACGGCAAAATGGAATGTTTGGTCACCCCAG |
| | | | TCTGATCTCAGCTGGAGGGAGTCAAATCACCAGAACCGCCAATAGGGCTG |
| | | | AGTTCAAGCACTTAATCCTCTCAGCTCTCTAGCTGTAGTGGTCGAAGCTC |
| | | | TGCCTAAGGGAAGAAGATGTGAAGATGATATGAGGATTTTCAATTGTTAT |
| | | | TTTTA |
| F5 | rs4656188 | 123 | GAGGGAACAGACACTCCAGTTGTAGTTTGTGCCCTGCAAAAGCAGACATC |
| | | | TGAGTGACCAGCAAACAAGCAGTGACAACTCATTGTGATAAATGGCATTA |
| | | | TACAGGTAACAACAGAGCTTAGAGATGGTGGATGTGAACTCAAAAACACA |
| | | | GGGATTGCTAAAGGATTAGGCCAAGAAGGGAACCAAGAAGGTAACCGACT |
| | | | KTTCTGGTCTGAGGGGTTTCCCAAGATTCAGGACTTTAACTATTAGAACT |
| | | | GGTAAAAAGTCCACGGCAAAATGGAATGTTTGGTCACCCCAGTCTGATCT |
| | | | CAGCTGGAGGGAGTCAAATCACCAGAACCGCCAATAGGGCTGAGTTCAAG |
| | | | CACTTAATCCTCTCAGCTCTCTAGCTGTAGTGGTCGAAGCTCTGCCTAAG |
| | | | GGAAGAAGATGTGAAGATGATATGAGGATTTTCAATTGTTATTTTTACTT |
| | | | ACTTTTTGATTGTTTTAATGACAAGTCAACGAAATCACTTT |
| F5 | rs1894697 | 124 | GCTGGAGGGAGTCAAATCACCAGAACCGCCAATAGGGCTGAGTTCAAGCA |
| | (at position | | CTTAATCCTCTCAGCTCTCTAGCTSTAGTGGTCGAAGCTCTGCCTAAGGG |
| | 75) | | AAGAAGATGTGAAGATGATATGAGGATTTTCAATTGTTATTTTTACTTAC |
| | | | TTTTTGATTGTTTTAATGACAAGTCAACGAAATCACTTTGGGGTTACACA |
| | | | CTCTCCTTAAAATGCAGTGTACAAGTCCTCATTATGCTGAGCCATTGGGA |
| | | | GCTTTTCATGGAAGGAGTAGTGATAACTGAATATAAAAACTTAGTTTTGC |
| | | | TCCTTCTTTGCTCCTACAGTCACTGGGAAAATGCTCATTTGCTCTGTGGG |
| | | | GAGACTCCATCCTTGGCTTTTAGGTTTCTGTTTTACATCTCAGATACATA |
| | | | ATCACTAGATACTAGATAATGGGCCTGAGAATCAGTTCCTTACCCACACA |
| | | | AAGGCTTGTTTTTTTTAGGAGACCATTGCAACAAAGCCAGGATTCCTTGC |
| | | | ATCACTAGGAGGCTTTGGTGGAAGCGTTTATCCATGCCAAAGAAATGCTT |
| | | | ATATTGAGTTCAGCAATTAACATATCTGTGTTAGCATTTATCATATGTAT |
| | | | TCAATTTATTCTGCTTTATAAGGCAGAGGGTTTTTACAAATGTTT |
| F5 | rs1894698 | 125 | CTGGAGGGAGTCAAATCACCAGAACCGCCAATAGGGCTGAGTTCAAGCAC |
| | (at position | | TTAATCCTCTCAGCTCTCTAGCTGTAGTGGTCGAAGCTCTGCCTAAGGGA |
| | 119) | | AGAAGATGTGAAGATGATRTGAGGATTTTCAATTGTTATTTTTACTTACT |
| | | | TTTTGATTGTTTTAATGACAAGTCAACGAAATCACTTTGGGGTTACACAC |
| | | | TCTCCTTAAAATGCAGTGTACAAGTCCTCATTATGCTGAGCCATTGGGAG |
| | | | CTTTTCATGGAAGGAGTAGTGATAACTGAATATAAAAACTTAGTTTTGCT |
| | | | CCTTCTTTGCTCCTACAGTCACTGGGAAAATGCTCATTTGCTCTGTGGGG |
| | | | AGACTCCATCCTTGGCTTTTAGGTTTCTGTTTTACATCTCAGATACATAA |
| | | | TCACTAGATACTAGATAATGGGCCTGAGAATCAGTTCCTTACCCACACAA |
| | | | AGGCTTGTTTTTTTTAGGAGACCATTGCAACAAAGCCAGGATTCCTTGCA |
| | | | TCACTAGGAGGCTTTGGT |
| F5 | rs1894699 | 126 | AGTCCTCATTATGCTGAGCCATTGGGAGCTTTTCATGGAAGGAGTAGTGA |
| | | | TAACTGAATATAAAAACTTAGTTTTGCTCCTTCTTTGCTCCTACAGTCAC |
| | | | TGGGAAAATGCTCATTTGCTCTGTGGGGAGACTCCATCCTTGGCTTTTAG |
| | | | GTTTCTGTTTTACATCTCAGATACATAATCACTAGATACTAGATAATGGG |
| | | | SCTGAGAATCAGTTCCTTACCCACACAAAGGCTTGTTTTTTTTAGGAGAC |
| | | | CATTGCAACAAAGCCAGGATTCCTTGCATCACTAGGAGGCTTTGGTGGAA |
| | | | GCGTTTATCCATGCCAAAGAAATGCTTATATTGAGTTCAGCAATTAACAT |
| | | | ATCTGTGTTAGCATTTATCATATGTATTCAATTTATTCTGCTTTATAAGG |
| | | | CAGAGGGTTTTTACAAATGTTT |
| F5 | rs1981491 | 127 | CATCACTAGGAGGCTTTGGTGGAAGCGTTTATCCATGCCAAAGAAATGCT |
| | | | TATATTGAGTTCAGCAATTAACATATCTGTGTTAGCATTTATCATATGTA |
| | | | TTCAATTTATTCTGCTTTATAAGGCAGAGGGTTTTTACAAATGTTTACTA |
| | | | TTCTTAGTTTCTACTGATGGTCTTTCCCTACCTTGTCCCATTTACCCCCA |
| | | | RTTATTCTTTTGGGAACTATAAATTCGTAAACTCTAAGCTCAGATCAATT |
| | | | TATTTTTATTTTTGTGCATATGGTTTTGCTTTT |
| F5 | rs7548857 | 128 | ATTTTAATTTTTTAATGTTTTTCCCTCAGGCTAGTATCATATACCCTGGA |
| | | | TCTTTAGTTGTAGGTCAGTTCTACCAGAGAGAAATAGAAGAGATGTGGTC |
| | | | TAAGTGGGAATTAGTTATTTTTTCTCTTAACGATTGCTATGATTTTGCCT |
| | | | TCTCATAAGTGCCCAAATGCAAATGGAAAGAGAAAATATTCTAGATGCCA |
| | | | YTGAATATTACTCTTCTGTCACCTTTTGAAATGCTGGTTTTTTTTGTGTG |
| | | | TTGAGGATTACTCCTAATCTGTTCATCTTTACTGCAATCTTCCTGCTGAT |
| | | | CCTTCCTGCTAGGGCTTGTCCTTGAAGAAGCCATGGTCATCATATAGAGG |
| | | | AACTGCAGAACTCGGAGAGAGTGGCTGAGCTGATACCTTAAGGTCTTATT |
| | | | CAAAGTACAGGCCAAGGTGAAACCTACTCAGGTCACTATACCTAACAAGG |
| | | | CTGCAC |
| F5 | rs6427202 | 129 | ATAATGATTTCAACTTGAAGAAATTCTTATTTTTTAAAATAGCATATTTA |
| | | | TCAAGTAATAATGGTTCTAGGGTTATTTAAAATCTGGTGCCACACTGTCT |
| | | | TCTTTGATACCTTCTTGCTTTGGGGAACAGGCTTTATTTCATCCAGCTTG |
| | | | GGAAGAAAACATTTGGTGACTCTCAAGACTCTGTACAAGTATCCATTTCC |
| | | | YCCAGTCTCAATAGAAGAGTATATAGATACGTTAGCTCAGTTGGTAAAAG |
| | | | CATCATGCCCCAAGTTTAATGCTGTCATCTTATTTGTATATGAAAGGGGG |
| | | | CATTAGATAATTGGGTGGAAGTATCAGCATTAATCCATTCTCACTATGAG |
| | | | AAAAACATGCTGCTGATGGTAAAATGCAAGAAGAGTCTTTTGTAAGTTTT |
| | | | AAGGTTTTTTTCAGTACCATCACAGTATCTTATATATACCAGGAAATGGA |
| | | | AAAAAA |
| F5 | rs9287093 | 130 | GTTGAAAACTTTGGGTGGTAGGTTTCAAACACACTCAAGTGACTTTACAA |
| | | | GAACTTTTATTGGCTGGGCTTCTTAGGAGCAAGCTTGGATTTTCATTTTA |
| | | | TCCCATGAGAATCCAGCCCATCTTTCATTAAAACAAACAACCACAGTGAG |
| | | | CCACAGCATGGGAAATAACGTGATGGACTCACCATCTGCCTATACATACA |
| | | | -/A/CG/G |
| | | | CTCAGGCCATACCTACATACATGCCCAATGTATTTACTCATTATACAAAG |
| | | | ACTTTTTTTCCATTTCCTGGTATATATAAGATACTGTGATGGTACTGAAA |
| | | | AAAACCTTAAAACTTACAAAAGACTCTTCTTGCATTTTACCATCAGCAGC |
| | | | ATGTTTTTCTCATAGTGAGAATGGATTAATGCTGATACTTCCACCCAATT |
| | | | ATCTAATGCCCCCTTTCATATACAAATAAGATGACAGCATTAAACTTGGG |
| | | | GCATG |
| F5 | rs1894700 | 131 | CTGAACAATTATTATTTAAATATAATCAGAGAATTGAATCGCCTTTGATT |
| | (at position | | AGTAAACCATCTCAGAATATAAGAATGGGTGATATCTTCTTTGTGGTSTT |
| | 98) | | TAATAAGACATACTTAGCCTATTAACATGAGATGGGAGGATAGTCAGAGC |
| | | | TGTTTATGTATTATTTTGTTTTGCTATTTCAGATCTTCTTTCCAACTGCT |
| | | | GTGATCCTCAGCTTTGTTTTATCTCTTTATTTCATCTTAGCCATCTGATT |
| | | | ATCTCAATTTTTTCCCCCGAGCCACAGCGTCTAACTCCCCTATGCAGGCT |
| | | | GGTTGTCTGTATGAATATGAAATGTGTCCATCCCCTGAAACTTAGATGTC |
| | | | TTACTTTGTACTTTGTGCACCCAAAGACACATCATTAACACCTCCATGTA |
| | | | TATTGGACTTTCCCTACTGATAGGAGAGGGAGAGGATGAAGAAATTGGTC |
| | | | TTCTTTATTGAAGAGAAGGAACTGAGATACTCAATATTAAAAGAAGTGTA |
| | | | GAGGGTTGGGTGAGGTTTTCAATAATAAAATGGAGCAAGTGAGGTCAGGA |
| | | | AGGGGAGAGGTGAGTGCCATAGAGAGCCAAGTGGAATGAACAGTTTCCTC |
| | | | TGCCTGAGGAATTCCTCATTTAATTAGGTGAAAGATTCCCTATCATCAG |
| F5 | rs5778621 | 132 | ATAAGAATGGGTGATATCTTCTTTGTGGTGTTTAATAAGACATACTTAGC |
| | | | CTATTAACATGAGATGGGAGGATAGTCAGAGCTGTTTATGTATTATTTTG |
| | | | TTTTGCTATTTCAGATCTTCTTTCCAACTGCTGTGATCCTCAGCTTTGTT |
| | | | TTATCTCTTTATTTCATCTTAGCCATCTGATTATCTCAATTTTTTCCCCC |
| | | | -/A/C |
| | | | GAGCCACAGCGTCTAACTCCCCTATGCAGGCTGGTTGTCTGTATGAATAT |
| | | | GAAATGTGTCCATCCCCTGAAACTTAGATGTCTTACTTTGTACTTTGTGC |
| | | | ACCCAAAGACACATCATTAACACCTCCATGTATATTGGACTTTCCCTACT |
| | | | GATAGGAGAGGGAGAGGATGAAGAAATTGGTCTTCTTTATTGAAGAGAAG |
| | | | GAACTGAGATACTCAATATTAAAAGAAGTGTAGAGGGTTGGGTGAGGTTT |
| | | | TCAATAATAAAATGGAGCAAGTGAGGTCAGGAAGGGGAGAGGTGAGTGCC |
| | | | ATAGAGAGCCAAGTGGAATGAACAGTTTCCTCTGCCTGAGGAATTCCTCA |
| | | | TTTAATTAGGTGAAAGATTCCCTATCATCAGCTGGTGACAGAATTTCTCT |
| F5 | rs7542281 | 133 | ACCCTGGATTGAGTGGCTTATAAACAACGGACACTTCTTCCTCACAGTTC |
| | | | TGGAGGCTGGAAGTTTAAAATCAGGGTGCCAGCATGGTTGGGTTCTGGTG |
| | | | CAGGGTCTCTTCCAGGTTGTAGACTGCCATCTTCTCCTTGTATCCTCACA |
| | | | TGGTAGAAAGAGGGTGAGTGAGAGGGTCCCTTTTATAAGGGCACTAATCT |
| | | | YATCATGAGGGCTCCATCCTCATTACCTAAAAATCTCCCAAAGGAGGGGG |
| | | | GAGGGATAGCATTGGGAGATATACCTAATGCTAGATGACGAGTTAGTGGG |
| | | | TGCAGCGCACCAGCATGGCACATGTATACATATGTAACTAACCTGCACAA |
| | | | TGTGCACATGTACCCTAAAACTTAAAGTATAATAAAAAAAAATCTCCCAA |
| | | | AGGCCCCACCTGCTAATGCCATCACACTGGAGGTTAGATTTCAGTATGTG |
| | | | AATTTTTGGAGGACACAAACTTCCAATCCATTGTAGTGATGTATCTATTC |
| | | | CAAAGGCGATGAAAGTAAATAAGACTTTTTTGGTAAAAGTACTTTTTTTT |
| | | | TTTTTTTTTGGTAATAAGTAAGACAAAGTACCTGCTCAAAATTATCAGCA |
| | | | AAATCAATAATTTTAAAGCAAGGGAAAAATAATGCATAGTCCTTACTTTC |
| | | | TAATCAGTCCTCGGCCCCTCAGTTTAGTTCA |
| F5 | rs2187954 | 134 | GTTCTGGAGGCTGGAAGTTTAAAATCAGGGTGCCAGCATGGTTGGGTTCT |
| | | | GGTGCAGGGTCTCTTCCAGGTTGTAGACTGCCATCTTCTCCTTGTATCCT |
| | | | CACATGGTAGAAAGAGGGTGAGTGAGAGGGTCCCTTTTATAAGGGCACTA |
| | | | ATCTCATCATGAGGGCTCCATCCTCATTACCTAAAAATCTCCCAAAGGAG |
| | | | VGGGGAGGGATAGCATTGGGAGATATACCTAATGCTAGATGACGAGTTAG |
| | | | TGGGTGCAGCGCACCAGCATGGCACATGTATACATATGTAACTAACCTGC |
| | | | ACAATGTGCACATGTACCCTAAAACTTAAAGTATAATAAAAAAAAATCTC |
| | | | CCAAAGGCCCCACCTGCTAATGCCATCACACTGGAGGTTAGATTTCAGTA |
| | | | TGTGAATTTTTGGAGGACACAAACTTCCAATCCATTGTAGTGATGTATCT |
| F5 | rs9332556 | 135 | GTCCTCCAAAAATTCACATACTGAAATCTAACCTCCAGTGTGATGGCATT |
| | | | AGCAGGTGGGGCCTTTGGGAGATTTTTTTTTATTATACTTTAAGTTTTAG |
| | | | GGTACATGTGCACATTGTGCAGGTTAGTTACATATGTATACATGTGCCAT |
| | | | GCTGGTGCGCTGCACCCACTAACTCGTCATCTAGCATTAGGTATATCTCC |
| | | | MAATGCTATCCCTCCCCCCTCCTTTGGGAGATTTTTAGGTAATGAGGATG |
| | | | GAGCCCTCATGATGAGATTAGTGCCCTTATAAAAGGGACCCTCTCACTCA |
| | | | CCCTCTTTCTACCATGTGAGGATACAAGGAGAAGATGGCAGTCTACAACC |
| | | | TGGAAGAGACCCTGCACCAGAACCCAACCATGCTGGCACCCTGATTTTAA |
| | | | ACTTCCAGCCTCCAGAACTGTGAGGAAGAAGTGTCCGTTGTTTATAAGCC |
| | | | ACTCAATCCAGGGTACTTTGTTAACAGCAACCCAAACTAAGAAAATCACC |
| | | | AATTTCTATCTTACTCACATGTGTTACCAGAACTCAAGCACACTTAAGAT |
| | | | CTATCTTATCTAAAGTAGAACATAAAGGAAAGAGGCTGTATTAATTGCAA |
| | | | CATGACTGGGAAAGAAAATACTTAACCAAAATGTGAACATGTATTCCCAC |
| | | | AGGCTATCAGAGCATTTGTTAGGAGCCAGAAATTTTA |
| F5 | rs2187955 | 136 | CTCCATCCTCATTACCTAAAAATCTCCCAAAGGAGGGGGGAGGGATAGCA |
| | | | TTGGGAGATATACCTAATGCTAGATGACGAGTTAGTGGGTGCAGCGCACC |
| | | | AGCATGGCACATGTATACATATGTAACTAACCTGCACAATGTGCACATGT |
| | | | ACCCTAAAACTTAAAGTATAATAAAAAAAAATCTCCCAAAGGCCCCACCT |
| | | | RCTAATGCCATCACACTGGAGGTTAGATTTCAGTATGTGAATTTTTGGAG |
| | | | GACACAAACTTCCAATCCATTGTAGTGATGTATCTATTCCAAAGGCGATG |
| | | | AAAGTAAATAAGACTTTTTTGGTAAAAGTACTTTTTTTTTTTTTTTTTGG |
| | | | TAATAAGTAAGACAAAGTACCTGCTCAAAATTATCAGCAAAATCAATAAT |
| | | | TTTAAAGCAAGGGAAAAATAATGCATAGTCCTTACTTTCTAATCAGTCCT |
| | | | CGGCCCCTCAGTTTAGTTCA |
| F5 | rs9332554 | 137 | GAAGAAATCTAGAGGTCAAACCTATCACAATTAGGGCACCAACATGTACC |
| | | | CTAGAGTTAACTAGGCAGAGCCCCTCAGGGGCCCAGGCCCTGTCTTCTTT |
| | | | ACATTTGTACTCTGGCATTTAGCACAGAGCCTGGAACCTAGTGGATGATG |
| | | | ATGGTCATGATCATGATGATATTAATCATTACTGAACTAAACTGAGGGGC |
| | | | YGAGGACTGATTAGAAAGTAAGGACTATGCATTATTTTTCCCTTGCTTTA |
| | | | AAATTATTGATTTTGCTGATAATTTTGAGCAGGTACTTTGTCTTACTTAT |
| | | | TACCAAAAAAAAAAAAAAAAAGTACTTTTACCAAAAAAGTCTTATTTACT |
| | | | TTCATCGCCTTTGGAATAGATACATCACTACAATGGATTGGAAGTTTGTG |
| | | | TCCTCCAAAAATTCACATACTGAAATCTAACCTCCAGTGTGATGGCATTA |
| | | | GCAGGTGGGGCCTTTGGGAGATTTTTTTTTATTATACTTTAAGTTTTAGG |
| | | | GTACATGTGCACATTGTGCAGGTTAGTTACATATGTATACATGTGCCATG |
| | | | CTGGTGCGCTGCACCCACTAACTCGTCATCTAGCATTAGGTATATCTCCC |
| | | | AATGCTATCCCTCCCCCCTCCTTTGGGAGATTTTTAGGTAATGAGGATGG |
| | | | AGCCCTCATGATGAGATTAGTGCCCTTATAAAAGGGACCCTCTCACTCAC |
| | | | C |
| F5 | rs9332553 | 138 | TCACCCTGCTATACCTAGCACAAGTGTTATAGCATCTTGGGCAATTTTTA |
| | | | GATACTTACATTGGTTGGTTCTCATTCCAGATTGACAGTGTGTTCTAATT |
| | | | GTAGCCCTAGAAGAAATCTAGAGGTCAAACCTATCACAATTAGGGCACCA |
| | | | ACATGTACCCTAGAGTTAACTAGGCAGAGCCCCTCAGGGGCCCAGGCCCT |
| | | | KTCTTCTTTACATTTGTACTCTGGCATTTAGCACAGAGCCTGGAACCTAG |
| | | | TGGATGATGATGGTCATGATCATGATGATATTAATCATTACTGAACTAAA |
| | | | CTGAGGGGCCGAGGACTGATTAGAAAGTAAGGACTATGCATTATTTTTCC |
| | | | CTTGCTTTAAAATTATTGATTTTGCTGATAATTTTGAGCAGGTACTTTGT |
| | | | CTTACTTATTACCAAAAAAAAAAAAAAAAAGTACTTTTACCAAAAAAGTC |
| | | | TTATTT |
| F5 | rs6670678 | 139 | TACTACTAAAAGTTGAATTATGAGCATCTTGAAACAGTGAATGATATAGA |
| | | | CTTGTACATAGGATATATTCTATAATTACACTGAGTGAGATGGCTAATGA |
| | | | GACAATTGGGGTGTAATTTTATCAATGCTTTTATTCTTTTCACTTCAAAT |
| | | | TATTTTACCTTTAGTCTAGAATAAAACAGGTTTGTTGTATCTTTGATTTT |
| | | | RCAACATACATTAATATAAAGTATAAAATACAAACAGCTATTAAGAGGAA |
| | | | GCATTTGTGAGATGCAGTTTTGGTGAATGTGATTTTGACTTTGTAATCAA |
| | | | AATAAAAAAAATTAAGCTCTAAACTGAAAAGAAGAGAAATGGACAGGGAC |
| | | | AACTATTGTGCTAGAGCACAAGAAGTCCTTGTTCAGCTGCTTGCTGGAAT |
| | | | AAAATCTTTACACAAGGGTAGCTCTTCATTTATATTTACTCAGTTCTGTT |
| | | | CATTTC |
| F5 | rs9332548 | 140 | ATAAATACAATACAAAGCTGACTATCGACTGAGCAGGAAATGAACAGAAC |
| | | | TGAGTAAATATAAATGAAGAGCTACCCTTGTGTAAAGATTTTATTCCAGC |
| | | | AAGCAGCTGAACAAGGACTTCTTGTGCTCTAGCACAATAGTTGTCCCTGT |
| | | | CCATTTCTCTTCTTTTCAGTTTAGAGCTTAATTTTTTTTATTTTGATTAC |
| | | | A/- |
| | | | AAGTCAAAATCACATTCACCAAAACTGCATCTCACAAATGCTTCCTCTTA |
| | | | ATAGCTGTTTGTATTTTATACTTTATATTAATGTATGTTGCAAAATCAAA |
| | | | GATACAACAAACCTGTTTTATTCTAGACTAAAGGTAAAATAATTTGAAGT |
| | | | GAAAAGAATAAAAGCATTGATAAAATTACACCCCAATTGTCTCATTAGCC |
| | | | ATCTCACTCAGTGTAATTATAGAATATATCCTATGTACAAGTCTATATCA |
| | | | TTCAC |
| F5 | rs2298907 | 141 | TGTAACTAGCATGCATCAAATTGACTTCAATGCTGCACCTTTGAGCAAAG |
| | | | TTTGTTGTTCAGTAAAATTTTCAAAGTCGTTTTGGTCAATTACCTTTAAA |
| | | | AAGCTTGTTTCATGGAGAATTTAATATTATTCTTTTCTTTTAGTTATATT |
| | | | CTCATATTTTAAATATATGAGTTGCATATGAATGTGATGTCACAAAATTA |
| | | | STTCTATAATATATAGCCATCTGCTGGTAAGCCAGCTCTCCAAATAAAAC |
| | | | ACGTTTTCTGCCAGACATTGTTCGTTGTATCTAAGTGTTGCCAAATTCTC |
| | | | TATACAAAAATTTGCTCTTCTAGATTTTTCCAAAACATTCCAATGCTTGA |
| | | | CTTAATGTAGGTGATTACATTTTTTCACTTCTCATCTGAATATTTTGGCC |
| | | | TTATGTAGAGACTTCCTTGAGTATATGATAAACACCTGAAACAAACTATA |
| | | | ACGGCT |
| F5 | rs2298905 | 142 | GTAGGGGGTTAAGATAGTATTACACTGGCTTGAGAAACCACAACAGAACA |
| | | | TAAGAGAACTCTGGATAACCCTTTTTGACAGAATGGACAAACGGTGATTT |
| | | | GTAAAAATGCTTGTAAGGACATTTCCTTTAATAATGTATTTAATAAGACT |
| | | | GTCTTAGATCAGGGAATAAAGTATCTGCAACTGTAACTAATCACTTAAAA |
| | | | RCTGTCACTAAAGGGTTTCAATTTGAGGTTAAATTTTCAGAAACTCTGTA |
| | | | ACTAGCATGCATCAAATTGACTTCAATGCTGCACCTTTGAGCAAAGTTTG |
| | | | TTGTTCAGTAAAATTTTCAAAGTCGTTTTGGTCAATTACCTTTAAAAAGC |
| | | | TTGTTTCATGGAGAATTTAATATTATTCTTTTCTTTTAGTTATATTCTCA |
| | | | TATTTTAAATATATGAGTTGCATATGAATGTGATGTCACAAAATTAGTTC |
| | | | TATAAT |
| F5 | rs9332542 | 143 | CCTCTTCCTTTACTGTTTATTTGTCCCTACATTCCCTACAGCCAGCAATT |
| | | | ATTTATGAGCAATTGCTTAAAATGCTCCCACAGCTTTCACTGGAGACATG |
| | | | ATTATTGGCATATAATATTGCCTCTGGTCCTATGAATCTAAGAAAGGTAA |
| | | | ATACATATTGGTAGGGAAAGTAAAATTTTACCTTGGAAATGAGATGCAAA |
| | | | YCTAGTAAGCAGATAGTATCTTTTATGGTAAAAGACAAGCCTTATAAGTT |
| | | | GATTTTATTATGCCCTCTGCATTAAAATAAAACATCTCCTCCTCCATGTC |
| | | | TGACACTCACCCTCACCTATAGCCTCCCTCTGTGGTTTGAGCTATTTTTT |
| | | | TGGGAGTGGGGATAAACTAACTGCATGCTGAGCCAGTTACATGAGAATCC |
| | | | AGATTCCTCTATCTCAGAGGTAGATATTTGAGAGATAGGTGGTAGGGGGT |
| | | | TAAGAT |
| F5 | rs9332538 | 144 | AATGGTAATAATAATCATGTCTCTGTCATACAGGTTTGTTGTCGTGAAAT |
| | | | CTTAGAATAATGACTGGCATATAACAGATAATAAATATTAGCCATGATAG |
| | | | TAAAGATAATTCTTCAATTAAAATGTGTTCAAATAATTTAGTATTTATCT |
| | | | TAAAACTTGTTAGTTTCAGAAAAAATCAGAAAGTAATTTTTAAATTTATT |
| | | | RTTTTCTATATAAACTGTTGTCAAACTCATACCCACTAAGGTATAAGTGA |
| | | | CTGATAATAGGTCAATGAACTACCTTCCTTAGGAGTACTTGCATTAGCAC |
| | | | TTAATCTTTGGCTTGAGGAAAAACAATGAAGCATTAACTGTGGCACTGAG |
| | | | ATTTCTTCCAAAGTGAATTTGGCAAGACTCTGGGTGAGGTAGTGGGAACA |
| F5 | rs9332537 | 145 | CCTCTGTGTGCCTCATCTTTCTCATCTGTAAAATGGTAATAATAATCATG |
| | | | TCTCTGTCATACAGGTTTGTTGTCGTGAAATCTTAGAATAATGACTGGCA |
| | | | TATAACAGATAATAAATATTAGCCATGATAGTAAAGATAATTCTTCAATT |
| | | | AAAATGTGTTCAAATAATTTAGTATTTATCTTAAAACTTGTTAGTTTCAG |
| | | | -/A |
| | | | AAAAATCAGAAAGTAATTTTTAAATTTATTGTTTTCTATATAAACTGTTG |
| | | | TCAAACTCATACCCACTAAGGTATAAGTGACTGATAATAGGTCAATGAAC |
| | | | TACCTTCCTTAGGAGTACTTGCATTAGCACTTAATCTTTGGCTTGAGGAA |
| | | | AAACAATGAAGCATTAACTGTGGCACTGAGATTTCTTCCAAAGTGAATTT |
| | | | GGCAAGACTCTGGGTGAGGTAGTGGGAACAGGAGTTTCTCCTATGTTCTT |
| | | | AAAAT |
| F5 | rs2227245 | 146 | CAAGTTTTAAGATAAATACTAAATTATTTGAACACATTTTAATTGAAGAA |
| | (at position | | TTATCTTTACTATCATGGCTAATATTTATTATCTGTTATATGCCAGTCAT |
| | 116) | | TATTCTAAGATTTCAYGACAACAAACCTGTATGACAGAGACATGATTATT |
| | | | ATTACCATTTTACAGATGAGAAAGATGAGGCACACAGAGGTTAAATAACT |
| | | | TCATAAAAGTCACTGAGCCAGGATTTGCACTTATTAGTCTAGTTCTAAAA |
| | | | CCTGCACATAAACCACTCTCCTACTCAATTATTCTCTCAAAGGTATGATG |
| | | | GCTGGAACATGTAGAAGGAAAGATATTTAAATGTGAACCATGAAAAGTCT |
| | | | GAAATTATTTTAAAATGTTCTCATACCAACAACTATTATAATATGGATAC |
| | | | AATTTTTTATACCAGTGCCTGATGGAACTCTACTATGCTTACAATGATCT |
| | | | GAACATCAGCATAATGGGATAATTAGAACCATATTAACATCAGGTACTTA |
| | | | CTATTCAGCGGCTGATACAATAACTTGCATGACTATTATCTTTATGATTA |
| | | | TTGCCATCATTGTCATCATTATTTATAGAGAGCTTATCCGATCCCAGG |
| F5 | rs5778622 | 147 | TCACGACAACAAACCTGTATGACAGAGACATGATTATTATTACCATTTTA |
| | | | CAGATGAGAAAGATGAGGCACACAGAGGTTAAATAACTTCATAAAAGTCA |
| | | | CTGAGCCAGGATTTGCACTTATTAGTCTAGTTCTAAAACCTGCACATAAA |
| | | | CCACTCTCCTACTCAATTATTCTCTCAAAGGTATGATGGCTGGAACATGT |
| | | | -/AGG |
| | | | AGAAGGAAAGATATTTAAATGTGAACCATGAAAAGTCTGAAATTATTTTA |
| | | | AAATGTTCTCATACCAACAACTATTATAATATGGATACAATTTTTTATAC |
| | | | CAGTGCCTGATGGAACTCTACTATGCTTACAATGATCTGAACATCAGCAT |
| | | | AATGGGATAATTAGAACCATATTAACATCAGGTACTTACTATTCAGCGGC |
| | | | TGATACAATAACTTGCATGACTATTATCTTTATGATTATTGCCATCATTG |
| | | | TCATCATTATTTATAGAGAGCTTATCCGATCCCAGGAACCATGTTTAGTA |
| | | | CTCTACCTAAGTGACTTCATTTAAATTTCAGGCAATCTTATGGGTGGTTA |
| | | | TAATCATTTCCATTTTATAGATGAGAAAACTGAGGCTCAGAGATGCTAAA |
| F5 | rs9332534 | 148 | CATGCAAGTTATTGTATCAGCCGCTGAATAGTAAGTACCTGATGTTAATA |
| | | | TGGTTCTAATTATCCCATTATGCTGATGTTCAGATCATTGTAAGCATAGT |
| | | | AGAGTTCCATCAGGCACTGGTATAAAAAATTGTATCCATATTATAATAGT |
| | | | TGTTGGTATGAGAACATTTTAAAATAATTTCAGACTTTTCATGGTTCACA |
| | | | -/TTTA |
| | | | AATATCTTTCCTTCTCCTACATGTTCCAGCCATCATACCTTTGAGAGAAT |
| | | | AATTGAGTAGGAGAGTGGTTTATGTGCAGGTTTTAGAACTAGACTAATAA |
| | | | GTGCAAATCCTGGCTCAGTGACTTTTATGAAGTTATTTAACCTCTGTGTG |
| | | | CCTCATCTTTCTCATCTGTAAAATGGTAATAATAATCATGTCTCTGTCAT |
| | | | ACAGGTTTGTTGTCGTGAAATCTTAGAATAATGACTGGCATATAACAGAT |
| | | | AATAA |
| F5 | rs2213870 | 149 | GAAAGATGAGGCACACAGAGGTTAAATAACTTCATAAAAGTCACTGAGCC |
| | | | AGGATTTGCACTTATTAGTCTAGTTCTAAAACCTGCACATAAACCACTCT |
| | | | CCTACTCAATTATTCTCTCAAAGGTATGATGGCTGGAACATGTAGAAGGA |
| | | | AAGATATTTAAATGTGAACCATGAAAAGTCTGAAATTATTTTAAAATGTT |
| | | | MTCATACCAACAACTATTATAATATGGATACAATTTTTTATACCAGTGCC |
| | | | TGATGGAACTCTACTATGCTTACAATGATCTGAACATCAGCATAATGGGA |
| | | | TAATTAGAACCATATTAACATCAGGTACTTACTATTCAGCGGCTGATACA |
| | | | ATAACTTGCATGACTATTATCTTTATGATTATTGCCATCATTGTCATCAT |
| | | | TATTTATAGAGAGCTTATCCGATCCCAGG |
| F5 | rs2213871 | 150 | ATAAACCACTCTCCTACTCAATTATTCTCTCAAAGGTATGATGGCTGGAA |
| | | | CATGTAGAAGGAAAGATATTTAAATGTGAACCATGAAAAGTCTGAAATTA |
| | | | TTTTAAAATGTTCTCATACCAACAACTATTATAATATGGATACAATTTTT |
| | | | TATACCAGTGCCTGATGGAACTCTACTATGCTTACAATGATCTGAACATC |
| | | | RGCATAATGGGATAATTAGAACCATATTAACATCAGGTACTTACTATTCA |
| | | | GCGGCTGATACAATAACTTGCATGACTATTATCTTTATGATTATTGCCAT |
| | | | CATTGTCATCATTATTTATAGAGAGCTTATCCGATCCCAGG |
| F5 | rs9332533 | 151 | GTGAGACTAGAAGCATTAATAGTATTAGACTTTTGGACATAGTGAAGTGG |
| | | | TTAAGAAAGTAAGCTTTAAAGGCAGACAAGTTAAAATTCTAGCTTTATCA |
| | | | TTTCTGATTCTACGACTTTAGCATCTCTGAGCCTCAGTTTTCTCATCTAT |
| | | | AAAATGGAAATGATTATAACCACCCATAAGATTGCCTGAAATTTAAATGA |
| | | | RGTCACTTAGGTAGAGTACTAAACATGGTTCCTGGGATCGGATAAGCTCT |
| | | | CTATAAATAATGATGACAATGATGGCAATAATCATAAAGATAATAGTCAT |
| | | | GCAAGTTATTGTATCAGCCGCTGAATAGTAAGTACCTGATGTTAATATGG |
| | | | TTCTAATTATCCCATTATGCTGATGTTCAGATCATTGTAAGCATAGTAGA |
| | | | GTTCCATCAGGCACTGGTATAAAAAATTGTATCCATATTATAATAGTTGT |
| | | | TGGTAT |
| F5 | rs9332531 | 152 | CTTTGCAGGCCATAAGATCTCTGTTGCAAGTACTCAACTCTGCCTTTTAG |
| | | | TACAAAAGCAGCCATAGACGATACCTATTTTTTTGGCTTTGGTTACAGAT |
| | | | GCCAGAACTAATTAACTTTGTTACCGTATTCTTTCCCTCAAGTCATACAC |
| | | | CAGAGTAAGACCGCCTCTGACAATACAATTTTATTTACAAAAACAGGAAA |
| | | | KTGGTTGGGTTTGGCCCATGAGCCACTGACATAGTGACATAGAACTAGCC |
| | | | TGATATAGGCTTCCTGGCACATAGATGACACTCAATAAGTGGTATTTGGT |
| | | | GGTGGTGAGACTAGAAGCATTAATAGTATTAGACTTTTGGACATAGTGAA |
| | | | GTGGTTAAGAAAGTAAGCTTTAAAGGCAGACAAGTTAAAATTCTAGCTTT |
| | | | ATCATTTCTGATTCTACGACTTTAGCATCTCTGAGCCTCAGTTTTCTCAT |
| | | | CTATAA |
| F5 | rs6691048 | 153 | GAGGGTAGTTACTTTAGATTTCTCTGACATGGGTGTGCTCAGAGACCTAG |
| | | | ATGAATTGAGGAACCAAATCACACAGCTCTCTGGCTCTGAGGCAGGAGTA |
| | | | GTCTTGATGTATCTAAGGAACAGTAAGAAAAACAGTACGGGAAGAAAGAA |
| | | | GAGATGAGGTGGATGGTAGGTGTGTAGAACAAGGGAGGCCCTATGGGCTA |
| | | | YGGTAAGAACTTGAATTTTCTTCTAAGTGAAATGGGACATCACTGGAGCA |
| | | | TTTTTCTTTTTTTTTTTGAGATGGAGTCTCGCTCTGTCTCCTAGGCTGGA |
| | | | GTGCAGTGATGCAATCTCAGGACCCACTGCAACCTCCGCCTCCTGGGTTC |
| | | | AAGCAATTCTCCTACCTCAACCTCCCAAGTAGCTGGGATTGCAGGCATGC |
| | | | ACCACCACACCCAGCTAATTTTTGTATTTTTAGTAGAGATGACTTTTCAC |
| | | | CATGTTGGCCAGGCTGGTCTCAAACTCCTGACCTCAGGTGATACACCCGC |
| | | | CTCAGCCTCCCAAAGTGCTGGGATTACAGGTGTGAGCCACCACGCCTCGT |
| | | | CTAAGTGAAATGGAGCATTTCTAAGTAAAATGAAAAACCACTTTGTACAG |
| | | | CAGAGACATAGTATAACGTGTATTAAAAGTTCATCCTAAAACAAAAAGTT |
| | | | CATCATAGCTGCTGTGTAGAGAATAGTCTGTGTAGGGACAAGAATGGAAG |
| | | | CAGAGAGATTAGGCAGTATAGGAAAGAGACTGCAGTGTACT |
| F5 | rs9332520 | 154 | AACATGGTGAAAAGTCATCTCTACTAAAAATACAAAAATTAGCTGGGTGT |
| | | | GGTGGTGCATGCCTGCAATCCCAGCTACTTGGGAGGTTGAGGTAGGAGAA |
| | | | TTGCTTGAACCCAGGAGGCGGAGGTTGCAGTGGGTCCTGAGATTGCATCA |
| | | | CTGCACTCCAGCCTAGGAGACAGAGCGAGACTCCATCTCAAAAAAAAAAA |
| | | | -/G/GGA |
| | | | AAATGCTCCAGTGATGTCCCATTTCACTTAGAAGAAAATTCAAGTTCTTA |
| | | | CCGTAGCCCATAGGGCCTCCCTTGTTCTACACACCTACCATCCACCTCAT |
| | | | CTCTTCTTTCTTCCCGTACTGTTTTTCTTACTGTTCCTTAGATACATCAA |
| | | | GACTACTCCTGCCTCAGAGCCAGAGAGCTGTGTGATTTGGTTCCTCAATT |
| | | | CATCTAGGTCTCTGAGCACACCCATGTCAGAGAAATCTAAAGTAACTACC |
| | | | CTCTCGCATCACCCTCTGTAATTGAAATGAACATCTTTCTTAAGCAAAGA |
| | | | AAGTTATTGCTAAGGAATGACAGTGGGTCAAAGTGGGCAAGACTTGGCCA |
| | | | GAACTATTTCTCCTACTCTGTTGACTTCTTTTGGGTCCAGGACAAGTGTT |
| F5 | rs9332516 | 155 | TAGAAAGTAAAACCTCATTCTTGATTTCTGCACCCATAACCTCCTGTCTC |
| | | | TGCTGTGTTCTCCCATTGCAGTAACTAATAACATCATTTACCCAGTTGTT |
| | | | CTGATAAAACACCTTGAGGTCATCCTTGACTTCTGTCTCTCACAAGCCAC |
| | | | ATGCAATCCATCAGCAAGTCTTGTTTGCCTTACCTACAAAAATGTCCAGA |
| | | | MTCCAACCACTACTCATCACGTTCTGCTACATGTGCTGGCCCAGTACACT |
| | | | GCAGTCTCTTTCCTATACTGCCTAATCTCTCTGCTTCCATTCTTGTCCCT |
| | | | ACACAGACTATTCTCTACACAGCAGCTATGATGAACTTTTTGTTTTAGGA |
| | | | TGAACTTTTAATACACGTTATACTATGTCTCTGCTGTACAAAGTGGTTTT |
| | | | TCATTTTACTTAGAAATGCTCCATTTCACTTAGACGAGGCGTGGTGGCTC |
| | | | ACACCT |
| F5 | rs9332513 | 156 | TGTTTTTTGAATATTTTTGATCCAAAATTGGTTGACTCCATGGATGTGTA |
| | | | ACCATGAATATGAACCATGGTCATGGAGGGCTGACTATATACAATTCCTC |
| | | | AAATTCTGACTCAGCCATCAACTTTTTCCCTAACTCCCCAGACAGAATTT |
| | | | GTGCTTCATCCTCTCTTCTTCTGTAATATTTTGCCCATATCTCAGCAGTC |
| | | | RCTCCTATCATACATCTATCTCCAAACTAGCTTGTGAGTTCCTTCAGTAC |
| | | | AGGGACTGTATTTGATCCAGGTAGCCAGAAACTTTCTAACACAGTGCCTG |
| | | | GCATGTAGAAAGTAAAACCTCATTCTTGATTTCTGCACCCATAACCTCCT |
| | | | GTCTCTGCTGTGTTCTCCCATTGCAGTAACTAATAACATCATTTACCCAG |
| | | | TTGTTCTGATAAAACACCTTGAGGTCATCCTTGACTTCTGTCTCTCACAA |
| | | | GCCACATGCAATCCATCAGCAAGTCTTGTTTGCCTTACCTACAAAAATGT |
| | | | CCAGACTCCAACCACTACTCATCACGTTCTGCTACATGTGCTGGCCCAGT |
| | | | ACACTGCAGTCTCTTTCCTATACTGCCTAATCTCTCTGCTTCCATTCTTG |
| | | | TCCCTACACAGACTATTCTCTACACAGCAGCTATGATGAACTTTTTGTTT |
| | | | TAGGATGAACTTTTAATACACGTTATACTATGTCTCTGCTGTACAAAGTG |
| | | | G |
| F5 | rs9332511 | 157 | TTGGCAGCATAGTCAGCACCCTGTTGGGCTCATTGGCAGCTGGGGTAGGT |
| | | | GGGAATAGTCAGTGGAAACTTCCATCAGGAGCCAAGACAGAGGGTAGCAG |
| | | | CTGGAAGAGGGGGGTCAGTCAGTGGAGTCCGACATGTCAGGAGTCAAAAG |
| | | | AGGTGTAGACATCTGGACTATTCTATAAAGATGGGGGCCTGAGCCTGCCC |
| | | | YGTGGAAGGAATAGGGAGAGCTTTGGCTCAGGAGTCCAGAGATCTGAGTT |
| | | | CTGATCCCAGCAATGCTACCAGATCACTGACTGACCTTGGGCAAGTCACC |
| | | | TTTGTCCCCCGATTCCTACCTCCCAGGCTTTGTTAACTAAAATAAGGACT |
| | | | GAACTTTGTGACCTCCACAGTCCTGTGTAAAGCTAACATAAAAACAAACA |
| | | | AAAAGCTTCAGGCACAAGATCAGAATAAACTCCAGGAGTAGGAGTTCAAG |
| | | | GAAAGGAATAGGACAACCGTGGGTGGCAAGACAGGGGTAGGGAAAGATAG |
| | | | GACCTCTGTCCCAGAAATCAGAGTTTACCATTGCTTCTGTAACCAGTTAC |
| | | | TGCAAACTTAGTGGCTAAAACAACACAAATTTATTCTTTTATGATTATAT |
| | | | ATCTGCAGATGAGAAGTCCAAAATGGGTTTTATTGGGCTAAAATCAAGGT |
| | | | GTTGGCAGAGCTGCATTCCTTCTGGAGGCTCTGGGGGAGAATTGTTTCCT |
| | | | T |
| F5 | rs9332510 | 158 | ATGCGGCACACAATTGGCAGCATAGTCAGCACCCTGTTGGGCTCATTGGC |
| | | | AGCTGGGGTAGGTGGGAATAGTCAGTGGAAACTTCCATCAGGAGCCAAGA |
| | | | CAGAGGGTAGCAGCTGGAAGAGGGGGGTCAGTCAGTGGAGTCCGACATGT |
| | | | CAGGAGTCAAAAGAGGTGTAGACATCTGGACTATTCTATAAAGATGGGGG |
| | | | YCTGAGCCTGCCCCGTGGAAGGAATAGGGAGAGCTTTGGCTCAGGAGTCC |
| | | | AGAGATCTGAGTTCTGATCCCAGCAATGCTACCAGATCACTGACTGACCT |
| | | | TGGGCAAGTCACCTTTGTCCCCCGATTCCTACCTCCCAGGCTTTGTTAAC |
| | | | TAAAATAAGGACTGAACTTTGTGACCTCCACAGTCCTGTGTAAAGCTAAC |
| | | | ATAAAAACAAACAAAAAGCTTCAGGCACAAGATCAGAATAAACTCCAGGA |
| | | | GTAGGAGTTCAAGGAAAGGAATAGGACAACCGTGGGTGGCAAGACAGGGG |
| | | | TAGGGAAAGATAGGACCTCTGTCCCAGAAATCAGAGTTTACCATTGCTTC |
| | | | TGTAACCAGTTACTGCAAACTTAGTGGCTAAAACAACACAAATTTATTCT |
| | | | TTTATGATTATATATCTGCAGATGAGAAGTCCAAAATGGGTTTTATTGGG |
| | | | CTAAAATCAAGGTGTTGGCAGAGCTGCATTCCTTCTGGAGGCTCTGGGGG |
| | | | A |
| F5 | rs9332500 | 159 | TGGGCATTGAAATAGGCATTGTGGATGCAATAGTGGACAAGACAGTCTCA |
| | | | TATCCTGGGTTCATGGAGATTATGGTCTAGTTTAATGAGCATAGATGTGC |
| | | | CTTCGCAAAATGATATTTTGCTTCCATTTGGCTCCCTAGATTCATTCAAA |
| | | | GGAACTATGTATGGCTGACTCTTTTGCATCTCTTTCCCGTCTATTCTTCT |
| | | | -/GTTT |
| | | | ATTTAGTGTGATATATACAATAGCCATAATTTTTAAAGAAAATCTCTTAT |
| | | | TTTTTCCTCATCTCTTCCTTTATCAAGAAGACTGGGTTGAATAGGGTTCT |
| | | | AATATCTTAACTATGATACCATATTCCGCAGGGATGATAGAGACCATCAA |
| | | | GGGAGAACTTCCTCCAGTTGAATCCCCTTGTCAGTAATTCACCTGTCCCT |
| | | | TTCCCCAATCAGATGAAGACCTGCTGTCTTTCCCAGAATCATCCTAGTCT |
| | | | GTCCT |
| F5 | rs3753305 | 160 | GAGTTGGCTATGTTGATATTCACCTAGTGAGTATCATAGCTCTAGGGGCA |
| | | | GGACAAGTTAGAACAAGTTCTTAGTTTAAGCAGCCAGATATATGGATTAG |
| | | | ATTTTTCATAAGAAAATGCTAGAGAGAAATGATATGATTTAGGGTTAAAC |
| | | | AATATGACAGTTTGTCTGGGTTGTGTTTCTATGGTTTTGACTCAACAATT |
| | | | SCTACCAGAGGAACGAATCTCCAGAACTTTGGAAACTTACCCACAGGATG |
| | | | AGGGGACAGAATGACCAGTCATGGTTCCCTGTGTACTCACCATGTGAATC |
| | | | AGACCTTTGCCCTATCTGGTTCTGCCTCCGGTATTTTGCTTGTTATTTTC |
| | | | AACGCTAACTACAGACAAGAGAAAAAACTCCCAAGCATGAACCCAGCATG |
| | | | CTACAAGAAAGCCAACAACCAAGTCTGCATTCTACTCATGAGCAGGCAAG |
| | | | ATTAGT |
| F7 | rs3093229 | 161 | CAACTTGCCTTGAGATGACAACCAAAGTTTTCCTGGTGTCCTCYACACTC |
| | (at position | | AAGAGTGACTGTGAGGCGGAGGGGCCCAGCCCTTCTTGCAGGCGGGAATG |
| | 44) | | AGTGGATGGGTGGATCAACAGAGGCTGCCACAGGAGAGAGGGAGGCCTGG |
| | | | CCTGGGAACAGAGCTGTGACCGTGCCCTTCCCCAGGGTAGGGGCTGAAGG |
| | | | ACCCTCCCATCCTAGTGACAGGGCCACAGCATGTCCAAGGAGGCCCCAGA |
| | | | GGAGGTCCCGGGAGTCCTGGGAGAGCCTGGTTAGCCTCCCTGAAGGGAG |
| F7 | rs3093230 | 162 | CAACTTGCCTTGAGATGACAACCAAAGTTTTCCTGGTGTCCTCCACACTC |
| | (at position | | AAGAGTGACTGTGAGGCGGAGGGGCCCAGCCCTTCTTGCAGGCGGGAATG |
| | 186) | | AGTGGATGGGTGGATCAACAGAGGCTGCCACAGGAGAGAGGGAGGCCTGG |
| | | | CCTGGGAACAGAGCTGTGACCGTGCCCTTCCCCAGRGTAGGGGCTGAAGG |
| | | | ACCCTCCCATCCTAGTGACAGGGCCACAGCATGTCCAAGGAGGCCCCAGA |
| | | | GGAGGTCCCGGGAGTCCTGGGAGAGCCTGGTTAGCCTCCCTGAAGGGAGG |
| | | | AAGTGGGGTTTTGTGAGAGGGATGGTGCAGCAGCCCCCACACCTGCTACT |
| | | | CCGTGTGGCCGGGTCCAGCCCCAGGCAAGGTTCCAGGCATGCCCCTGGGA |
| | | | CAGACGTGGGAGGGAGACCAGCAGGCAGGTCCCCCTCAGGG |
| F7 | rs762635 | 163 | ACTTGAGGTCAGGAGTTCGAAACCAGCCTGGTCAACACGGTGAAACCCCA |
| | | | TCTCTGCTAAAAAAAAAAAATATATATATATAAATTAGCCAGGCATGGTG |
| | | | ACGTGCACCTGTGGTCCCAGCTACTCAGGAGGCTGAGGCACAAGAATCAC |
| | | | TTGAACCCGGGAGGTGGAGGTTGCAGTGAGATTGCACCAGTGCACTCTCC |
| | | | MGCCTGGCAACAGAGCAAGACTCTGTCTCAAACAAACAAAACAAAACAAA |
| | | | CAAAAAGACGTAAGATGTGGACCGCTGGAGAATGGGGGTGCTGCCTGCAG |
| | | | TCAAAACGGAGTGGGGGTGCCCAGCTCAGGGCCAGAATGATCCTATTCCC |
| | | | GGCACTTCTCAGTGAGGCTCTGTGGCTCACCTAAGAAACCAGCCTCCCTT |
| | | | GCAGGCAACGGCCTAGCTGGCCTGGTCTGGAGGCTCTCTTCAAATATTTA |
| | | | CATCCACA |
| F7 | rs762636 | 164 | TGAAACCCCATCTCTGCTAAAAAAAAAAAATATATATATATAAATTAGCC |
| | | | AGGCATGGTGACGTGCACCTGTGGTCCCAGCTACTCAGGAGGCTGAGGCA |
| | | | CAAGAATCACTTGAACCCGGGAGGTGGAGGTTGCAGTGAGATTGCACCAG |
| | | | TGCACTCTCCAGCCTGGCAACAGAGCAAGACTCTGTCTCAAACAAACAAA |
| | | | RCAAAACAAACAAAAAGACGTAAGATGTGGACCGCTGGAGAATGGGGGTG |
| | | | CTGCCTGCAGTCAAAACGGAGTGGGGGTGCCCAGCTCAGGGCCAGAATGA |
| | | | TCCTATTCCCGGCACTTCTCAGTGAGGCTCTGTGGCTCACCTAAGAAACC |
| | | | AGCCTCCCTTGCAGGCAACGGCCTAGCTGGCCTGGTCTGGAGGCTCTCTT |
| | | | CAAATATTTACATCCACA |
| F7 | rs510317 | 165 | GGACCGCTGGAGAATGGGGGTGCTGCCTGCAGTCAAAACGGAGTGGGGGT |
| | | | GCCCAGCTCAGGGCCAGAATGATCCTATTCCCGGCACTTCTCAGTGAGGC |
| | | | TCTGTGGCTCACCTAAGAAACCAGCCTCCCTTGCAGGCAACGGCCTAGCT |
| | | | GGCCTGGTCTGGAGGCTCTCTTCAAATATTTACATCCACACCCAAGATAC |
| | | | DGTCTTGAGATTTGACTCGCATGATTGCTATGGGACAAGTTTTCATCTGC |
| | | | AGTTTAAATCTGTTTCCCAACTTACATTAGGGGTTTGGAATTCTAGATCG |
| | | | TATTTGAAGTGTTGGTGCCACACACACCTTAACACCTGCACGCTGGCAAC |
| | | | AAAACCGTCCGCTCTGCAGCAC |
| F7 | rs3093237 | 166 | GATGGGGTGTGGAGGATCGGGGGTGGGGATGGCGTGTGGGGTGTGGGGGA |
| | | | TGGGCCGTGGGGGGGTGGGGCCTGGGAAACAGCATGTGGGGCATGGGGTG |
| | | | TGGGGGTGAGGTGTGGGAAAGTGTGTGGGGTGTGGGGGATGGGGCATGGA |
| | | | AAGGGCGTGTGGGGTGCAGGGGATGGGGCATGGAGGTGTGGGGGATGGGG |
| | | | YGTGTGGGGTGTCGGGGATGGGGCATGTGGGGTGTGGGGGATGGGGCATG |
| | | | GAAAGGGCGTGTGGGGTGCAGAGGATGGGGCATGGGGGGGTGGGGATGGC |
| | | | GAGTGGGGCTGGGGCCTGGGAATGGTGAGTGGGGCATGGGGATGGCGAGT |
| | | | AGGGGGTGTGGCGTGAGGATGGCTAGTGGGGCGTGGGGATGGCGTGTGGG |
| | | | GATGGCGAGTGGGGGGTGGGCTGTGAGGGACAGTGCCTGGGATGTGGGGC |
| | | | TGCAGCCCTAGCTCACAGCATGGCCTTATGACCCCGGCCACCTTCCTGCC |
| | | | CCAGGCGGGGTCGCTA |
| F10 | rs483743 | 167 | TATTGCTATTAGGAAAACACATATGCATGCATTTCTTCTAGATTATCATC |
| | | | TAAGAGTGGCTTCTCCAGAGAGAGACGACTGAATTAAAGGTTATCAACAA |
| | | | GTTCCAATTCCAGATAAGATGAAGAAATCACATTCCACACTGCCTCTCCC |
| | | | ACTGAGTGTAGCTCCAAAACATGGATAGAATGCATGTAGCAGCTATTTGA |
| | | | SGACCCTAAAAAGTAAATCGCAGTGTATTGCAGAATAAGACTACAATTAG |
| | | | ATGTATGATATGATACAACTGGCTGTGAGTTTATCATTTTTTCCTCCAGT |
| | | | CTTCCAGACATCACTTGACCTGAATCTAATGGACATTTATAGGATTCTCA |
| | | | ACAATAGCAAAGTACACTTTCCTTCCACATATGGAAAATTCCTCAAGGTA |
| | | | GACTATATCCTGTGTCTTAAAGCATACCTCAATAAAAAGATTGAACTCAC |
| | | | ATAAAGTATGTTTTCTGACCATAATGGAATTAAAGTAAAAATTACTAACA |
| | | | GAAAAATAACTGGAAACTTCCCTAAGTACTCGGAAATTAAGTCACACATG |
| | | | TATAAATAATCTGTGAGTCAAAGAGAAAATTTTAAGGGGAGTAAGAAAGT |
| F10 | rs483949 | 168 | ATTTAAAAATAAAATGTTAACCTAAAAACCAATAGTCATGGTCTCGGCCA |
| | | | GCGCCTCGCCGAGTTGCAGTGAGCTGAGATCGTGCCCTCCCACGCCCGCA |
| | | | GCCCGCGTCCTGCCTTGGCCTCCGTAGTCGCTGAGAGCCACAGCCTAGAG |
| | | | CGCCAGCGCGCAGGCGCACAACTGACGCCAGGCCACGAACCCAGTACTGC |
| | | | KCCTGCACAGCAGAAGCACTAGCACTGAGGCCGGGCCGCGAACCCGGCAC |
| | | | TGCGCCTGCGCAGCAAAAGGACACGCACTGAGGCCAGGCCGCGAACCCAG |
| | | | CACGGTGCCTGCGCAGCAGGAAGACCGGCATCCACACCGGACGACGAACC |
| | | | CAGCATCGCGCCTGCGCAGTAGGAGGAGAGCAATGCCACCAGGCCGCGAT |
| | | | TGCGCAGCCGCAGCAGCCCCGCGCGGAAGACGCTACCCTCCTCTCCCCCG |
| | | | AAGAGG |
| F10 | rs3211753 | 169 | AAAAACAAACAAACAAAAACAAGAAAAAGGACCTATGTTGGAAATGGAAG |
| | | | AGAGGGGACATCACTACAGAAACTGTAGATGTTAAATGTATAATAAGAAA |
| | | | ATACTTTGAACAACTCTGCATATATAAATTTGCATGAGATTTGAACTTGG |
| | | | ATGAAATGAGCCTATTCTTCAATACCACAAGCCACCAAAACATACACAAG |
| | | | RTGAAAGAGATACCTGCCAATTCAATTCTTAATTTAAAACCTTCTGAAAA |
| | | | AGTAATGTTCAGGTACAGATGGTTTCACTGGTAGAATTTTACCAAACATT |
| | | | TCAAAAAGAACACCAATTCTATACAACTCTTCCAGAACATAGAAGAGGGA |
| | | | ACACTTCTTAGTTTGTCTTAGGCCAGCATTACCCTGATGTCAAAACCAGA |
| | | | CAAATACTGAAAACAAAAACCACCCTACGTAACAATATCTCTCATGAATC |
| | | | TAGACATAAAAATCCTCAACAAAATATTAGCAAACGGTGCAGCAATATAT |
| | | | TTTTAAAAGAGTAATAATACACCATGACCAAGTGAGTTTTTCTGGGGCAC |
| | | | ACATGACTGGCTCAATATTTAAAAATAATTATGTAATCCACCATATAAAC |
| | | | AAAAGAGAACATCCACATAATCATGTCAATTGATGCAACAAACAAATCTG |
| | | | GCAAAATTTAACATCCATTTATGATTTTATAAAAAACCTATCAGCAGAAT |
| | | | ATGAATAGGAGGGAATTTTATGAACATAATAAAGTTCATCTACAAAGAGT |
| | | | CTACAGTTGATATTATACTTAAAGGTGAAAACTGAAGGTTTTCTCCCTGA |
| F10 | rs473950 | 170 | CATGGCTGTAGGAGGGAGAAGAATGAGAGCCGAGCAAAAGGGGAATCCTC |
| | | | TTAAAAAAAATCAGATCTCATGAGAACATACTCCCACGAGAACAGCATGG |
| | | | AGGAACCACCCTCACGATTCAGTTACCTCCCACTTGGTCCCTCTCACTAC |
| | | | ACATGGGGATTATGGGAACTACAATTCAAGATGAGATTTGGGTGGGGACA |
| | | | RAGCCAAACCATATCAATGCTCCTAAAATTTGCAAATGAGTGTAACAAGG |
| | | | TCACAGAATACAAGGTCAGCACATGTGTTAATCACATTTTTATGTAATAG |
| | | | CAATGCACAGTTATTTGTAAGCCAAAAATTTTTAAATGCCATTTACAATT |
| | | | GCTTCAAAGAAAATTATATACTTATATGTAAAGCTAATAAAACATATACA |
| | | | GGATCTTTATCCCAAAATCTACAAAATTCCAATGAAAGTATTTAAACAGA |
| | | | CCTAAATAAATAGAGACACATACAGTGTTCATGGATTGAAAGACTCAACA |
| | | | TATTAAGATATCAATTTTCGGCCGGGCGCGGTGGCTCATGCCTGTAATCC |
| F10 | rs3211758 | 171 | TGATTAATAAAAGAAAAAAGTCATAAATTGGACTTTATCAAAATTAAAAC |
| | | | CTTTTGCACTTCAGAAATAAACACTGTTAAGAGGATGAAAACACAAGCTA |
| | | | CAAACTAAGAGAAAATATTTGCAAATCACATATCCAACAAAGGAATCATA |
| | | | TTCGGAATATATAAAGAAATCTTAACAGATCAGAAGAAGAAAATAAACAC |
| | | | YCAGTTAAACAAAAGACCTTAACAGCCAACTCGCCAAAGAGGATATATGG |
| | | | ATAGAAAATAAACATGTGAGAAGATACTCAACATTATTAGCTCTTACAGA |
| | | | AATGCAGATAAAAACCACAATAAGAACGACTATATACTCATAGAGTAAAA |
| | | | AACACTGACACAGAACAGCGCTGGTTAAGACACGGAGAAAGCAGAACTTT |
| | | | GATACACTGCTCGTGGGAATGCAAAATGGCACGGCCACTTTGAAAAGGAA |
| F10 | rs2251102 | 172 | ACTCTTTTACAATAACTATTATGGAAACACCCTGAGGGAAAAAGTCTTAA |
| | | | ATGAGTAAAATGAGAACAAAAGCAGAAAGGAAATTCAGGAACTATTTTCA |
| | | | AAATTCAAATATCCTGATGAACCCCTCTCACTGAAGGTTAAAACAGAAAA |
| | | | AATACTGACCTTTGTATTTATTCCAGAATTCATTCTAAAAGGAAAAAAGA |
| | | | RATTTAAAAATATATTAGGGAAAACAGAGACATTTTATACCAATAACAAC |
| | | | CATAATTAAGTGACTCAAGCTGAATAGGATATTTTCCCCAATGGAAGTCA |
| | | | CTTTTAGGAATGAATTGTTCTAGAACTATTCAATCTAAAGAGGAAAGCTA |
| | | | TTCAGATCTTCTGCATCTGTGAAGATGTGGCTTCAGTCATCTTAAATGAA |
| | | | CCATCTTTGTTGAATATTGAAAATATTTTCAAGTTAATTACCTAGAAACA |
| F10 | rs776897 | 173 | CAGGTAACAGTGACACCAAGAGGACAGGACTGAGCCCTGGGCTCCGGGCC |
| | | | CAGGTGGTTCAAACATGAAGACCATGAGGTTTGGAAACAGACCCATTATT |
| | | | TCTGTAAGCCAGATCTGCTGTTTAACCTCAGCTTCCCCATCTGACAAATG |
| | | | GGACCAACACTATTGCCTGACTGCTTGGGTGATCCCTGGAGCACTTTGCA |
| | | | YGATGCCTGGCCCACCGCAGGCCCTCAGTCTGCATTGGGACTGTGGGGGG |
| | | | ATCCAGTGCAAGGGCTCAAAGCACCAGGGCAGGCAAAGGGCAGAGCTGGC |
| | | | CCGAGGAACTGGAGCTAAGGTGCGGGGCTGGGATAGGAGTCAGGGGACGC |
| | | | TCAGGCTCTGAGCTCCTTTTACCAGGACCAGTGTTCATTGAACGTAGTTT |
| | | | TTCTTTTCCTTGATGAATGTGGACAACAGGCGGCCAGAGGGCAGTGAGCA |
| SERPINE1 | rs2227676 | 174 | ATGAGGACTGGGATGAACTGGTGGCTGGGTGTGGGGAAAATGGAAGTGAA |
| | | | GGAAGGCCAAAAGAGACAGAGAAGGCCTGGCGCGGCGACTCACGCCTATA |
| | | | ATCCCAGCACTTTGGGAGGCTGAGAAGGGGGATTGCTTGAGGCCAGAAGT |
| | | | TGAATACCAGTCTGGGCAGCATAGCAAGACCCTGCCTCTACAAAAAAAAA |
| | | | WTTTTTTTTAATTAGCCAGGCTTGGTGACATGCATCTGTAGTCTACTCAA |
| | | | GAAGCTGAGGTGAGGCCAGGCACGGTGGCTCACGCCTGTATTCCCAGCAC |
| | | | TTTGGGAGGTCAAGGCGGGTGGATGACCTGAGGTCAGGAGTTCAAGACCA |
| | | | GCCTGGCCAACATGGTGAAACCCCATCTGTATAAAAATACAAAAATTAGC |
| | | | TGGGCATGATAGCAGGTGCCTGTAATTCCAGCTACTCAGGAGGCTGAGGT |
| SERPINE1 | rs2227681 | 175 | GTGAAACCCCATCTGTATAAAAATACAAAAATTAGCTGGGCATGATAGCA |
| | | | GGTGCCTGTAATTCCAGCTACTCAGGAGGCTGAGGTGGGAGAATCTATTG |
| | | | AACCCGGGAGGGGGAGGTTGCAGTGAGCCGAGATCATGCCATTGCACTCC |
| | | | AGCCTGGGCGACAGAGTGAGACTCCTTCTCAAAACAAACAAACAAACAAA |
| | | | -/CAAA |
| | | | CAAAATACAGAAGCTGAGGCGGGAGGAACATTTGAACCGGATTCGGAGGC |
| | | | TGCAGTGAGCTATGATTGCACCACTGCGCTCCAGTCTGTGTGACAGTGAG |
| | | | ACCCTGTCTCTTACACACACACACACACACACACACACACATGCACACAC |
| | | | ACAGAGAGAGAGAAATTAGAAGATACTGAATTGGCAGAAGAGAAGGGAAA |
| | | | TAGAAATTAAAATACTGAATAGGGGAGCAGTGAACAGGGGATACCCAAAA |
| | | | GCCAA |
| SERPINE1 | rs2227683 | 176 | TGATAGCAGGTGCCTGTAATTCCAGCTACTCAGGAGGCTGAGGTGGGAGA |
| | | | ATCTATTGAACCCGGGAGGGGGAGGTTGCAGTGAGCCGAGATCATGCCAT |
| | | | TGCACTCCAGCCTGGGCGACAGAGTGAGACTCCTTCTCAAAACAAACAAA |
| | | | CAAACAAACAAACAAAATACAGAAGCTGAGGCGGGAGGAACATTTGAACC |
| | | | RGATTCGGAGGCTGCAGTGAGCTATGATTGCACCACTGCGCTCCAGTCTG |
| | | | TGTGACAGTGAGACCCTGTCTCTTACACACACACACACACACACACACAT |
| | | | GCACACACACAGAGAGAGAGAAATTAGAAGATACTGAATTGGCAGAAGAG |
| | | | AAGGGAAATAGAAATTAAAATACTGAATAGGGGAGCAGTGAACAGGGGAT |
| | | | ACCCAAAAGCCAAGAGCGAGAGAGAGCCTGGCTTCCAGAAATAGTGGAGA |
| | | | AGCCAGGAGAACTAGGTGAAAACCCAGTGCTGGGTTGCCATCAGCGAGAG |
| SERPINA5 | rs2069973 | 177 | TCTGCACCTCCTCTCCCTCCTTCCTCTCCCCGTCATCCCTAAATCTTGTC |
| | (at position | | CTCGAGCCACTGCCACCCTGTGTAAACCCTCATGCCCAGTCTTGSGGGTG |
| | 95) | | CCATCCCTTCTCTTTGAAGCTGAATGGACCAAACATACCCATTGAGTGTT |
| | | | GGGTGGGGACATCTCTGGAAAGTCAGCACCTGGACCAGCTCCACCCCTCT |
| | | | CTGAGGACACCTTCTTTCCCTTTCAGAACAAAGAACAGCCACCATGCAGC |
| | | | TCTTCCTCCTCTTGTGCCTGGTGCTTCTCAGCCCTCAGGGGGCCTCCCTT |
| | | | CACCGCCACCACCCCCGGGAGATGAAGAAGAGAGTCGAGGACCTCCATGT |
| | | | AGGTGCCACGGTGGCCCCCAGCAGCAGAAGGGACTTTACCTTTGACCTCT |
| | | | ACAGGGCCTTGGCTTCCGCTGCCCCCAGCCAGAGCATCTTCTTCTCCCCT |
| | | | GTGAGCATCTCCATGAGCCTGGCCATGCTCTCCCTGGGGGCTGGGTCCAG |
| | | | CACAAAGATGCAGATCCTGGAGGGCCTGGGCCTCAACCTCCAGAAAAGCT |
| | | | CAGAGAAGGAGCTGCACAGAGGCTTTCAGCAGCTCCTTCAGGAAC |
| SERPINA5 | rs2069974 | 178 | TCCATATCCCATCCTCCAAAATGTGTCCCTTGATGTGGATGGGTAGACAG |
| | | | GATTCCTGCCCTGGCAGCCAGACCCCTGCCTTGGGTCTGCACCTCCTCTC |
| | | | CCTCCTTCCTCTCCCCGTCATCCCTAAATCTTGTCCTCGAGCCACTGCCA |
| | | | CCCTGTGTAAACCCTCATGTCCAGTCTTGGGGGTGCCATCCCTTCTCTTT |
| | | | RAAGCTGAATGGACCAAACATACCCATTGAGTGTTGGGTGGGGACATCTC |
| | | | TGGAAAGTCAGCACCTGGACCAGCTCCACCCCTCTCTGAGGACACCTTCT |
| | | | TTCCCTTTCAGAACAAAGAACAGCCACCATGCAGCTCTTCCTCCTCTTGT |
| | | | GCCTGGTGCTTCTCAGCCCTCAGGGGGCCTCCCTTCACCGCCACCACCCC |
| SERPINA5 | rs6115 | 179 | AACAGCCACCATGCAGCTCTTCCTCCTCTTGTGCCTGGTGCTTCTCAGCC |
| | | | CTCAGGGGGCCTCCCTTCACCGCCACCACCCCCGGGAGATGAAGAAGAGA |
| | | | GTCGAGGACCTCCATGTAGGTGCCACGGTGGCCCCCAGCAGCAGAAGGGA |
| | | | CTTTACCTTTGACCTCTACAGGGCCTTGGCTTCCGCTGCCCCCAGCCAGA |
| | | | RCATCTTCTTCTCCCCTGTGAGCATCTCCATGAGCCTGGCCATGCTCTCC |
| | | | CTGGGGGCTGGGTCCAGCACAAAGATGCAGATCCTGGAGGGCCTGGGCCT |
| | | | CAACCTCCAGAAAAGCTCAGAGAAGGAGCTGCACAGAGGCTTTCAGCAGC |
| | | | TCCTTCAGGAACTCAACCAGCCCAGAGATGGCTTC |
| SERPINA5 | rs6112 | 180 | CTGAGCCTCGGCAATGCCCTTTTCACCGACCTGGTGGTAGACCTGCAGGA |
| | (at position | | CACCTTCGTAAGTGCCATGAAGACGCTGTACCTGGCAGACACTTTCCCYA |
| | 99) | | CCAACTTTAGGGACTCTGCAGGGGCCATGAAGCAGATCAATGATTATGTG |
| | | | GCAAAGCAAACGAAGGGCAAGATTGTGGACTTGCTTAAGAACCTCGATAG |
| | | | CAATGCGGTCGTGATCATGGTGAATTACATCTTCTTTAAAGGTAAGGCCC |
| | | | TTGGGCCCAAACCTGCACTTTCTTTGGCTTTTCTGCTGCTTTTATCTAAA |
| | | | GAATACCCAATTCCCTCACATACATAAAAGACGGGGAGTACGTTAAGTTC |
| | | | TTTTGGGTGCCTGTTGAGAAAAATTAAGTAAACAAGCAGCCAGAGAAGGT |
| SERPINA5 | rs2066969 | 181 | TTCCCAATGGGAAAAACCATTCATTTCCAGGATCCATACTAACTTCTTTC |
| | | | TAAAATTTAAATCAAAATATTGGAATGAAAGTGCAAACAGAGAAGTTCAC |
| | | | CCAGATATCAGGTAGCATTCACAGCCAGCCACATTTTTCACCCTCTTCAC |
| | | | TTGGAGATTTGGTCTTGAGTAAAACGTTAGAGAATCAGAGAACATCAGGG |
| | | | RTCCAGGGCCTCTGAAGATGTGAAAACCAACCTCCTTGTTTTGCAAATGT |
| | | | GGAAGGAAAAGTCCCACGAAAAGTCCAAGAATGTGCCCAATGTTATAAAG |
| | | | AGACTTGCCTTCATATTCAAGAGGTTCAACAGTCACTGCTCTGGGGCTGC |
| | | | CATAAAGATGGTCTCCGCTGGCTATCTTTACTGTCT |
| SERPINA5 | rs6107 | 182 | CCTTTTCCCTTTCCAGGCAGCTCGAGCTTTACCTTCCCAAATTCTCCATT |
| | | | GAGGGCTCCTATCAGCTGGAGAAAGTCCTCCCCAGTCTGGGGATCAGTAA |
| | | | CGTCTTCACCTCCCATGCTGATCTGTCCGGCATCAGCAACCACTCAAATA |
| | | | TCCAGGTGTCTGAGGTGGGTTCAGAAGCTCCTATGCATCTGCTTCCCAAG |
| | | | RTCTATTCTGTTCTATTCTTTCTATTCTACTCTACCCCATTTCATTCCAT |
| | | | TCCATTCCACTCAACTCCACTCCACTCCACTCCACTCCAGTTCACTCTAT |
| | | | TCAATTCCACTCCACTCCACTCCAGTTCACTTTATTCAATTCCACTCCAC |
| | | | TCCACTCCAGTTCACTCTATTCAGTTCCACTCCACTCCACTCCACTCCAG |
| | | | TTCACTCTATTCCATTCCACTCCATTCCACTCCTCCACTCCTCTCATCCA |
| | | | CTCCACTCTACTCCTCCACTCCACATCTCCACTCCACTCCTCCACTCCAC |
| SERPINA5 | rs6109 | 183 | ATTACACCTTGCTCAAAGATGCCATGAGAATTCAATGACAGACACATGCG |
| | | | AAGTCACCCCCCAGCACAGTGCCTGGGGCAGAGTAGCTGCTCCATTGTTC |
| | | | CATTTCCTACTTGCTCCATGGCTCAGTTGAACAGATACTTAGAGGTTGAT |
| | | | GCCCATAGGCAGAAGCTTTGCCATTTGCTATGATGACTTCACCTGCCCCT |
| | | | RGTGGCCTGGTGATGCCTGGTGTCTCCCCTGCAGATGGTGCACAAAGCTG |
| | | | TGGTGGAGGTGGACGAGTCGGGAACCAGAGCAGCGGCAGCCACGGGGACA |
| | | | ATATTCACTTTCAGGTCGGCCCGCCTGAACTCTCAGAGGCTAGTGTTCAA |
| | | | CAGGCCCTTTCTGATGTTCATTGTGGATAACAACATCCTCTTCCTTGGCA |
| SERPINA5 | rs6116 | 184 | TTTCCTACTTGCTCCATGGCTCAGTTGAACAGATACTTAGAGGTTGATGC |
| | | | CCATAGGCAGAAGCTTTGCCATTTGCTATGATGACTTCACCTGCCCCTGG |
| | | | TGGCCTGGTGATGCCTGGTGTCTCCCCTGCAGATGGTGCACAAAGCTGTG |
| | | | GTGGAGGTGGACGAGTCGGGAACCAGAGCAGCGGCAGCCACGGGGACAAT |
| | | | MTTCACTTTCAGGTCGGCCCGCCTGAACTCTCAGAGGCTAGTGTTCAACA |
| | | | GGCCCTTTCTGATGTTCATTGTGGATAACAACATCCTCTTCCTTGGCAAA |
| | | | GTGAACCGCCCCTGAGGTGGGGCTTCTCCTGAAATCTACAGGCCTCAGGG |
| | | | TGGGAGATGAAGGGGGCTATGCTATGGCCCATCTGTATGCTGGTAGCTAG |
| SERPINA5 | rs6108 | 185 | GAACCAGAGCAGCGGCAGCCACGGGGACAATATTCACTTTCAGGTCGGCC |
| | | | CGCCTGAACTCTCAGAGGCTAGTGTTCAACAGGCCCTTTCTGATGTTCAT |
| | | | TGTGGATAACAACATCCTCTTCCTTGGCAAAGTGAACCGCCCCTGAGGTG |
| | | | GGGCTTCTCCTGAAATCTACAGGCCTCAGGGTGGGAGATGAAGGGGGCTA |
| | | | WGCTATGGCCCATCTGTATGCTGGTAGCTAGTGATTTACACAGGTTTAGT |
| | | | TGACTAATGAGGCATTACAAATAATATTACTCTATGATGATTGCTTCCAC |
| | | | CCACACGACTGCAACATACAGGTGCCTTGGGGAAATGTGGAGAACATTCA |
| | | | ATCTTGCCGTCACTATTCATCAATGAAGATTAACACTGAGATCCAGAGAG |
| | | | GCTGGATGACTTGCTCAAGTTCACCAGCATGGTAGTGGCAAAGAGAGGTC |
| | | | CAGAGTCCTGGCCCTTGATGCCCAGCTCAGTGCCACAAAGCTCAATAGGA |
| | | | GGGATGTTCCAGTGGATGAGGGCCACCAGGAAGCACAGGTCCAAGGC |
| SERPINA5 | rs938 | 186 | GGGAGATGAAGGGGGCTAAGCTATGGCCCATCTGTATGCTGGTAGCTAGT |
| | | | GATTTACACAGGTTTAGTTGACTAATGAGGCATTACAAATAATATTACTC |
| | | | TATGATGATTGCTTCCACCCACACGACTGCAACATACAGGTGCCTTGGGG |
| | | | AAATGTGGAGAACATTCAATCTTGCCGTCACTATTCATCAATGAAGATTA |
| | | | RCACTGAGATCCAGAGAGGCTGGATGACTTGCTCAAGTTCACCAGCATGG |
| | | | TAGTGGCAAAGAGAGGTCCAGAGTCCTGGCCCTTGATGCCCAGCTCAGTG |
| | | | CCACAAAGCTCAATAGGAGGGATGTTCCAGTGGATGAGGGCCACCAGGAA |
| | | | GCACAGGTCCAAGGCTGGTCCCACACTTATCAGCAGCAACAACTGTCAGT |
| | | | TCATCCTGCATGGGAAAAATGTTGGAATGGGAGTCTGAAATGGGGCTACT |
| | | | GTTTCAGTCCTAATGTGCTGTGTGACATTGGGACAACACTTTCCCTCTCT |
| | | | GGACCTCAGTTTCCCTCTGTATACAAGGATCAGATTCTTGCTGTGACCCA |
| | | | AGAACTCCTGAAATCATATAGAAAGGCTGGGGTGGGCCCTGTCATTCGT |
| SERPINA5 | rs1050013 | 187 | TTCCACCCACACGACTGCAACATACAGGTGCCTTGGGGAAATGTGGAGAA |
| | | | CATTCAATCTTGCCGTCACTATTCATCAATGAAGATTAACACTGAGATCC |
| | | | AGAGAGGCTGGATGACTTGCTCAAGTTCACCAGCATGGTAGTGGCAAAGA |
| | | | GAGGTCCAGAGTCCTGGCCCTTGATGCCCAGCTCAGTGCCACAAAGCTCA |
| | | | RTAGGAGGGATGTTCCAGTGGATGAGGGCCACCAGGAAGCACAGGTCCAA |
| | | | GGCTGGTCCCACACTTATCAGCAGCAACAACTGTCAGTTCATCCTGCATG |
| | | | GGAAAAATGTTGGAATGGGAGTCTGAAATGGGGCTACTGTTTCAGTCCTA |
| | | | ATGTGCTGTGTGACATTGGGACAACACTTTCCCTCTCTGGACCTCAGTTT |
| | | | CCCTCTGTATACAAGGATCAGATTCTTGCTGTGACCCAAGAACTCCTGAA |
| | | | ATCATATAGAAAGGCTGGGGTGGGCCCTGTCATTCGTGGTTGATTTCAA |
| SERPINA5 | rs9113 | 188 | AGGTCCAGAGTCCTGGCCCTTGATGCCCAGCTCAGTGCCACAAAGCTCAA |
| | | | TAGGAGGGATGTTCCAGTGGATGAGGGCCACCAGGAAGCACAGGTCCAAG |
| | | | GCTGGTCCCACACTTATCAGCAGCAACAACTGTCAGTTCATCCTGCATGG |
| | | | GAAAAATGTTGGAATGGGAGTCTGAAATGGGGCTACTGTTTCAGTCCTAA |
| | | | YGTGCTGTGTGACATTGGGACAACACTTTCCCTCTCTGGACCTCAGTTTC |
| | | | CCTCTGTATACAAGGATCAGATTCTTGCTGTGACCCAAGAACTCCTGAAA |
| | | | TCATATAGAAAGGCTGGGGTGGGCCCTGTCATTCGTGGTTGATTTCAATA |
| | | | CACTCAAGTGCCATTCATCCTTTAAGAAAAACATCTGGATATCAAGGTGG |
| | | | AAATGGCCCATTTAATGATTGATTATATCATTTTGTGGATATAGTTATAA |
| | | | TCTGATGG |
| SERPINA5 | rs7070 | 189 | TAGAAAGGCTGGGGTGGGCCCTGTCATTCGTGGTTGATTTCAATACACTC |
| | | | AAGTGCCATTCATCCTTTAAGAAAAACATCTGGATATCAAGGTGGAAATG |
| | | | GCCCATTTAATGATTGATTATATCATTTTGTGGATATAGTTATAATCTGA |
| | | | TGGGCCTGGCTGGGAGTGGAAGAAGGGAAGCCTTTTGCAAATAGTAGAGT |
| | | | RTCAGTTGCAGGTGCCAATGACTAACTTTTTGAATTCTATGTTGGCATTA |
| | | | ACAATAAAGCATTTTGCAAACACTGGTTATAACTGTCTTTATGGAGGCAG |
| | | | CTCTGGGAATGGTGACATTGATAGCTTACCATGCTCCAGGCCGGGTGCCT |
| | | | GGCCCTTCACCTGGATGGTCGCATTTGCCCCTCATAAGACTCCCATGAAG |
| | | | AAAGGCACCACTGTTATCCCATCTGTTATTCACAGATGGGAAAGGCAAGG |
| | | | CTTGAAGTGGTTAGGTGGCTTACCCAGTCACATATCTTCTAAGTGGTGCA |
| | | | GCCAGAATTTGGCGGGGGGAGTGCGACCAAGAACCCTACACTCAGTCCTG |
| | | | TGCTCTGTGCTGTGGAGGAGAGATGACCAGGAGCAGAAACTTCATT |
| SERPINA5 | rs2069995 | 190 | AAGGCACCACTGTTATCCCATCTGTTATTCACAGATGGGAAAGGCAAGGC |
| | | | TTGAAGTGGTTAGGTGGCTTACCCAGTCACATATCTTCTAAGTGGTGCAG |
| | | | CCAGAATTTGGCGGGGGGAGTGCGACCAAGAACCCTACACTCAGTCCTGT |
| | | | GCTCTGTGCTGTGGAGGAGAGATGACCAGGAGCAGAAACTTCATTCAGGG |
| | | | RCATCTCAGGCACCAGCTCCCCCATGAGCCAGCTAAGTTCCCTCCCTCCC |
| | | | TTCACCAAGCACCATGTGTTTCCTCATGTGCCGAATGAAGAGGATTAGAT |
| | | | ACTCAAGAATGGAATGAGTGGGTGAGTGAGTCCTTCGCTGCACCCAAGTC |
| | | | TGATTTTCTGTGCGCCTGCTCACCCCACCCTGCATGTTCTAAGCATGCTT |
| SERPINA5 | rs2069996 | 191 | ATCTTCTAAGTGGTGCAGCCAGAATTTGGCGGGGGGAGTGCGACCAAGAA |
| | | | CCCTACACTCAGTCCTGTGCTCTGTGCTGTGGAGGAGAGATGACCAGGAG |
| | | | CAGAAACTTCATTCAGGGACATCTCAGGCACCAGCTCCCCCATGAGCCAG |
| | | | CTAAGTTCCCTCCCTCCCTTCACCAAGCACCATGTGTTTCCTCATGTGCC |
| | | | RAATGAAGAGGATTAGATACTCAAGAATGGAATGAGTGGGTGAGTGAGTC |
| | | | CTTCGCTGCACCCAAGTCTGATTTTCTGTGCGCCTGCTCACCCCACCCTG |
| | | | CATGTTCTAAGCATGCTTCCATAAGGCTGTGCCCCACCCTCTGATTCTAG |
| | | | AGTCTGGACTGTATCAGAGGTGAGTGCCTACTAGAGGTAACAAGGTCAGG |
| IL6 | rs2069825 | 192 | TTTCATTTTCACACCAAAGAATCCCACCGCGGCAGAGGACCACCGTCTCT |
| | (at position | | -/CT |
| | 51) | | GTTTAGACAATCGGTGAAGAATGGATGACCTCACTTTCCCCAACAGGCGG |
| IL6 | rs1800797 | 193 | CAAACCTCTGGCACAGAGAGCAAAGTCCTCACTGGGAGGATTCCCAAGGG |
| | (at position | | GTCACTTGGGAGAGGGCAGGGCAGCAGCCAACCTCCTCTAAGTGGGCTGA |
| | 196) | | AGCAGGTGAAGAAAGTGGCAGAAGCCACGCGGTGGCAAAAAGGAGTCACA |
| | | | CACTCCACCTGGAGACGCCTTGAAGTAACTGCACGAAATTTGAGGRTGGC |
| | | | CAGGCAGTTCTACAACAGCCSCTCACAGGGAGAGCCAGAACACAGAAGAA |
| | | | CTCAGATGACTGGTAGTATTACCTTCTTCATAATCCCAGGCTTGGGGGGC |
| | | | TGCGATGGAGTCAGAGGAAACTCAGTTCAGAACATCTTTGGTTTTTACAA |
| | | | ATACAAATTAACTGGAACGCTAAATTCTAGCCTGTTAATCTGGTCACTG |
| IL6 | rs2069832 | 194 | ATCTCAGCCCTGAGAAAGGAGGTGGGTAGGCTTGGCGATGGGGTTGAAGG |
| | | | GCCCGGTGCGCATGCGTTCCCCTTGCCCCTGCGTGTGGCCGGGGGCTGCC |
| | | | TGCATTAGGAGGTCTTTGCTGGGTTCTAGAGCACTGTAGATTTGAGGCCA |
| | | | ACGGGGCCGACTAGACTGACTTCTGTATTTATCCTTTGCTGGTGTCAGGA |
| | | | RGTTCCTTTCCTTTCTGGAAAATGCAGAATGGGTCTGAAATCCATGCCCA |
| | | | CCTTTGGCATGAGCTGAGGGTTATTGCTTCTCAGGGCTTCCTTTTCCCTT |
| | | | TCCAAAAAATTAGGTCTGTGAAGCTCCTTTTTGTCCCCCGGGCTTTGGAA |
| | | | GGACTAGAAAAGTGCCACCTGAAAGGCATGTTCAGCTTCTCAGAGCAGTT |
| IL6 | rs2069833 | 195 | GGTCTGAAATCCATGCCCACCTTTGGCATGAGCTGAGGGTTATTGCTTCT |
| | | | CAGGGCTTCCTTTTCCCTTTCCAAAAAATTAGGTCTGTGAAGCTCCTTTT |
| | | | TGTCCCCCGGGCTTTGGAAGGACTAGAAAAGTGCCACCTGAAAGGCATGT |
| | | | TCAGCTTCTCAGAGCAGTTGCAGTACTTTTTGGTTATGTAAACTCAATGG |
| | | | YTAGGATTCCTCAAAGCCATTCCAGCTAAGATTCATACCTCAGAGCCCAC |
| | | | CAAAGTGGCAAATCATAAATAGGTTAAAGCATCTCCCCACTTTCAATGCA |
| | | | AGGTATTTTGGTCCTGTTTGGTAGAAAGAAAAGAACACAGGAGGGGAGAT |
| | | | TGGGAGCCCACACTCGAATTCTGGTTCTGCCAAACCAGCCTTGTGATCTT |
| | | | GGGTAAATTCCCTACCACCTCTGGACTCCATCAGTAAAATTGGGCGTGGA |
| | | | CTAGGTGATCTCATAGATCCTTCCTGCTGGAACATTCTATGGCTTGAATT |
| | | | ATATTCTCCTAATTATTGTCAAAATTGCTGTTATTAAGTATCTACTGTGT |
| IL6 | rs1474348 | 196 | TACTTAATCCTGAGTCTCAGTTTCCTTATCTCCAAAAACCTTCCTTGCAA |
| | | | ATTTGTTTTGAAGATTAGACACAATATTTATTTAAAGTGCCTGGCACACA |
| | | | GTAGATACTTAATAACAGCAATTTTGACAATAATTAGGAGAATATAATTC |
| | | | AAGCCATAGAATGTTCCAGCAGGAAGGATCTATGAGATCACCTAGTCCAC |
| | | | SCCCAATTTTACTGATGGAGTCCAGAGGTGGTAGGGAATTTACCCAAGAT |
| | | | CACAAGGCTGGTTTGGCAGAACCAGAATTCGAGTGTGGGCTCCCAATCTC |
| | | | CCCTCCTGTGTTCTTTTCTTTCTACCAAACAGGACCAAAATACCTTGCAT |
| | | | TGAAAGTGGGGAGATGCTTTAACCTATTTATGATTTGCCACTTTGGTGGG |
| | | | CTCTGAGGTATGAATCTTAGCTGGAATGGCTTTGAGGAATCCTAGCCATT |
| | | | GAGTTTACATAACCAAAAAGTACTGCAACTGCTCTGAGAAGCTGAACATG |
| IL6 | rs1474347 | 197 | TCTTGTTACATGTCTGGGAAAGAATACCAGAATTGTTATCACCTAAGTGT |
| | | | CCCTAAAACAAACACCACTAGAGGGCCTTTTCATTGTTCAACCACAGCCA |
| | | | GGAAAGTCTCTAAGAAAAATGAAGCTACAACTCATTGGCATCCTGGCAAG |
| | | | CAAATTCCAGTGGAGTGGGGGCACACTTGGGTTCAGTTCCAAGCTCACCT |
| | | | KTGACTTTAGGTGTGTTACTTAATCCTGAGTCTCAGTTTCCTTATCTCCA |
| | | | AAAACCTTCCTTGCAAATTTGTTTTGAAGATTAGACACAATATTTATTTA |
| | | | AAGTGCCTGGCACACAGTAGATACTTAATAACAGCAATTTTGACAATAAT |
| | | | TAGGAGAATATAATTCAAGCCATAGAATGTTCCAGCAGGAAGGATCTATG |
| | | | AGATCACCTAGTCCACGCCCAATTTTACTGATGGAGTCCAGAGGTGGTAG |
| | | | GGAATTTACCCAAGATCACAAGGCTGGTTTGGCAGAACCAGAATTCGAGT |
| | | | GTGGGCTCCCAATCTCCCCTCCTGTGTTCTTTTCTTTCTACCAAACAGGA |
| IL6 | rs1554606 | 198 | ATCCAGGCAGCAACAAAAAGTGGGTAAATGTAAAGAATGTTATGTAAATT |
| | | | TCATGAGGAGGCCAACTTCAAGCTTTTTTAAAGGCAGTTTATTCTTGGAC |
| | | | AGGTATGGCCAGAGATGGTGCCACTGTGGTGAGATTTTAACAACTGTCAA |
| | | | ATGTTTAAAACTCCCACAGGTTTAATTAGTTCATCCTGGGAAAGGTACTC |
| | | | KCAGGGCCTTTTCCCTCTCTGGCTGCCCCTGGCAGGGTCCAGGTCTGCCC |
| | | | TCCCTCCCTGCCCAGCTCATTCTCCACAGTGAGATAACCTGCACTGTCTT |
| | | | CTGATTATTTATCAAAGGGAGTTTCCAGCTCAGCATACACAAGGCAGAGA |
| | | | GTGCAGACAGAACATCAAGGGGACAATTCAGAGAAGGATCC |
| IL6 | rs2069845 | 199 | CCCTGGGCATCTTCTTGTGGTGTGGAGTCTGACTTAGCAAGCCTCGGGTG |
| | | | GGTTTGAGGGTCAAATTTCTACCAGGCTTATATCCCTGGTGATGCTGCAG |
| | | | AATTCCAGGACCACACTTGGAGGTTTAAGGCCTTCCACAAGTTACTTATC |
| | | | CCATATGGTGGGTCTATGGAAAGGTGTTTCCCAGTCCTCTTTACACCACC |
| | | | RGATCAGTGGTCTTTCAACAGATCCTAAAGGGATGGTGAGAGGGAAACTG |
| | | | GAGAAAAGTATCAGATTTAGAGGCCACTGAAGAACCCATATTAAAATGCC |
| | | | TTTAAGTATGGGCTCTTCATTCATATACTAAATATGAACTATGTGCCAGG |
| | | | CATTATTTCATATGACAGAATACAAACAAATAAGATAGTGATGCTGGTCA |
| | | | GGCTTGGTGGCTCATGCCTGTATTCCCTAAACTTTGGGAGCCTAAGGTGA |
| IL6 | rs1818879 | 200 | GTTCTGCCTCCCTCTTCTGGGTTCCTAAAGCACTGCACCTATCTACCTGT |
| | | | CAAAGCATCTACCACATTGTACCACACCTTAAAATCAATGGTTTTTTTCT |
| | | | TCTCAGCCAGCATGTGGATGCCTCAATAAAGCAGACTCCTTTCATGACCT |
| | | | AAAACTAATTTCAGGGGGGAAAAAAAGACGAGCTGGGCGCAGTGGCTCAC |
| | | | RCCTATAATCCCAGCACTTTGGGAGGCTGAGGCGGGAGGATCACTTGAGG |
| | | | TCAGAAGACCAGCCTGGCCAACATGGCAAAACCCCGTCTCTACTAAAAAT |
| | | | ACAAAAATTAGCTGGGCGTGGTGGCGCACCTATAATCCCAGCTACTCAGG |
| | | | AAGCTGAGACATGATAATCGCTTGAGCCTGGGAGGTAGAGCCTGGGGCTG |
| | | | CACTCCATCCTGGGCAACAGAGGGAGATTCTGTCTCAAAAAATAATAATA |
| | | | ATAATATAAATAAATAAATAATTTTTTTAAAAAAAGACTCTTTCCTATAT |
| IL10 | rs1554286 | 201 | TCTCACTCACCTTTGGCTCCTGCCCTTAGGGTTACCTGGGTTGCCAAGCC |
| | | | TTGTCTGAGATGATCCAGTTTTACCTGGAGGAGGTGATGCCCCAAGCTGA |
| | | | GAACCAAGACCCAGACATCAAGGCGCATGTGAACTCCCTGGGGGAGAACC |
| | | | TGAAGACCCTCAGGCTGAGGCTACGGCGCTGTGTAAGTAGCAGATCAGTT |
| | | | YTTTCCCTTGCAGCTGCCCCCAAAATACCATCTCCTACAGACCAGCAGGG |
| | | | ACACTCACATCCACAGACACAGCAAAGACACAGACTGGCAGAGCTAGCTG |
| | | | TAAATGAGGAAAGACTCCTGGAGTCAGATCTCTTGCTCATTTCTCTTTGA |
| | | | GCAGGCGTTGGGGGTGGCTGCTAGGCATTTACATGTGAAATTTGCAAACA |
| | | | GCTTTCCTGTTATTTGTGAGTCATTTGTGGGTTATTAACTACTCCCCTCT |
| | | | CTCTTCATAAAAGGAGCCCAGAGCTTCAGTCAGGCCTCCACTGCCTCTTT |
| IL10 | rs1518111 | 202 | TCTCTAAATGAAAGGGCATCAAAAAGACCGCATTTCAGTTATTTCCCCAA |
| | | | ACCTCAAGTTCATTCTCCTTTTGTTCTTCCTGCAGCAAATGAAGGATCAG |
| | | | CTGGACAACTTGTTGTTAAAGGAGTCCTTGCTGGAGGACTTTAAGGTGAG |
| | | | AGCAGGGGCGGGGTGCTGGGGGAGTGTGCAGCATGATTAAGGGAAGGGAG |
| | | | RCTCTGCTTCCTGATTGCAGGGAATTGGGTTTGTTTCCTTCGCTTTGAAA |
| | | | AGGAGAAGTGGGAAGATGTTAACTCAGCACATCCAGCAGCCAGAGGGTTT |
| | | | ACAAAGGGCTCAGTCCTTCGGGGAGGCTTCTGGTGAAGGAGGATCGCTAG |
| | | | AACCAAGCTGTCCTCTTAAGCTAGTTGCAGCAGCCCCTCCTCCCAGCCAC |
| | | | CTCCGCCAATCTCTCACTCACCTTTGGCTCCTGCCCTTAGGGTTACCTGG |
| | | | GTTGCCAAGCCTTGTCTGAGATGATCCAGTTTTACCTGGAGGAGGTGATG |
| | | | CCCCAAGCTGAGAACCAAGACCCAGACATCAAGGCGCATG |
| IL10 | rs1518110 | 203 | TCCTCTGTTTTTAAAACTCCCCTTTTGATTTTTTTGGGCCAGAGCCAATT |
| | (at position | | TKATTTAAAAAAAAAAATCTCTAAATGAAAGGGCATCAAAAAGACCGCAT |
| | 52) | | TTCAGTTATTTCCCCAAACCTCAAGTTCATTCTCCTTTTGTTCTTCCTGC |
| | | | AGCAAATGAAGGATCAGCTGGACAACTTGTTGTTAAAGGAGTCCTTGCTG |
| | | | GAGGACTTTAAGGTGAGAGCAGGGGCGGGGTGCTGGGGGAGTGTGCAGCA |
| | | | TGATTAAGGGAAGGGAGACTCTGCTTCCTGATTGCAGGGAATTGGGTTTG |
| | | | TTTCCTTCGCTTTGAAAAGGAGAAGTGGGAAGATGTTAACTCAGCACATC |
| | | | CAGCAGCCAGAGGGTTTACAAAGGGCTCAGTCCTTCGGGGAGGCTTCTGG |
| | | | TGAAGGAGGATCGCTAGAACCAAGCTGTCCTCTTAAGCTAGTTGCAGCAG |
| IL10 | rs3024490 | 204 | CCAACACCTATTCCCCCAAACTTAAATTCTTAAGAGAATCCTAGATCAAG |
| | | | CCATGGGTTTGGTGAGTTAAGCTAAGCCAGATGATACAGTAAATGTGCAG |
| | | | GAAACCTGCCTTATAAAGTAAATGCGTTCTCTCTCGTGCTGAGAAACTTA |
| | | | TAAGATCCTGCTGGCGCTCTATACTTTATTGGCTAGGAGAAGTAAAGAAA |
| | | | KGTCTGATTCGAGGTGAAGATGCTCCCCATGCCTTGCAGCAGGGAAATTT |
| | | | AAATTGCCTCTGCTTAGAGCGTTTCCAGACCTGAAAGACCAGTGGTTTAG |
| | | | GGAAGCACTCTACATGAGGGAAACCTGCATTAGAAGGAGCTTCTTAATCC |
| | | | CTGGGATCTTTCCAAGCTAAACTGGATGTCTACAGTGGGGAGAAAGAAAA |
| | | | GCAGAGAACAGGACATGAGGGGGGCTCAAGGCCCCGAAGGGTTGACATAG |
| | | | GTGTCC |
| IL10 | rs1800871 | 205 | GACTTCTTTTCCTTGTTATTTCAACTTCTTCCACCCCATCTTTTAAACTT |
| | | | TAGACTCCAGCCACAGAAGCTTACAACTAAAAGAAACTCTAAGGCCAATT |
| | | | TAATCCAAGGTTTCATTCTATGTGCTGGAGATGGTGTACAGTAGGGTGAG |
| | | | GAAACCAAATTCTCAGTTGGCACTGGTGTACCCTTGTACAGGTGATGTAA |
| | | | YATCTCTGTGCCTCAGTTTGCTCACTATAAAATAGAGACGGTAGGGGTCA |
| | | | TGGTGAGCACTACCTGACTAGCATATAAGAAGCTTTCAGCAAGTGCAGAC |
| | | | TACTCTTACCCACTTCCCCCAAGCACAGTTGGGGTGGGGGACAGCTGAAG |
| | | | AGGTGGAAACATGTGCCTGAGAATCCTAATGAAATCGGGGTAAAGGAGCC |
| TNFRSF1A | rs1800693 | 206 | TATCAAGAGACAGCAAAAATATTTGTAAAGAAAGGATGTCCAACAATCTG |
| | | | TGTGGTTGTTTTTCTGTGTTCCTCCAATGGTAGGGCCTCTGTTCACCAGT |
| | | | GCCGTCTCTTCTTTTAGCTGTAAGAAAAGCCTGGAGTGCACGAAGTTGTG |
| | | | CCTACCCCAGATTGAGAATGTTAAGGGCACTGAGGACTCAGGTGAGGAGA |
| | | | RGTGACCTGGTGCCCATGCTCACCTGCCCTCTCCCTCTTCTTGCCCCCAC |
| | | | CCGTCCATCCATCCCACCCATCCATCTATCCCTGCGGCCCCCCTCTGCCC |
| | | | GCTCCTCTGACCAACACCTGCTTTGTCTGCAGGCACCACAGTGCTGTTGC |
| | | | CCCTGGTCATTTTCTTTGGTCTTTGCCTTTTATCCCTCCTCTTCATTGGT |
| | | | TTAATGTATCGCTACCAACGGTGGAAGTCCAAGCTCTACTCCATTGGTGA |
| TNFRSF1A | rs4149587 | 207 | GGCTCAGCCTCCACCTCCAGGGCTCAAGCCATCCTCTTGCCTTAGCCTCC |
| | | | TGAGTAGCTGGGATTAGAGGCACACACCACTACACCCAGCTAATGTTTTA |
| | | | CTTTTTGTAGAGACAGGGTCCTACTATATTGCCCAGGCTGGCCTCGGACT |
| | | | CCTGGGCTCAAGCGATCTTCCGCCTCAGCCTCCCAAAGTGCTAGGATTAC |
| | | | SGGCATGAGCCACCACGCCTGGCCTGGGCCTTAGATTTCTTATATTTAAA |
| | | | GTAAGCATAATGACATTCATTTGGTGAATTTGTGAGAACCAAAAACAAAG |
| | | | AAACAAACAAAACCTACAACACGTCTGACACAAAACTATTTATTTTCCAT |
| | | | TAATCTTCTTTTTTTTTTTTTTTTTTTTTTTTGACACAGAGTCCTGCTCT |
| | | | GTCGCCCAGGCTGGAATGCAGTGGCGCGATCTCGGCTCACTGCAACCTCT |
| TNFRSF1A | rs1800692 | 208 | CGTGCACCTCTCCTGTGAGCGCAGCTCTCCTGAGGCCAAGCCCTCTCCCC |
| | | | ACCCCAGGGGTTGGCCCCTTCCCCATGCGGTGGCACTTCCTTTCCTTCCC |
| | | | CCTCCTGTATTCTGTGGGTCTGACAACCAACTCCTCTCTGGCCGCCCCCA |
| | | | CCCTGTCCCTCGTCACTTCCTCTGTCCTGTGGGGTGGGGGTGCAGGCGCT |
| | | | YCTCCTTTAGCTGTGCCGCACTTCTCCCTACAGGCCAGGAGAAACAGAAC |
| | | | ACCGTGTGCACCTGCCATGCAGGTTTCTTTCTAAGAGAAAACGAGTGTGT |
| | | | CTCCTGTAGTAAGTGAGTATCTCTGAGAGCTGCTGGGCACTGGATGGTGG |
| | | | CATGGGTTGGGACGGGTGACTGGTGGGAACCATTAGCTGGGCAACAGATG |
| | | | CCAGGATGCCCCAGAGTGCTCAGGGTCCTACTGGCTGAGTAGGAGACACT |
| TNFRSF1A | rs887477 | 209 | GGGCTCTGGAGGCTTTCCTGTATTGCCAGTGGGCTTGGGGAGGGTCTGTG |
| | | | GAGACTCAGAACTGGCCTTGTTTCCTAAGGATTGTCTGGGGACCCCAGGG |
| | | | AGGCCCCCAAACCCAGCACAACTGGTCAGAACCAGCCAGGCTGTGGGAAT |
| | | | GCGGTGAACCCAGGGTGGGAGGGCAGCCTTGGCTTGCTTCCTGCTGGGAC |
| | | | KGGGGAGTGTTGGGGGATGGAGTGAGAGCTCACGGAATGGGTTTAGCTGT |
| | | | TGGAGACTTGTTGAACTGGGAGGAGGAGCTGGGGCGGGGCCTCAGCTAAA |
| | | | GGCCGCTGAGGGGCTAGGAGGAGCCAAGTGGCCCTCAGGGAAGGGAGGGC |
| | | | ACAGACCTGATGGGCGGAAGCCAGGGTCGAGGGAGACTTCCCTTCGGGAT |
| | | | GGAATGGGGAGAGGGAGGCATTTCCCGGAACATGTGGGCCAAGTGGGACA |
| TNFRSF1A | rs1860545 | 210 | AAGACATTTTTTGATCTCTCATCTTATAAGGTTCGTGGTCACTTTGGGGA |
| | (at position | | GATCATATCTGTCACCCAACATAACCATATTATGATAAGAGCCAAAAGTA |
| | 175) | | GATAGGGTCAGTTCACGTGCTTCGAGTTCACAGGGACTATGGGTCTAAGG |
| | | | AGCCGGGGTGGAGGAAACAGACATYGTCAATGGTGGCTTCACGGGAGGGA |
| | | | GATGGGATCTCAACTGGGCCCTTGGAGGAGAAGCTGCCACGACCTCCCCC |
| | | | AACACCTTGACATTAAATGAACAGACACATGAATGAGGGGGAAAGGAAGA |
| | | | CTAATTGGGTCCCTGCAAGGTGGCTGGATCGGGGTCAGACCACAAGGCCG |
| | | | ATCTCAGCGTCGCCTCCCCACTCTGCAGCCCCAGCACAGGAAGTCACACT |
| | | | TTAAAGCCTCCTCTGGCGGAAATTGTGGGGGAGTTGGAGGGGTGTTGGGC |
| | | | CACCCCCTCAACTGTCTCTCCACAGGCACCCCAGCTTCCTGCCCTTCTGC |
| | | | TCCAGGCTGGAGTCTGGGCCTAAAGAGCTCACCTCCTGTTTCTCCTGTTT |
| TNFRSF1A | rs4149581 | 211 | TTGTGTGTGGGGAGGTGGGGGGATGGTCTGAAAACTCTCCCCCGGAGATA |
| | | | AATATATTCCTACCAGGGGTGCTGTCTCCTCACCTCCCTCTTTGGGAATC |
| | | | ACTGGCTTCTACTAGAGTGGAAGACAGATGTATCATTAGATCGATCAGTT |
| | | | GATCCATATTTATCTGCTCCCAGTCTGGAGGTCTGGTTCTGGGAGCTGAG |
| | | | RGGACACCAGGGGAGGATAAGACACTTTCTGACCAAGACATTTTTTTGAT |
| | | | CTCTCATCTTATAAGGTTCGTGGTCACTTTGGGGAGATCATATCTGTCAC |
| | | | CCAACATAACCATATTATGATAAGAGCCAAAAGTAGATAGGGTCAGTTCA |
| | | | CGTGCTTCGAGTTCACAGGGACTATGGGTCTAAGGAGCCGGGGTGGAGGA |
| | | | AACAGACATCGTCAATGGTGGCTTCACGGGAGGGAGATGGGATCTCAACT |
| TNFRSF1A | rs4149580 | 212 | TGTGCTTGTGTGTGGGGAGGTGGGGGGATGGTCTGAAAACTCTCCCCCGG |
| | | | AGATAAATATATTCCTACCAGGGGTGCTGTCTCCTCACCTCCCTCTTTGG |
| | | | GAATCACTGGCTTCTACTAGAGTGGAAGACAGATGTATCATTAGATCGAT |
| | | | CAGTTGATCCATATTTATCTGCTCCCAGTCTGGAGGTCTGGTTCTGGGAG |
| | | | YTGAGAGGACACCAGGGGAGGATAAGACACTTTCTGACCAAGACATTTTT |
| | | | TTGATCTCTCATCTTATAAGGTTCGTGGTCACTTTGGGGAGATCATATCT |
| | | | GTCACCCAACATAACCATATTATGATAAGAGCCAAAAGTAGATAGGGTCA |
| | | | GTTCACGTGCTTCGAGTTCACAGGGACTATGGGTCTAAGGAGCCGGGGTG |
| | | | GAGGAAACAGACATCGTCAATGGTGGCTTCACGGGAGGGAGATGGGATCT |
| TNFRSF1A | rs4149576 | 213 | TAGGTTGTAGCAAATAGAAAGCACTCAATAAAGTTTTTATATTGCTGTGA |
| | | | CTAGTAGTAATTACTGGGTGGCTACCTGTGTTGGGAAAACAGAGGGTAAA |
| | | | GGTAGCCTGAACAGGTAAAGGGAAGTGCCTGCGTCCTGGGGTGCTTCAGC |
| | | | CCAGGTGGGATTATGTCTCCTAAGGGACAGAAGCCTGGCCTGGAGCTGGA |
| | | | RGAAAGGGAAAACAAAGGGAATGCAACATCCTTCTGAATTTCTCACCATT |
| | | | CAGTGGGCAATGCAGAGCTCACAGTGTGTGTGTGTGTGTGTGTGTGTGTG |
| | | | TGTGTGTGAGAGAGAGAGAGAGAGAGAGAGAGAAGTGGGGTAGGGGAGTA |
| | | | GGGAAGAATGATACAGGAGAGACTGTGGCAAAGCAAACAGGATTTTGCTG |
| | | | CTCTCAAAGAGCTTACAGCCTAGTAACCAAGATGGCTTACAGTGAAAAAT |
| TNFRSF1A | rs767455 | 214 | GCGCAGCCCCTACTCCAAAAGGCGGATGAATGGGGAACCCCACACTGGCA |
| | | | GTGGCTGAGGTTAGGACCTGCAGGCCTGAGGCTGGCGCCAGGACCAGGCC |
| | | | CGGGCAGGAGAGGCTCGGCCCCCTCCCGGAGAGGGCCCACGCCAGCCGGA |
| | | | AGGTGCCTCGCCCACCAGCCCACTCTTCCCTTTGTCCCTGGTCTCACCAG |
| | | | YGGCAGCAGCAGGTCAGGCACGGTGGAGAGGCCCATGCCAGACAGCTATG |
| | | | GCCTCTCACTCCCCCATTTGGGCTCATGGCAGTGTGGCAGCGGCAGTGCT |
| | | | GGGGCTTCCCGGGACTCGGTCTGTCCAGGACGTCCCAAGTGCNCTTGGGT |
| | | | GACAGTTGAGGGTTGAGACTCGGGCATAGAGATCACGGCCTGGTCCCAGT |
| | | | GATCTTGAACCCCAAAGGCCAGAACTGGAGCCTCAGTCCAGAGAATTC |
| TNFRSF1A | rs4149570 | 215 | GGATCAGTAAATTCCCAAGAAAGAGGGAGACTAGGAGGCTAGTGAAGAAC |
| | | | TNGGAGTAAAGGGGAGGATTACTAAGGGACATGGAGTACCTATCATGTGT |
| | | | CGGACGCTTATYTATATCTCTCCCATCTGAACAAATCCTTACAGGAACCC |
| | | | CAGGAGACAGGTTATCTCCACTCTGCAAATTGGAAAACAGATCCAGACAG |
| | | | KTTCAGTTATGTGTCTGAGAAGTTCATTTRTGTGTCCAAGACACATTCTT |
| | | | AGCTAAAAAGCTAAGCATTCTGAATTGGAACCCAGAGAATTTGACTCCCA |
| | | | GACTCTGGATCTTTTCACTGCTGTGATCCATCTGGGAAAGGCTAGTGATG |
| | | | TGGGCAAGGGGCTTATTGCCCCTTGGTGTTTGGTTGGGAGTGGTCGGATT |
| | | | GGTGGGTTGGGGGCACAAGGCAGCCAGMTCTGGGACTCCTGTGCTTGTGA |
| | | | CTGGACTACAAAGAGTTAAAGAACGTTGGGCCTCCTCCTCCCGCCTCCTG |
| TNFRSF1A | rs4149569 | 216 | GGGGTCTGACTCAGTGACAGAAAAAGTGGCAGTGTGTCTCTCATAGCCAA |
| | | | AGGGGCCCTTGGACCGGCAGTCGGGAGTCTGGGGTTCTCTGTTGGCTCTG |
| | | | CCTCCTGGCACATTGGGTTTCTGGACCTCAGTTTTCTCCTCTATAAAACC |
| | | | GGGCAGTTGGGTGGGCACGGTGGCTCACACCTGTAATCCTAGCACTTTAG |
| | | | SAGGCTGAGGTGGGCAGATCATTTGGGCCCAGGAGTTCAAGACCTGCCTG |
| | | | TGTAACATGGTGAGACCCTGTCTCTACAAAAAATACAAAAATTACCCAGG |
| | | | CGTGGTGGTATGCACCTATAGTCCCAGCTGCTTGGGAGGCTGAGGTGGGA |
| | | | GGATTACTTGAACCTGGGAGGTCGAGGCTGCAGTGAGCTGCGATGGTACC |
| VEGF | rs865577 | 217 | TTTCTCCACCCCCAAAGGAATGCAAACCAGGGAAGGGAGGGGAGATCCCA |
| | | | TTAGGCTGAGCCCTCTGTGCCTCCAGCTCACACAGGAAGGGTCACAGTTC |
| | | | CCACAAATGGGACATGTCTATATAGGAAATGACACTAAATGTCCACTCTC |
| | | | CCCTGGGAGCTAGGGGAAACAAGGGACACTTCCCCCAACACCTAGGATCC |
| | | | VTGAACACTGTCTTCCTGCTCTGTGCGCACGACTCCTTCTCCAAATAAAA |
| | | | TTTTACTGGAAAGAGCAGAAGAAAAAGGCAACAAGTCCTACTTCTAGCAG |
| | | | AGACCTGAACAGCGGAGAGTCCTCACGAAACTGAGGGTGAACCTCGTGGT |
| | | | GCCCAGCTCTTTCTTTCTTGATCCTTATATTCCTGTGCCCCTTCCCCTTC |
| | | | CTCCCCACAGTTCTGAAGAAAAAGGAATTAGGCCATCCACCCATCCCCTG |
| VEGF | rs833068 | 218 | AAGTAGGACTTGTTGCCTTTTTCTTCTGCTCTTTCCAGTAAAATTTTATT |
| | | | TGGAGAAGGAGTCGTGCGCACAGAGCAGGAAGACAGTGTTCACGGATCCT |
| | | | AGGTGTTGGGGGAAGTGTCCCTTGTTTCCCCTAGCTCCCAGGGGAGAGTG |
| | | | GACATTTAGTGTCATTTCCTATATAGACATGTCCCATTTGTGGGAACTGT |
| | | | RACCCTTCCTGTGTGAGCTGGAGGCACAGAGGGCTCAGCCTAATGGGATC |
| | | | TCCCCTCCCTTCCCTGGTTTGCATTCCTTTGGGGGTGGAGAAAACCCCAC |
| | | | TTGACTATGTTCGGGTGCTGTGAACTTCCCTCCCAGGCCAGCAGAGGGCT |
| | | | GGCTGTAGCTCCCAGGCGCCCCGCCCCCCTGCCCAACCCCGAGTCCGCCT |
| | | | GCCTTTTGTTCCGTTGTGGTTTGGATCCTCCCATTTCTCTGGGGACACCC |
| VEGF | rs833069 | 219 | CCAGGGTGTCCCCAGAGAAATGGGAGGATCCAAACCACAACGGAACAAAA |
| | | | GGCAGGCGGACTCGGGGTTGGGCAGGGGGGCGGGGCGCCTGGGAGCTACA |
| | | | GCCAGCCCTCTGCTGGCCTGGGAGGGAAGTTCACAGCACCCGAACATAGT |
| | | | CAAGTGGGGTTTTCTCCACCCCCAAAGGAATGCAAACCAGGGAAGGGAGG |
| | | | RGAGATCCCATTAGGCTGAGCCCTCTGTGCCTCCAGCTCACACAGGAAGG |
| | | | GTCACAGTTCCCACAAATGGGACATGTCTATATAGGAAATGACACTAAAT |
| | | | GTCCACTCTCCCCTGGGAGCTAGGGGAAACAAGGGACACTTCCCCCAACA |
| | | | CCTAGGATCCGTGAACACTGTCTTCCTGCTCTGTGCGCACGACTCCTTCT |
| | | | CCAAATAAAATTTTACTGGAAAGAGCAGAAGAAAAAGGCAACAAGTCCTA |
| VEGF | rs833070 | 220 | CCTCCCCAGAGGTGGAGAGCACAGGCCACAGTCAGTGGTGGGGAGAGCCA |
| | | | GGGTGTCCCCAGAGAAATGGGAGGATCCAAACCACAACGGAACAAAAGGC |
| | | | AGGCGGACTCGGGGTTGGGCAGGGGGGCGGGGCGCCTGGGAGCTACAGCC |
| | | | AGCCCTCTGCTGGCCTGGGAGGGAAGTTCACAGCACCCGAACATAGTCAA |
| | | | RTGGGGTTTTCTCCACCCCCAAAGGAATGCAAACCAGGGAAGGGAGGGGA |
| | | | GATCCCATTAGGCTGAGCCCTCTGTGCCTCCAGCTCACACAGGAAGGGTC |
| | | | ACAGTTCCCACAAATGGGACATGTCTATATAGGAAATGACACTAAATGTC |
| | | | CACTCTCCCCTGGGAGCTAGGGGAAACAAGGGACACTTCCCCCAACACCT |
| VEGF | rs3024991 | 221 | CTGACCTAAATCTGGCGTGGCTGGGTAGTGGCCAGCAGTGGTGATGCCCA |
| | | | GCCTGTTCTGCCTCCTCCTTCCCCACCCCAGGAGCCCTTTCCTTGGCCTA |
| | | | GGACCTGGCTTCTCAGCCACTGACCGGCCCCCTGCTTCCAGTGCGCCACT |
| | | | TACCCCTTCCAGCTTCCCAGTGGTCTCTGGTCTGGGAGAGGCAGGACAAA |
| | | | -/T |
| | | | GGTCTTTGTTTGCTGGAGAAAAGGTTGTCTGCGATAAATAAGGAAAACCA |
| | | | CGAAAGCCTGGTTGTTGGAGTGTACGTGTGTGCTCCCCCAGGCAGTGGAG |
| | | | GCCAGCCCTCCTTGGAGGGGCGGCTGCCTGATGAAGGATGCGGGTGAGGT |
| | | | TCCCCGCCTCCACCTCCCATGGGACTTGGGGATTCATTCCAAGGGGAAGC |
| | | | TTTTTGGGGGAATTCCTACCCCAGGTCTTTTTACCCTCAGTTACCAACCC |
| VEGF | rs735286 (at | 222 | ACTTACTACATCCTGAGTACTGTGTACAGTAGTCCACAGCTATCATTTCA |
| | position | | CACAAGTTTCTCCACATGGTACTATTAGACACTATTGAGATTCCATTTTA |
| | 118) | | CAGATGGGGAACAGGAGRCTCAAAGAGGCTAAGTAAGTTGCCCCAAGGCC |
| | | | GCACAGCTAGTAAGTAAAGGAGTCAGAATTAGCTGACATCAAAGTGTTCC |
| | | | CAAGCCTATATTAGGCAAAAACAGAGGAGGCACCTTTCAGGAGGAGGCAC |
| | | | CTTTCCCCCTGCCAGTCCTCTTCCCCAGACATGAGCTGAGAAGGTGGTGG |
| | | | GCATCAGCACAGGGGCTGGGCCCTCCTGGAACCCACAGGTGGCAGTGGGC |
| | | | GGACACGCTGTGCCAGCCCTGCCAGCCACTGATAACCCCGCCCAAGAGGG |
| | | | CAAACTGCTTGCATCATGGAAAAAACAGTGCTGCCACTGTAGCCACGAAA |
| VEGF | rs3024997 | 223 | GGCCCAGGATTCAGTTCAGCTGTCACAGTGAGGTGGCGGGATCAGATGTG |
| | | | GCAGGCCATGTCCCTTGGAACTTGAGTACATCGTGTGATCTCTGGAATGA |
| | | | AAACAGGCCTTCACCAGTGTTGATGGTGGAAAGCTTAGGGAAGTGCTTCA |
| | | | AACACAGTAGGAGGGACTTACGTTAGATTTTGGAAGGACTTGCCTGATTC |
| | | | RGAAGCTCCAAAGAGTGGCATTACAGAGCTGGGTGGAGAGAGGGGCTAGC |
| | | | CATCTTTTGTGTCGCCCACCGGGCTCATGTGTCATCGCCTCTCATGCAGT |
| | | | GGTGAAGTTCATGGATGTCTATCAGCGCAGCTACTGCCATCCAATCGAGA |
| | | | CCCTGGTGGACATCTTCCAGGAGTACCCTGATGAGATCGAGTACATCTTC |
| | | | AAGCCATCCTGTGTGCCCCTGATGCGATGCGGGGGCTGCTGCAATGACGA |
| VEGF | rs3024998 | 224 | CTGAGGAGTCCAACATCACCATGCAGGTGGGCATCTTTGGGAAGTGGGGC |
| | | | AAGGGGGGGATAGGGAGGGGGGTAACACTTTGGGAACAGGTGGTCCCAGG |
| | | | TCGTTTCCTGGCTAGATTTGCCTTGTCTGGCTCCTGCCCCTGAGTTGCAC |
| | | | AGGGGAGGTATGGTGGGGTCTTGCCTTCTGTGGAGAAGATGCTTCATTCC |
| | | | YAGCCCAGGTTCCCAGCAAGCCCCAACCATCTCCTTCTCCCTGATGGTTG |
| | | | CCCATGGGCTCAGGAGGGGACAGATGGATGCCTGTGTCAGGAGCCCCTCT |
| | | | CTCCCTCTCTTGGAGAGAGTCCTGAGTGCCCCCCCTTCTTGGGGGCTTTG |
| | | | TTTGGGAAGCTGGATGAGCCTGGTCCATGGAGAGTTTAAAAAGTCTTTTG |
| | | | GTGTTACCTGGTAATGGGGCACATCTCAGCCCAGATAGGGTGGGAGGGAG |
| VEGF | rs3025006 | 225 | CTTTTGGTGGCTGCTGTGACGGTGCAGTTGGATGCGAGGCCGGCTGGAGG |
| | | | GTGGTTTCTCAGTGCATGCCCTCCTGTAGGCGGCAGGCGGCAGACACACA |
| | | | GCCCTCTTGGCCAGGGAGAAAAAGTTGAATGTTGGTCATTTTCAGAGGCT |
| | | | TGTGAGTGCTCCGTGTTAAGGGGCAGGTAGGATGGGGTGGGGGACAAGGT |
| | | | YTGGCGGCAGTAACCCTTCAAGACAGGGTGGGCGGCTGGCATCAGCAAGA |
| | | | GCTTGCAGGGAAAGAGAGACTGAGAGAGAGCACCTGTGCCCTGCCCTTTC |
| | | | CCCCACACCATCTTGTCTGCCTCCAGTGCTGTGCGGACATTGAAGCCCCC |
| | | | ACCAGGCCTCAACCCCTTGCCTCTTCCCTCAGCTCCCAGCTTCCAGAGCG |
| | | | AGGGGATGCGGAAACCTTCCTTCCACCCTTTGGTGCTTTCTCCTAAGGGG |
| VEGF | rs3025007 | 226 | GTTGAATGTTGGTCATTTTCAGAGGCTTGTGAGTGCTCCGTGTTAAGGGG |
| | | | CAGGTAGGATGGGGTGGGGGACAAGGTTTGGCGGCAGTAACCCTTCAAGA |
| | | | CAGGGTGGGCGGCTGGCATCAGCAAGAGCTTGCAGGGAAAGAGAGACTGA |
| | | | GAGAGAGCACCTGTGCCCTGCCCTTTCCCCCACACCATCTTGTCTGCCTC |
| | | | YAGTGCTGTGCGGACATTGAAGCCCCCACCAGGCCTCAACCCCTTGCCTC |
| | | | TTCCCTCAGCTCCCAGCTTCCAGAGCGAGGGGATGCGGAAACCTTCCTTC |
| | | | CACCCTTTGGTGCTTTCTCCTAAGGGGGACAGACTTGCCCTCTCTGGTCC |
| | | | CTTCTCCCCCTCCTTTCTTCCCTGTGACAGACATCCTGAGGTGTGTTCTC |
| | | | TTGGGCTTGGCAGGCATGGAGAGCTCTGGTTCTCTTGAAGGGGACAGGCT |
| VEGF | rs3025009 | 227 | GGCGGCAGTAACCCTTCAAGACAGGGTGGGCGGCTGGCATCAGCAAGAGC |
| | | | TTGCAGGGAAAGAGAGACTGAGAGAGAGCACCTGTGCCCTGCCCTTTCCC |
| | | | CCACACCATCTTGTCTGCCTCCAGTGCTGTGCGGACATTGAAGCCCCCAC |
| | | | CAGGCCTCAACCCCTTGCCTCTTCCCTCAGCTCCCAGCTTCCAGAGCGAG |
| | | | RGGATGCGGAAACCTTCCTTCCACCCTTTGGTGCTTTCTCCTAAGGGGGA |
| | | | CAGACTTGCCCTCTCTGGTCCCTTCTCCCCCTCCTTTCTTCCCTGTGACA |
| | | | GACATCCTGAGGTGTGTTCTCTTGGGCTTGGCAGGCATGGAGAGCTCTGG |
| | | | TTCTCTTGAAGGGGACAGGCTACAGCCTGCCCCCCTTCCTGTTTCCCCAA |
| | | | ATGACTGCTCTGCCATGGGGAGAGTAGGGGGCTCGCCTGGGCTCGGAAGA |
| PROC | rs971207 | 228 | CGTGCAGCGTCCTCCTCCATGTAGCCTGGCTGCGTTTTTCTCTGACGTTG |
| | | | TCCGGCGTGCATCGCATTTCCCTCTTTACCCCCTTGCTTCCTTGAGGAGA |
| | | | GAACAGAATCCCGATTCTGCCTTCTTCTATATTTTCCTTTTTATGCATTT |
| | | | TAATCAAATTTATATATGTATGAAACTTTAAAAATCAGAGTTTTACAACT |
| | | | YTTACATTTCAGCATGCTGTTCCTTGGCATGGGTCCTTTTTTCATTCATT |
| | | | TTCATTAAAAGGTGGACCCTTTTAATGTGGAAATTCCTATCTTCTGCCTC |
| | | | TAGGGACATTTATCACTTATTTCTTCTACAATCTCCCCTTTACTTCCTCT |
| | | | ATTTTCTCTTTCTGGACCTCCCATTATTCAGACCTCTTTCCTCTAGTTTT |
| | | | ATTGTCTCTTCTATTTCCCATCTCTTTGACTTTGTGTTTTCTTTCAGGGA |
| PROC | rs973760 | 229 | CAGCAACCCTGGTACCTGGTTAGGAACGCAGACCCTCTGCCCCCATCCTC |
| | | | CCAACTCTGAAAAACACTGGCTTAGGGAAAGGCGCGATGCTCAGGGGTCC |
| | | | CCCAAAGCCCGCAGGCAGAGGGAGTGATGGGACTGGAAGGAGGCCGAGTG |
| | | | ACTTGGTGAGGGATTCGGGTCCCTTGCATGCCAGAGGCTGCTGTGGGAGC |
| | | | RGACAGTCGCGAGAGCAGCACTGCAGCTGCATGGGGAGAGGGTGTTGCTC |
| | | | CAGGGACGTGGGATGGAGGCTGGGCGCGGGCGGGTGGCGCTGGAGGGCGG |
| | | | GGGAGGGGCAGGGAGCACCAGCTCCTAGCAGCCAACGACCATCGGGCGTC |
| | | | GATCCCTGTTTGTCTGGAAGCCCTCCCCTCCCCTGCCCGCTCACCCGCTG |
| PROC | rs1158867 | 230 | TTAGCTAATATTCTCAGCCCAGTCATCAGACCGGCAGAGGCAGCCACCCC |
| | | | ACTGTCCCCAGGGAGGACACAAACATCCTGGCACCCTCTCCACTGCATTC |
| | | | TGGAGCTGCTTTCTAGGCAGGCAGTGTGAGCTCAGCCCCACGTAGAGCGG |
| | | | GCAGCCGAGGCCTTCTGAGGCTATGTCTCTAGCGAACAAGGACCCTCAAT |
| | | | YCCAGCTTCCGCCCTGACGGCCAGCACACAGGGACAGCCCTTTCATTCCG |
| | | | CTTCCACCTGGGGGTGCAGGCAGAGCAGCAGCGGGGGTAGGCACTGCCCG |
| | | | GAGCTCANAAGTCCTCCTCAGACAGGTGCCAGTGCC |
| PROC | rs1518759 | 231 | GAGGCTGAGGTGGGAGGATTGCTTGAGCTTGGGAGTTTGAGACTAGCCTG |
| | | | GGCAACACAGTGAGACCCTGTCTCTATTTTTAAAAAAAGTAAAAAAAGAT |
| | | | CTAAAAATTTAACTTTTTATTTTGAAATAATTAGATATTTCCAGGAAGCT |
| | | | GCAAAGAAATGCCTGGTGGGCCTGTTGGCCTGTGGGTTTCCTGCAAGGCC |
| | | | KTGGGAAGGCCCTGTCATTGGCAGAACCCCAGATCGTGAGGGCTTTCCTT |
| | | | TTAGGCTGCTTTCTAAGAGGACTCCTCCAAGCTCTTGGAGGATGGAAGAC |
| | | | GCTCACCCATGGTGTTCGGCCCCTCAGAGCAGGGTGGGGCAGGGGAGCTG |
| | | | GTGCCTGTGCAGGCTGTGGACATTTGCATGACTCCCTGTGGTCAGCTAAG |
| PROC | rs1799809 | 232 | TATTTTAGATTTGACGAAATATGGAATATTACCTGTTGTGCTGATCTTGG |
| | | | GCAAACTATAATATCTCTGGGCAAAAATGTCCCCATCTGAAAAACAGGGA |
| | | | CAACGTTCCTCCCTCAGCCAGCCACTATGGGGCTAAAATGAGACCACATC |
| | | | TGTCAAGGGTTTTGCCCTCACCTCCCTCCCTGCTGGACGGCATCCTTGGT |
| | | | RGGCAGAGGTGGGCTTCGGGCAGAACAAGCCGTGCTGAGCTAGGACCAGG |
| | | | AGTGCTAGTGCCACTGTTTGTCTATGGAGAGGGAGGCCTCAGTGCTGAGG |
| | | | GCCAAGCAAATATTTGTGGTTATGGATTAACTCGAACTCCAGGCTGTCAT |
| | | | GGCGGCAGGACGGCGAACTTGCAGTATCTCCACGACCCGCCCCTGTGAGT |
| PROC | rs1799810 | 233 | CCTCACCTCCCTCCCTGCTGGACGGCATCCTTGGTGGGCAGAGGTGGGCT |
| | | | TCGGGCAGAACAAGCCGTGCTGAGCTAGGACCAGGAGTGCTAGTGCCACT |
| | | | GTTTGTCTATGGAGAGGGAGGCCTCAGTGCTGAGGGCCAAGCAAATATTT |
| | | | GTGGTTATGGATTAACTCGAACTCCAGGCTGTCATGGCGGCAGGACGGCG |
| | | | WACTTGCAGTATCTCCACGACCCGCCCCTGTGAGTCCCCCTCCAGGCAGG |
| | | | TCTATGAGGGGTGTGGAGGGAGGGCTGCCCCCGGGAGAAGAGAGCTAGGT |
| | | | GGTGATGAGGGCTGAATCCTCCAGCCAGGGTGCTCAACAAGCCTGAGCTT |
| | | | GGGGTGAAAGGACACAAGGCCCTCCACAGGCCAGGCCTGGCAGCCACAGT |
| PROC | rs2069901 | 234 | CCACCACAGCCCAGCATGGTGTGGTGCCTCAGCAGGAGGCATCTGGTTAC |
| | | | AATCAACACAAGCTGTTCCAGCCAATTTAAAGAAACTTCAGGAGGAATAG |
| | | | GGTTTTAGGAGGGCATGGGGACCCTCCTGCACCCGAAGCCAGGATGTGCC |
| | | | ACCAATCATAAGGAGGCAGGGGCCTCCTTCCGCTGCTCCCTGGGACTCTC |
| | | | YAGGTGTCCGTGGCCTCAGTCCCCCTCTGCACACCTGCATCTTCCTTCTC |
| | | | ATCAGCTTCCTCTGCTTTAAGCGTAAACATGGATGCCCAGGACCTGGCCT |
| | | | CAATCTTCCGAGTCTGGTACTTATGGTGTACTGACAGTGTGAGACCCTAC |
| | | | TCCTCTGATCAATCCCCTGGGTTGGTGACTTCCCTGTGCAATCAATGGAA |
| PROC | rs2069902 | 235 | GGCCTCCTTCCGCTGCTCCCTGGGACTCTCCAGGTGTCCGTGGCCTCAGC |
| | (at position | | SCCCCTCTGCACACCTGCATCTTCCTTCTCATCAGCTTCCTCTGCTTTAA |
| | 51) | | G |
| PROC | rs2069912 | 236 | CCCCTTTCCTGGTCTCCACAGCCAACGGGAGGAGGCCATGATTCTTGGGG |
| | | | AGGTCCGCAGGACACATGGGCCCCTAAAGCCACACCAGGCTGTTGGTTTC |
| | | | ATTTGTGCCTTTATAGAGCTGTTTATCTGCTTGGGACCTGCACCTCCACC |
| | | | CTTTCCCAAGGTGCCCTCAGCTCAGGCATACCCTCCTCTAGGATGCCTTT |
| | | | YCCCCCATCCCTTCTTGCTCACACCCCCAACTTGATCTCTCCCTCCTAAC |
| | | | TGTGCCCTGCACCCAAGACAGACACTTCACAGAGCCCAGGAGACACCTGG |
| | | | GGACCCTTCCTGGGTGATAGGTCTGTCTATCCTCCAGGTGTCCCTGCCCA |
| | | | AGGGGAGAAGCATGGGGAATACTTGGTTGGGGGAGGAGAGGAAGACTGGG |
| PROC | rs2069913 | 237 | GGCCCCTAAAGCCACACCAGGCTGTTGGTTTCATTTGTGCCTTTATAGAG |
| | | | CTGTTTATCTGCTTGGGACCTGCACCTCCACCCTTTCCCAAGGTGCCCTC |
| | | | AGCTCAGGCATACCCTCCTCTAGGATGCCTTTTCCCCCATCCCTTCTTGC |
| | | | TCACACCCCCAACTTGATCTCTCCCTCCTAACTGTGCCCTGCACCCAAGA |
| | | | SAGACACTTCACAGAGCCCAGGAGACACCTGGGGACCCTTCCTGGGTGAT |
| | | | AGGTCTGTCTATCCTCCAGGTGTCCCTGCCCAAGGGGAGAAGCATGGGGA |
| | | | ATACTTGGTTGGGGGAGGAGAGGAAGACTGGGGGGATGTGTCAAGATGGG |
| | | | GCTGCACGTGGTGTACTGGCAGAAGAGTGAGAGGATTTAACTTGGCAGCC |
| PROC | rs2069914 | 238 | ACACCAGGCTGTTGGTTTCATTTGTGCCTTTATAGAGCTGTTTATCTGCT |
| | | | TGGGACCTGCACCTCCACCCTTTCCCAAGGTGCCCTCAGCTCAGGCATAC |
| | | | CCTCCTCTAGGATGCCTTTTCCCCCATCCCTTCTTGCTCACACCCCCAAC |
| | | | TTGATCTCTCCCTCCTAACTGTGCCCTGCACCCAAGACAGACACTTCACA |
| | | | RAGCCCAGGAGACACCTGGGGACCCTTCCTGGGTGATAGGTCTGTCTATC |
| | | | CTCCAGGTGTCCCTGCCCAAGGGGAGAAGCATGGGGAATACTTGGTTGGG |
| | | | GGAGGAGAGGAAGACTGGGGGGATGTGTCAAGATGGGGCTGCACGTGGTG |
| | | | TACTGGCAGAAGAGTGAGAGGATTTAACTTGGCAGCCTTTACAGCAGCAG |
| PROC | rs2069922 | 239 | TTTCCCTGCTTCCTTTCTTCCTGGCGTCCCCGCCTTCCTCCGGGCGCCCC |
| | (at position | | -/C |
| | 51) | | TGCGCACCTGGGGCCACCTCCTGGAGCGCAAGCCCAGTGGTGGCTCCGCT |
| PROC | rs2069928 | 240 | CTGAAACGAGACACAGAAGACCAAGAAGACCAAGTAGATCCGCGGCTCAT |
| | | | TGATGGGAAGATGACCAGGCGGGGAGACAGCCCCTGGCAGGTGGGAGGCG |
| | | | AGGCAGCACCGGCTGCTCACGTGCTGGGTCCGGGATCACTGAGTCCATCC |
| | | | TGGCAGCTATGCTCAGGGTGCAGAAACCGAGAGGGAAGCGCTGCCATTGC |
| | | | KTTTGGGGGATGATGAAGGTGGGGGATGCTTCAGGGAAAGATGGACGCAA |
| | | | CCTGAGGGGAGAGGAGCAGCCAGGGTGGGTGAGGGGAGGGGCATGGGGGC |
| | | | ATGGAGGGGTCTGCAGGAGGGAGGGTTACAGTTTCTAAAAAGAGCTGGAA |
| | | | AGACACTGCTCTGCTGGCGGGATTTTAGGCAGAAGCCCTGCTGATGGGAG |
| PROC | rs2069933 | 241 | CTCCCTGGCAGTGCCGTGTTCTGGGGGTCCTCCTCTCTGGGTCTCACTGC |
| | | | CCCTGGGGTCTCTCCAGCTACCTTTGCTCCACGTTCCTTTGTGGCTCTGG |
| | | | TCTGTGTCTGGGGTTTCCAGGGGTCTCGGGCTTCCCTGCTGCCCATTCCT |
| | | | TCTCTGGTCTCACGGCTCCGTGACTCCTGAAAACCAACCAGCATCCTACC |
| | | | YCTTTGGGATTGACACCTGTTGGCCACTCCTTCTGGCAGGAAAAGTCACC |
| | | | GTTGATAGGGTTCCACGGCATAGACAGGTGGCTCCGCGCCAGTGCCTGGG |
| | | | ACGTGTGGGTGCACAGTCTCCGGGTGAACCTTCTTCAGGCCCTCTGCCCA |
| | | | GGCCTGCAGGGGCACAGCAGTGGGTGGGCCTCAGGAAAGTGCCACTGGGG |
| PROCR | rs2069940 | 242 | TGTCCACTAATAAATTATGACCTCAGTTTCAAAAAGATTGCTTTAGGTAA |
| | (at position | | SCAATCATCTTCTGAGATTTATACAGATTGCTCATAATTCTCTCCTATTT |
| | 51) | | T |
| SERPINE1 | rs2227631 | 243 | CCTGGTGCCAAAAACGTTGAGGACCACTGCTCCACAGAATCTATCGGTCA |
| | | | CTCTTCCTCCCCTCACCCCCTTGCCCTAAAAGCACACCCTGCAAACCTGC |
| | | | CATGAATTGACACTCTGTTTCTATCCCTTTTCCCCTTGTGTCTGTGTCTG |
| | | | GAGGAAGAGGATAAAGGACAAGCTGCCCCAAGTCCTAGCGGGCAGCTCGA |
| | | | RGAAGTGAAACTTACACGTTGGTCTCCTGTTTCCTTACCAAGCTTTTACC |
| | | | ATGGTAACCCCTGGTCCCGTTCAGCCACCACCACCCCACCCAGCACACCT |
| | | | CCAACCTCAGCCAGACAAGGTTGTTGACACAAGAGAGCCCTCAGGGGCAC |
| | | | AGAGAGAGTCTGGACACGTGGGGAGTCAGCCGTGTATCATCGGAGGCGGC |

An “allele” is defined as any one or more alternative forms of a given gene. In a diploid cell or organism the members of an allelic pair (i.e. the two alleles of a given gene) occupy corresponding positions (loci) on a pair of homologous chromosomes and if these alleles are genetically identical the cell or organism is said to be “homozygous”, but if genetically different the cell or organism is said to be “heterozygous” with respect to the particular gene.

A “gene” is an ordered sequence of nucleotides located in a particular position on a particular chromosome that encodes a specific functional product and may include untranslated and untranscribed sequences in proximity to the coding regions (5′ and 3′ to the coding sequence). Such non-coding sequences may contain regulatory sequences needed for transcription and translation of the sequence or introns etc. or may as yet to have any function attributed to them beyond the occurrence of the SNP of interest. For Example, the sequences identified in TABLES 1C and 1D.

A “genotype” is defined as the genetic constitution of an organism, usually in respect to one gene or a few genes or a region of a gene relevant to a particular context (i.e. the genetic loci responsible for a particular phenotype).

TABLE 1E
below shows a genotype correlation for protein C pathway
associated gene SNPs with values representing an indication
of responsiveness to treatment of an inflammatory condition
with activated protein C or protein C like compound.

| | | Responsiveness To |
|---|---|---|
| Polymorphism | Genotype | Treatment^{∞} |
| rs1800791 | A | IR |
| rs1800791 | G | NAR |
| rs3136516 | G | IR |
| rs3136516 | GG | IR |
| rs3136516 | A | NAR |
| rs253073 | G | IR |
| rs253073 | GG | IR |
| rs253073 | A | NAR |
| rs2227750 | GG | IR |
| rs2227750 | C | NAR |
| rs1361600 | GG | IR |
| rs1361600 | A | NAR |
| rs9332575 | G | IR |
| rs9332575 | A | NAR |
| rs4656687 | T | IR |
| rs4656687 | C | NAR |
| rs9332630 | A | IR |
| rs9332630 | G | NAR |
| rs9332546 | A | IR |
| rs9332546 | G | NAR |
| rs2774030 | AG | IR |
| rs2026160 | C | IR |
| rs2026160 | A | NAR |
| rs3211719 | G | IR |
| rs3211719 | A | NAR |
| rs3093261 | T | IR |
| rs3093261 | C | NAR |
| rs1799889 | G | IR |
| rs1799889 | — | NAR |
| rs1050813 | A | IR |
| rs1050813 | AG | IR |
| rs1050813 | GG | NAR |
| rs2069972 | TT | IR |
| rs2069972 | C | NAR |
| rs2069840 | C | IR |
| rs2069840 | G | NAR |
| rs1800795 | G | IR |
| rs1800795 | C | NAR |
| rs1800872 | A | IR |
| rs1800872 | C | NAR |
| rs2243154 | AA | IR |
| rs2243154 | AG | IR |
| rs2243154 | GG | NAR |
| rs4149577 | CT | IR |
| rs1413711 | AA | IR |
| rs1413711 | G | NAR |
| rs2069895 | AG | IR |
| rs2069898 | CT | IR |
| rs2069904 | AG | IR |
| rs1799808 | CT | IR |
| rs2069910 | C | IR |
| rs2069910 | CT | IR |
| rs2069915 | AG | IR |
| rs2069916 | CT | IR |
| rs2069918 | A | IR |
| rs2069918 | AA | IR |
| rs2069919 | AG | IR |
| rs2069920 | CT | IR |
| rs2069924 | CT | IR |
| rs5937 | CT | IR |
| rs2069931 | CT | IR |
| rs777556 | C | IR |
| rs1033797 | C | IR |
| rs1033799 | A | IR |
| rs2295888 | G | IR |
| rs867186 | AG | IR |
| rs867186 | G | IR |

^{∞}Improved Response (IR); No Response or Adverse Response(NAR).
A “phenotype” is defined as the observable characters of an organism.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A “transition” is the replacement of one purine by another purine or one pyrimidine by another pyrimidine. A “transversion” is the replacement of a purine by a pyrimidine or vice versa. Single nucleotide polymorphisms can also arise from a deletion (represented by “−” or “del”) of a nucleotide or an insertion (represented by “+” or “ins” or “I”) of a nucleotide relative to a reference allele. Furthermore, a person of skill in the art would appreciate that an insertion or deletion within a given sequence could alter the relative position and therefore the position number of another polymorphism within the sequence. Furthermore, although an insertion or deletion may by some definitions not qualify as a SNP as it may involve the deletion of or insertion of more than a single nucleotide at a given position, as used herein such polymorphisms are also called SNPs as they generally result from an insertion or deletion at a single site within a given sequence.

A “systemic inflammatory response syndrome” or (SIRS) is defined as including both septic (i.e. sepsis or septic shock) and non-septic systemic inflammatory response (i.e. post operative). “SIRS” is further defined according to ACCP (American College of Chest Physicians) guidelines as the presence of two or more of A) temperature >38° C. or <36° C., B) heart rate >90 beats per minute, C) respiratory rate >20 breaths per minute, and D) white blood cell count >12,000 per mm³ or <4,000 mm³. In the following description, the presence of two, three, or four of the “SIRS” criteria were scored each day over the 28 day observation period.

“Sepsis” is defined as the presence of at least two “SIRS” criteria and known or suspected source of infection. Septic shock was defined as sepsis plus one new organ failure by Brussels criteria plus need for vasopressor medication.

Subject outcome or prognosis as used herein refers the ability of a subject to recover from an inflammatory condition and may be used to determine the efficacy of a treatment regimen, for example the administration of activated protein C or protein C like compound. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Assessing subject outcome, prognosis, or response of a subject to activated protein C or protein C like compound or protein C like compound administration may be accomplished by various methods. For Example, an “APACHE II” score is defined as Acute Physiology And Chronic Health Evaluation and herein was calculated on a daily basis from raw clinical and laboratory variables. Vincent et al. (Vincent J L. Ferreira F. Moreno R. Scoring systems for assessing organ dysfunction and survival. Critical Care Clinics. 16:353-366, 2000) summarize APACHE score as follows “First developed in 1981 by Knaus et al., the APACHE score has become the most commonly used survival prediction model in ICUs worldwide. The APACHE II score, a revised and simplified version of the original prototype, uses a point score based on initial values of 12 routine physiologic measures, age, and previous health status to provide a general measure of severity of disease. The values recorded are the worst values taken during the subject's first 24 hours in the ICU. The score is applied to one of 34 admission diagnoses to estimate a disease-specific probability of mortality (APACHE II predicted risk of death). The maximum possible APACHE II score is 71, and high scores have been well correlated with mortality. The APACHE II score has been widely used to stratify and compare various groups of critically ill subjects, including subjects with sepsis, by severity of illness on entry into clinical trials.” Furthermore, the criteria or indication for administering activated vasopressin (XIGRIS™-drotrecogin alfa (activated)) in the United States is an APACHE II score of ≧25. In Europe, the criteria or indication for administering activated protein C or protein C like compound is an APACHE II score of ≧25 or 2 new organ system failures.

“Activated protein C” as used herein includes Drotrecogin alfa (activated) which is sold as XIGRIS™ by Eli Lilly and Company. Drotrecogin alfa (activated) is a serine protease glycoprotein of approximately 55 kilodalton molecular weight and having the same amino acid sequence as human plasma-derived Activated Protein C. The protein consists of a heavy chain and a light chain linked by a disulfide bond. XIGRIS™, Drotecogin alfa (activated) is currently indicated for the reduction of mortality in adult subjects with severe sepsis (sepsis associated with acute organ dysfunction) who have a high risk of death (e.g., as determined by an APACHE II score of greater >25 or having 2 or more organ system failures).

XIGRIS™ is available in 5 mg and 20 mg single-use vials containing sterile, preservative-free, lyophilized drug. The vials contain 5.3 mg and 20.8 mg of drotrecogin alfa (activated), respectively. The 5 and 20 mg vials of XIGRIS™ also contain 40.3 and 158.1 mg of sodium chloride, 10.9 and 42.9 mg of sodium citrate, and 31.8 and 124.9 mg of sucrose, respectively. XIGRIS™ is recommended for intravenous administration at an infusion rate of 24 mcg/kg/hr for a total duration of infusion of 96 hours. Dose adjustment based on clinical or laboratory parameters is not recommended. If the infusion is interrupted, it is recommended that when restarted the infusion rate should be 24 mcg/kg/hr. Dose escalation or bolus doses of drotrecogin alfa are not recommended. XIGRIS™ may be reconstituted with Sterile Water for Injection and further diluted with sterile normal saline injection. These solutions must be handled so as to minimize agitation of the solution (Product information. XIGRIS™, Drotecogin alfa (activated), Eli Lilly and Company, November 2001).

Drotrecogin alfa (activated) is a recombinant form of human Activated Protein C, which may be produced using a human cell line expressing the complementary DNA for the inactive human Protein C zymogen, whereby the cells secrete protein into the fermentation medium. The protein may be enzymatically activated by cleavage with thrombin and subsequently purified. Methods, DNA compounds and vectors for producing recombinant activated human protein C are described in U.S. Pat. Nos. 4,775,624; 4,992,373; 5,196,322; 5,270,040; 5,270,178; 5,550,036; 5,618,714 all of which are incorporated herein by reference.

Treatment of sepsis using activated protein C or protein C like compound in combination with a bactericidal and endotoxin neutralizing agent is described in U.S. Pat. No. 6,436,397;methods for processing protein C is described in U.S. Pat. No. 6,162,629; protein C derivatives are described in U.S. Pat. Nos. 5,453,373 and 6,630,138; glycosylation mutants are described in U.S. Pat. No. 5,460,953; and Protein C formulations are described in U.S. Pat. Nos. 6,630,137, 6,436,397, 6,395,270 and 6,159,468, all of which are incorporated herein by reference.

A “Brussels score” score is a method for evaluating organ dysfunction as compared to a baseline.

If the Brussels score is 0 (i.e. moderate, severe, or extreme), then organ failure was recorded as present on that particular day (see TABLE 2A below). In the following description, to correct for deaths during the observation period, days alive and free of organ failure (DAF) were calculated as previously described. For example, acute lung injury was calculated as follows. Acute lung injury is defined as present when a subject meets all of these four criteria. 1) Need for mechanical ventilation, 2) Bilateral pulmonary infiltrates on chest X-ray consistent with acute lung injury, 3) PaO₂/FiO₂ ratio is less than 300, 4) No clinical evidence of congestive heart failure or if a pulmonary artery catheter is in place for clinical purposes, a pulmonary capillary wedge pressure less than 18 mm Hg (1). The severity of acute lung injury is assessed by measuring days alive and free of acute lung injury over a 28 day observation period. Acute lung injury is recorded as present on each day that the person has moderate, severe or extreme dysfunction as defined in the Brussels score. Days alive and free of acute lung injury is calculated as the number of days after onset of acute lung injury that a subject is alive and free of acute lung injury over a defined observation period (28 days). Thus, a lower score for days alive and free of acute lung injury indicates more severe acute lung injury. The reason that days alive and free of acute lung injury is preferable to simply presence or absence of acute lung injury, is that acute lung injury has a high acute mortality and early death (within 28 days) precludes calculation of the presence or absence of acute lung injury in dead subjects. The cardiovascular, renal, neurologic, hepatic and coagulation dysfunction were similarly defined as present on each day that the person had moderate, severe or extreme dysfunction as defined by the Brussels score. Days alive and free of steroids are days that a person is alive and is not being treated with exogenous corticosteroids (e.g. hydrocortisone, prednisone, methylprednisolone). Days alive and free of pressors are days that a person is alive and not being treated with intravenous vasopressors (e.g. dopamine, norepinephrine, epinephrine, phenylephrine). Days alive and free of an International Normalized Ratio (INR)>1.5 are days that a person is alive and does not have an INR>1.5.

TABLE 2A
Brussels Organ Dysfunction Scoring System

| | Free of Organ | | Clinically Significant | | |
|---|---|---|---|---|---|
| | Dysfunction | | Organ Dysfunction | | |
| ORGANS | Normal | Mild | Moderate | Severe | Extreme |
| DAF ORGAN | 1 | | 0 | | |
| DYSFUNCTION | | | | | |
| SCORE | | | | | |
| Cardiovascular | >90 | ≦90 | ≦90 | ≦90 plus | ≦90 plus |
| Systolic BP | | Responsive | Unresponsive to fluid | pH ≦7.3 | pH ≦7.2 |
| (mmHg) | | to fluid | | | |
| Pulmonary | >400 | 400-301 | 300-201 | 200-101 | ≦100 |
| PₐO₂/F_{I}O₂ | | | Acute lung injury | ARDS | Severe |
| (mmHg) | | | | | ARDS |
| Renal | <1.5 | 1.5-1.9 | 2.0-3.4 | 3.5-4.9 | ≧5.0 |
| Creatinine | | | | | |
| (mg/Dl) | | | | | |
| Hepatic | <1.2 | 1.2-1.9 | 2.0-5.9 | 6.0-11.9 | ≧12 |
| Bilirubin | | | | | |
| (mg/dL) | | | | | |
| Hematologic | >120 | 120-81 | 80-51 | 50-21 | ≦20 |
| Platelets | | | | | |
| (×10⁵/mm³) | | | | | |
| Neurologic | 15 | 14-13 | 12-10 | 9-6 | ≦5 |
| (Glascow Score) | | | | | |

Round Table Conference on Clinical Trials for the Treatment of Sepsis Brussels, Mar. 12-14, 1994.

Analysis of variance (ANOVA) is a standard statistical approach to test for statistically significant differences between sets of measurements.

The Fisher exact test is a standard statistical approach to test for statistically significant differences between rates and proportions of characteristics measured in different groups.

### 2. General Methods

One aspect of the invention may involve the identification of subjects or the selection of subjects that are either at risk of developing and inflammatory condition or the identification of subjects who already have an inflammatory condition. For example, subjects who have undergone major surgery or scheduled for or contemplating major surgery may be considered as being at risk of developing an inflammatory condition. Furthermore, subjects may be determined as having an inflammatory condition using diagnostic methods and clinical evaluations known in the medical arts. An inflammatory condition, may be selected from the group consisting of: sepsis, septicemia, pneumonia, septic shock, systemic inflammatory response syndrome (SIRS), Acute Respiratory Distress Syndrome (ARDS), acute lung injury, aspiration pneumanitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for subjects undergoing major surgery or dialysis, subjects who are immunocompromised, subjects on immunosuppressive agents, subjects with HIV/AIDS, subjects with suspected endocarditis, subjects with fever, subjects with fever of unknown origin, subjects with cystic fibrosis, subjects with diabetes mellitus, subjects with chronic renal failure, subjects with acute renal failure, oliguria, subjects with acute renal dysfunction, glomerulo-nephritis, interstitial-nephritis, acute tubular necrosis (ATN), subjects with bronchiectasis, subjects with chronic obstructive lung disease, chronic bronchitis, emphysema, or asthma, subjects with febrile neutropenia, subjects with meningitis, subjects with septic arthritis, subjects with urinary tract infection, subjects with necrotizing fasciitis, subjects with other suspected Group A streptococcus infection, subjects who have had a splenectomy, subjects with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic carinii, pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

Once a subject is identified as being at risk for developing or having an inflammatory condition or is to be administered activated protein C, then genetic sequence information may be obtained from the subject. Or alternatively genetic sequence information may already have been obtained from the subject. For example, a subject may have already provided a biological sample for other purposes or may have even had their genetic sequence determined in whole or in part and stored for future use. Genetic sequence information may be obtained in numerous different ways and may involve the collection of a biological sample that contains genetic material. Particularly, genetic material, containing the sequence or sequences of interest. Many methods are known in the art for collecting bodily samples and extracting genetic material from those samples. Genetic material can be extracted from blood, tissue and hair and other samples. There are many known methods for the separate isolation of DNA and RNA from biological material. Typically, DNA may be isolated from a biological sample when first the sample is lysed and then the DNA is isolated from the lysate according to any one of a variety of multi-step protocols, which can take varying lengths of time. DNA isolation methods may involve the use of phenol (Sambrook, J. et al., “Molecular Cloning”, Vol. 2, pp. 9.14-9.23, Cold Spring Harbor Laboratory Press (1989) and Ausubel, Frederick M. et al., “Current Protocols in Molecular Biology”, Vol. 1, pp. 2.2.1-2.4.5, John Wiley & Sons, Inc. (1994)). Typically, a biological sample is lysed in a detergent solution and the protein component of the lysate is digested with proteinase for 12-18 hours. Next, the lysate is extracted with phenol to remove most of the cellular components, and the remaining aqueous phase is processed further to isolate DNA. In another method, described in Van Ness et al. (U.S. Pat. No. 5,130,423), non-corrosive phenol derivatives are used for the isolation of nucleic acids. The resulting preparation is a mix of RNA and DNA.

Other methods for DNA isolation utilize non-corrosive chaotropic agents. These methods, which are based on the use of guanidine salts, urea and sodium iodide, involve lysis of a biological sample in a chaotropic aqueous solution and subsequent precipitation of the crude DNA fraction with a lower alcohol. The final purification of the precipitated, crude DNA fraction can be achieved by any one of several methods, including column chromatography (Analects, (1994) Vol 22, No. 4, Pharmacia Biotech), or exposure of the crude DNA to a polyanion-containing protein as described in Koller (U.S. Pat. No. 5,128,247).

Yet another method of DNA isolation, which is described by Botwell, D. D. L. (Anal. Biochem. (1987) 162:463-465) involves lysing cells in 6M guanidine hydrochloride, precipitating DNA from the lysate at acid pH by adding 2.5 volumes of ethanol, and washing the DNA with ethanol.

Numerous other methods are known in the art to isolate both RNA and DNA, such as the one described by CHOMCZYNSKI (U.S. Pat. No. 5,945,515), whereby genetic material can be extracted efficiently in as little as twenty minutes. EVANS and HUGH (U.S. Pat. No. 5,989,431) describe methods for isolating DNA using a hollow membrane filter.

Once a subject's genetic material has been obtained from the subject it may then be further be amplified by Reverse Transcription Polymerase Chain Reaction (RT-PCR), Polymerase Chain Reaction (PCR), Transcription Mediated Amplification (TMA), Ligase chain reaction (LCR), Nucleic Acid Sequence Based Amplification (NASBA) or other methods known in the art, and then further analyzed to detect or determine the presence or absence of one or more polymorphisms or mutations in the sequence of interest, provided that the genetic material obtained contains the sequence of interest. Particularly, a person may be interested in determining the presence or absence of a mutation in a protein C pathway associated gene sequence, as described in TABLES 1A-D. The sequence of interest may also include other mutations, or may also contain some of the sequence surrounding the mutation of interest.

Detection or determination of a nucleotide identity, or the presence of one or more single nucleotide polymorphism(s) (SNP typing), may be accomplished by any one of a number methods or assays known in the art. Many DNA typing methodologies are useful detection of SNPs. The majority of SNP genotyping reactions or assays can be assigned to one of four broad groups (sequence-specific hybridization, primer extension, oligonucleotide ligation and invasive cleavage). Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, etc.

In general, sequence-specific hybridization involves a hybridization probe, which is capable of distinguishing between two DNA targets differing at one nucleotide position by hybridization. Usually probes are designed with the polymorphic base in a central position in the probe sequence, whereby under optimized assay conditions only the perfectly matched probe target hybrids are stable and hybrids with a one base mismatch are unstable. A strategy which couples detection and sequence discrimination is the use of a “molecular beacon”, whereby the hybridization probe (molecular beacon) has 3′ and 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Similarly, primer extension reactions (i.e. mini sequencing, nucleotide-specific extensions, or simple PCR amplification) are useful in sequence discrimination reactions. For example, in mini sequencing a primer anneals to its target DNA immediately upstream of the SNP and is extended with a single nucleotide complementary to the polymorphic site. Where the nucleotide is not complementary, no extension occurs.

Oligonucleotide ligation assays require two sequence-specific probes and one common ligation probe per SNP. The common ligation probe hybridizes adjacent to a sequence-specific probe and when there is a perfect match of the appropriate sequence-specific probe, the ligase joins both the sequence-specific and the common probes. Where there is not a perfect match the ligase is unable to join the sequence-specific and common probes. Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

Alternatively, an invasive cleavage method requires an oligonucleotide called an Invader™ probe and sequence-specific probes to anneal to the target DNA with an overlap of one nucleotide. When the sequence-specific probe is complementary to the polymorphic base, overlaps of the 3′ end of the invader oligonucleotide form a structure that is recognized and cleaved by a Flap endonuclease releasing the 5′ arm of the allele specific probe.

5′ exonuclease activity or TaqMan™ assay (Applied Biosystems) is based on the 5′ nuclease activity of Taq polymerase that displaces and cleaves the oligonucleotide probes hybridized to the target DNA generating a fluorescent signal. It is necessary to have two probes that differ at the polymorphic site wherein one probe is complementary to the ‘normal’ sequence and the other to the mutation of interest. These probes have different fluorescent dyes attached to the 5′ end and a quencher attached to the 3′ end when the probes are intact the quencher interacts with the fluorophor by fluorescence resonance energy transfer (FRET) to quench the fluorescence of the probe. During the PCR annealing step the hybridization probes hybridize to target DNA. In the extension step the 5′ fluorescent dye is cleaved by the 5′ nuclease activity of Taq polymerase, leading to an increase in fluorescence of the reporter dye. Mismatched probes are displaced without fragmentation. The presence of a mutation in a sample is determined by measuring the signal intensity of the two different dyes.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput genotyping array (BORTOLIN S. et al. Clinical Chemistry (2004) 50(11): 2028-36). This method amplifies genomic DNA by PCR followed by sequence-specific primer extension with universally tagged genotyping primers. The products are then sorted on a Tag-It array and detected using the Luminex xMAP system.

Mutation detection methods may include but are not limited to the following:

Restriction Fragment Length Polymorphism (RFLP) strategy—An RFLP gel-based analysis can be used to indicate the presence or absence of a specific mutation at polymorphic sites within a gene.

Briefly, a short segment of DNA (typically several hundred base pairs) is amplified by PCR. Where possible, a specific restriction endonuclease is chosen that cuts the short DNA segment when one polymorphism is present but does not cut the short DNA segment when the polymorphism is not present, or vice versa. After incubation of the PCR amplified DNA with this restriction endonuclease, the reaction products are then separated using gel electrophoresis. Thus, when the gel is examined the appearance of two lower molecular weight bands (lower molecular weight molecules travel farther down the gel during electrophoresis) indicates that the DNA sample had a polymorphism was present that permitted cleavage by the specific restriction endonuclease. In contrast, if only one higher molecular weight band is observed (at the molecular weight of the PCR product) then the initial DNA sample had the polymorphism that could not be cleaved by the chosen restriction endonuclease. Finally, if both the higher molecular weight band and the two lower molecular weight bands are visible then the DNA sample contained both polymorphisms, and therefore the DNA sample, and by extension the subject providing the DNA sample, was heterozygous for this polymorphism;

Sequencing—For example the Maxam-Gilbert technique for sequencing (MAXAM A M. and GILBERT W. Proc. Natl. Acad. Sci. USA (1977) 74(4):560-564) involves the specific chemical cleavage of terminally labelled DNA. In this technique four samples of the same labeled DNA are each subjected to a different chemical reaction to effect preferential cleavage of the DNA molecule at one or two nucleotides of a specific base identity. The conditions are adjusted to obtain only partial cleavage, DNA fragments are thus generated in each sample whose lengths are dependent upon the position within the DNA base sequence of the nucleotide(s) which are subject to such cleavage. After partial cleavage is performed, each sample contains DNA fragments of different lengths, each of which ends with the same one or two of the four nucleotides. In particular, in one sample each fragment ends with a C, in another sample each fragment ends with a C or a T, in a third sample each ends with a G, and in a fourth sample each ends with an A or a G. When the products of these four reactions are resolved by size, by electrophoresis on a polyacrylamide gel, the DNA sequence can be read from the pattern of radioactive bands. This technique permits the sequencing of at least 100 bases from the point of labeling. Another method is the dideoxy method of sequencing was published by SANGER et al. (Proc. Natl. Acad. Sci. USA (1977) 74(12):5463-5467). The Sanger method relies on enzymatic activity of a DNA polymerase to synthesize sequence-dependent fragments of various lengths. The lengths of the fragments are determined by the random incorporation of dideoxynucleotide base-specific terminators. These fragments can then be separated in a gel as in the Maxam-Gilbert procedure, visualized, and the sequence determined. Numerous improvements have been made to refine the above methods and to automate the sequencing procedures. Similarly, RNA sequencing methods are also known. For example, reverse transcriptase with dideoxynucleotides have been used to sequence encephalomyocarditis virus RNA (ZIMMERN D. and KAESBERG P. Proc. Natl. Acad. Sci. USA (1978) 75(9):4257-4261). MILLS D R. and KRAMER F R. (Proc. Natl. Acad. Sci. USA (1979) 76(5):2232-2235) describe the use of Q13 replicase and the nucleotide analog inosine for sequencing RNA in a chain-termination mechanism. Direct chemical methods for sequencing RNA are also known (PEATTIE D A. Proc. Natl. Acad. Sci. USA (1979) 76(4):1760-1764). Other methods include those of Donis-Keller et al. (1977, Nucl. Acids Res. 4:2527-2538), SIMONCSITS A. et al. (Nature (1977) 269(5631):833-836), AXELROD V D. et al. (Nucl. Acids Res. (1978) 5(10):3549-3563), and KRAMER F R. and MILLS D R. (Proc. Natl. Acad. Sci. USA (1978) 75(11):5334-5338). Nucleic acid sequences can also be read by stimulating the natural fluoresce of a cleaved nucleotide with a laser while the single nucleotide is contained in a fluorescence enhancing matrix (U.S. Pat. No. 5,674,743); In a mini sequencing reaction, a primer that anneals to target DNA adjacent to a SNP is extended by DNA polymerase with a single nucleotide that is complementary to the polymorphic site. This method is based on the high accuracy of nucleotide incorporation by DNA polymerases. There are different technologies for analyzing the primer extension products. For example, the use of labeled or unlabeled nucleotides, ddNTP combined with dNTP or only ddNTP in the mini sequencing reaction depends on the method chosen for detecting the products;

Probes used in hybridization can include double-stranded DNA, single-stranded DNA and RNA oligonucleotides, and peptide nucleic acids. Hybridization methods for the identification of single nucleotide polymorphisms or other mutations involving a few nucleotides are described in the U.S. Pat. Nos. 6,270,961; 6,025,136; and 6,872,530. Suitable hybridization probes for use in accordance with the invention include oligonucleotides and PNAs from about 10 to about 400 nucleotides, alternatively from about 20 to about 200 nucleotides, or from about 30 to about 100 nucleotides in length.

A template-directed dye-terminator incorporation with fluorescent polarization-detection (TDI-FP) method is described by FREEMAN B D. et al. (J Mol Diagnostics (2002) 4(4):209-215) for large scale screening;

Oligonucleotide ligation assay (OLA) is based on ligation of probe and detector oligonucleotides annealed to a polymerase chain reaction amplicon strand with detection by an enzyme immunoassay (VILLAHERMOSA ML. J Hum Virol (2001) 4(5):238-48; ROMPPANEN E L. Scand J Clin Lab Invest (2001) 61(2):123-9; IANNONE M A. et al. Cytometry (2000) 39(2):131-40);

Ligation-Rolling Circle Amplification (L-RCA) has also been successfully used for genotyping single nucleotide polymorphisms as described in QI X. et al. Nucleic Acids Res (2001) 29(22):E116;

5′ nuclease assay has also been successfully used for genotyping single nucleotide polymorphisms (AYDIN A. et al. Biotechniques (2001) (4):920-2, 924, 926-8.);

Polymerase proofreading methods are used to determine SNPs identities, as described in WO 0181631;

Detection of single base pair DNA mutations by enzyme-amplified electronic transduction is described in PATOLSKY F et al. Nat. Biotech. (2001) 19(3):253-257;

Gene chip technologies are also known for single nucleotide polymorphism discrimination whereby numerous polymorphisms may be tested for simultaneously on a single array (EP 1120646 and GILLES P N. et al. Nat. Biotechnology (1999) 17(4):365-70);

Matrix assisted laser desorption ionization time of flight (MALDI-TOF) mass spectroscopy is also useful in the genotyping single nucleotide polymorphisms through the analysis of microsequencing products (HAFF L A. and SMIRNOV I P. Nucleic Acids Res. (1997) 25(18):3749-50; HAFF L A. and SMIRNOV I P. Genome Res. (1997) 7:378-388; SUN X. et al. Nucleic Acids Res. (2000) 28 e68; BRAUN A. et al. Clin. Chem. (1997) 43:1151-1158; LITTLE DP. et al. Eur. J. Clin. Chem. Clin. Biochem. (1997) 35:545-548; FEI Z. et al. Nucleic Acids Res. (2000) 26:2827-2828; and BLONDAL T. et al. Nucleic Acids Res. (2003) 31(24):e155).

Sequence-specific PCR methods have also been successfully used for genotyping single nucleotide polymorphisms (HAWKINS J R. et al. Hum Mutat (2002) 19(5):543-553). Alternatively, a Single-Stranded Conformational Polymorphism (SSCP) assay or a Cleavase Fragment Length Polymorphism (CFLP) assay may be used to detect mutations as described herein.

Alternatively, if a subject's sequence data is already known, then obtaining may involve retrieval of the subjects nucleic acid sequence data (for example from a database), followed by determining or detecting the identity of a nucleic acid or genotype at a polymorphic site by reading the subject's nucleic acid sequence at the one or more polymorphic sites.

Once the identity of a polymorphism(s) is determined or detected an indication may be obtained as to subject response to activated protein C or protein C like compound or protein C like compound administration based on the genotype (the nucleotide at the position) of the polymorphism of interest. As described herein, polymorphisms in protein C pathway associated gene sequences, may be used to predict a subject's response to activated protein C or protein C like compound treatment. Methods for predicting a subject's response to activated protein C or protein C like compound treatment may be useful in making decisions regarding the administration of activated protein C.

Methods of treatment of an inflammatory condition in a subject having an improved response polymorphism in a protein C pathway associated gene are described herein. An improved response may include an improvement subsequent to administration of said therapeutic agent, whereby the subject has an increased likelihood of survival, reduced likelihood of organ damage or organ dysfunction (Brussels score), an improved APACHE II score, days alive and free of pressors, inotropes, and reduced systemic dysfunction (cardiovascular, respiratory, ventilation, CNS, coagulation [INR>1.5], renal and/or hepatic).

As described above genetic sequence information or genotype information may be obtained from a subject wherein the sequence information contains one or more polymorphic sites in a protein C pathway associated gene sequence. Also, as previously described the sequence identity of one or more polymorphisms in a protein C pathway associated gene sequence of one or more subjects may then be detected or determined. Furthermore, subject response to administration of activated protein C or protein C like compound may be assessed as described above. For example, the APACHE II scoring system or the Brussels score may be used to assess a subject's response to treatment by comparing subject scores before and after treatment. Once subject response has been assessed, subject response may be correlated with the sequence identity of one or more polymorphism(s). The correlation of subject response may further include statistical analysis of subject outcome scores and polymorphism(s) for a number of subjects.

### Cohort Description

All patients admitted to the ICU of St. Paul's Hospital (Vancouver, BC, Canada) were screened for inclusion. The ICU is a mixed medical-surgical ICU in a tertiary care, university-affiliated teaching hospital. Severe sepsis was defined as the presence of at least two systemic inflammatory response syndrome criteria and a known or suspected source of infection plus at least one new organ dysfunction by Brussels criteria (at least moderate, severe or extreme). From this cohort we identified XIGRIS™-treated subjects who were critically ill patients who had severe sepsis, no XIGRIS™ contraindications (e.g. platelet count >30,000, International normalization ration (INR)<3.0) and were treated with XIGRIS™. Control subjects were critically ill patients who had severe sepsis (at least 2 of 4 SIRS criteria, known or suspected infection, and APACHE II ≧25), a platelet count >30,000, INR <3.0, bilirubin <20 mmol/L and were not treated with XIGRIS™. Accordingly, the control group (untreated with XIGRIS™) is comparable to the XIGRIS™-treated group.

### Genotyping

Discarded whole blood samples, stored at 4° C., were collected from the hospital laboratory. The buffy coat was extracted and the samples were transferred to 1.5 mL cryotubes, bar coded and cross-referenced with a unique patient number and stored at −80° C. DNA was extracted from the buffy coat using a QIAamp DNA Midi kit (Qiagen, Mississauga, ON, Canada). Single nucleotide polymorphisms in fibrinogen B beta polypeptide (FGB), coagulation factor II (F2), coagulation factor II receptor (F2R), coagulation factor 111 (F3), coagulation factor V (F5), coagulation factor VII (F7), coagulation factor X (F10), plasminogen activator inhibitor type I (SERPINE1), protein C inhibitor (SERPINA5), interleukin 6 (IL6), interleukin 10 (IL10), interleukin 12A (IL12A), tumor necrosis factor alpha receptor-1 (TNFRSF1A), vascular endothelial growth factor (VEGF), protein C (PROC) and protein C receptor (PROCR) genes were genotyped. TABLE 1A gives the full name of each of these genes and provides a complete list of the 40 haplotype tagged polymorphisms that were genotyped. TABLE 1C gives the flanking sequences for each of the polymorphisms listed in TABLE 1A.

### Clinical Phenotype

Our primary outcome variable was 28-day mortality. Secondary outcome variables were organ dysfunctions (TABLE 2C). Baseline demographics recorded were age, gender, admission APACHE II score (KNAUS W A. et al. Crit. Care Med (1985) 13:818-829), and medical or surgical diagnosis on admission to the ICU (based on the APACHE III diagnostic codes) (KNAUS W A. et al. Chest (1991) 100:1619-1636) (TABLE 2B). After meeting the inclusion criteria, data were recorded for each 24-hour period (8 am to 8 am) for 28-days after ICU admission or until hospital discharge to evaluate organ dysfunction and the intensity of SIRS (Systemic Inflammatory Response Syndrome) and sepsis. Raw clinical and laboratory variables were recorded using the worst or most abnormal variable for each 24-hour period with the exception of Glasgow Coma Score, for which the best possible score for each 24-hour period was recorded. Missing data on the date of admission was assigned a normal value and missing data after day one was substituted by carrying forward the previous day's value. When data collection for each patient was complete, all patient identifiers were removed from all records and the patient file was assigned a unique random number linked with the blood samples. The completed raw data file was used to calculate descriptive and severity of illness scores using standard definitions as described below.

TABLE 2B
Baseline characteristics key.

| Baseline Characteristic | Description |
|---|---|
| AGE | Age, in years |
| SEX/GENDER | % Male |
| APACHE II | APACHE II score |
| SURGICAL | % Surgical admissions |
| SS.ADMIT | % Patients with septic shock upon admission |
| SS.ANY | % Patients with septic shock anytime during |
| | admission |

TABLE 2C
Secondary outcome variables key.
Secondary Outcome Description

Day alive and free of cardiovascular dysfunction
Days alive and free of use of vasopressors
Days alive and free of inotropic agents
Days alive and free of acute lung injury
Days alive and free of respiratory dysfunction
Days alive and free of use of mechanical ventilators
Days alive and free of acute renal dysfunction
Days alive and free of any of renal dysfunction
Days alive and free of renal support
Days alive and free of coagulation dysfunction
Days alive and free of INR >1.5
Days alive and free of neurological dysfunction
Days alive and free of acute hepatic dysfunction
Days alive and free of ¾ SIRS criteria

Organ dysfunction was evaluated at baseline and daily using the Brussels score (SIBBALD W J. and VINCENT J L. Chest (1995) 107(2):522-7) (TABLE 2A). If the Brussels score was moderate, severe, or extreme dysfunction then organ dysfunction was recorded as present on that day. To correct for deaths during the observation period, we calculated the days alive and free of organ dysfunction (RUSSELL J A. et al. Crit. Care Med (2000) 28(10):3405-11 and BERNARD G R. et al. Chest (1997) 112(1):164-72). For example, the severity of cardiovascular dysfunction was assessed by measuring days alive and free of cardiovascular dysfunction over a 28-day observation period. Days alive and free of cardiovascular dysfunction was calculated as the number of days after inclusion that a patient was alive and free of cardiovascular dysfunction over 28-days. Thus, a lower score for days alive and free of cardiovascular dysfunction indicates more cardiovascular dysfunction. The reason that days alive and free of cardiovascular dysfunction is preferable to simply presence or absence of cardiovascular dysfunction is that severe sepsis has a high acute mortality so that early death (within 28-days) precludes calculation of the presence or absence of cardiovascular dysfunction in dead patients. Organ dysfunction has been evaluated in this way in observational studies [34] and in randomized controlled trials of new therapy in sepsis, acute respiratory distress syndrome (BERNARD G R. et al. N Engl J Med (1997) 336(13):912-8) and in critical care (HEBERT P C. et al. N Engl J Med (1999) 340(6):409-17).

To further evaluate cardiovascular, respiratory, and renal function we also recorded, during each 24 hour period, vasopressor support, mechanical ventilation, and renal support, respectively. Vasopressor use was defined as dopamine >5 μg/kg/min or any dose of norepinephrine, epinephrine, vasopressin, or phenylephrine. Mechanical ventilation was defined as need for intubation and positive airway pressure (i.e. T-piece and mask ventilation were not considered ventilation). Renal support was defined as hemodialysis, peritoneal dialysis, or any continuous renal support mode (e.g. continuous veno-venous hemodialysis).

We also scored the presence of three or four of the SIRS criteria each day over the 28-day observation period as a cumulative measure of the severity of SIRS. SIRS was considered present when subjects met at least two of four SIRS criteria. The SIRS criteria were 1) fever (>38° C.) or hypothermia (<35.5° C.), 2) tachycardia (>100 beats/min in the absence of beta blockers, 3) tachypnea (>20 breaths/min) or need for mechanical ventilation, and 4) leukocytosis (total leukocyte count >11,000/μL).

Haplotype Determination and Selection of htSNPs

We used two steps to determine haplotypes and then haplotype clades of the study genes. We inferred haplotypes using PHASE software using un-phased Caucasian genotype data (from http://pga.mbt.washington.edu/) (STEPHENS M. et al. Am J Hum Genet (2001) 68(4):978-89). We then used MEGA 2 to infer a phylogenetic tree so that we could identify major haplotype clades (KUMAR S. et al. Bioinformatics (2001) 17:1244-1245). Haplotypes were sorted according to this phylogenetic tree and this haplotype structure was inspected to choose SNPs that tagged each major haplotype Glade, so-called haplotype tag SNPs (htSNPs) (not shown). Polymorphisms genotyped are listed in TABLE 1A. Polymorphisms included in the Linkage analysis are listed in TABLE 1B with all flanking sequences in TABLES 1C and 1D.

### Statistical Analysis

Baseline characteristics age, gender, APACHE II, and percent surgical patients were recorded in both groups and compared using a chi-squared or Kruskal-Wallis test where appropriate. For each SNP of each gene the 28 day survival rate (%) for patients who were treated with XIGRIS™ (activated protein C) was compared to control patients who were not treated with XIGRIS™ using a chi-squared test. We considered a by-genotype effect to be significant when two criteria were fulfilled. First, we required an increase of >20% in 28-day survival rate in the XIGRIS™ treated group compared to the control group. Second, we required that p<0.1 for this comparison. When both criteria were met we considered the polymorphism allele or genotype which predicted increased 28-day survival with XIGRIS™ treatment to be an “Improved Response Polymorphism” (IRP). Organ dysfunction results were only considered for polymorphisms that were an IRP and were compared between XIGRIS™-treated patients and matched controls using a Kruskal-Wallis test.

### Results

### Baseline Characteristics

Baseline characteristics for the XIGRIS™-treated patients (N=49) and the matched controls (N=250) are given in TABLE 3. These are typical of subjects who have severe sepsis with regards to age, sex and APACHE II score.

TABLE 3
Baseline characteristics (Age, Gender, % Surgical, APACHE II) for XIGRIS ™-treated
patients matched control patients (not treated with XIGRIS ™). Data are shown as 25
percentile/median/75 percentile. Statistical analysis was conducted using a chi-squared or
Kruskal-Wallis test (F) where appropriate.

| | Matched | | | | | |
|---|---|---|---|---|---|---|
| | Controls | XIGRIS ™-Treated | TOTAL | | | |
| | (N = 250) | Patients (N = 49) | (N = 299) | Test Statistic | D.F. | P-VALUE |
| AGE | 51/63/73 | 38/52/67 | 49/62/72 | F = 10.45 | 1.297 | 0.00137 |
| SEX | 65%(163) | 57%(28) | 64%(191) | Chisquare = 1.15 | 1 | 0.283 |
| APACHEII | 27/29/33.75 | 23/32/37 | 26/29/34 | F = 0.18 | 1.297 | 0.674 |
| SURGICAL | 22%(55) | 29%(14) | 23%(69) | Chisquare = 1.0 | 1 | 0.318 |
| SS.ADMIT | 83%(208) | 90%(44) | 84%(252) | Chisquare = 1.35 | 1 | 0.246 |
| SS.ANY | 88%(219) | 92%(45) | 88%(264) | Chisquare = 0.71 | 1 | 0.399 |

D.F., degrees of freedom.

### Survival

Overall, 47 SNP allele or genotype IRPs were identified involving 40 SNPs (TABLE 4). Twenty-eight day Survival by each of the 47 IRPs is given in TABLE 5. For patients with a given IRP allele or genotype, survival is greater for the XIGRIS™-treated patients compared to the matched controls by at least 20% (P<0.1 for each IRP).

TABLE 4
Sample size (N) for TABLES 5 to 18. When the improved response
polymorphism (IRP) is an allele, N represents the number of alleles
genotyped. When the IRP is a genotype, N represents the
number of individuals genotyped.

| | | | N |
|---|---|---|---|
| | | N Matched | XIGRIS ™-Treated |
| SNP | IRP | Controls | Patients or Alleles |
| FGB.155840914.G/A | A | 55 | 8 |
| F2.46717332.G/A | G | 231 | 42 |
| F2.46717332.G/A | GG | 67 | 8 |
| F2R.76059983.A/G | G | 182 | 32 |
| F2R.76059983.A/G | GG | 39 | 7 |
| F2R.76049220.G/C | GG | 128 | 30 |
| F3.94719939.A/G | GG | 42 | 10 |
| F5.166258759.A/G | G | 34 | 9 |
| F5.166236816.T/C | T | 207 | 30 |
| F5.166227911.A/G | A | 157 | 24 |
| F5.166269905.G/A | A | 107 | 21 |
| F7.112808416.A/G | AG | 81 | 17 |
| F10.112840894.A/C | C | 91 | 13 |
| F10.112825510.A/G | G | 81 | 17 |
| F10.112824083.T/C | T | 119 | 21 |
| SERPINE1.100363146.4G/5G | I | 169 | 25 |
| SERPINE1.100375050.G/A | A | 65 | 8 |
| SERPINE1.100375050.G/A | AG | 45 | 8 |
| SERPINA5.94123294.C/T | TT | 56 | 8 |
| IL6.22541812.C/G | C | 52 | 4 |
| IL6.22539885.G/C | G | 93 | 5 |
| IL10.203334802.C/A | A | 59 | 5 |
| IL12A.161198944.G/A | A | 30 | 7 |
| IL12A.161198944.G/A | AG | 30 | 7 |
| TNFRSF1A.6317783.T/C | CT | 88 | 15 |
| VEGF.43848656.G/A | AA | 38 | 4 |
| PROC.127890298.A/G | AG | 74 | 15 |
| PROC.127890457.T/C | CT | 78 | 16 |
| PROC.127892009.G/A | AG | 75 | 16 |
| PROC.127892092.C/T | CT | 90 | 16 |
| PROC.127894204.T/C | C | 214 | 46 |
| PROC.127894204.T/C | CT | 82 | 16 |
| PROC.127894608.G/A | AG | 83 | 16 |
| PROC.127894645.C/T | CT | 84 | 17 |
| PROC.127895556.G/A | A | 88 | 21 |
| PROC.127895556.G/A | AA | 13 | 4 |
| PROC.127895783.G/A | AG | 77 | 15 |
| PROC.127895876.T/C | CT | 84 | 17 |
| PROC.127899224.C/T | CT | 84 | 17 |
| PROC.127901000.T/C | CT | 79 | 11 |
| PROC.127901799.C/T | CT | 84 | 17 |
| PROC.127975205.T/C | C | 133 | 21 |
| PROCR.33183348.T/C | C | 50 | 8 |
| PROCR.33183694.C/A | A | 46 | 8 |
| PROCR.33186524.A/G | G | 35 | 7 |
| PROCR.33228215.A/G | G | 43 | 10 |
| PROCR.33228215.A/G | AG | 37 | 8 |

TABLE 5
28-day survival of XIGRIS ™-treated patients and matched controls (patients not
treated with XIGRIS ™) by different improved response polymorphisms (IRP) in the coagulation,
fibrinolysis and inflammation pathways in a cohort of critically ill patients who had severe sepsis
and no XIGRIS ™ contraindications. Data is presented for both IRP and non-IRP patients.
The chi square tests and the reported P-values correspond to the comparison of IRP Matched Controls
to IRP XIGRIS ™-treated patients only (Column A versus Column B). 28-day survival is given as
% survival (N survived/N total).

| | | 28-Day Survival | | | | | | |
|---|---|---|---|---|---|---|---|---|
| | | | | | D | | | |
| | | A | B | C | non-IRP | | | |
| | | IRP | IRP | non-IRP | XIGRIS ™- | | | |
| | | Matched | XIGRIS ™- | Matched | Treated | A vs B | | |
| SNP | IRP | Controls | Treated Patients | Controls | Patients | Chi-square | D.F. | P-VALUE |
| FGB.155840914.G/A | A | 53% | 88% (7/8) | 53% | 56% (27/48) | 3.45 | 1 | 0.0633 |
| | | (29/55) | | (169/319) | | | | |
| F2.46717332.G/A | G | 46% | 67% (28/42) | 58% | 57% (26/46) | 5.89 | 1 | 0.0153 |
| | | (107/231) | | (115/197) | | | | |
| F2.46717332.G/A | GG | 39% | 75% (6/8) | 58% | 58% (21/36) | 3.83 | 1 | 0.0504 |
| | | (26/67) | | (85/147) | | | | |
| F2R.76059983.A/G | G | 49% | 72% (23/32) | 54% | 57% (31/54) | 5.49 | 1 | 0.0191 |
| | | (90/182) | | (140/258) | | | | |
| F2R.76059983.A/G | GG | 36% | 71% (5/7) | 56% | 61% (22/36) | 3.09 | 1 | 0.0788 |
| | | (14/39) | | (101/181) | | | | |
| F2R.76049220.G/C | GG | 47% | 67% (20/30) | 61% | 54% (7/13) | 3.81 | 1 | 0.051 |
| | | (60/128) | | (54/89) | | | | |
| F3.94719939.A/G | GG | 48% | 80% (8/10) | 53% | 56% (18/32) | 3.41 | 1 | 0.0649 |
| | | (20/42) | | (91/173) | | | | |
| F5.166258759.A/G | G | 56% | 89% (8/9) | 52% | 58% (26/45) | 3.32 | 1 | 0.0685 |
| | | (19/34) | | (163/314) | | | | |
| F5.166236816.T/C | T | 53% | 73% (22/30) | 53% | 50% (12/24) | 4.53 | 1 | 0.0333 |
| | | (109/207) | | (73/139) | | | | |
| F5.166227911.A/G | A | 55% | 75% (18/24) | 49% | 53% (16/30) | 3.28 | 1 | 0.0702 |
| | | (87/157) | | (89/183) | | | | |
| F5.166269905.G/A | A | 54% | 76% (16/21) | 51% | 55% (18/33) | 3.48 | 1 | 0.0622 |
| | | (58/107) | | (124/241) | | | | |
| F7.112808416.A/G | AG | 42% | 65% (11/17) | 61% | 60% (6/10) | 2.92 | 1 | 0.0873 |
| | | (34/81) | | (56/92) | | | | |
| F10.112840894.A/C | C | 46% | 77% (10/13) | 54% | 59% (24/41) | 4.31 | 1 | 0.0379 |
| | | (42/91) | | (138/255) | | | | |
| F10.112825510.A/G | G | 41% | 71% (12/17) | 56% | 59% (22/37) | 5.04 | 1 | 0.0248 |
| | | (33/81) | | (149/267) | | | | |
| F10.112824083.T/C | T | 47% | 71% (15/21) | 55% | 61% (19/31) | 4.24 | 1 | 0.0395 |
| | | (56/119) | | (124/227) | | | | |
| SERPINE1.100363146.4G/5G | I | 44% | 68% (17/25) | 59% | 59% (17/29) | 4.87 | 1 | 0.0273 |
| | | (75/169) | | (99/169) | | | | |
| SERPINE1.100375050.G/A | A | 48% | 88% (7/8) | 53% | 59% (27/46) | 4.52 | 1 | 0.0334 |
| | | (31/65) | | (151/283) | | | | |
| SERPINE1.100375050.G/A | AG | 51% | 88% (7/8) | 51% | 88% (7/8) | 3.66 | 1 | 0.0557 |
| | | (23/45) | | (23/45) | | | | |
| SERPINA5.94123294.C/T | TT | 52% | 88% (7/8) | 52% | 56% (19/34) | 3.63 | 1 | 0.0568 |
| | | (29/56) | | (80/155) | | | | |
| IL6.22541812.C/G | C | 58% | 100% (4/4) | 60% | 100% (2/2) | 2.79 | 1 | 0.095 |
| | | (30/52) | | (12/20) | | | | |
| IL6.22539885.G/C | G | 49% | 100% (5/5) | 49% | 100% (3/3) | 4.86 | 1 | 0.0276 |
| | | (46/93) | | (18/37) | | | | |
| IL.10.203334802.C/A | A | 47% | 100% (5/5) | 45% | 64% (7/11) | 5.1 | 1 | 0.024 |
| | | (28/59) | | (62/139) | | | | |
| IL12A.161198944.G/A | A | 50% | 86% (6/7) | 54% | 58% (34/59) | 2.95 | 1 | 0.0859 |
| | | (15/30) | | (203/378) | | | | |
| IL12A.161198944.G/A | AG | 50% | 86% (6/7) | 54% | 54% (14/26) | 2.95 | 1 | 0.0859 |
| | | (15/30) | | (94/174) | | | | |
| TNFRSF1A.6317783.T/C | CT | 47% | 73% (11/15) | 60% | 50% (6/12) | 3.67 | 1 | 0.0555 |
| | | (41/88) | | (46/77) | | | | |
| VEGF.43848656.G/A | AA | 53% | 100% (4/4) | 54% | 59% (13/22) | 3.32 | 1 | 0.0686 |
| | | (20/38) | | (70/129) | | | | |
| PROC.127890298.A/G | AG | 57% | 80% (12/15) | 49% | 50% (13/26) | 2.82 | 1 | 0.0929 |
| | | (42/74) | | (68/139) | | | | |
| PROC.127890457.T/C | CT | 58% | 81% (13/16) | 49% | 46% (12/26) | 3.12 | 1 | 0.0774 |
| | | (45/78) | | (68/139) | | | | |
| PROC.127892009.G/A | AG | 55% | 81% (13/16) | 49% | 50% (14/28) | 3.86 | 1 | 0.0494 |
| | | (41/75) | | (68/140) | | | | |
| PROC.127892092.C/T | CT | 51% | 81% (13/16) | 51% | 53% (16/30) | 5 | 1 | 0.0253 |
| | | (46/90) | | (74/144) | | | | |
| PROC.127894204.T/C | C | 53% | 74% (34/46) | 51% | 48% (20/42) | 6.87 | 1 | 0.00879 |
| | | (113/214) | | (105/206) | | | | |
| PROC.127894204.T/C | CT | 50% | 75% (12/16) | 53% | 54% (15/28) | 3.37 | 1 | 0.0664 |
| | | (41/82) | | (68/128) | | | | |
| PROC.127894608.G/A | AG | 53% | 88% (14/16) | 52% | 46% (12/26) | 6.58 | 1 | 0.0103 |
| | | (44/83) | | (67/129) | | | | |
| PROC.127894645.C/T | CT | 52% | 82% (14/17) | 51% | 48% (13/27) | 5.19 | 1 | 0.0227 |
| | | (44/84) | | (67/132) | | | | |
| PROC.127895556.G/A | A | 51% | 71% (15/21) | 52% | 59% (41/69) | 2.82 | 1 | 0.093 |
| | | (45/88) | | (181/346) | | | | |
| PROC.127895556.G/A | AA | 46% (6/13) | 100% (4/4) | 52% | 59% (24/41) | 3.66 | 1 | 0.0557 |
| | | | | (107/204) | | | | |
| PROC.127895783.G/A | AG | 56% | 80% (12/15) | 49% | 48% (13/27) | 3.05 | 1 | 0.0809 |
| | | (43/77) | | (67/138) | | | | |
| PROC.127895876.T/C | CT | 51% | 82% (14/17) | 52% | 48% (13/27) | 5.58 | 1 | 0.0181 |
| | | (43/84) | | (67/129) | | | | |
| PROC.127899224.C/T | CT | 52% | 82% (14/17) | 51% | 50% (14/28) | 5.19 | 1 | 0.0227 |
| | | (44/84) | | (65/127) | | | | |
| PROC.127901000.T/C | CT | 56% | 82% (9/11) | 49% | 52% (16/31) | 2.72 | 1 | 0.099 |
| | | (44/79) | | (67/137) | | | | |
| PROC.127901799.C/T | CT | 54% | 82% (14/17) | 51% | 46% (12/26) | 4.82 | 1 | 0.0281 |
| | | (45/84) | | (66/130) | | | | |
| PROC.127975205.T/C | C | 53% | 76% (16/21) | 51% | 57% (36/63) | 3.84 | 1 | 0.0501 |
| | | (71/133) | | (145/283) | | | | |
| PROCR.33183348.T/C | C | 52% | 88% (7/8) | 52% | 61% (49/80) | 3.54 | 1 | 0.0598 |
| | | (26/50) | | (202/390) | | | | |
| PROCR.33183694.C/A | A | 52% | 88% (7/8) | 53% | 58% (45/78) | 3.48 | 1 | 0.0622 |
| | | (24/46) | | (198/374) | | | | |
| PROCR.33186524.A/G | G | 51% | 86% (6/7) | 52% | 59% (48/81) | 2.8 | 1 | 0.0943 |
| | | (18/35) | | (208/401) | | | | |
| PROCR.33228215.A/G | G | 51% | 90% (9/10) | 52% | 61% (51/84) | 5.04 | 1 | 0.0248 |
| | | (22/43) | | (216/417) | | | | |
| PROCR.33228215.A/G | AG | 43% | 88% (7/8) | 53% | 59% (23/39) | 5.16 | 1 | 0.0232 |
| | | (16/37) | | (103/193) | | | | |

D.F., degrees of freedom.

### Organ Dysfunctions of IRP Patients

Significant improvements (P<0.1) in days alive and free of different organ dysfunctions were observed when comparing XIGRIS™-treated patients to the matched controls with a specific IRP allele or genotype (TABLES 6-18). This indicates that for IRP individuals, XIGRIS™ treatment results in improvement in the function of several organ systems including the cardiovascular (and cardiovascular support by vasopressor and inotrope medications), respiratory (plus respiratory support with mechanical ventilation and acute lung injury), renal (and renal support using a form of dialysis), coagulation (and prolonged INR>1.5) and the central nervous systems plus less clinical evidence of inflammation (more days alive and free of 3 of 4 SIRS criteria).

Significant improvements in days alive and free of cardiovascular dysfunction were noted when comparing XIGRIS™-treated patients and the matched controls for 28 of the IRPs (TABLE 6). Significant improvements in days alive and free of vasopressors were noted when comparing XIGRIS™-treated patients and the matched controls for 13 of the IRPs (TABLE 7). Significant improvements in days alive and free of inotropic agents were noted when comparing XIGRIS™-treated patients and the matched controls for 23 of the IRPs (TABLE 8).

TABLE 6
Days alive and free of cardiovascular dysfunction by several polymorphisms in the
coagulation, fibrinolysis and inflammation pathways in a cohort of critically ill patients who had
severe sepsis and no XIGRIS ™ contraindications. More days alive and free of organ dysfunction
indicates improved organ function. Statistical analysis was conducted using a Kruskal-Wallis test
(F). Data is presented as 25^{th} percentile/median/75^{th} percentile.

| | | Days Alive and Free of Cardiovascular | | | | |
|---|---|---|---|---|---|---|
| | | Dysfunction | | | | |
| | | | XIGRIS ™-Treated | | | |
| SNP | IRP | Matched Controls | Patients | F | D.F. | P |
| F5.166258759.A/G | G | 3/12.5/23 | 15/27/27 | 6.69 | 1.41 | 0.0134 |
| F5.166236816.T/C | T | 0/15/24 | 9.5/22/26 | 4.12 | 1.235 | 0.0435 |
| F5.166227911.A/G | A | 2/16/24 | 10.5/22/26 | 2.95 | 1.179 | 0.0875 |
| F5.166269905.G/A | A | 1.5/15/23.5 | 11/22/26 | 3.89 | 1.126 | 0.0509 |
| F10.112840894.A/C | C | 0.50/9/23 | 20/25/27 | 4.39 | 1.102 | 0.0386 |
| F10.112825510.A/G | G | 0/8/22 | 9/24/26 | 5.04 | 1.96 | 0.027 |
| F10.112824083.T/C | T | 1/13/23 | 9/24/26 | 5.24 | 1.138 | 0.0236 |
| SERPINE1.100375050.G/A | A | 0/16/24 | 22.75/25.5/26.25 | 4.83 | 1.71 | 0.0313 |
| SERPINE1.100375050.G/A | AG | 0/16/24 | 22.75/25.5/26.25 | 4.05 | 1.51 | 0.0495 |
| IL6.22541812.C/G | C | 1.75/18/26 | 26/26.5/27.25 | 5.75 | 1.54 | 0.0200 |
| IL6.22539885.G/C | G | 1/9/25 | 11/27/27 | 3.79 | 1.96 | 0.0546 |
| TNFRSF1A.6317783.T/C | CT | 1/9/23 | 10/22/26 | 3.2 | 1.101 | 0.0767 |
| VEGF.43848656.G/A | AA | 2/13/22.75 | 22.75/24.5/26.25 | 3.47 | 1.40 | 0.0698 |
| PROC.127890298.A/G | AG | 3/18/25 | 20/25/26 | 3.52 | 1.87 | 0.0641 |
| PROC.127890457.T/C | CT | 3/18.5/25 | 22/25/26.25 | 4.29 | 1.92 | 0.0412 |
| PROC.127892009.G/A | AG | 2.5/18/24.5 | 22/25/26.25 | 5.41 | 1.89 | 0.0222 |
| PROC.127894204.T/C | C | 1.25/14/24 | 3.5/23/26 | 2.79 | 1.258 | 0.096 |
| PROC.127894608.G/A | AG | 0.5/14/24 | 14.5/24.5/26 | 3.89 | 1.97 | 0.0514 |
| PROC.127894645.C/T | CT | 0/14/24 | 13/24/26 | 3.09 | 1.99 | 0.0818 |
| PROC.127895556.G/A | AA | 4/9/18 | 20.75/24.5/25.25 | 3.6 | 1.15 | 0.0773 |
| PROC.127895783.G/A | AG | 3/18/25 | 20/25/26 | 3.94 | 1.90 | 0.0503 |
| PROC.127895876.T/C | CT | 0/10.5/24 | 13/24/26 | 3.4 | 1.99 | 0.0682 |
| PROC.127899224.C/T | CT | 0/13/24 | 13/24/26 | 3.49 | 1.99 | 0.0647 |
| PROC.127901000.T/C | CT | 2.5/18/24.5 | 20/25/26 | 3.3 | 1.88 | 0.0727 |
| PROC.127901799.C/T | CT | 0/14.5/24 | 13/24/25 | 2.78 | 1.99 | 0.0986 |
| PROC.127975205.T/C | C | 1/14/24 | 11/24/26 | 3.28 | 1.152 | 0.072 |
| PROCR.33228215.A/G | G | 1.5/12/23.5 | 22.5/24.5/26 | 6.07 | 1.51 | 0.0172 |
| PROCR.33228215.A/G | AG | 1/6/25 | 20.25/25.5/26.25 | 4.31 | 1.43 | 0.0439 |

IRP, improved response polymorphism.
D.F., degrees of freedom.

TABLE 7
Days alive and free of vasopressors by several polymorphisms in the coagulation,
fibrinolysis and inflammation pathways in a cohort of critically ill patients who had severe sepsis
and no XIGRIS ™ contraindications. More days alive and free of organ dysfunction indicates
improved organ function. Statistical analysis was conducted using a Kruskal-Wallis test (F). Data
is presented as 25^{th} percentile/median/75^{th} percentile.

| | | Days Alive and Free of Vassopressors | | | | |
|---|---|---|---|---|---|---|
| | | | XIGRIS ™-Treated | | | |
| SNP | IRP | Matched Controls | Patients | F | D.F. | P |
| F5.166258759.A/G | G | 3.25/18.5/25.75 | 17/27/28 | 3.3 | 1.41 | 0.0764 |
| F10.112840894.A/C | C | 2/15/25.5 | 24/25/28 | 3.45 | 1.102 | 0.0663 |
| SERPINE1.100375050.G/A | A | 2/20/26 | 25/26/27 | 3.43 | 1.71 | 0.0683 |
| SERPINE1.100375050.G/A | AG | 1/20/26 | 25/26/27 | 2.96 | 1.51 | 0.0912 |
| IL6.22541812.C/G | C | 1.75/20.5/27 | 27/27.5/28 | 6.15 | 1.54 | 0.0163 |
| IL6.22539885.G/C | G | 1/17/26 | 17/28/28 | 4.2 | 1.96 | 0.0432 |
| IL10.203334802.C/A | A | 0/12/23 | 26/28/28 | 11.21 | 1.62 | 0.00139 |
| VEGF.43848656.G/A | AA | 2/17/25.75 | 25.75/26/26.5 | 3.66 | 1.40 | 0.0631 |
| PROC.127892009.G/A | AG | 3.5/21/26 | 24.25/26/27 | 3.16 | 1.89 | 0.0787 |
| PROC.127894608.G/A | AG | 2/18/26 | 21.75/25/26 | 3.26 | 1.97 | 0.0743 |
| PROC.127975205.T/C | C | 2/18/26 | 17/25/26 | 2.81 | 1.152 | 0.0955 |
| PROCR.33228215.A/G | G | 1.5/19/26 | 26/26/27 | 4.2 | 1.51 | 0.0456 |
| PROCR.33228215.A/G | AG | 1/15/26 | 23.75/26.5/27.25 | 3.89 | 1.43 | 0.055 |

IRP, improved response polymorphism.
D.F., degrees of freedom.

TABLE 8
Days alive and free of inotropic agents by several polymorphisms in the
coagulation, fibrinolysis and inflammation pathways in a cohort of critically
ill patients who had severe sepsis and no XIGRIS ™ contraindications.

| | | Days Alive and Free | | | | |
|---|---|---|---|---|---|---|
| | | of Inotropic Agents | | | | |
| | | | XIGRIS ™-Treated | | | |
| SNP | IRP | Matched Controls | Patients | F | D.F. | P |
| F2.46717332.G/A | GG | 3/13/28 | 21.25/27/28 | 3.27 | 1.73 | 0.0746 |
| F5.166258759.A/G | G | 4.25/26/28 | 28/28/28 | 5.62 | 1.41 | 0.0225 |
| F5.166236816.T/C | T | 4/24/28 | 16.25/28/28 | 4.3 | 1.235 | 0.0392 |
| F5.166227911.A/G | A | 6/26/28 | 20.75/28/28 | 3.99 | 1.179 | 0.0474 |
| F10.112825510.A/G | G | 2/21/28 | 14/28/28 | 3.36 | 1.96 | 0.0698 |
| SERPINE1.100375050.G/A | A | 5/22/28 | 27.5/28/28 | 4.42 | 1.71 | 0.039 |
| SERPINE1.100375050.G/A | AG | 7/26/28 | 27.5/28/28 | 3.08 | 1.51 | 0.0852 |
| IL6.22539885.G/C | G | 2/22/28 | 28/28/28 | 4.65 | 1.96 | 0.0335 |
| IL10.203334802.C/A | A | 4.5/16/28 | 28/28/28 | 5.7 | 1.62 | 0.0201 |
| TNFRSF1A.6317783.T/C | CT | 5.75/22/28 | 18.5/28/28 | 3.1 | 1.101 | 0.0811 |
| VEGF.43848656.G/A | AA | 4.25/24.5/28 | 28/28/28 | 3.97 | 1.40 | 0.0531 |
| PROC.127892009.G/A | AG | 5.5/26/28 | 27.5/28/28 | 3.48 | 1.89 | 0.0652 |
| PROC.127892092.C/T | CT | 4.25/25/28 | 27.25/28/28 | 4.51 | 1.104 | 0.0361 |
| PROC.127894204.T/C | C | 5/26/28 | 13.75/28/28 | 2.74 | 1.258 | 0.099 |
| PROC.127894608.G/A | AG | 4/26/28 | 27.5/28/28 | 4.56 | 1.97 | 0.0352 |
| PROC.127894645.C/T | CT | 4/25/28 | 26/28/28 | 4.17 | 1.99 | 0.0438 |
| PROC.127895876.T/C | CT | 3.75/23.5/28 | 26/28/28 | 4.54 | 1.99 | 0.0356 |
| PROC.127899224.C/T | CT | 4/25/28 | 26/28/28 | 4.27 | 1.99 | 0.0413 |
| PROC.127901000.T/C | CT | 5.5/26/28 | 28/28/28 | 3.21 | 1.88 | 0.0765 |
| PROC.127901799.C/T | CT | 4/25/28 | 28/28/28 | 5.46 | 1.99 | 0.0215 |
| PROC.127975205.T/C | C | 4/26/28 | 25/28/28 | 3.86 | 1.152 | 0.0513 |
| PROCR.33228215.A/G | G | 3/24/28 | 28/28/28 | 4.42 | 1.51 | 0.0405 |
| PROCR.33228215.A/G | AG | 2/19/28 | 26.75/28/28 | 4.3 | 1.43 | 0.0442 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

Significant improvements in days alive and free of acute lung injury were noted when comparing XIGRIS™-treated patients and the matched controls for 3 of the IRPs (TABLE 9). Significant improvements in days alive and free of respiratory dysfunction were noted when comparing XIGRIS™-treated patients and the matched controls for 16 of the IRPs (TABLE 10). Significant improvements in days alive and free of mechanical ventilator use were noted when comparing XIGRIS™-treated patients and the matched controls for 29 of the IRPs (TABLE 11).

TABLE 9
Days alive and free of acute lung injury by several polymorphisms in the
coagulation, fibrinolysis and inflammation pathways in a cohort of critically
ill patients who had severe sepsis and no XIGRIS ™ contraindications.

| | | Days Alive and Free of Acute Lung | | | | |
|---|---|---|---|---|---|---|
| | | Injury | | | | |
| | | | XIGRIS ™-Treated | | | |
| SNP | IRP | Matched Controls | Patients | F | D.F. | P |
| PROCR.33183348.T/C | C | 2.25/8/27 | 1.5/2/6.75 | 4.71 | 1.56 | 0.0343 |
| PROCR.33183694.C/A | A | 3/11.5/27 | 1.5/2/6.75 | 4.94 | 1.52 | 0.0306 |
| PROCR.33186524.A/G | G | 2.5/15/27 | 2/2/7.5 | 3.1 | 1.40 | 0.086 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism. D.F., degrees of freedom.

TABLE 10
Days alive and free of respiratory dysfunction by several polymorphisms in the
coagulation, fibrinolysis and inflammation pathways in a cohort of critically ill
patients who had severe sepsis and no XIGRIS ™ contraindications.

| | | Days Alive and Free of Respiratory | | | | |
|---|---|---|---|---|---|---|
| | | Dysfunction | | | | |
| | | | XIGRIS ™-Treated | | | |
| SNP | IRP | Matched Controls | Patients | F | D.F. | P |
| F2R.76059983.A/G | G | 0/3/20 | 3.5/19/22 | 5.13 | 1.212 | 0.0245 |
| F3.94719939.A/G | GG | 0/2.5/19.5 | 19.25/22.5/24 | 3.85 | 1.50 | 0.0553 |
| F5.166236816.T/C | T | 0/3/22 | 4/20/23 | 4.54 | 1.235 | 0.0341 |
| F5.166227911.A/G | A | 0/7/22 | 4/19.5/22.25 | 2.75 | 1.179 | 0.099 |
| F10.112840894.A/C | C | 0/2/21.5 | 19/21/24 | 4.71 | 1.102 | 0.0324 |
| F10.112825510.A/G | G | 0/1/18 | 4/20/23 | 4.64 | 1.96 | 0.0338 |
| F10.112824083.T/C | T | 0/3/19 | 4/19/23 | 4.77 | 1.138 | 0.0306 |
| IL10.203334802.C/A | A | 0/1/15.5 | 19/23/24 | 8.16 | 1.62 | 0.00583 |
| TNFRSF1A.6317783.T/C | CT | 0/2/20.25 | 4/19/22.5 | 3.67 | 1.101 | 0.0581 |
| VEGF.43848656.G/A | AA | 0/2/18 | 16/21/22.5 | 2.98 | 1.40 | 0.0921 |
| PROC.127890298.A/G | AG | 0/8.5/20 | 10/20/23 | 3.02 | 1.87 | 0.0856 |
| PROC.127890457.T/C | CT | 0/9/20.75 | 12/20/23.25 | 3.31 | 1.92 | 0.072 |
| PROC.127892009.G/A | AG | 0/7/20 | 12/20/23.25 | 4.41 | 1.89 | 0.0386 |
| PROC.127894204.T/C | C | 0/6/20 | 1/15/22.75 | 3.1 | 1.258 | 0.0794 |
| PROC.127895783.G/A | AG | 0/8/20 | 10/20/22.5 | 3.18 | 1.90 | 0.078 |
| PROC.127975205.T/C | C | 0/4/20 | 4/14/23 | 3.02 | 1.152 | 0.0844 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

TABLE 11
Days alive and free of mechanical ventilator use by several polymorphisms in the
coagulation, fibrinolysis and inflammation pathways in a cohort of critically ill
patients who had severe sepsis and no XIGRIS ™ contraindications.

| | | Days Alive and Free of Mechanical | | | | |
|---|---|---|---|---|---|---|
| | | Ventilator Use | | | | |
| | | | XIGRIS ™-Treated | | | |
| SNP | IRP | Matched Controls | Patients | F | D.F. | P |
| F2.46717332.G/A | G | 0/0/18 | 0.25/4/19 | 3.03 | 1.271 | 0.083 |
| F2R.76059983.A/G | G | 0/1/18 | 1.75/18.5/22 | 7.76 | 1.212 | 0.00581 |
| F2R.76059983.A/G | GG | 0/0/8.5 | 7/19/21 | 3.95 | 1.44 | 0.0531 |
| F3.94719939.A/G | GG | 0/0/19.5 | 19.25/22/24 | 5.06 | 1.50 | 0.0289 |
| F5.166236816.T/C | T | 0/1/21 | 3/17.5/22 | 5.66 | 1.235 | 0.0181 |
| F5.166227911.A/G | A | 0/2/21 | 3/17.5/22 | 3.77 | 1.179 | 0.0539 |
| F5.166269905.G/A | A | 0/2/20 | 3/17/22 | 3.22 | 1.126 | 0.0751 |
| F10.112840894.A/C | C | 0/0/19.5 | 17/20/24 | 5.27 | 1.102 | 0.0237 |
| F10.112825510.A/G | G | 0/0/15 | 3/20/23 | 5.76 | 1.96 | 0.0184 |
| F10.112824083.T/C | T | 0/2/18 | 3/19/23 | 4.73 | 1.138 | 0.0314 |
| SERPINE1.100363146.4G/5G | I | 0/1/18 | 1/17/22 | 4.04 | 1.192 | 0.0459 |
| SERPINE1.100375050.G/A | AG | 0/6/21 | 15.25/21/24.25 | 3 | 1.51 | 0.0893 |
| IL10.203334802.C/A | A | 0/0/11 | 19/23/23 | 9.94 | 1.62 | 0.00249 |
| IL6.22541812.C/G | C | 0/1/18.25 | 22/23.5/24 | 3.92 | 1.54 | 0.0527 |
| IL6.22539885.G/C | G | 0/1/18 | 7/19/23 | 3.28 | 1.96 | 0.0733 |
| TNFRSF1A.6317783.T/C | CT | 0/0.5/17.25 | 3/17/22 | 4.38 | 1.101 | 0.039 |
| VEGF.43848656.G/A | AA | 0/0.5/18 | 15.25/20.5/22.25 | 3.25 | 1.40 | 0.079 |
| PROC.127890298.A/G | AG | 0/6/18 | 9/20/23 | 4.26 | 1.87 | 0.0421 |
| PROC.127890457.T/C | CT | 0/7/18.75 | 11.5/20/22.5 | 4.74 | 1.92 | 0.032 |
| PROC.127892009.G/A | AG | 0/4/18 | 11.5/20/22.5 | 5.77 | 1.89 | 0.0184 |
| PROC.127894204.T/C | C | 0/4/18 | 1/15/22 | 5.23 | 1.258 | 0.023 |
| PROC.127894608.G/A | AG | 0/1/21 | 4/19.5/22 | 3.12 | 1.97 | 0.0807 |
| PROC.127894645.C/T | CT | 0/0/18.5 | 4/19/22 | 3.73 | 1.99 | 0.0563 |
| PROC.127895783.G/A | AG | 0/5/18 | 9/20/22 | 4.43 | 1.90 | 0.038 |
| PROC.127895876.T/C | CT | 0/1/20 | 4/19/22 | 3.16 | 1.99 | 0.0784 |
| PROC.127899224.C/T | CT | 0/0.5/20 | 4/19/22 | 3.15 | 1.99 | 0.079 |
| PROC.127901000.T/C | CT | 0/5/18 | 9/20/22 | 3.95 | 1.88 | 0.0499 |
| PROC.127901799.C/T | CT | 0/1/18.5 | 4/17/20 | 3.04 | 1.99 | 0.0843 |
| PROC.127975205.T/C | C | 0/3/18 | 2/14/22 | 3.63 | 1.152 | 0.0586 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

Significant improvements in days alive and free of acute renal dysfunction were noted when comparing XIGRIS™-treated patients and the matched controls for 23 of the IRPs (TABLE 12). Significant improvements in days alive and free of any renal dysfunction were noted when comparing XIGRIS™-treated patients and the matched controls for 32 of the IRPs (TABLE 13). Significant improvements in days alive and free of renal support with any form of dialysis were noted when comparing XIGRIS™-treated patients and the matched controls for 19 of the IRPs (TABLE 14).

TABLE 12
Days alive and free of acute renal dysfunction by several polymorphisms in the
coagulation, fibrinolysis and inflammation pathways in a cohort of critically ill
patients who had severe sepsis and no XIGRIS ™ contraindications.

| | | Days Alive and Free of | | | | |
|---|---|---|---|---|---|---|
| | | Acute Renal Dysfunction | | | | |
| | | | XIGRIS ™-Treated | | | |
| SNP | IRP | Matched Controls | Patients | F | D.F. | P |
| F2.46717332.G/A | G | 2/11/26.5 | 5.5/17/28 | 4.31 | 1.271 | 0.0387 |
| F2R.76059983.A/G | G | 1.25/12/27 | 4.5/25.5/28 | 4.27 | 1.212 | 0.0401 |
| F2R.76059983.A/G | GG | 1/7/20.5 | 14/28/28 | 3.25 | 1.44 | 0.0781 |
| F5.166258759.A/G | G | 3/15.5/28 | 15/28/28 | 3.63 | 1.41 | 0.0636 |
| F5.166236816.T/C | T | 2/10/27 | 6.75/27/28 | 6.99 | 1.235 | 0.00875 |
| F5.166227911.A/G | A | 2/15/27 | 13.5/27/28 | 6.17 | 1.179 | 0.0139 |
| F10.112825510.A/G | G | 2/13/26 | 14/27/28 | 5.87 | 1.96 | 0.0172 |
| SERPINA5.94123294.C/T | TT | 2/15.5/27 | 24/28/28 | 6.53 | 1.62 | 0.0131 |
| IL6.22541812.C/G | C | 2/11.5/26 | 0/0/6.75 | 3.11 | 1.54 | 0.0836 |
| TNFRSF1A.6317783.T/C | CT | 2/12/27.25 | 13/27/28 | 4.73 | 1.101 | 0.0319 |
| VEGF.43848656.G/A | AA | 3/10.5/28 | 27/27.5/28 | 3.21 | 1.40 | 0.0809 |
| PROC.127894204.T/C | C | 2/14/27.75 | 4/25.5/28 | 3.28 | 1.258 | 0.0714 |
| PROC.127894204.T/C | CT | 1/13/27 | 2.5/27.5/28 | 2.92 | 1.96 | 0.091 |
| PROC.127894608.G/A | AG | 1/10/27 | 19.5/28/28 | 6.27 | 1.97 | 0.014 |
| PROC.127894645.C/T | CT | 2/13/27 | 3/28/28 | 3.75 | 1.99 | 0.0558 |
| PROC.127895876.T/C | CT | 1/10/27 | 3/28/28 | 4.69 | 1.99 | 0.0327 |
| PROC.127899224.C/T | CT | 1/13/27.25 | 3/28/28 | 3.55 | 1.99 | 0.0623 |
| PROC.127901000.T/C | CT | 2/16/27.5 | 21/28/28 | 4.65 | 1.88 | 0.0338 |
| PROC.127901799.C/T | CT | 1.75/13/27.25 | 25/28/28 | 6.48 | 1.99 | 0.0125 |
| PROC.127975205.T/C | C | 1/12/28 | 12/19/28 | 2.8 | 1.152 | 0.0962 |
| PROCR.33183348.T/C | C | 2/5.5/25.5 | 17.25/23.5/28 | 3.11 | 1.56 | 0.0834 |
| PROCR.33183694.C/A | A | 2/5.5/23.75 | 17.25/23.5/28 | 3.46 | 1.52 | 0.0687 |
| PROCR.33228215.A/G | G | 2/7/27.5 | 21.25/28/28 | 3.54 | 1.51 | 0.0657 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

TABLE 13
Days alive and free of any renal dysfunction by several polymorphisms in the
coagulation, fibrinolysis and inflammation pathways in a cohort of critically ill
patients who had severe sepsis and no XIGRIS ™ contraindications.

| | | Days Alive and Free of Any Renal | | | | |
|---|---|---|---|---|---|---|
| | | Dysfunction | | | | |
| | | | XIGRIS ™-Treated | | | |
| SNP | IRP | Matched Controls | Patients | F | D.F. | P |
| F2.46717332.G/A | G | 1/6/25 | 3.5/14.5/28 | 5.41 | 1.271 | 0.0208 |
| F2R.76059983.A/G | G | 1/8.5/24.75 | 4.5/22/28 | 6.23 | 1.212 | 0.0133 |
| F2R.76059983.A/G | GG | 1/5/18 | 14/28/28 | 3.83 | 1.44 | 0.0567 |
| F2R.76049220.G/C | GG | 0/6.5/21.25 | 3.5/16.5/28 | 5.12 | 1.156 | 0.025 |
| F5.166236816.T/C | T | 1/8/26 | 6.75/16.5/28 | 7.35 | 1.235 | 0.00719 |
| F5.166227911.A/G | A | 1/12/27 | 13.5/16.5/28 | 5.07 | 1.179 | 0.0256 |
| F10.112825510.A/G | G | 1/8/25 | 14/27/28 | 6.67 | 1.96 | 0.0113 |
| SERPINE1.100375050.G/A | A | 0/7/26 | 12.25/28/28 | 3.95 | 1.71 | 0.0506 |
| SERPINE1.100375050.G/A | AG | 1/12/27 | 12.25/28/28 | 2.94 | 1.51 | 0.0924 |
| SERPINA5.94123294.C/T | TT | 1/13.5/27 | 13.5/28/28 | 4.86 | 1.62 | 0.0313 |
| IL12A.161198944.G/A | A | 1/3.5/16.25 | 15.5/28/28 | 5.19 | 1.35 | 0.0289 |
| IL12A.161198944.G/A | AG | 1/3.5/16.25 | 15.5/28/28 | 5.19 | 1.35 | 0.0289 |
| TNFRSF1A.6317783.T/C | CT | 0/7.5/20.5 | 13/18/27 | 6.99 | 1.101 | 0.0095 |
| VEGF.43848656.G/A | AA | 0/6/27 | 27/27.5/28 | 4.4 | 1.40 | 0.0424 |
| PROC.127890298.A/G | AG | 0.25/8.5/24.75 | 2/28/28 | 4.47 | 1.87 | 0.0375 |
| PROC.127890457.T/C | CT | 1/8.5/24.75 | 2.5/27.5/28 | 4.41 | 1.92 | 0.0386 |
| PROC.127892009.G/A | AG | 1/9/25.5 | 2.5/27.5/28 | 4.07 | 1.89 | 0.0467 |
| PROC.127892092.C/T | CT | 0/9.5/27 | 2.5/27.5/28 | 3.37 | 1.104 | 0.0693 |
| PROC.127894204.T/C | C | 1/10.5/26 | 3.25/19/28 | 6.44 | 1.258 | 0.0117 |
| PROC.127894204.T/C | CT | 0/9/26 | 1.75/27.5/28 | 4.32 | 1.96 | 0.0404 |
| PROC.127894608.G/A | AG | 0.5/8/26 | 19.5/28/28 | 8.87 | 1.97 | 0.00366 |
| PROC.127894645.C/T | CT | 1/9/26.25 | 3/28/28 | 5.66 | 1.99 | 0.0193 |
| PROC.127895556.G/A | A | 0/5/22.25 | 2/19/28 | 5.32 | 1.107 | 0.023 |
| PROC.127895783.G/A | AG | 1/9/25 | 9/28/28 | 5.53 | 1.90 | 0.0208 |
| PROC.127895876.T/C | CT | 0/8/26 | 3/28/28 | 6.98 | 1.99 | 0.0096 |
| PROC.127899224.C/T | CT | 0.75/9.5/27 | 3/28/28 | 5.62 | 1.99 | 0.0197 |
| PROC.127901000.T/C | CT | 1/9/26 | 21/28/28 | 7.97 | 1.88 | 0.00587 |
| PROC.127901799.C/T | CT | 1/9.5/27 | 25/28/28 | 8.66 | 1.99 | 0.00405 |
| PROC.127975205.T/C | C | 0/7/26 | 10/19/28 | 6.4 | 1.152 | 0.0125 |
| PROCR.33183348.T/C | C | 0/3/22 | 13.5/18.5/21.25 | 2.94 | 1.56 | 0.0918 |
| PROCR.33183694.C/A | A | 0/3/21.25 | 13.5/18.5/21.25 | 3.48 | 1.52 | 0.0677 |
| PROCR.33228215.A/G | G | 0/3/23 | 15/23.5/28 | 3.92 | 1.51 | 0.0533 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

TABLE 14
Days alive and free of renal support by several polymorphisms in the
coagulation, fibrinolysis and inflammation pathways in a cohort of critically ill
patients who had severe sepsis and no XIGRIS ™ contraindications.

| | | Days Alive and Free of Renal Support | | | | |
|---|---|---|---|---|---|---|
| | | | XIGRIS ™-Treated | | | |
| SNP | IRP | Matched Controls | Patients | F | D.F. | P |
| F2R.76059983.A/G | G | 1/9/28 | 4.25/27.5/28 | 4.13 | 1.212 | 0.0433 |
| F10.112825510.A/G | G | 2/13/28 | 5/28/28 | 3.19 | 1.96 | 0.0771 |
| SERPINE1.100375050.G/A | A | 2/15/28 | 22/28/28 | 2.99 | 1.71 | 0.088 |
| SERPINE1.100375050.G/A | AG | 2/20/28 | 22/28/28 | 2.8 | 1.51 | 0.100 |
| IL10.203334802.C/A | A | 2/15/27.5 | 15/28/28 | 3.36 | 1.62 | 0.0714 |
| IL12A.161198944.G/A | A | 1/4.5/25.25 | 21.5/28/28 | 3.83 | 1.35 | 0.0583 |
| IL12A.161198944.G/A | AG | 1/4.5/25.25 | 21.5/28/28 | 3.83 | 1.35 | 0.0583 |
| VEGF.43848656.G/A | AA | 2.25/14/28 | 28/28/28 | 5.07 | 1.40 | 0.0299 |
| PROC.127890298.A/G | AG | 1/15/28 | 13/28/28 | 4.33 | 1.87 | 0.0405 |
| PROC.127890457.T/C | CT | 1/15/28 | 19/28/28 | 5.44 | 1.92 | 0.0219 |
| PROC.127892009.G/A | AG | 1/15/28 | 19/28/28 | 4.67 | 1.89 | 0.0335 |
| PROC.127894608.G/A | AG | 1/10/28 | 7.5/28/28 | 4.02 | 1.97 | 0.0478 |
| PROC.127894645.C/T | CT | 1/12/28 | 6/28/28 | 3.18 | 1.99 | 0.0777 |
| PROC.127895783.G/A | AG | 1/15/28 | 14.5/28/28 | 4.91 | 1.90 | 0.0293 |
| PROC.127895876.T/C | CT | 1/9.5/28 | 6/28/28 | 3.73 | 1.99 | 0.0563 |
| PROC.127899224.C/T | CT | 1/13/28 | 6/28/28 | 2.83 | 1.99 | 0.0954 |
| PROC.127901000.T/C | CT | 1/15/28 | 28/28/28 | 6.21 | 1.88 | 0.0146 |
| PROC.127901799.C/T | CT | 1/14/28 | 9/28/28 | 5.02 | 1.99 | 0.0273 |
| PROC.127975205.T/C | C | 1/11/28 | 9/28/28 | 3.01 | 1.152 | 0.085 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

Significant improvements in days alive and free of coagulation dysfunction (as measured by the Brussels hematologic platelet count) were noted when comparing XIGRIS™-treated patients and the matched controls for the IL10.203334802.C/A and PROC.127895556.G/A IRP (TABLE 15). Significant improvements in days alive and free of INR>1.5 were noted when comparing XIGRIS™-treated patients and the matched controls for 43 of the IRPs (TABLE 16).

TABLE 15
Days alive and free of coagulation dysfunction (as measured by the
Brussels hematologic platelet count)by several polymorphisms in the
coagulation, fibrinolysis and inflammation pathways in a cohort of
critically ill patients who had severe sepsis and no XIGRIS ™
contraindications.

| | | Days Alive and Free of | | | | |
|---|---|---|---|---|---|---|
| | | Coagulation Dysfunction | | | | |
| | | | XIGRIS ™-Treated | | | |
| SNP | IRP | Matched Controls | Patients | F | D.F. | P |
| IL10.203334802.C/A | A | 4/20/28 | 27/28/28 | 3.42 | 1.62 | 0.0692 |
| PROC.127895556.G/A | AA | 7/15/25 | 23.25/27.5/28 | 3.35 | 1.15 | 0.087 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

TABLE 16
Days alive and free of INR > 1.5 by several polymorphisms in the coagulation,
fibrinolysis and inflammation pathways in a cohort of critically ill patients who
had severe sepsis and no XIGRIS ™ contraindications.

| | | Days Alive and Free of INR > 1.5 | | | | |
|---|---|---|---|---|---|---|
| | | | XIGRIS ™- | | | |
| SNP | IRP | Matched Controls | Treated Patients | F | D.F. | P |
| FGB.155840914.G/A | A | 9/23/28 | 27.75/28/28 | 4.04 | 1.61 | 0.0488 |
| F2.46717332.G/A | G | 2/15/28 | 5.25/27.5/28 | 6.45 | 1.271 | 0.0117 |
| F2.46717332.G/A | GG | 2/9/27.5 | 5.75/28/28 | 3.38 | 1.73 | 0.0699 |
| F3.94719939.A/G | GG | 1.25/18/26 | 10.5/28/28 | 4.45 | 1.50 | 0.0399 |
| F2R.76059983.A/G | G | 3/20/28 | 5/28/28 | 4.63 | 1.212 | 0.0325 |
| F2R.76049220.G/C | GG | 2.75/17.5/28 | 5/27/28 | 3.06 | 1.156 | 0.0824 |
| F5.166258759.A/G | G | 7.25/26.5/28 | 28/28/28 | 4.49 | 1.41 | 0.0401 |
| F5.166236816.T/C | T | 3/23/28 | 17.25/28/28 | 6.94 | 1.235 | 0.00901 |
| F5.166227911.A/G | A | 6/25/28 | 23.75/28/28 | 5.81 | 1.179 | 0.017 |
| F5.166269905.G/A | A | 5.5/26/28 | 27/28/28 | 4.95 | 1.126 | 0.0279 |
| F7.112808416.A/G | AG | 2/10/28 | 4/28/28 | 4.08 | 1.96 | 0.0463 |
| F10.112840894.A/C | C | 3/18/28 | 27/28/28 | 4.56 | 1.102 | 0.0352 |
| F10.112825510.A/G | G | 2/12/27 | 14/28/28 | 8.09 | 1.96 | 0.00545 |
| F10.112824083.T/C | T | 3.5/21/28 | 14/28/28 | 8.63 | 1.138 | 0.00387 |
| SERPINE1.100363146.4G/5G | I | 3/16/28 | 4/28/28 | 5.75 | 1.192 | 0.0174 |
| SERPINE1.100375050.G/A | A | 7/23/28 | 28/28/28 | 6.02 | 1.71 | 0.0166 |
| SERPINE1.100375050.G/A | AG | 8/23/28 | 28/28/28 | 5.55 | 1.51 | 0.0223 |
| SERPINA5.94123294.C/T | TT | 2.75/20.5/28 | 22.25/28/28 | 3.71 | 1.62 | 0.0587 |
| IL6.22541812.C/G | C | 5/26/28 | 28/28/28 | 4.06 | 1.54 | 0.049 |
| IL6.22539885.G/C | G | 3/19/28 | 28/28/28 | 7.02 | 1.96 | 0.00941 |
| IL10.203334802.C/A | A | 4.5/15/27 | 28/28/28 | 9.22 | 1.62 | 0.0035 |
| TNFRSF1A.6317783.T/C | CT | 6.75/21.5/28 | 20.5/28/28 | 4.32 | 1.101 | 0.0402 |
| VEGF.43848656.G/A | AA | 3.25/22/28 | 27.75/28/28 | 3.33 | 1.40 | 0.0755 |
| PROC.127890298.A/G | AG | 5.25/23.5/28 | 26.5/28/28 | 3.41 | 1.87 | 0.0681 |
| PROC.127890457.T/C | CT | 6.5/24.5/28 | 26.75/28/28 | 3.91 | 1.92 | 0.0508 |
| PROC.127892009.G/A | AG | 4.5/22/28 | 26.75/28/28 | 4.35 | 1.89 | 0.04 |
| PROC.127892092.C/T | CT | 3/22.5/28 | 26/28/28 | 3.64 | 1.104 | 0.0593 |
| PROC.127894204.T/C | C | 4/21.5/28 | 7/28/28 | 7.44 | 1.258 | 0.00682 |
| PROC.127894204.T/C | CT | 2/20.5/28 | 21/27.5/28 | 3.51 | 1.96 | 0.064 |
| PROC.127894608.G/A | AG | 2/21/28 | 26/28/28 | 5.93 | 1.97 | 0.0167 |
| PROC.127894645.C/T | CT | 2/22.5/28 | 26/28/28 | 3.3 | 1.99 | 0.0722 |
| PROC.127895556.G/A | A | 4/21.5/28 | 6/28/28 | 4.57 | 1.107 | 0.0348 |
| PROC.127895556.G/A | AA | 8/23/28 | 28/28/28 | 6.11 | 1.15 | 0.0259 |
| PROC.127895783.G/A | AG | 5/23/28 | 26.5/28/28 | 3.41 | 1.90 | 0.0682 |
| PROC.127895876.T/C | CT | 2/20.5/28 | 26/28/28 | 4.7 | 1.99 | 0.0325 |
| PROC.127899224.C/T | CT | 2/22/28 | 26/28/28 | 3.77 | 1.99 | 0.0551 |
| PROC.127901799.C/T | CT | 2/22/28 | 26/28/28 | 3.56 | 1.99 | 0.0622 |
| PROC.127975205.T/C | C | 4/21/28 | 26/28/28 | 8.76 | 1.152 | 0.00358 |
| PROCR.33183348.T/C | C | 2/21.5/28 | 28/28/28 | 6.3 | 1.56 | 0.015 |
| PROCR.33183694.C/A | A | 2.25/21.5/28 | 28/28/28 | 6.03 | 1.52 | 0.0174 |
| PROCR.33186524.A/G | G | 2/21/28 | 28/28/28 | 4.52 | 1.40 | 0.0398 |
| PROCR.33228215.A/G | G | 2/21/28 | 28/28/28 | 7.58 | 1.51 | 0.00817 |
| PROCR.33228215.A/G | AG | 1/16/28 | 28/28/28 | 7.68 | 1.43 | 0.0082 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

Significant improvements in days alive and free of neurological dysfunction were noted when comparing XIGRIS™-treated patients and the matched controls for 11 of the IRPs (TABLE 17).

TABLE 17
Days alive and free of neurological dysfunction by several polymorphisms
in the coagulation, fibrinolysis and inflammation pathways in a cohort of
critically ill patients who had severe sepsis and no XIGRIS ™
contraindications.

| | | Days Alive and Free of Neurological | | | | |
|---|---|---|---|---|---|---|
| | | Dysfunction | | | | |
| | | | XIGRIS ™-Treated | | | |
| SNP | IRP | Matched Controls | Patients | F | D.F. | P |
| FGB.155840914.G/A | A | 4.5/18/25 | 26/27/27 | 4.68 | 1.61 | 0.0345 |
| F2R.76059983.A/G | G | 3/15/26 | 8.5/25/26.25 | 3.56 | 1.212 | 0.0606 |
| IL10.203334802.C/A | A | 2/15/26.5 | 25/26/28 | 4.57 | 1.62 | 0.0365 |
| IL12A.161198944.G/A | A | 2.25/18/25.75 | 24/25/27 | 3.18 | 1.35 | 0.0832 |
| IL12A.161198944.G/A | AG | 2.25/18/25.75 | 24/25/27 | 3.18 | 1.35 | 0.0832 |
| PROC.127894608.G/A | AG | 2.5/15/26.5 | 23.75/25/27 | 4.11 | 1.97 | 0.0454 |
| PROC.127894645.C/T | CT | 2.75/19/26.25 | 23/25/27 | 3.16 | 1.99 | 0.0785 |
| PROC.127895876.T/C | CT | 2/15/26.25 | 23/25/27 | 3.86 | 1.99 | 0.0522 |
| PROC.127899224.C/T | CT | 2/19/26 | 23/25/27 | 3.73 | 1.99 | 0.0564 |
| PROC.127901799.C/T | CT | 2/20/26.25 | 23/25/27 | 3.49 | 1.99 | 0.0646 |
| PROCR.33228215.A/G | AG | 2/14/25 | 23.5/25/27 | 3.49 | 1.43 | 0.0684 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

Significant improvements in days alive and free of ¾ SIRS criteria were noted when comparing XIGRIS™-treated patients and the matched controls for 3 of the IRPs (TABLE 18).

TABLE 18
Days alive and free of ¾ SIRS criteria by several polymorphisms
in the coagulation, fibrinolysis and inflammation pathways in a cohort of
critically ill patients who had severe sepsis and no XIGRIS ™
contraindications.

| | | Days Alive and Free of | | | | |
|---|---|---|---|---|---|---|
| | | ¾SIRS Criteria | | | | |
| | | | XIGRIS ™-Treated | | | |
| SNP | IRP | Matched Controls | Patients | F | D.F. | P |
| F3.94719939.A/G | GG | 0/5.5/19.75 | 7/22/23 | 2.92 | 1.50 | 0.0935 |
| IL6.22541812.C/G | C | 0.75/9/24.25 | 23.5/26/26 | 3.05 | 1.54 | 0.0862 |
| IL10.203334802.C/A | A | 1/5/11.5 | 16/16/22 | 4.67 | 1.62 | 0.0346 |

More days alive and free of organ dysfunction indicates improved organ function. Statistical analysis was conducted using a Kruskal-Wallis test (F).
Data is presented as 25^{th} percentile/median/75^{th} percentile.
IRP, improved response polymorphism.
D.F., degrees of freedom.

### Organ Dysfunctions of IRP Patients Compared to Those of Non-IRP Patients

Organ dysfunctions were also compared between IRP patients and patients having alleles/genotypes other than the IRP (TABLEs 20-33; sample sizes in TABLE 19) for all IRP SNPs. Results are reported as the difference in median days alive and free of a given organ dysfunction between both (1) IRP patients and non-IRP patients in the matched-control group and (2) IRP XIGRIS™-treated patients and non-IRP XIGRIS™-treated patients. In virtually every case the average difference in days alive and free of different organ dysfunctions in XIGRIS™-treated patients is greater than the difference in matched controls. Furthermore, the IRP patients have fewer days alive and free than the non-TRP patients when they are not treated with XIGRIS™. In contrast, the IRP patients have more days alive and free than the non-IRP patients when they are treated with XIGRIS™. This confirms that the IRP genotype identifies patients who respond particularly well to XIGRIS™.

TABLE 19
Improved response polymorphism (IRP) description and sample
size (N) for TABLES 20 to 32.

| | | | Matched | | XIGRIS ™-Treated | |
|---|---|---|---|---|---|---|
| | | | Controls | | Patients | |
| SNP | IRP | non-IRP | N IRP | N non-IRP | N IRP | N non-IRP |
| FGB.155840914.G/A | A | G | 55 | 319 | 8 | 48 |
| F2.46717332.G/A | G | A | 231 | 197 | 42 | 46 |
| F2.46717332.G/A | GG | AG/AA | 67 | 147 | 8 | 36 |
| F2R.76059983.A/G | G | A | 182 | 258 | 32 | 54 |
| F2R.76059983.A/G | GG | AG/AA | 39 | 181 | 7 | 36 |
| F2R.76049220.G/C | GG | GC/CC | 128 | 89 | 30 | 13 |
| F3.94719939.A/G | GG | AG/AA | 42 | 173 | 10 | 32 |
| F5.166258759.A/G | G | A | 34 | 314 | 9 | 45 |
| F5.166236816.T/C | T | C | 207 | 139 | 30 | 24 |
| F5.166227911.A/G | A | G | 157 | 183 | 24 | 30 |
| F5.166269905.G/A | A | G | 107 | 241 | 21 | 33 |
| F7.112808416.A/G | AG | AA/GG | 81 | 92 | 17 | 10 |
| F10.112840894.A/C | C | A | 91 | 255 | 13 | 41 |
| F10.112825510.A/G | G | A | 81 | 267 | 17 | 37 |
| F10.112824083.T/C | T | C | 119 | 227 | 21 | 31 |
| SERPINE1.100363146.4G/5G | I | D | 169 | 169 | 25 | 29 |
| SERPINE1.100375050.G/A | A | G | 65 | 283 | 8 | 46 |
| SERPINE1.100375050.G/A | AG | AA/GG | 45 | 129 | 8 | 19 |
| SERPINA5.94123294.C/T | TT | CT/CC | 56 | 155 | 8 | 34 |
| IL6.22541812.C/G | C | G | 52 | 20 | 4 | 2 |
| IL6.22539885.G/C | G | C | 93 | 37 | 5 | 3 |
| IL10.203334802.C/A | A | C | 59 | 139 | 5 | 11 |
| IL12A.161198944.G/A | A | G | 30 | 378 | 7 | 59 |
| IL12A.161198944.G/A | AG | AA/GG | 30 | 174 | 7 | 26 |
| TNFRSF1A.6317783.T/C | CT | CC/TT | 88 | 77 | 15 | 12 |
| VEGF.43848656.G/A | AA | AG/GG | 38 | 129 | 4 | 22 |
| PROC.127890298.A/G | AG | AA/GG | 74 | 139 | 15 | 26 |
| PROC.127890457.T/C | CT | CC/TT | 78 | 139 | 16 | 26 |
| PROC.127892009.G/A | AG | AA/GG | 75 | 140 | 16 | 28 |
| PROC.127892092.C/T | CT | CC/TT | 90 | 144 | 16 | 30 |
| PROC.127894204.T/C | C | T | 214 | 206 | 46 | 42 |
| PROC.127894204.T/C | CT | CC/TT | 82 | 128 | 16 | 28 |
| PROC.127894608.G/A | AG | AA/GG | 83 | 129 | 16 | 26 |
| PROC.127894645.C/T | CT | CC/TT | 84 | 132 | 17 | 27 |
| PROC.127895556.G/A | A | G | 88 | 346 | 21 | 69 |
| PROC.127895556.G/A | AA | AG/GG | 13 | 204 | 4 | 41 |
| PROC.127895783.G/A | AG | AA/GG | 77 | 138 | 15 | 27 |
| PROC.127895876.T/C | CT | CC/TT | 84 | 129 | 17 | 27 |
| PROC.127899224.C/T | CT | CC/TT | 84 | 127 | 17 | 28 |
| PROC.127901000.T/C | CT | CC/TT | 79 | 137 | 11 | 31 |
| PROC.127901799.C/T | CT | CC/TT | 84 | 130 | 17 | 26 |
| PROC.127975205.T/C | C | T | 133 | 283 | 21 | 63 |
| PROCR.33183348.T/C | C | T | 50 | 390 | 8 | 80 |
| PROCR.33183694.C/A | A | C | 46 | 374 | 8 | 78 |
| PROCR.33186524.A/G | G | A | 35 | 401 | 7 | 81 |
| PROCR.33228215.A/G | AG | AA/GG | 37 | 193 | 8 | 39 |
| PROCR.33228215.A/G | G | A | 43 | 417 | 10 | 84 |

When the IRP is an allele, N represents the number of alleles genotyped.
When the IRP is a genotype, N represents the number of individuals genotyped.

For cardiovascular dysfunction (TABLE 20), on average matched-control patients having the IRP allele/genotype do worse than patients having alleles/genotypes other than the IRP (−1.3 days alive and free of cardiovascular dysfunction). In contrast, on average, XIGRIS™-treated patients having the IRP allele/genotype do better than patients having alleles/genotypes other than the IRP (+8.7 days alive and free of cardiovascular dysfunction). Clearly, the IRP patients benefit the most from XIGRIS™ treatment in terms of improvements of days alive and free of cardiovascular dysfunction.

TABLE 20
Difference in median days alive and free of cardiovascular dysfunction
between improved response polymorphism (IRP) and non-IRP patients
by treatment (control or XIGRIS ™).

| | | Matched Controls | | | XIGRIS ™-Treated Patients | | |
|---|---|---|---|---|---|---|---|
| | | Median | Median | | Median | Median | |
| SNP | IRP | IRP | IRP | DIFFERENCE | non-IRP | non-IRP | DIFFERENCE |
| FGB.155840914.G/A | A | 16 | 14 | 2 | 22.5 | 15 | 7.5 |
| F2.46717332.G/A | G | 9 | 17 | −8 | 15 | 14 | 1 |
| F2.46717332.G/A | GG | 3 | 17 | −14 | 14.5 | 15.5 | −1 |
| F3.94719939.A/G | GG | 8.5 | 14 | −5.5 | 24.5 | 14 | 10.5 |
| F2R.76059983.A/G | G | 14 | 14 | 0 | 16 | 14 | 2 |
| F2R.76059983.A/G | GG | 5 | 16 | −11 | 14 | 17.5 | −3.5 |
| F2R.76049220.G/C | GG | 9 | 18 | −9 | 15 | 22 | 7 |
| F5.166258759.A/G | G | 12.5 | 14 | −1.5 | 27 | 15 | 12 |
| F5.166236816.T/C | T | 15 | 13 | 2 | 22 | 9.5 | 12.5 |
| F5.166227911.A/G | A | 16 | 9 | 7 | 22 | 13 | 9 |
| F5.166269905.G/A | A | 15 | 14 | 1 | 22 | 15 | 7 |
| F7.112808416.A/G | AG | 7 | 17.5 | −10.5 | 22 | 15.5 | 6.5 |
| F10.112840894.A/C | C | 9 | 14 | −5 | 25 | 15 | 10 |
| F10.112825510.A/G | G | 8 | 16 | −8 | 24 | 15 | 9 |
| F10.112824083.T/C | T | 13 | 15 | −2 | 24 | 15 | 9 |
| SERPINE1.100363146.4G/5G | I | 9 | 16 | −7 | 22 | 15 | 7 |
| SERPINE1.100375050.G/A | A | 16 | 14 | 2 | 25.5 | 15 | 10.5 |
| SERPINE1.100375050.G/A | AG | 16 | 14 | 2 | 25.5 | 11 | 14.5 |
| SERPINA5.94123294.C/T | TT | 15.5 | 12 | 3.5 | 18.5 | 14 | 4.5 |
| IL6.22541812.C/G | C | 18 | 19.5 | −1.5 | 26.5 | 27.5 | −1 |
| IL6.22539885.G/C | G | 9 | 13 | −4 | 27 | 26 | 1 |
| IL10.203334802.C/A | A | 7 | 9 | −2 | 27 | 26 | 1 |
| IL12A.161198944.G/A | A | 15 | 14 | 1 | 22 | 15 | 7 |
| IL12A.161198944.G/A | GA | 15 | 14 | 1 | 22 | 13 | 9 |
| TNFRSF1A.6317783.T/C | CT | 9 | 19 | −10 | 22 | 7.5 | 14.5 |
| VEGF.43848656.G/A | AA | 13 | 16 | −3 | 24.5 | 15 | 9.5 |
| PROC.127890298.A/G | AG | 18 | 9 | 9 | 25 | 8.5 | 16.5 |
| PROC.127890457.T/C | CT | 18.5 | 9 | 9.5 | 25 | 8.5 | 16.5 |
| PROC.127892009.G/A | AG | 18 | 9 | 9 | 25 | 8.5 | 16.5 |
| PROC.127892092.C/T | CT | 14.5 | 11 | 3.5 | 19 | 14.5 | 4.5 |
| PROC.127894204.T/C | C | 14 | 13 | 1 | 23 | 8.5 | 14.5 |
| PROC.127894204.T/C | CT | 9 | 14.5 | −5.5 | 19 | 12.5 | 6.5 |
| PROC.127894608.G/A | AG | 14 | 14 | 0 | 24.5 | 8.5 | 16 |
| PROC.127894645.C/T | CT | 14 | 12 | 2 | 24 | 9 | 15 |
| PROC.127895556.G/A | A | 9 | 15 | −6 | 15 | 15 | 0 |
| PROC.127895556.G/A | AA | 9 | 14.5 | −5.5 | 24.5 | 15 | 9.5 |
| PROC.127895783.G/A | AG | 18 | 9 | 9 | 25 | 8 | 17 |
| PROC.127895876.T/C | CT | 10.5 | 14 | −3.5 | 24 | 9 | 15 |
| PROC.127899224.C/T | CT | 13 | 14 | −1 | 24 | 10 | 14 |
| PROC.127901000.T/C | CT | 18 | 9 | 9 | 25 | 9 | 16 |
| PROC.127901799.C/T | CT | 14.5 | 13.5 | 1 | 24 | 8.5 | 15.5 |
| PROC.127975205.T/C | C | 14 | 14 | 0 | 24 | 14 | 10 |
| PROCR.33183348.T/C | C | 11.5 | 14 | −2.5 | 23 | 15 | 8 |
| PROCR.33183694.C/A | A | 11.5 | 14 | −2.5 | 23 | 14 | 9 |
| PROCR.33186524.A/G | G | 15 | 14 | 1 | 22 | 15 | 7 |
| PROCR.33228215.A/G | AG | 6 | 14 | −8 | 25.5 | 14 | 11.5 |
| PROCR.33228215.A/G | G | 12 | 14 | −2 | 24.5 | 15 | 9.5 |
| AVERAGE | | | | −1.3 | | | 8.7 |
| DIFFERENCE | | | | | | | |

Data is shown for several polymorphisms in the coagulation, fibrinolysis and inflammation pathways in a cohort of critically ill patients who had severe sepsis and no XIGRIS ™ contraindications.
DIFFERENCE = median days alive and free of cardiovascular dysfunction of patients having the IRP minus median days alive and free of cardiovascular dysfunction of patients having the non-IRP allele/genotype, within (1) Matched Controls and (2) XIGRIS ™-Treated Patients.

For days alive a free of use of vasopressors (TABLE 21), on average matched-control patients having the IRP allele/genotype do worse than patients having alleles/genotypes other than the IRP (−1.3 days alive and free of use of vasopressors). In contrast, on average, XIGRIS™-treated patients having the IRP allele/genotype do better than patients having alleles/genotypes other than the IRP (+6.5 days alive and free of use of vasopressors). Clearly, the IRP patients benefit the most from XIGRIS™ treatment in terms of improvements of days alive and free of use of vasopressors.

TABLE 21
Difference in median days alive and free of use of vasopressors between
improved response polymorphism (IRP) and non-IRP patients by treatment
(control or XIGRIS ™).

| | | Matched Controls | | | XIGRIS ™-Treated Patients | | |
|---|---|---|---|---|---|---|---|
| | | Median | Median | | Median | Median | |
| SNP | IRP | IRP | non-IRP | DIFFERENCE | IRP | non-IRP | DIFFERENCE |
| FGB.155840914.G/A | A | 18 | 20 | −2 | 24.5 | 21 | 3.5 |
| F2.46717332.G/A | G | 14 | 21 | −7 | 22.5 | 23 | −0.5 |
| F2.46717332.G/A | GG | 7 | 20 | −13 | 22 | 23 | −1 |
| F2R.76059983.A/G | G | 18 | 19 | −1 | 24 | 17 | 7 |
| F2R.76059983.A/G | GG | 11 | 20 | −9 | 23 | 22.5 | 0.5 |
| F2R.76049220.G/C | GG | 17.5 | 20 | −2.5 | 22.5 | 25 | −2.5 |
| F3.94719939.A/G | GG | 16.5 | 19 | −2.5 | 24.5 | 17 | 7.5 |
| F5.166258759.A/G | G | 18.5 | 18 | 0.5 | 27 | 21 | 6 |
| F5.166236816.T/C | T | 19 | 18 | 1 | 25 | 14 | 11 |
| F5.166227911.A/G | A | 20 | 12 | 8 | 25.5 | 19 | 6.5 |
| F5.166269905.G/A | A | 19 | 18 | 1 | 24 | 21 | 3 |
| F7.112808416.A/G | AG | 10 | 22 | −12 | 25 | 20.5 | 4.5 |
| F10.112840894.A/C | C | 15 | 18 | −3 | 25 | 17 | 8 |
| F10.112825510.A/G | G | 13 | 20 | −7 | 25 | 21 | 4 |
| F10.112824083.T/C | T | 18 | 19 | −1 | 25 | 21 | 4 |
| SERPINE1.100363146.4G/5G | I | 13 | 19 | −6 | 24 | 21 | 3 |
| SERPINE1.100375050.G/A | A | 20 | 18 | 2 | 26 | 19 | 7 |
| SERPINE1.100375050.G/A | AG | 20 | 18 | 2 | 26 | 17 | 9 |
| SERPINA5.94123294.C/T | TT | 18 | 18 | 0 | 24 | 21.5 | 2.5 |
| IL6.22541812.C/G | C | 20.5 | 20.5 | 0 | 27.5 | 28 | −0.5 |
| IL6.22539885.G/C | G | 17 | 18 | −1 | 28 | 27 | 1 |
| IL10.203334802.C/A | A | 12 | 13 | −1 | 28 | 26 | 2 |
| IL12A.161198944.G/A | A | 18 | 19 | −1 | 25 | 21 | 4 |
| IL12A.161198944.G/A | GA | 18 | 19 | −1 | 25 | 17 | 8 |
| TNFRSF1A.6317783.T/C | CT | 13.5 | 22 | −8.5 | 24 | 10.5 | 13.5 |
| VEGF.43848656.G/A | AA | 17 | 20 | −3 | 26 | 19 | 7 |
| PROC.127890298.A/G | AG | 21 | 17 | 4 | 26 | 13 | 13 |
| PROC.127890457.T/C | CT | 21.5 | 17 | 4.5 | 26 | 10.5 | 15.5 |
| PROC.127892009.G/A | AG | 21 | 16.5 | 4.5 | 26 | 13 | 13 |
| PROC.127892092.C/T | CT | 19 | 17.5 | 1.5 | 24.5 | 20 | 4.5 |
| PROC.127894204.T/C | C | 18 | 19 | −1 | 24 | 11 | 13 |
| PROC.127894204.T/C | CT | 17.5 | 19 | −1.5 | 24.5 | 17 | 7.5 |
| PROC.127894608.G/A | AG | 18 | 19 | −1 | 25 | 10.5 | 14.5 |
| PROC.127894645.C/T | CT | 18 | 18.5 | −0.5 | 25 | 11 | 14 |
| PROC.127895556.G/A | A | 17 | 19 | −2 | 24 | 23 | 1 |
| PROC.127895556.G/A | AA | 17 | 19 | −2 | 25 | 22 | 3 |
| PROC.127895783.G/A | AG | 21 | 16.5 | 4.5 | 26 | 11 | 15 |
| PROC.127895876.T/C | CT | 18 | 19 | −1 | 25 | 11 | 14 |
| PROC.127899224.C/T | CT | 18 | 19 | −1 | 25 | 14 | 11 |
| PROC.127901000.T/C | CT | 21 | 16 | 5 | 25 | 15 | 10 |
| PROC.127901799.C/T | CT | 19 | 18 | 1 | 25 | 10.5 | 14.5 |
| PROC.127975205.T/C | C | 18 | 19 | −1 | 25 | 21 | 4 |
| PROCR.33183348.T/C | C | 18 | 19 | −1 | 26 | 23 | 3 |
| PROCR.33183694.C/A | A | 18 | 19 | −1 | 26 | 21.5 | 4.5 |
| PROCR.33186524.A/G | G | 18 | 19 | −1 | 26 | 22 | 4 |
| PROCR.33228215.A/G | AG | 15 | 19 | −4 | 26.5 | 22 | 4.5 |
| PROCR.33228215.A/G | G | 19 | 18 | 1 | 26 | 22.5 | 3.5 |
| AVERAGE | | | | −1.3 | | | 6.5 |
| DIFFERENCE | | | | | | | |

Data is shown for several polymorphisms in the coagulation, fibrinolysis and inflammation pathways in a cohort of critically ill patients who had severe sepsis and no XIGRIS ™ contraindications.
DIFFERENCE = median days alive and free of use of vasopressors of patients having the IRP minus median days alive and free of use of vasopressors of patients having the non-IRP allele/genotype, within (1) Matched Controls and (2) XIGRIS ™-Treated Patients.

For days alive a free of inotropic agents (TABLE 22), on average matched-control patients having the IRP allele/genotype do worse than patients having alleles/genotypes other than the IRP (−1.8 days alive and free of use of inotropic agents). In contrast, on average, XIGRIS™-treated patients having the IRP allele/genotype do better than patients having alleles/genotypes other than the IRP (+5.3 days alive and free of use of inotropic agents). Clearly, the IRP patients benefit the most from XIGRIS™ treatment in terms of improvements of days alive and free of use of inotropic agents.

TABLE 22
Difference in median days alive and free of inotropic agents between
improved response polymorphism (IRP) and non-IRP patients by
treatment (control or XIGRIS ™).

| | | Matched Controls | | | XIGRIS ™-Treated Patients | | |
|---|---|---|---|---|---|---|---|
| | | Median | Median | | Median | Median | |
| SNP | IRP | IRP | non-IRP | DIFFERENCE | IRP | non-IRP | DIFFERENCE |
| FGB.155840914.G/A | A | 24 | 26 | −2 | 28 | 25 | 3 |
| F2.46717332.G/A | G | 21 | 27 | −6 | 27 | 27 | 0 |
| F2.46717332.G/A | GG | 13 | 27 | −14 | 27 | 27 | 0 |
| F2R.76059983.A/G | G | 24 | 26 | −2 | 28 | 27 | 1 |
| F2R.76059983.A/G | GG | 17 | 26 | −9 | 28 | 27 | 1 |
| F2R.76049220.G/C | GG | 23 | 28 | −5 | 28 | 23 | 5 |
| F3.94719939.A/G | GG | 23.5 | 26 | −2.5 | 27 | 26.5 | 0.5 |
| F5.166258759.A/G | G | 26 | 25 | 1 | 28 | 25 | 3 |
| F5.166236816.T/C | T | 24 | 26 | −2 | 28 | 16.5 | 11.5 |
| F5.166227911.A/G | A | 26 | 23 | 3 | 28 | 24 | 4 |
| F5.166269905.G/A | A | 26 | 25 | 1 | 28 | 23 | 5 |
| F7.112808416.A/G | AG | 15 | 27.5 | −12.5 | 26 | 26.5 | −0.5 |
| F10.112840894.A/C | C | 22 | 26 | −4 | 28 | 25 | 3 |
| F10.112825510.A/G | G | 21 | 26 | −5 | 28 | 25 | 3 |
| F10.112824083.T/C | T | 23 | 26 | −3 | 28 | 25 | 3 |
| SERPINE1.100363146.4G/5G | I | 21 | 27 | −6 | 26 | 25 | 1 |
| SERPINE1.100375050.G/A | A | 22 | 25 | −3 | 28 | 25 | 3 |
| SERPINE1.100375050.G/A | AG | 26 | 25 | 1 | 28 | 23 | 5 |
| SERPINA5.94123294.C/T | TT | 25 | 26 | −1 | 28 | 25 | 3 |
| IL6.22541812.C/G | C | 28 | 28 | 0 | 27 | 28 | −1 |
| IL6.22539885.G/C | G | 22 | 22 | 0 | 28 | 26 | 2 |
| IL10.203334802.C/A | A | 16 | 19 | −3 | 28 | 26 | 2 |
| IL12A.161198944.G/A | A | 22 | 26 | −4 | 28 | 25 | 3 |
| IL12A.161198944.G/A | GA | 22 | 26 | −4 | 28 | 24 | 4 |
| TNFRSF1A.6317783.T/C | CT | 22 | 28 | −6 | 28 | 14 | 14 |
| VEGF.43848656.G/A | AA | 24.5 | 26 | −1.5 | 28 | 25 | 3 |
| PROC.127890298.A/G | AG | 27 | 23 | 4 | 28 | 18.5 | 9.5 |
| PROC.127890457.T/C | CT | 27 | 23 | 4 | 28 | 13 | 15 |
| PROC.127892009.G/A | AG | 26 | 23 | 3 | 28 | 18.5 | 9.5 |
| PROC.127892092.C/T | CT | 25 | 26 | −1 | 28 | 20.5 | 7.5 |
| PROC.127894204.T/C | C | 26 | 23 | 3 | 28 | 14 | 14 |
| PROC.127894204.T/C | CT | 24.5 | 26 | −1.5 | 28 | 24 | 4 |
| PROC.127894608.G/A | AG | 26 | 26 | 0 | 28 | 12 | 16 |
| PROC.127894645.C/T | CT | 25 | 26 | −1 | 28 | 14 | 14 |
| PROC.127895556.G/A | A | 26 | 25.5 | 0.5 | 28 | 26 | 2 |
| PROC.127895556.G/A | AA | 26 | 26 | 0 | 28 | 26 | 2 |
| PROC.127895783.G/A | AG | 26 | 23 | 3 | 28 | 14 | 14 |
| PROC.127895876.T/C | CT | 23.5 | 26 | −2.5 | 28 | 14 | 14 |
| PROC.127899224.C/T | CT | 25 | 26 | −1 | 28 | 16 | 12 |
| PROC.127901000.T/C | CT | 26 | 23 | 3 | 28 | 23 | 5 |
| PROC.127901799.C/T | CT | 25 | 26 | −1 | 28 | 12 | 16 |
| PROC.127975205.T/C | C | 26 | 25 | 1 | 28 | 26 | 2 |
| PROCR.33183348.T/C | C | 25.5 | 25.5 | 0 | 28 | 26 | 2 |
| PROCR.33183694.C/A | A | 25.5 | 26 | −0.5 | 28 | 26 | 2 |
| PROCR.33186524.A/G | G | 26 | 25 | 1 | 28 | 26 | 2 |
| PROCR.33228215.A/G | AG | 19 | 26 | −7 | 28 | 26 | 2 |
| PROCR.33228215.A/G | G | 24 | 26 | −2 | 28 | 26 | 2 |
| AVERAGE | | | | −1.8 | | | 5.3 |
| DIFFERENCE | | | | | | | |

Data is shown for several polymorphisms in the coagulation, fibrinolysis and inflammation pathways in a cohort of critically ill patients who had severe sepsis and no XIGRIS ™ contraindications.
DIFFERENCE = median days alive and free of use of inotropic agents of patients having the IRP minus median days alive and free of use of inotropic agents of patients having the non-IRP allele/genotype, within (1) Matched Controls and (2) XIGRIS ™-Treated Patients.

For days alive a free of acute lung injury (TABLE 23), on average matched-control patients having the IRP allele/genotype do the same as patients having alleles/genotypes other than the IRP (0.2 days alive and free of use of acute lung injury). In contrast, on average, XIGRIS™-treated patients having the IRP allele/genotype do better than patients having alleles/genotypes other than the IRP (+4.2 days alive and free of use of acute lung injury). Clearly, the IRP patients benefit the most from XIGRIS™ treatment in terms of improvements of days alive and free of use of acute lung injury.

TABLE 23
Difference in median days alive and free of acute lung injury between improved
response polymorphism (IRP) and non-IRP patients by treatment (control or
XIGRIS ™). Data is shown for several polymorphisms in the coagulation,
fibrinolysis and inflammation pathways in a cohort of critically ill patients who
had severe sepsis and no XIGRIS ™ contraindications.
DIFFERENCE = median days alive and free of acute lung injury of
patients having the IRP minus median days alive and free of acute lung injury
patients having the non-IRP allele/genotype., within (1) Matched
Controls and (2) XIGRIS ™-Treated Patients.

| | | Matched Controls | | | XIGRIS ™-Treated Patients | | |
|---|---|---|---|---|---|---|---|
| | | Median | Median | | Median | Median | |
| SNP | IRP | IRP | non-IRP | DIFFERENCE | IRP | non-IRP | DIFFERENCE |
| FGB.155840914.G/A | A | 17 | 11 | 6 | 11 | 8.5 | 2.5 |
| F2.46717332.G/A | G | 8 | 17 | −9 | 5.5 | 14 | −8.5 |
| F2.46717332.G/A | GG | 6 | 16 | −10 | 6 | 13 | −7 |
| F2R.76059983.A/G | G | 11 | 10 | 1 | 17 | 5 | 12 |
| F2R.76059983.A/G | GG | 8 | 12 | −4 | 17 | 8.5 | 8.5 |
| F2R.76049220.G/C | GG | 11 | 11 | 0 | 10.5 | 4 | 6.5 |
| F3.94719939.A/G | GG | 4.5 | 11 | −6.5 | 21.5 | 5.5 | 16 |
| F5.166258759.A/G | G | 16 | 11 | 5 | 22 | 9 | 13 |
| F5.166236816.T/C | T | 9 | 15 | −6 | 15 | 6.5 | 8.5 |
| F5.166227911.A/G | A | 12 | 11 | 1 | 14 | 8 | 6 |
| F5.166269905.G/A | A | 15 | 9 | 6 | 16 | 5 | 11 |
| F7.112808416.A/G | AG | 6 | 16.5 | −10.5 | 6 | 11.5 | −5.5 |
| F10.112840894.A/C | C | 11 | 11 | 0 | 22 | 8 | 14 |
| F10.112825510.A/G | G | 5 | 14 | −9 | 18 | 8 | 10 |
| F10.112824083.T/C | T | 12 | 11 | 1 | 14 | 9 | 5 |
| SERPINE1.100363146.4G/5G | I | 9 | 12 | −3 | 8 | 14 | −6 |
| SERPINE1.100375050.G/A | A | 11 | 12 | −1 | 18 | 8.5 | 9.5 |
| SERPINE1.100375050.G/A | AG | 17 | 11 | 6 | 18 | 8 | 10 |
| SERPINA5.94123294.C/T | TT | 10.5 | 9 | 1.5 | 5 | 11 | −6 |
| IL6.22541812.C/G | C | 22 | 22 | 0 | 16 | 15.5 | 0.5 |
| IL6.22539885.G/C | G | 11 | 15 | −4 | 9 | 16 | −7 |
| IL10.203334802.C/A | A | 7 | 15 | −8 | 3 | 16 | −13 |
| IL12A.161198944.G/A | A | 17 | 12 | 5 | 17 | 9 | 8 |
| IL12A.161198944.G/A | AG | 17 | 11 | 6 | 17 | 8.5 | 8.5 |
| TNFRSF1A.6317783.T/C | CT | 9.5 | 15 | −5.5 | 17 | 3.5 | 13.5 |
| VEGF.43848656.G/A | AA | 9 | 14 | −5 | 24 | 8.5 | 15.5 |
| PROC.127890298.A/G | AG | 15 | 8 | 7 | 17 | 5.5 | 11.5 |
| PROC.127890457.T/C | CT | 15.5 | 7 | 8.5 | 19.5 | 5.5 | 14 |
| PROC.127892009.G/A | AG | 15 | 7.5 | 7.5 | 19.5 | 5.5 | 14 |
| PROC.127892092.C/T | CT | 10.5 | 10.5 | 0 | 10.5 | 7 | 3.5 |
| PROC.127894204.T/C | C | 13.5 | 8 | 5.5 | 9 | 8 | 1 |
| PROC.127894204.T/C | CT | 13.5 | 8.5 | 5 | 10 | 8.5 | 1.5 |
| PROC.127894608.G/A | AG | 13 | 9 | 4 | 16.5 | 5.5 | 11 |
| PROC.127894645.C/T | CT | 9.5 | 10 | −0.5 | 16 | 6 | 10 |
| PROC.127895556.G/A | A | 11.5 | 9 | 2.5 | 9 | 12 | −3 |
| PROC.127895556.G/A | AA | 9 | 10.5 | −1.5 | 13.5 | 8 | 5.5 |
| PROC.127895783.G/A | AG | 15 | 7.5 | 7.5 | 17 | 6 | 11 |
| PROC.127895876.T/C | CT | 11 | 9 | 2 | 16 | 6 | 10 |
| PROC.127899224.C/T | CT | 10 | 11 | −1 | 16 | 7 | 9 |
| PROC.127901000.T/C | CT | 15 | 8 | 7 | 17 | 6 | 11 |
| PROC.127901799.C/T | CT | 9.5 | 11 | −1.5 | 17 | 5.5 | 11.5 |
| PROC.127975205.T/C | C | 11 | 9 | 2 | 9 | 12 | −3 |
| PROCR.33183348.T/C | C | 8 | 10 | −2 | 2 | 14 | −12 |
| PROCR.33183694.C/A | A | 11.5 | 10.5 | 1 | 2 | 10.5 | −8.5 |
| PROCR.33186524.A/G | G | 15 | 10 | 5 | 2 | 12 | −10 |
| PROCR.33228215.A/G | AG | 5 | 12 | −7 | 4.5 | 12 | −7.5 |
| PROCR.33228215.A/G | G | 14 | 11 | 3 | 2.5 | 12 | −9.5 |
| AVERAGE | | | | 0.2 | | | 4.2 |
| DIFFERENCE | | | | | | | |

For respiratory dysfunction (TABLE 24), on average matched-control patients having the IRP allele/genotype do worse than patients having alleles/genotypes other than the IRP (−0.2 days alive and free of respiratory dysfunction). In contrast, on average, XIGRIS™-treated patients having the IRP allele/genotype do better than patients having alleles/genotypes other than the IRP (+8.4 days alive and free of respiratory dysfunction). Clearly, the IRP patients benefit the most from XIGRIS™ treatment in terms of improvements of days alive and free of respiratory dysfunction.

TABLE 24
Difference in median days alive and free of respiratory dysfunction between
improved response polymorphism (IRP) and non-IRP patients by treatment
(control or XIGRIS ™). Data is shown for several polymorphisms in
the coagulation, fibrinolysis and inflammation pathways in a cohort of
critically ill patients who had severe sepsis and no XIGRIS ™
contraindications. DIFFERENCE = median days alive and free of
respiratory dysfunction of patients having the IRP minus median days alive
and free of respiratory dysfunction of patients having the non-IRP
allele/genotype, within (1) Matched Controls and (2) XIGRIS ™-
Treated Patients.

| | | Matched Controls | | | XIGRIS ™-Treated Patients | | |
|---|---|---|---|---|---|---|---|
| | | Median | Median | | Median | Median | |
| SNP | IRP | IRP | non-IRP | DIFFERENCE | IRP | non-IRP | DIFFERENCE |
| FGB.155840914.G/A | A | 9 | 5 | 4 | 19.5 | 9 | 10.5 |
| F2.46717332.G/A | G | 2 | 8 | −6 | 6 | 14.5 | −8.5 |
| F2.46717332.G/A | GG | 1 | 8 | −7 | 5.5 | 14.5 | −9 |
| F2R.76059983.A/G | G | 3 | 7 | −4 | 19 | 4 | 15 |
| F2R.76059983.A/G | GG | 0 | 8 | −8 | 19 | 5.5 | 13.5 |
| F2R.76049220.G/C | GG | 5.5 | 4 | 1.5 | 15.5 | 4 | 11.5 |
| F3.94719939.A/G | GG | 2.5 | 6 | −3.5 | 22.5 | 4 | 18.5 |
| F5.166258759.A/G | G | 6.5 | 4.5 | 2 | 20 | 17 | 3 |
| F5.166236816.T/C | T | 3 | 6 | −3 | 20 | 7 | 13 |
| F5.166227911.A/G | A | 7 | 4 | 3 | 19.5 | 13 | 6.5 |
| F5.166269905.G/A | A | 9 | 3 | 6 | 20 | 9 | 11 |
| F7.112808416.A/G | AG | 2 | 9 | −7 | 17 | 14 | 3 |
| F10.112840894.A/C | C | 2 | 5 | −3 | 21 | 5 | 16 |
| F10.112825510.A/G | G | 1 | 7 | −6 | 20 | 9 | 11 |
| F10.112824083.T/C | T | 3 | 5 | −2 | 19 | 17 | 2 |
| SERPINE1.100363146.4G/5G | I | 2 | 5 | −3 | 17 | 17 | 0 |
| SERPINE1.100375050.G/A | A | 8 | 4 | 4 | 21.5 | 9 | 12.5 |
| SERPINE1.100375050.G/A | AG | 8 | 4 | 4 | 21.5 | 5 | 16.5 |
| SERPINA5.94123294.C/T | TT | 6.5 | 3 | 3.5 | 7.5 | 9.5 | −2 |
| IL6.22541812.C/G | C | 8 | 7 | 1 | 24 | 21.5 | 2.5 |
| IL6.22539885.G/C | G | 3 | 7 | −4 | 19 | 24 | −5 |
| IL10.203334802.C/A | A | 1 | 1 | 0 | 23 | 20 | 3 |
| IL12A.161198944.G/A | A | 11.5 | 5 | 6.5 | 19 | 9 | 10 |
| IL12A.161198944.G/A | GA | 11.5 | 4 | 7.5 | 19 | 7 | 12 |
| TNFRSF1A.6317783.T/C | CT | 2 | 10 | −8 | 19 | 9 | 10 |
| VEGF.43848656.G/A | AA | 2 | 8 | −6 | 21 | 13 | 8 |
| PROC.127890298.A/G | AG | 8.5 | 2 | 6.5 | 20 | 4 | 16 |
| PROC.127890457.T/C | CT | 9 | 2 | 7 | 20 | 3 | 17 |
| PROC.127892009.G/A | AG | 7 | 2 | 5 | 20 | 4 | 16 |
| PROC.127892092.C/T | CT | 3 | 4.5 | −1.5 | 15.5 | 4 | 11.5 |
| PROC.127894204.T/C | C | 6 | 3 | 3 | 15 | 4 | 11 |
| PROC.127894204.T/C | CT | 3 | 5.5 | −2.5 | 15.5 | 4.5 | 11 |
| PROC.127894608.G/A | AG | 4 | 4 | 0 | 20 | 4 | 16 |
| PROC.127894645.C/T | CT | 2 | 6 | −4 | 20 | 4 | 16 |
| PROC.127895556.G/A | A | 3.5 | 3 | 0.5 | 9 | 6 | 3 |
| PROC.127895556.G/A | AA | 5 | 3 | 2 | 16 | 5 | 11 |
| PROC.127895783.G/A | AG | 8 | 2 | 6 | 20 | 4 | 16 |
| PROC.127895876.T/C | CT | 3 | 5 | −2 | 20 | 4 | 16 |
| PROC.127899224.C/T | CT | 2 | 6 | −4 | 20 | 4 | 16 |
| PROC.127901000.T/C | CT | 7 | 2 | 5 | 20 | 4 | 16 |
| PROC.127901799.C/T | CT | 2.5 | 5.5 | −3 | 17 | 4 | 13 |
| PROC.127975205.T/C | C | 4 | 3 | 1 | 14 | 4 | 10 |
| PROCR.33183348.T/C | C | 3 | 4 | −1 | 5 | 14 | −9 |
| PROCR.33183694.C/A | A | 3 | 5 | −2 | 5 | 11.5 | −6.5 |
| PROCR.33186524.A/G | G | 3 | 4 | −1 | 6 | 14 | −8 |
| PROCR.33228215.A/G | AG | 3 | 6 | −3 | 19 | 6 | 13 |
| PROCR.33228215.A/G | G | 8 | 4 | 4 | 12.5 | 9 | 3.5 |
| AVERAGE | | | | −0.2 | | | 8.4 |
| DIFFERENCE | | | | | | | |

For days alive and free of use of mechanical ventilators (TABLE 25), on average matched-control patients having the IRP allele/genotype do worse than patients having alleles/genotypes other than the IRP (−0.5 days alive and free of use of mechanical ventilators). In contrast, on average, XIGRIS™-treated patients having the IRP allele/genotype do better than patients having alleles/genotypes other than the IRP (+8.8 days alive and free of use of mechanical ventilators). Clearly, the IRP patients benefit the most from XIGRIS™ treatment in terms of improvements of days alive and free of use of mechanical ventilators.

TABLE 25
Difference in median days alive and free of mechanical ventilator use between
improved response polymorphism (IRP) and non-IRP patients by treatment
(control or XIGRIS ™). Data is shown for several polymorphisms in the
coagulation, fibrinolysis and inflammation pathways in a cohort of critically ill
patients who had severe sepsis and no XIGRIS ™ contraindications.
DIFFERENCE = median days alive and free of use of mechanical
ventilator of patients having the IRP minus median days alive and free of use
of mechanical ventilator of patients having the non-IRP allele/genotype, within
(1) Matched Controls and (2) XIGRIS ™-Treated Patients.

| | | Matched Controls | | | XIGRIS ™-Treated Patients | | |
|---|---|---|---|---|---|---|---|
| | | Median | Median | | | Median | |
| SNP | IRP | IRP | non-IRP | DIFFERENCE | Median IRP | non-IRP | DIFFERENCE |
| FGB.155840914.G/A | A | 7 | 2 | 5 | 18 | 7 | 11 |
| F2.46717332.G/A | G | 0 | 6 | −6 | 4 | 14.5 | −10.5 |
| F2.46717332.G/A | GG | 0 | 6 | −6 | 4 | 14.5 | −10.5 |
| F2R.76059983.A/G | G | 1 | 4 | −3 | 18.5 | 3.5 | 15 |
| F2R.76059983.A/G | GG | 0 | 5 | −5 | 19 | 4 | 15 |
| F2R.76049220.G/C | GG | 2 | 3 | −1 | 15.5 | 4 | 11.5 |
| F3.94719939.A/G | GG | 0 | 3 | −3 | 22 | 3 | 19 |
| F5.166258759.A/G | G | 1 | 2 | −1 | 20 | 17 | 3 |
| F5.166236816.T/C | T | 1 | 4 | −3 | 17.5 | 4 | 13.5 |
| F5.166227911.A/G | A | 2 | 2 | 0 | 17.5 | 12 | 5.5 |
| F5.166269905.G/A | A | 2 | 1 | 1 | 17 | 7 | 10 |
| F7.112808416.A/G | AG | 0 | 7 | −7 | 17 | 12 | 5 |
| F10.112840894.A/C | C | 0 | 2 | −2 | 20 | 4 | 16 |
| F10.112825510.A/G | G | 0 | 4 | −4 | 20 | 7 | 13 |
| F10.112824083.T/C | T | 2 | 1 | 1 | 19 | 17 | 2 |
| SERPINE1.100363146.4G/5G | I | 1 | 2 | −1 | 17 | 17 | 0 |
| SERPINE1.100375050.G/A | A | 7 | 1 | 6 | 21 | 7 | 14 |
| SERPINE1.100375050.G/A | AG | 6 | 1 | 5 | 21 | 3 | 18 |
| SERPINA5.94123294.C/T | TT | 4 | 1 | 3 | 5.5 | 8.5 | −3 |
| IL6.22541812.C/G | C | 1 | 5.5 | −4.5 | 23.5 | 21 | 2.5 |
| IL6.22539885.G/C | G | 1 | 7 | −6 | 19 | 24 | −5 |
| IL10.203334802.C/A | A | 0 | 0 | 0 | 23 | 20 | 3 |
| IL12A.161198944.G/A | A | 7 | 2 | 5 | 19 | 7 | 12 |
| IL12A.161198944.G/A | GA | 7 | 2 | 5 | 19 | 5 | 14 |
| TNFRSF1A.6317783.T/C | CT | 0.5 | 6 | −5.5 | 17 | 8.5 | 8.5 |
| VEGF.43848656.G/A | AA | 0.5 | 4 | −3.5 | 20.5 | 12 | 8.5 |
| PROC.127890298.A/G | AG | 6 | 1 | 5 | 20 | 2.5 | 17.5 |
| PROC.127890457.T/C | CT | 7 | 1 | 6 | 20 | 1.5 | 18.5 |
| PROC.127892009.G/A | AG | 4 | 0.5 | 3.5 | 20 | 2.5 | 17.5 |
| PROC.127892092.C/T | CT | 1 | 2 | −1 | 15.5 | 3 | 12.5 |
| PROC.127894204.T/C | C | 4 | 1 | 3 | 15 | 3 | 12 |
| PROC.127894204.T/C | CT | 1 | 3 | −2 | 15.5 | 3 | 12.5 |
| PROC.127894608.G/A | AG | 1 | 2 | −1 | 19.5 | 2.5 | 17 |
| PROC.127894645.C/T | CT | 0 | 3.5 | −3.5 | 19 | 3 | 16 |
| PROC.127895556.G/A | A | 2.5 | 1 | 1.5 | 7 | 4 | 3 |
| PROC.127895556.G/A | AA | 3 | 1 | 2 | 15 | 4 | 11 |
| PROC.127895783.G/A | AG | 5 | 1 | 4 | 20 | 2 | 18 |
| PROC.127895876.T/C | CT | 1 | 3 | −2 | 19 | 3 | 16 |
| PROC.127899224.C/T | CT | 0.5 | 4 | −3.5 | 19 | 3 | 16 |
| PROC.127901000.T/C | CT | 5 | 1 | 4 | 20 | 3 | 17 |
| PROC.127901799.C/T | CT | 1 | 3 | −2 | 17 | 2.5 | 14.5 |
| PROC.127975205.T/C | C | 3 | 2 | 1 | 14 | 3 | 11 |
| PROCR.33183348.T/C | C | 0 | 2 | −2 | 3.5 | 14 | −10.5 |
| PROCR.33183694.C/A | A | 0 | 3 | −3 | 3.5 | 10.5 | −7 |
| PROCR.33186524.A/G | G | 0 | 2 | −2 | 4 | 14 | −10 |
| PROCR.33228215.A/G | AG | 0 | 3 | −3 | 18.5 | 4 | 14.5 |
| PROCR.33228215.A/G | G | 3 | 2 | 1 | 11 | 7 | 4 |
| AVERAGE | | | | −0.5 | | | 8.8 |
| DIFFERENCE | | | | | | | |

For acute renal dysfunction (TABLE 26), on average matched-control patients having the IRP allele/genotype do worse than patients having alleles/genotypes other than the IRP (−2.7 days alive and free of acute renal dysfunction). In contrast, on average, XIGRIS™-treated patients having the IRP allele/genotype do better than patients having alleles/genotypes other than the IRP (+12.2 days alive and free of acute renal dysfunction). Clearly, the IRP patients benefit the most from XIGRIS™ treatment in terms of improvements of days alive and free of acute renal dysfunction.

TABLE 26
Difference in median days alive and free of acute renal dysfunction between
improved response polymorphism (IRP) and non-IRP patients by treatment
(control or XIGRIS ™). Data is shown for several polymorphisms in the
coagulation, fibrinolysis and inflammation pathways in a cohort of critically ill
patients who had severe sepsis and no XIGRIS ™ contraindications.
DIFFERENCE = median days alive and free of acute renal dysfunction
of patients having the IRP minus median days alive and free of acute renal
dysfunction of patients having the non-IRP allele/genotype, within (1)
Matched Controls and (2) XIGRIS ™-Treated Patients.

| | | Matched Controls | | | XIGRIS ™-Treated Patients | | |
|---|---|---|---|---|---|---|---|
| | | Median | Median | | Median | Median | |
| SNP | IRP | IRP | non-IRP | DIFFERENCE | IRP | non-IRP | DIFFERENCE |
| FGB.155840914.G/A | A | 18 | 13 | 5 | 27.5 | 12 | 15.5 |
| F2.46717332.G/A | G | 11 | 19 | −8 | 17 | 20 | −3 |
| F2.46717332.G/A | GG | 6 | 19 | −13 | 16 | 20 | −4 |
| F2R.76059983.A/G | G | 12 | 16 | −4 | 25.5 | 13.5 | 12 |
| F2R.76059983.A/G | GG | 7 | 16 | −9 | 28 | 14.5 | 13.5 |
| F2R.76049220.G/C | GG | 11.5 | 22 | −10.5 | 22 | 15 | 7 |
| F3.94719939.A/G | GG | 12.5 | 14 | −1.5 | 20.5 | 16.5 | 4 |
| F5.166258759.A/G | G | 15.5 | 12.5 | 3 | 28 | 12 | 16 |
| F5.166236816.T/C | T | 10 | 15 | −5 | 27 | 4.5 | 22.5 |
| F5.166227911.A/G | A | 15 | 12 | 3 | 27 | 5 | 22 |
| F5.166269905.G/A | A | 16 | 12 | 4 | 26 | 5 | 21 |
| F7.112808416.A/G | AG | 8 | 17.5 | −9.5 | 15 | 13 | 2 |
| F10.112840894.A/C | C | 12 | 13 | −1 | 26 | 12 | 14 |
| F10.112825510.A/G | G | 13 | 13 | 0 | 27 | 12 | 15 |
| F10.112824083.T/C | T | 13 | 13 | 0 | 26 | 12 | 14 |
| SERPINE1.100363146.4G/5G | I | 9 | 18 | −9 | 12 | 14 | −2 |
| SERPINE1.100375050.G/A | A | 12 | 13 | −1 | 28 | 12 | 16 |
| SERPINE1.100375050.G/A | AG | 12 | 13 | −1 | 28 | 12 | 16 |
| SERPINA5.94123294.C/T | TT | 15.5 | 13 | 2.5 | 28 | 13.5 | 14.5 |
| IL6.22541812.C/G | C | 11.5 | 9.5 | 2 | 0 | 13.5 | −13.5 |
| IL6.22539885.G/C | G | 10 | 13 | −3 | 12 | 0 | 12 |
| IL10.203334802.C/A | A | 11 | 9 | 2 | 27 | 0 | 27 |
| IL12A.161198944.G/A | A | 12 | 14.5 | −2.5 | 28 | 12 | 16 |
| IL12A.161198944.G/A | GA | 12 | 15.5 | −3.5 | 28 | 12 | 16 |
| TNFRSF1A.6317783.T/C | CT | 12 | 16 | −4 | 27 | 1 | 26 |
| VEGF.43848656.G/A | AA | 10.5 | 14 | −3.5 | 27.5 | 12 | 15.5 |
| PROC.127890298.A/G | AG | 15.5 | 13 | 2.5 | 28 | 12.5 | 15.5 |
| PROC.127890457.T/C | CT | 15.5 | 13 | 2.5 | 27.5 | 12 | 15.5 |
| PROC.127892009.G/A | AG | 16 | 12.5 | 3.5 | 27.5 | 12.5 | 15 |
| PROC.127892092.C/T | CT | 14 | 12 | 2 | 27.5 | 13.5 | 14 |
| PROC.127894204.T/C | C | 14 | 15 | −1 | 25.5 | 12.5 | 13 |
| PROC.127894204.T/C | CT | 13 | 15.5 | −2.5 | 27.5 | 13 | 14.5 |
| PROC.127894608.G/A | AG | 10 | 17 | −7 | 28 | 12 | 16 |
| PROC.127894645.C/T | CT | 13 | 15 | −2 | 28 | 12 | 16 |
| PROC.127895556.G/A | A | 10 | 15.5 | −5.5 | 19 | 25 | −6 |
| PROC.127895556.G/A | AA | 14 | 14.5 | −0.5 | 22.5 | 15 | 7.5 |
| PROC.127895783.G/A | AG | 16 | 12 | 4 | 28 | 12 | 16 |
| PROC.127895876.T/C | CT | 10 | 18 | −8 | 28 | 12 | 16 |
| PROC.127899224.C/T | CT | 13 | 16 | −3 | 28 | 12.5 | 15.5 |
| PROC.127901000.T/C | CT | 16 | 13 | 3 | 28 | 12 | 16 |
| PROC.127901799.C/T | CT | 13 | 15 | −2 | 28 | 12 | 16 |
| PROC.127975205.T/C | C | 12 | 15 | −3 | 19 | 15 | 4 |
| PROCR.33183348.T/C | C | 5.5 | 14 | −8.5 | 23.5 | 20 | 3.5 |
| PROCR.33183694.C/A | A | 5.5 | 15 | −9.5 | 23.5 | 14 | 9.5 |
| PROCR.33186524.A/G | G | 5 | 14 | −9 | 28 | 15 | 13 |
| PROCR.33228215.A/G | AG | 5 | 14 | −9 | 28 | 14 | 14 |
| PROCR.33228215.A/G | G | 7 | 13 | −6 | 28 | 14.5 | 13.5 |
| AVERAGE | | | | −2.7 | | | 12.2 |
| DIFFERENCE | | | | | | | |

For any renal dysfunction (TABLE 27), on average matched-control patients having the IRP allele/genotype do worse than patients having alleles/genotypes other than the IRP (−1.9 days alive and free of any renal dysfunction). In contrast, on average, XIGRIS™-treated patients having the IRP allele/genotype do better than patients having alleles/genotypes other than the IRP (+10.1 days alive and free of any renal dysfunction). Clearly, the IRP patients benefit the most from XIGRIS™ treatment in terms of improvements of days alive and free of any renal dysfunction.

TABLE 27
Difference in median days alive and free of any renal dysfunction between
improved response polymorphism (IRP) and non-IRP patients by treatment
(control or XIGRIS ™). Data is shown for several polymorphisms in
the coagulation, fibrinolysis and inflammation pathways in a cohort of
critically ill patients who had severe sepsis and no XIGRIS ™
contraindications. DIFFERENCE = median days alive and free of any
renal dysfunction of patients having the IRP minus median days alive and
free of any renal dysfunction of patients having the non-IRP allele/genotype,
within (1) Matched Controls and (2) XIGRIS ™-Treated Patients.

| | | Matched Controls | | | XIGRIS ™-Treated Patients | | |
|---|---|---|---|---|---|---|---|
| | | | Median | | | Median | |
| SNP | IRP | Median IRP | non-IRP | DIFFERENCE | Median IRP | non-IRP | DIFFERENCE |
| FGB.155840914.G/A | A | 8 | 9 | −1 | 14 | 12 | 2 |
| F2.46717332.G/A | G | 6 | 12 | −6 | 14.5 | 14.5 | 0 |
| F2.46717332.G/A | GG | 5 | 12 | −7 | 15.5 | 14.5 | 1 |
| F2R.76059983.A/G | G | 8.5 | 9 | −0.5 | 22 | 13 | 9 |
| F2R.76059983.A/G | GG | 5 | 10 | −5 | 28 | 14 | 14 |
| F2R.76049220.G/C | GG | 6.5 | 14 | −7.5 | 16.5 | 15 | 1.5 |
| F3.94719939.A/G | GG | 8.5 | 9 | −0.5 | 20.5 | 14 | 6.5 |
| F5.166258759.A/G | G | 12.5 | 9 | 3.5 | 15 | 12 | 3 |
| F5.166236816.T/C | T | 8 | 11 | −3 | 16.5 | 4.5 | 12 |
| F5.166227911.A/G | A | 12 | 7 | 5 | 16.5 | 5 | 11.5 |
| F5.166269905.G/A | A | 13 | 8 | 5 | 15 | 5 | 10 |
| F7.112808416.A/G | AG | 4 | 13 | −9 | 14 | 13 | 1 |
| F10.112840894.A/C | C | 8 | 10 | −2 | 18 | 12 | 6 |
| F10.112825510.A/G | G | 8 | 9 | −1 | 27 | 12 | 15 |
| F10.112824083.T/C | T | 11 | 9 | 2 | 15 | 12 | 3 |
| SERPINE1.100363146.4G/5G | I | 5 | 13 | −8 | 12 | 14 | −2 |
| SERPINE1.100375050.G/A | A | 7 | 9 | −2 | 28 | 12 | 16 |
| SERPINE1.100375050.G/A | AG | 12 | 9 | 3 | 28 | 12 | 16 |
| SERPINA5.94123294.C/T | TT | 13.5 | 8 | 5.5 | 28 | 13 | 15 |
| IL6.22541812.C/G | C | 7.5 | 4 | 3.5 | 0 | 13.5 | −13.5 |
| IL6.22539885.G/C | G | 4 | 9 | −5 | 12 | 0 | 12 |
| IL10.203334802.C/A | A | 10 | 6 | 4 | 27 | 0 | 27 |
| IL12A.161198944.G/A | A | 3.5 | 9 | −5.5 | 28 | 12 | 16 |
| IL12A.161198944.G/A | GA | 3.5 | 9 | −5.5 | 28 | 12 | 16 |
| TNFRSF1A.6317783.T/C | CT | 7.5 | 12 | −4.5 | 18 | 1 | 17 |
| VEGF.43848656.G/A | AA | 6 | 9 | −3 | 27.5 | 12 | 15.5 |
| PROC.127890298.A/G | AG | 8.5 | 10 | −1.5 | 28 | 12 | 16 |
| PROC.127890457.T/C | CT | 8.5 | 10 | −1.5 | 27.5 | 11 | 16.5 |
| PROC.127892009.G/A | AG | 9 | 9 | 0 | 27.5 | 12 | 15.5 |
| PROC.127892092.C/T | CT | 9.5 | 7 | 2.5 | 27.5 | 13 | 14.5 |
| PROC.127894204.T/C | C | 10.5 | 9 | 1.5 | 19 | 12 | 7 |
| PROC.127894204.T/C | CT | 9 | 10 | −1 | 27.5 | 13 | 14.5 |
| PROC.127894608.G/A | AG | 8 | 11 | −3 | 28 | 11 | 17 |
| PROC.127894645.C/T | CT | 9 | 10.5 | −1.5 | 28 | 12 | 16 |
| PROC.127895556.G/A | A | 5 | 10 | −5 | 19 | 14 | 5 |
| PROC.127895556.G/A | AA | 14 | 8.5 | 5.5 | 22.5 | 14 | 8.5 |
| PROC.127895783.G/A | AG | 9 | 7 | 2 | 28 | 10 | 18 |
| PROC.127895876.T/C | CT | 8 | 12 | −4 | 28 | 12 | 16 |
| PROC.127899224.C/T | CT | 9.5 | 10 | −0.5 | 28 | 12 | 16 |
| PROC.127901000.T/C | CT | 9 | 9 | 0 | 28 | 12 | 16 |
| PROC.127901799.C/T | CT | 9.5 | 9.5 | 0 | 28 | 11 | 17 |
| PROC.127975205.T/C | C | 7 | 10 | −3 | 19 | 14 | 5 |
| PROCR.33183348.T/C | C | 3 | 9.5 | −6.5 | 18.5 | 14 | 4.5 |
| PROCR.33183694.C/A | A | 3 | 10 | −7 | 18.5 | 14 | 4.5 |
| PROCR.33186524.A/G | G | 3 | 10 | −7 | 18 | 14 | 4 |
| PROCR.33228215.A/G | AG | 2 | 10 | −8 | 18.5 | 14 | 4.5 |
| PROCR.33228215.A/G | G | 3 | 9 | −6 | 23.5 | 14 | 9.5 |
| AVERAGE | | | | −1.9 | | | 10.1 |
| DIFFERENCE | | | | | | | |

For days alive and free of renal support (TABLE 28), on average matched-control patients having the IRP allele/genotype do worse than patients having alleles/genotypes other than the IRP (−2 days alive and free of renal support). In contrast, on average, XIGRIS™-treated patients having the IRP allele/genotype do better than patients having alleles/genotypes other than the IRP (+14.8 days alive and free of renal support). Clearly, the IRP patients benefit the most from XIGRIS™ treatment in terms of improvements of days alive and free of renal support.

TABLE 28
Difference in median days alive and free of renal support between improved
response polymorphism (IRP) and non-IRP patients by treatment (control or
XIGRIS ™). Data is shown for several polymorphisms in the coagulation,
fibrinolysis and inflammation pathways in a cohort of critically ill patients who
had severe sepsis and no XIGRIS ™ contraindications.
DIFFERENCE = median days alive and free of renal support of patients
having the IRP minus median days alive and free of renal support of patients
having the non-IRP allele/genotype, within (1) Matched Controls and (2)
XIGRIS ™-Treated Patients.

| | | Matched Controls | | | XIGRIS ™-Treated Patients | | |
|---|---|---|---|---|---|---|---|
| | | Median | Median | | | Median | |
| SNP | IRP | IRP | non-IRP | DIFFERENCE | Median IRP | non-IRP | DIFFERENCE |
| FGB.155840914.G/A | A | 13 | 15 | −2 | 8 | 9.5 | −1.5 |
| F2.46717332.G/A | G | 12 | 19 | −7 | 10 | 14 | −4 |
| F2.46717332.G/A | GG | 6 | 19 | −13 | 19 | 12 | 7 |
| F2R.76059983.A/G | G | 9 | 15 | −6 | 27.5 | 9.5 | 18 |
| F2R.76059983.A/G | GG | 7 | 15 | −8 | 28 | 10 | 18 |
| F2R.76049220.G/C | GG | 11 | 22 | −11 | 17.5 | 14 | 3.5 |
| F3.94719939.A/G | GG | 15.5 | 13 | 2.5 | 27 | 9.5 | 17.5 |
| F5.166258759.A/G | G | 21 | 12 | 9 | 28 | 9 | 19 |
| F5.166236816.T/C | T | 13 | 13 | 0 | 27 | 7 | 20 |
| F5.166227911.A/G | A | 21 | 9 | 12 | 27.5 | 7 | 20.5 |
| F5.166269905.G/A | A | 16 | 12 | 4 | 26 | 5 | 21 |
| F7.112808416.A/G | AG | 6 | 17 | −11 | 5 | 14.5 | −9.5 |
| F10.112840894.A/C | C | 11 | 13 | −2 | 26 | 9 | 17 |
| F10.112825510.A/G | G | 13 | 13 | 0 | 28 | 5 | 23 |
| F10.112824083.T/C | T | 16 | 12 | 4 | 26 | 5 | 21 |
| SERPINE1.100363146.4G/5G | I | 8 | 16 | −8 | 10 | 14 | −4 |
| SERPINE1.100375050.G/A | A | 15 | 13 | 2 | 28 | 9 | 19 |
| SERPINE1.100375050.G/A | AG | 20 | 12 | 8 | 28 | 5 | 23 |
| SERPINA5.94123294.C/T | TT | 17.5 | 12 | 5.5 | 28 | 10 | 18 |
| IL6.22541812.C/G | C | 15 | 15 | 0 | 7.5 | 21.5 | −14 |
| IL6.22539885.G/C | G | 5 | 15 | −10 | 15 | 0 | 15 |
| IL10.203334802.C/A | A | 15 | 6 | 9 | 28 | 2 | 26 |
| IL12A.161198944.G/A | A | 4.5 | 14 | −9.5 | 28 | 10 | 18 |
| IL12A.161198944.G/A | GA | 4.5 | 14.5 | −10 | 28 | 8.5 | 19.5 |
| TNFRSF1A.6317783.T/C | CT | 12 | 15 | −3 | 26 | 3.5 | 22.5 |
| VEGF.43848656.G/A | AA | 14 | 13 | 1 | 28 | 7 | 21 |
| PROC.127890298.A/G | AG | 15 | 13 | 2 | 28 | 7 | 21 |
| PROC.127890457.T/C | CT | 15 | 13 | 2 | 28 | 5.5 | 22.5 |
| PROC.127892009.G/A | AG | 15 | 12.5 | 2.5 | 28 | 7 | 21 |
| PROC.127892092.C/T | CT | 15 | 11 | 4 | 28 | 9 | 19 |
| PROC.127894204.T/C | C | 15 | 13 | 2 | 27 | 8 | 19 |
| PROC.127894204.T/C | CT | 13 | 15 | −2 | 28 | 10 | 18 |
| PROC.127894608.G/A | AG | 10 | 15 | −5 | 28 | 6.5 | 21.5 |
| PROC.127894645.C/T | CT | 12 | 15.5 | −3.5 | 28 | 8 | 20 |
| PROC.127895556.G/A | A | 5.5 | 15 | −9.5 | 26 | 10 | 16 |
| PROC.127895556.G/A | AA | 11 | 13 | −2 | 27 | 10 | 17 |
| PROC.127895783.G/A | AG | 15 | 12 | 3 | 28 | 6 | 22 |
| PROC.127895876.T/C | CT | 9.5 | 16 | −6.5 | 28 | 8 | 20 |
| PROC.127899224.C/T | CT | 13 | 15 | −2 | 28 | 6.5 | 21.5 |
| PROC.127901000.T/C | CT | 15 | 12 | 3 | 28 | 8 | 20 |
| PROC.127901799.C/T | CT | 14 | 14.5 | −0.5 | 28 | 6.5 | 21.5 |
| PROC.127975205.T/C | C | 11 | 14 | −3 | 28 | 9 | 19 |
| PROCR.33183348.T/C | C | 5.5 | 13 | −7.5 | 14.5 | 12 | 2.5 |
| PROCR.33183694.C/A | A | 5.5 | 13.5 | −8 | 14.5 | 10 | 4.5 |
| PROCR.33186524.A/G | G | 15 | 13 | 2 | 1 | 14 | −13 |
| PROCR.33228215.A/G | AG | 2 | 15 | −13 | 21.5 | 10 | 11.5 |
| PROCR.33228215.A/G | G | 3 | 13 | −10 | 28 | 10 | 18 |
| AVERAGE | | | | −2 | | | 14.8 |
| DIFFERENCE | | | | | | | |

For coagulation dysfunction (as measured by the Brussels hematologic platelet count) (TABLE 29), on average matched-control patients having the IRP allele/genotype do worse than patients having alleles/genotypes other than the IRP (−1.6 days alive and free of coagulation dysfunction). In contrast, on average, XIGRIS™-treated patients having the IRP allele/genotype do better than patients having alleles/genotypes other than the IRP (+9 days alive and free of coagulation dysfunction). Clearly, the IRP patients benefit the most from XIGRIS™ treatment in terms of improvements of days alive and free of coagulation dysfunction.

TABLE 29
Difference in median days alive and free of coagulation dysfunction (as
measured by the Brussels hematologic platelet count) between improved
response polymorphism (IRP) and non-IRP patients by treatment (control
or XIGRIS ™). Data is shown for several polymorphisms in the
coagulation, fibrinolysis and inflammation pathways in a cohort of critically
ill patients who had severe sepsis and no XIGRIS ™ contraindications.
DIFFERENCE = median days alive and free of coagulation dysfunction
of patients having the IRP minus median days alive and free of coagulation
dysfunction of patients having the non-IRP allele/genotype, within (1)
Matched Controls and (2) XIGRIS ™-Treated Patients.

| | | Matched Controls | | | XIGRIS ™-Treated Patients | | |
|---|---|---|---|---|---|---|---|
| | | | Median | | | Median | |
| SNP | IRP | Median IRP | non-IRP | DIFFERENCE | Median IRP | non-IRP | DIFFERENCE |
| FGB.155840914.G/A | A | 22 | 23 | −1 | 27.5 | 20 | 7.5 |
| F2.46717332.G/A | G | 19 | 25 | −6 | 20 | 25.5 | −5.5 |
| F2.46717332.G/A | GG | 11 | 25 | −14 | 16 | 25.5 | −9.5 |
| F2R.76059983.A/G | G | 22 | 23.5 | −1.5 | 27 | 20 | 7 |
| F2R.76059983.A/G | GG | 12 | 24 | −12 | 24 | 21.5 | 2.5 |
| F2R.76049220.G/C | GG | 20 | 26 | −6 | 22 | 23 | −1 |
| F3.94719939.A/G | GG | 15 | 23 | −8 | 27.5 | 17 | 10.5 |
| F5.166258759.A/G | G | 17.5 | 22 | −4.5 | 28 | 20 | 8 |
| F5.166236816.T/C | T | 23 | 21 | 2 | 25.5 | 11 | 14.5 |
| F5.166227911.A/G | A | 25 | 20 | 5 | 24 | 16 | 8 |
| F5.166269905.G/A | A | 22 | 23 | −1 | 24 | 20 | 4 |
| F7.112808416.A/G | AG | 13 | 25.5 | −12.5 | 23 | 21 | 2 |
| F10.112840894.A/C | C | 21 | 23 | −2 | 28 | 14 | 14 |
| F10.112825510.A/G | G | 16 | 23 | −7 | 27 | 20 | 7 |
| F10.112824083.T/C | T | 22 | 23 | −1 | 27 | 20 | 7 |
| SERPINE1.100363146.4G/5G | I | 20 | 24 | −4 | 23 | 20 | 3 |
| SERPINE1.100375050.G/A | A | 23 | 22 | 1 | 27.5 | 17 | 10.5 |
| SERPINE1.100375050.G/A | AG | 23 | 22 | 1 | 27.5 | 12 | 15.5 |
| SERPINA5.94123294.C/T | TT | 23 | 22 | 1 | 17.5 | 23.5 | −6 |
| IL6.22541812.C/G | C | 28 | 28 | 0 | 27.5 | 28 | −0.5 |
| IL6.22539885.G/C | G | 20 | 20 | 0 | 28 | 27 | 1 |
| IL10.203334802.C/A | A | 20 | 15 | 5 | 28 | 27 | 1 |
| IL12A.161198944.G/A | A | 19.5 | 23 | −3.5 | 28 | 14 | 14 |
| IL12A.161198944.G/A | GA | 19.5 | 23 | −3.5 | 28 | 13 | 15 |
| TNFRSF1A.6317783.T/C | CT | 20.5 | 25 | −4.5 | 27 | 12.5 | 14.5 |
| VEGF.43848656.G/A | AA | 21.5 | 24 | −2.5 | 28 | 17 | 11 |
| PROC.127890298.A/G | AG | 24.5 | 19 | 5.5 | 28 | 11 | 17 |
| PROC.127890457.T/C | CT | 25.5 | 19 | 6.5 | 28 | 10 | 18 |
| PROC.127892009.G/A | AG | 24 | 18.5 | 54 | 28 | 11 | 17 |
| PROC.127892092.C/T | CT | 23 | 20 | 3 | 27.5 | 13 | 14.5 |
| PROC.127894204.T/C | C | 22.5 | 22.5 | 0 | 25.5 | 14 | 11.5 |
| PROC.127894204.T/C | CT | 22.5 | 22 | 0.5 | 27 | 11 | 16 |
| PROC.127894608.G/A | AG | 23 | 21 | 2 | 27.5 | 10 | 17.5 |
| PROC.127894645.C/T | CT | 23 | 20.5 | 2.5 | 27 | 10 | 17 |
| PROC.127895556.G/A | A | 17.5 | 23 | −5.5 | 23 | 20 | 3 |
| PROC.127895556.G/A | AA | 15 | 23 | −8 | 27.5 | 20 | 7.5 |
| PROC.127895783.G/A | AG | 24 | 18.5 | 5.5 | 28 | 10 | 18 |
| PROC.127895876.T/C | CT | 22.5 | 21 | 1.5 | 27 | 10 | 17 |
| PROC.127899224.C/T | CT | 23.5 | 21 | 2.5 | 27 | 11 | 16 |
| PROC.127901000.T/C | CT | 24 | 20 | 4 | 28 | 12 | 16 |
| PROC.127901799.C/T | CT | 23 | 21 | 2 | 28 | 10 | 18 |
| PROC.127975205.T/C | C | 23 | 22 | 1 | 27 | 20 | 7 |
| PROCR.33183348.T/C | C | 20.5 | 22.5 | −2 | 27 | 20 | 7 |
| PROCR.33183694.C/A | A | 20.5 | 23 | −2.5 | 27 | 20 | 7 |
| PROCR.33186524.A/G | G | 20 | 23 | −3 | 27 | 20 | 7 |
| PROCR.33228215.A/G | AG | 15 | 23 | −8 | 27.5 | 20 | 7.5 |
| PROCR.33228215.A/G | G | 15 | 23 | −8 | 27 | 20 | 7 |
| AVERAGE | | | | −1.6 | | | 9 |
| DIFFERENCE | | | | | | | |

For days alive and free of INR>1.5 (TABLE 30), on average matched-control patients having the IRP allele/genotype do worse than patients having alleles/genotypes other than the IRP (−1.7 days alive and free of INR>1.5). In contrast, on average, XIGRIS™-treated patients having the IRP allele/genotype do better than patients having alleles/genotypes other than the IRP (+5.4 days alive and free of INR>1.5). Clearly, the IRP patients benefit the most from XIGRIS™ treatment in terms of improvements of days alive and free of INR>1.5.

TABLE 30
Difference in median days alive and free of INR >1.5 between
improved response polymorphism (IRP) and non-IRP patients by
treatment (control or XIGRIS ™). Data is shown for several
polymorphisms in the coagulation, fibrinolysis and inflammation
pathways in a cohort of critically ill patients who had severe sepsis
and no XIGRIS ™ contraindications. DIFFERENCE =
median days alive and free of INR >1.5 of patients having the IRP
minus median days alive and free of INR >1.5 of patients having
the non-IRP allele/genotype, within (1) Matched Controls and
(2) XIGRIS ™-Treated Patients.

| | | Matched Controls | | | XIGRIS ™-Treated Patients | | |
|---|---|---|---|---|---|---|---|
| | | | Median | | Median | Median | |
| SNP | IRP | Median IRP | non-IRP | DIFFERENCE | IRP | non-IRP | DIFFERENCE |
| FGB.155840914.G/A | A | 23 | 23 | 0 | 28 | 27.5 | 0.5 |
| F2.46717332.G/A | G | 15 | 25 | −10 | 27.5 | 26.5 | 1 |
| F2.46717332.G/A | GG | 9 | 24 | −15 | 28 | 26.5 | 1.5 |
| F2R.76059983.A/G | G | 20 | 23 | −3 | 28 | 26.5 | 1.5 |
| F2R.76059983.A/G | GG | 9 | 23 | −14 | 26 | 27 | −1 |
| F2R.76049220.G/C | GG | 17.5 | 26 | −8.5 | 27 | 27 | 0 |
| F3.94719939.A/G | GG | 18 | 23 | −5 | 28 | 26 | 2 |
| F5.166258759.A/G | G | 26.5 | 22 | 4.5 | 28 | 27 | 1 |
| F5.166236816.T/C | T | 23 | 22 | 1 | 28 | 16.5 | 11.5 |
| F5.166227911.A/G | A | 25 | 19 | 6 | 28 | 27.5 | 0.5 |
| F5.166269905.G/A | A | 26 | 22 | 4 | 28 | 27 | 1 |
| F7.112808416.A/G | AG | 10 | 26 | −16 | 28 | 27 | 1 |
| F10.112840894.A/C | C | 18 | 23 | −5 | 28 | 28 | 0 |
| F10.112825510.A/G | G | 12 | 25 | −13 | 28 | 28 | 0 |
| F10.112824083.T/C | T | 21 | 23 | −2 | 28 | 28 | 0 |
| SERPINE1.100363146.4G/5G | I | 16 | 25 | −9 | 28 | 27 | 1 |
| SERPINE1.100375050.G/A | A | 23 | 22 | 1 | 28 | 27.5 | 0.5 |
| SERPINE1.100375050.G/A | AG | 23 | 21 | 2 | 28 | 27 | 1 |
| SERPINA5.94123294.C/T | TT | 20.5 | 22 | −1.5 | 28 | 26 | 2 |
| IL6.22541812.C/G | C | 26 | 26.5 | −0.5 | 28 | 28 | 0 |
| IL6.22539885.G/C | G | 19 | 26 | −7 | 28 | 28 | 0 |
| IL10.203334802.C/A | A | 15 | 16 | −1 | 28 | 28 | 0 |
| IL12A.161198944.G/A | A | 20 | 23 | −3 | 28 | 27 | 1 |
| IL12A.161198944.G/A | GA | 20 | 23 | −3 | 28 | 27 | 1 |
| TNFRSF1A.6317783.T/C | CT | 21.5 | 25 | −3.5 | 28 | 16.5 | 11.5 |
| VEGF.43848656.G/A | AA | 22 | 23 | −1 | 28 | 28 | 0 |
| PROC.127890298.A/G | AG | 23.5 | 19 | 4.5 | 28 | 12 | 16 |
| PROC.127890457.T/C | CT | 24.5 | 19 | 5.5 | 28 | 9 | 19 |
| PROC.127892009.G/A | AG | 22 | 18.5 | 3.5 | 28 | 12 | 16 |
| PROC.127892092.C/T | CT | 22.5 | 21 | 1.5 | 28 | 20.5 | 7.5 |
| PROC.127894204.T/C | C | 21.5 | 21 | 0.5 | 28 | 14 | 14 |
| PROC.127894204.T/C | CT | 20.5 | 22.5 | −2 | 27.5 | 18.5 | 9 |
| PROC.127894608.G/A | AG | 21 | 22 | −1 | 28 | 9 | 19 |
| PROC.127894645.C/T | CT | 22.5 | 20 | 2.5 | 28 | 10 | 18 |
| lPROC.127895556.G/A | A | 21.5 | 21 | 0.5 | 28 | 26 | 2 |
| PROC.127895556.G/A | AA | 23 | 21 | 2 | 28 | 26 | 2 |
| PROC.127895783.G/A | AG | 23 | 17.5 | 5.5 | 28 | 10 | 18 |
| PROC.127895876.T/C | CT | 20.5 | 21 | −0.5 | 28 | 10 | 18 |
| PROC.127899224.C/T | CT | 22 | 21 | 1 | 28 | 12 | 16 |
| PROC.127901000.T/C | CT | 24 | 19 | 5 | 28 | 14 | 14 |
| PROC.127901799.C/T | CT | 22 | 21 | 1 | 28 | 9 | 19 |
| PROC.127975205.T/C | C | 21 | 21 | 0 | 28 | 26 | 2 |
| PROCR.33183348.T/C | C | 21.5 | 21 | 0.5 | 28 | 27 | 1 |
| PROCR.33183694.C/A | A | 21.5 | 22 | −0.5 | 28 | 26 | 2 |
| PROCR.33186524.A/G | G | 21 | 21 | 0 | 28 | 26 | 2 |
| PROCR.33228215.A/G | AG | 16 | 23 | −7 | 28 | 26 | 2 |
| PROCR.33228215.A/G | G | 21 | 22 | −1 | 28 | 27 | 1 |
| AVERAGE DIFFERENCE | | | | −1.7 | | | 5.4 |

For neurological dysfunction (TABLE 31), on average matched-control patients having the IRP allele/genotype do worse than patients having alleles/genotypes other than the IRP (−2.1 days alive and free of neurological dysfunction). In contrast, on average, XIGRIS™-treated patients having the IRP allele/genotype do better than patients having alleles/genotypes other than the IRP (+7.3 days alive and free of neurological dysfunction). Clearly, the IRP patients benefit the most from XIGRIS™ treatment in terms of improvements of days alive and free of neurological dysfunction.

TABLE 31
Difference in median days alive and free of neurological dysfunction
between improved response polymorphism (IRP) and non-IRP
patients by treatment (control or XIGRIS ™). Data is shown for
several polymorphisms in the coagulation, fibrinolysis and inflammation
pathways in a cohort of critically ill patients who had severe sepsis and
no XIGRIS ™ contraindications. DIFFERENCE = median
days alive and free of neurological dysfunction of patients having the
IRP minus median days alive and free of neurological dysfunction of
patients having the non-IRP allele/genotype, within (1) Matched Controls
and (2) XIGRIS ™-Treated Patients.

| | | Matched Controls | | | XIGRIS ™-Treated Patients | | |
|---|---|---|---|---|---|---|---|
| | | Median | Median | | | Median | |
| SNP | IRP | IRP | non-IRP | DIFFERENCE | Median IRP | non-IRP | DIFFERENCE |
| FGB.155840914.G/A | A | 18 | 19 | −1 | 27 | 19 | 8 |
| F2.46717332.G/A | G | 14 | 22 | −8 | 23 | 23 | 0 |
| F2.46717332.G/A | GG | 8 | 21 | −13 | 23.5 | 23 | 0.5 |
| F2R.76059983.A/G | G | 15 | 19 | −4 | 25 | 22.5 | 2.5 |
| F2R.76059983.A/G | GG | 8 | 20 | −12 | 25 | 23 | 2 |
| F2R.76049220.G/C | GG | 15 | 22 | −7 | 24.5 | 22 | 2.5 |
| F3.94719939.A/G | GG | 17 | 18 | −1 | 24.5 | 22.5 | 2 |
| F5.166258759.A/G | G | 22 | 16 | 6 | 26 | 19 | 7 |
| F5.166236816.T/C | T | 18 | 16 | 2 | 25 | 3.5 | 21.5 |
| F5.166227911.A/G | A | 19 | 15 | 4 | 24 | 5 | 19 |
| F5.166269905.G/A | A | 23 | 15 | 8 | 25 | 19 | 6 |
| F7.112808416.A/G | AG | 10 | 23 | −13 | 23 | 14 | 9 |
| F10.112840894.A/C | C | 14 | 20 | −6 | 26 | 22 | 4 |
| F10.112825510.A/G | G | 11 | 20 | −9 | 25 | 22 | 3 |
| F10.112824083.T/C | T | 14 | 20 | −6 | 23 | 22 | 1 |
| SERPINE1.100363146.4G/5G | I | 14 | 21 | −7 | 23 | 22 | 1 |
| SERPINE1.100375050.G/A | A | 14 | 19 | −5 | 25.5 | 20.5 | 5 |
| SERPINE1.100375050.G/A | AG | 22 | 16 | 6 | 25.5 | 9 | 16.5 |
| SERPINA5.94123294.C/T | TT | 20.5 | 16 | 4.5 | 24.5 | 22.5 | 2 |
| IL6.22541812.C/G | C | 21.5 | 22.5 | −1 | 26 | 27 | −1 |
| IL6.22539885.G/C | G | 16 | 15 | 1 | 26 | 26 | 0 |
| IL10.203334802.C/A | A | 15 | 15 | 0 | 26 | 25 | 1 |
| IL12A.161198944.G/A | A | 18 | 19 | −1 | 25 | 22 | 3 |
| IL12A.161198944.G/A | GA | 18 | 19 | −1 | 25 | 14 | 11 |
| TNFRSF1A.6317783.T/C | CT | 14.5 | 22 | −7.5 | 23 | 15 | 8 |
| VEGF.43848656.G/A | AA | 17.5 | 19 | −1.5 | 26.5 | 20.5 | 6 |
| PROC.127890298.A/G | AG | 20.5 | 16 | 4.5 | 25 | 10 | 15 |
| PROC.127890457.T/C | CT | 20.5 | 16 | 4.5 | 25 | 8 | 17 |
| PROC.127892009.G/A | AG | 20 | 15.5 | 4.5 | 25 | 10 | 15 |
| PROC.127892092.C/T | CT | 17 | 17 | 0 | 24.5 | 15.5 | 9 |
| PROC.127894204.T/C | C | 18 | 19 | −1 | 24 | 11 | 13 |
| PROC.127894204.T/C | CT | 15 | 19 | −4 | 24.5 | 15 | 9.5 |
| PROC.127894608.G/A | AG | 15 | 18 | −3 | 25 | 6.5 | 18.5 |
| PROC.127894645.C/T | CT | 19 | 16 | 3 | 25 | 7 | 18 |
| PROC.127895556.G/A | A | 10 | 19.5 | −9.5 | 23 | 23 | 0 |
| PROC.127895556.G/A | AA | 10 | 19 | −9 | 21.5 | 23 | −1.5 |
| PROC.127895783.G/A | AG | 21 | 15.5 | 5.5 | 25 | 9 | 16 |
| PROC.127895876.T/C | CT | 15 | 18 | −3 | 25 | 7 | 18 |
| PROC.127899224.C/T | CT | 19 | 16 | 3 | 25 | 8 | 17 |
| PROC.127901000.T/C | CT | 21 | 16 | 5 | 25 | 11 | 14 |
| PROC.127901799.C/T | CT | 20 | 16 | 4 | 25 | 6.5 | 18.5 |
| PROC.127975205.T/C | C | 16 | 18 | −2 | 24 | 23 | 1 |
| PROCR.33183348.T/C | C | 10 | 19 | −9 | 24 | 23 | 1 |
| PROCR.33183694.C/A | A | 10 | 19.5 | −9.5 | 24 | 23 | 1 |
| PROCR.33186524.A/G | G | 14 | 18 | −4 | 24 | 23 | 1 |
| PROCR.33228215.A/G | AG | 14 | 19 | −5 | 25 | 23 | 2 |
| PROCR.33228215.A/G | G | 16 | 18 | −2 | 24.5 | 23 | 1.5 |
| AVERAGE | | | | −2.1 | | | 7.3 |
| DIFFERENCE | | | | | | | |

For acute hepatic dysfunction (TABLE 32), on average matched-control patients having the IRP allele/genotype do worse than patients having alleles/genotypes other than the IRP (−2.3 days alive and free of acute hepatic dysfunction). In contrast, on average, XIGRIS™-treated patients having the IRP allele/genotype do better than patients having alleles/genotypes other than the IRP (+8 days alive and free of acute hepatic dysfunction). Clearly, the IRP patients benefit the most from XIGRIS™ treatment in terms of improvements of days alive and free of acute hepatic dysfunction.

TABLE 32
Difference in median days alive and free of acute hepatic dysfunction
between improved response polymorphism (IRP) and non-IRP patients
by treatment (control or XIGRIS ™). Data is shown for several
polymorphisms in the coagulation, fibrinolysis and inflammation
pathways in a cohort of critically ill patients who had severe sepsis and
no XIGRIS ™ contraindications. DIFFERENCE = median days
alive and free of acute hepatic dysfunction of patients having the IRP
minus median days alive and free of acute hepatic dysfunction of patients
having the non-IRP allele/genotype, within (1) Matched Controls and (2)
XIGRIS ™-Treated Patients.

| | | Matched Controls | | | XIGRIS ™-Treated Patients | | |
|---|---|---|---|---|---|---|---|
| | | Median | Median | | Median | Median | |
| SNP | IRP | IRP | non-IRP | DIFFERENCE | IRP | non-IRP | DIFFERENCE |
| FGB.155840914.G/A | A | 28 | 28 | 0 | 28 | 19 | 9 |
| F2.46717332.G/A | G | 20 | 28 | −8 | 25.5 | 26 | −0.5 |
| F2.46717332.G/A | GG | 13 | 28 | −15 | 19.5 | 26 | −6.5 |
| F2R.76059983.A/G | G | 23.5 | 28 | −4.5 | 28 | 25.5 | 2.5 |
| F2R.76059983.A/G | GG | 15 | 28 | −13 | 28 | 25.5 | 2.5 |
| F2R.76049220.G/C | GG | 22.5 | 28 | −5.5 | 27.5 | 24 | 3.5 |
| F3.94719939.A/G | GG | 23.5 | 28 | −4.5 | 28 | 16.5 | 11.5 |
| F5.166258759.A/G | G | 28 | 26 | 2 | 28 | 19 | 9 |
| F5.166236816.T/C | T | 28 | 26 | 2 | 28 | 8.5 | 19.5 |
| F5.166227911.A/G | A | 28 | 20 | 8 | 28 | 12 | 16 |
| F5.166269905.G/A | A | 28 | 26 | 2 | 27 | 19 | 8 |
| F7.112808416.A/G | AG | 15 | 28 | −13 | 23 | 25 | −2 |
| F10.112840894.A/C | C | 23 | 28 | −5 | 28 | 19 | 9 |
| F10.112825510.A/G | G | 17 | 28 | −11 | 24 | 24 | 0 |
| F10.112824083.T/C | T | 22 | 28 | −6 | 26 | 24 | 2 |
| SERPINE1.100363146.4G/5G | I | 21 | 28 | −7 | 28 | 19 | 9 |
| SERPINE1.100375050.G/A | A | 26 | 26 | 0 | 28 | 21 | 7 |
| SERPINE1.100375050.G/A | AG | 28 | 26 | 2 | 28 | 14 | 14 |
| SERPINA5.94123294.C/T | TT | 28 | 26 | 2 | 28 | 16.5 | 11.5 |
| IL6.22541812.C/G | C | 28 | 23.5 | 4.5 | 28 | 27 | 1 |
| IL6.22539885.G/C | G | 19 | 17 | 2 | 27 | 28 | −1 |
| IL10.203334802.C/A | A | 17 | 18 | −1 | 26 | 23 | 3 |
| IL12A.161198944.G/A | A | 20 | 28 | −8 | 28 | 23 | 5 |
| IL12A.161198944.G/A | GA | 20 | 28 | −8 | 28 | 16.5 | 11.5 |
| TNFRSF1A.6317783.T/C | CT | 22.5 | 28 | −5.5 | 27 | 5 | 22 |
| VEGF.43848656.G/A | AA | 20 | 28 | −8 | 27 | 21 | 6 |
| PROC.127890298.A/G | AG | 28 | 23 | 5 | 28 | 11 | 17 |
| PROC.127890457.T/C | CT | 28 | 23 | 5 | 28 | 9 | 19 |
| PROC.127892009.G/A | AG | 28 | 22 | 6 | 28 | 11 | 17 |
| PROC.127892092.C/T | CT | 24.5 | 27 | −2.5 | 28 | 16.5 | 11.5 |
| PROC.127894204.T/C | C | 28 | 25 | 3 | 28 | 13 | 15 |
| PROC.127894204.T/C | CT | 25 | 28 | −3 | 28 | 16.5 | 11.5 |
| PROC.127894608.G/A | AG | 26 | 28 | −2 | 28 | 11 | 17 |
| PROC.127894645.C/T | CT | 24.5 | 27 | −2.5 | 28 | 12 | 16 |
| PROC.127895556.G/A | A | 19 | 28 | −9 | 19 | 28 | −9 |
| PROC.127895556.G/A | AA | 14 | 28 | −14 | 23 | 28 | −5 |
| PROC.127895783.G/A | AG | 28 | 22 | 6 | 28 | 10 | 18 |
| PROC.127895876.T/C | CT | 24.5 | 28 | −3.5 | 28 | 12 | 16 |
| PROC.127899224.C/T | CT | 24.5 | 28 | −3.5 | 28 | 13 | 15 |
| PROC.127901000.T/C | CT | 28 | 23 | 5 | 28 | 12 | 16 |
| PROC.127901799.C/T | CT | 27 | 27 | 0 | 28 | 11 | 17 |
| PROC.127975205.T/C | C | 28 | 26 | 2 | 27 | 24 | 3 |
| PROCR.33183348.T/C | C | 28 | 26 | 2 | 27.5 | 27.5 | 0 |
| PROCR.33183694.C/A | A | 28 | 28 | 0 | 27.5 | 23.5 | 4 |
| PROCR.33186524.A/G | G | 28 | 26 | 2 | 28 | 24 | 4 |
| PROCR.33228215.A/G | AG | 20 | 28 | −8 | 25.5 | 27 | −1.5 |
| PROCR.33228215.A/G | G | 28 | 26 | 2 | 28 | 26.5 | 1.5 |
| AVERAGE | | | | −2.3 | | | 8 |
| DIFFERENCE | | | | | | | |

For days alive and free of ¾ SIRS criteria (TABLE 33), on average matched-control patients having the IRP allele/genotype do worse than patients having alleles/genotypes other than the IRP (−1 days alive and free of ¾ SIRS criteria). In contrast, on average, XIGRIS™-treated patients having the IRP allele/genotype do better than patients having alleles/genotypes other than the IRP (+7.6 days alive and free of ¾ SIRS criteria). The IRP patients benefit the most from XIGRIS™ treatment in terms of improvements of days alive and free of ¾ SIRS criteria.

TABLE 33
Difference in median days alive and free of ¾ SIRS criteria
between improved response polymorphism (IRP) and non-IRP patients
by treatment (control or XIGRIS ™). Data is shown for several
polymorphisms in the coagulation, fibrinolysis and inflammation
pathways in a cohort of critically ill patients who had severe sepsis and
no XIGRIS ™ contraindications. DIFFERENCE = median
days alive and free of ¾ SIRS criteria of patients having the IRP
minus median days alive and free of ¾ SIRS criteria of patients
having the non-IRP allele/genotype, within (1) Matched Controls and
(2) XIGRIS ™-Treated Patients.

| | | Matched Controls | | | XIGRIS ™-Treated Patients | | |
|---|---|---|---|---|---|---|---|
| | | Median | Median | | | Median | |
| SNP | IRP | non-IRP | IRP | DIFFERENCE | Median IRP | non-IRP | DIFFERENCE |
| FGB.155840914.G/A | A | 10 | 9 | 1 | 18 | 2 | 16 |
| F2.46717332.G/A | G | 6 | 12 | −6 | 5 | 2 | 3 |
| F2.46717332.G/A | GG | 3 | 12 | −9 | 4.5 | 3 | 1.5 |
| F2R.76059983.A/G | G | 9 | 9 | 0 | 8 | 2.5 | 5.5 |
| F2R.76059983.A/G | GG | 8 | 9 | −1 | 8 | 3.5 | 4.5 |
| F2R.76049220.G/C | GG | 9 | 8 | 1 | 6 | 2 | 4 |
| F3.94719939.A/G | GG | 5.5 | 9 | −3.5 | 22 | 2 | 20 |
| F5.166258759.A/G | G | 9.5 | 8 | 1.5 | 20 | 4 | 16 |
| F5.166236816.T/C | T | 9 | 9 | 0 | 19.5 | 1 | 18.5 |
| F5.166227911.A/G | A | 10 | 7 | 3 | 19.5 | 1.5 | 18 |
| F5.166269905.G/A | A | 10 | 8 | 2 | 7 | 2 | 5 |
| F7.112808416.A/G | AG | 4 | 12 | −8 | 4 | 4 | 0 |
| F10.112840894.A/C | C | 6 | 9 | −3 | 20 | 2 | 18 |
| F10.112825510.A/G | G | 5 | 10 | −5 | 4 | 7 | −3 |
| F10.112824083.T/C | T | 8 | 9 | −1 | 2 | 11 | −9 |
| SERPINE1.100363146.4G/5G | I | 6 | 9 | −3 | 7 | 4 | 3 |
| SERPINE1.100375050.G/A | A | 7 | 9 | −2 | 21 | 2 | 19 |
| SERPINE1.100375050.G/A | AG | 10 | 8 | 2 | 21 | 1 | 20 |
| SERPINA5.94123294.C/T | TT | 8 | 9 | −1 | 5 | 3 | 2 |
| IL6.22541812.C/G | C | 9 | 11 | −2 | 26 | 21 | 5 |
| IL6.22539885.G/C | G | 5 | 9 | −4 | 16 | 26 | −10 |
| IL10.203334802.C/A | A | 5 | 5 | 0 | 16 | 20 | −4 |
| IL12A.161198944.G/A | A | 8.5 | 9 | −0.5 | 16 | 4 | 12 |
| IL12A.161198944.G/A | GA | 8.5 | 9 | −0.5 | 16 | 3 | 13 |
| TNFRSF1A.6317783.T/C | CT | 9 | 10 | −1 | 4 | 5.5 | −1.5 |
| VEGF.43848656.G/A | AA | 6.5 | 10 | −3.5 | 20 | 3 | 17 |
| PROC.127890298.A/G | AG | 12 | 6 | 6 | 20 | 2 | 18 |
| PROC.127890457.T/C | CT | 12 | 6 | 6 | 20.5 | 2 | 18.5 |
| PROC.127892009.G/A | AG | 12 | 6 | 6 | 20.5 | 2 | 18.5 |
| PROC.127892092.C/T | CT | 8 | 8.5 | −0.5 | 7.5 | 3 | 4.5 |
| PROC.127894204.T/C | C | 9 | 7.5 | 1.5 | 6 | 2 | 4 |
| PROC.127894204.T/C | CT | 9 | 7.5 | 1.5 | 5.5 | 3 | 2.5 |
| PROC.127894608.G/A | AG | 9 | 9 | 0 | 16 | 2 | 14 |
| PROC.127894645.C/T | CT | 8.5 | 9 | −0.5 | 11 | 2 | 9 |
| PROC.127895556.G/A | A | 5 | 9 | −4 | 4 | 6 | −2 |
| PROC.127895556.G/A | AA | 4 | 9 | −5 | 5 | 3 | 2 |
| PROC.127895783.G/A | AG | 12 | 6 | 6 | 20 | 2 | 18 |
| PROC.127895876.T/C | CT | 7.5 | 9 | −1.5 | 11 | 2 | 9 |
| PROC.127899224.C/T | CT | 8.5 | 9 | −0.5 | 11 | 2 | 9 |
| PROC.127901000.T/C | CT | 12 | 6 | 6 | 21 | 2 | 19 |
| PROC.127901799.C/T | CT | 8.5 | 9 | −0.5 | 8 | 2 | 6 |
| PROC.127975205.T/C | C | 9 | 9 | 0 | 6 | 2 | 4 |
| PROCR.33183348.T/C | C | 3.5 | 9 | −5.5 | 4.5 | 5 | −0.5 |
| PROCR.33183694.C/A | A | 4 | 9 | −5 | 4.5 | 2 | 2.5 |
| PROCR.33186524.A/G | G | 4 | 9 | −5 | 3 | 4 | −1 |
| PROCR.33228215.A/G | AG | 4 | 9 | −5 | 11 | 3 | 8 |
| PROCR.33228215.A/G | G | 7 | 9 | −2 | 5 | 6 | −1 |
| AVERAGE | | | | −1 | | | 7.6 |
| DIFFERENCE | | | | | | | |

Overall, there is marked improvement in days alive and free of different organ dysfunctions for the IRP individuals compared to the non-IRP individuals, but importantly, this improvement is only seen when the individuals are treated with XIGRIS™.

We report that polymorphisms within fibrinogen B beta polypeptide (FGB), coagulation factor II (F2), coagulation factor II receptor (F2R), coagulation factor III (F3), coagulation factor V (F5), coagulation factor VII (F7), coagulation factor X (F10), plasminogen activator inhibitor type 1 (SERPINE1), protein C inhibitor (SERPINA5), interleukin 6 (IL6), interleukin 10 (IL10), interleukin 12A (IL12A), tumor necrosis factor alpha receptor-1 (TNFRSF1A), vascular endothelial growth factor (VEGF), protein C (PROC) and protein C receptor (PROCR) genes predict enhanced response to XIGRIS™ treatment.

### Linkage Disequilibrium Analysis

Polymorphisms found to be in linkage disequilibrium with the polymorphisms identified as having an improved response association with XIGRIS™ are listed in TABLE 1B. Polymorphisms in linkage disequilibrium with those listed in TABLE 1A were identified using the LD-select algorithm which analyzes patterns of linkage disequilibrium between polymorphic SNPs across all gene regions of interest (CARLSON C S. et al. Am. J. Hum. Genet. (2004) 74:106-120), r²≧0.5/minor allele frequency (MAF)=0.05. The binning algorithm used in LD-select identified all SNPs that exceed the r² threshold of ≧0.5 with our IRP SNPs. A minimum minor allele frequency of 0.05 was used throughout the analysis.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

1. A method for identifying a subject having an improved response polymorphism in a protein C pathway-associated gene, the method comprising determining a genotype of said subject at one or more polymorphic sites in the subject's protein C pathway-associated gene sequences or a combination thereof, wherein said genotype is indicative of the subject's response to administration of activated protein C or protein C-like compound.

2. The method of claim 1, wherein the polymorphic site is rs1800791; rs3136516; rs253073; rs2227750; rs1361600; rs9332575; rs4656687; rs9332630; rs9332546; rs2774030; rs2026160; rs3211719; rs3093261; rs1799889; rs1050813; rs2069972; rs2069840; rs1800795; rs1800872; rs2243154; rs4149577; rs1413711; rs2069895; rs2069898; rs2069904; rs1799808; rs2069910; rs2069915; rs2069916; rs2069918; rs2069919; rs2069920; rs2069924; rs5937; rs2069931; rs777556; rs1033797; rs1033799; rs2295888; rs867186; or one or more polymorphic sites in linkage disequilibrium (LD) with any of said sites.

3. The method of claim 1, wherein the improved response polymorphism is rs1800791A; rs3136516G; rs3136516GG; rs253073G; rs253073GG; rs2227750GG; rs1361600GG; rs9332575G; rs4656687T; rs9332630A; rs9332546A; rs2774030AG; rs2026160C; rs3211719G; rs3093261T; rs1799889G; rs1050813A; rs1050813AG; rs2069972TT; rs2069840C; rs1800795G; rs1800872A; rs2243154A; rs2243154AG; rs4149577CT; rs1413711AA; rs2069895AG; rs2069898CT; rs2069904AG; rs1799808CT; rs2069910C; rs2069910CT; rs2069915AG; rs2069916CT; rs2069918A; rs2069918AA; rs2069919AG; rs2069920CT; rs2069924CT; rs5937CT; rs2069931CT; rs777556C; rs1033797C; rs1033799A; rs2295888G; rs867186AG; rs867186G; or one or more polymorphic sites in LD with any of said sites.

4. The method of claim 2, wherein the one or more polymorphic sites in LD is selected from the polymorphic sites listed in TABLE 1B.

5. The method of claim 1, further comprising comparing the genotype so determined with known genotypes which are known to be indicative of the subject's response to administration of activated protein C or a protein C like compound.

6. The method of claim 1, further comprising obtaining protein C pathway associated gene sequence for the subject.

7. The method of claim 1, wherein the genotype is determined using a nucleic acid sample from the subject.

8. The method of claim 7, further comprising obtaining the nucleic acid sample from the subject.

9. The method of claim 1, wherein said genotype is determined using one or more of the following techniques:
(a) restriction fragment length analysis;
(b) sequencing;
(c) micro-sequencing assay;
(d) hybridization;
(e) invader assay;
(f) gene chip hybridization assays;
(g) oligonucleotide ligation assay;
(h) ligation rolling circle amplification;
(i) 5′ nuclease assay;
(j) polymerase proofreading methods;
(k) allele specific PCR;
(l) matrix assisted laser desorption ionization time of flight (MALDI-TOF) mass spectroscopy;
(m) ligase chain reaction assay;
(n) enzyme-amplified electronic transduction;
(o) single base pair extension assay; and
(p) reading sequence data.

10. The method of claim 1, wherein the subject is critically ill with an inflammatory condition.

11. The method of claim 10, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for a subject undergoing major surgery or dialysis or who is otherwise immunocompromised, a subject on immunosuppressive agents, a subject with HIV/AIDS, a subject with suspected endocarditis, a subject with fever, a subject with fever of unknown origin, a subject with cystic fibrosis, a subject with diabetes mellitus, a subject with chronic renal failure, acute renal failure, oliguria, acute renal dysfunction, glomerulo-nephritis, interstitial-nephritis, or acute tubular necrosis (ATN), a subject with bronchiectasis, a subject with chronic obstructive lung disease, chronic bronchitis, emphysema, or asthma, a subject with febrile neutropenia, a subject with meningitis, a subject with septic arthritis, a subject with urinary tract infection, a subject with necrotizing fasciitis, a subject with other suspected Group A streptococcus infection, a splenectomized subject a subject with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

12. The method of claim 10, wherein the inflammatory condition is SIRS; sepsis; or septic shock.

13. The method of claim 1, further comprising selective administration of activated protein C or protein C like compound, wherein a subject has one or more improved response polymorphisms in the subject's protein C pathway-associated gene sequences.

14. The method of claim 1, further comprising selectively not administering activated protein C or protein C like compound, wherein a subject does not have one or more improved response polymorphisms in the subject's protein C pathway associated gene sequences.

15. A method for selecting a group of subjects for determining the efficacy of a candidate drug known or suspected of being useful for the treatment of an inflammatory condition, the method comprising
(a) determining a genotype at one or more polymorphic sites in a protein C pathway-associated gene sequence for each subject, wherein said genotype is indicative of the subject's response to the candidate drug, and
(b) sorting subjects based on their genotype.

16. The method of claim 15 further comprising, administering the candidate drug to the subjects or a subset of subjects and determining each subject's ability to recover from the inflammatory condition.

17. The method of claim 16, further comprising comparing the subject's response to the candidate drug based on genotype of the subject.

18. (canceled)

19. A method of selecting a subject expected to be responsive to treatment of an inflammatory condition by administration of an activated protein C or protein C-like compound, comprising identifying a subject who has an improved response polymorphism in his or her protein C pathway-associated gene sequence, wherein a subject so identified is selected for treatment of the inflammatory condition with the activated protein C or protein C-like compound.

20.-21. (canceled)

22. The method of claim 19, further comprising determining the subject's APACHE II score as an assessment of subject risk.

23. The method of claim 19, further comprising determining the number of organ system failures for the subject as an assessment of subject risk.

24. The method of claim 22, wherein the subject's APACHE II score of >25 is indicative of an increased risk.

25. The method of claim 23, wherein 2 or more organ system failures are indicative of increased subject risk.

26. The method of claim 19, wherein the inflammatory condition is selected from the group consisting of: sepsis, septicemia, pneumonia, septic shock, systemic inflammatory response syndrome (SIRS), Acute Respiratory Distress Syndrome (ARDS), acute lung injury, aspiration pneumonitis, infection, pancreatitis, bacteremia, peritonitis, abdominal abscess, inflammation due to trauma, inflammation due to surgery, chronic inflammatory disease, ischemia, ischemia-reperfusion injury of an organ or tissue, tissue damage due to disease, tissue damage due to chemotherapy or radiotherapy, and reactions to ingested, inhaled, infused, injected, or delivered substances, glomerulonephritis, bowel infection, opportunistic infections, and for a subject undergoing major surgery or dialysis or, subjects who is otherwise immunocompromised, a subject on immunosuppressive agents, a subject with HIV/AIDS, a subject with suspected endocarditis, a subject with fever, a subject with fever of unknown origin, a subject with cystic fibrosis, a subject with diabetes mellitus, a subject with chronic renal failure, acute renal failure, oliguria, acute renal dysfunction, glomerulo-nephritis, interstitial-nephritis, or acute tubular necrosis (ATN), a subject with bronchiectasis, a subject with chronic obstructive lung disease, chronic bronchitis, emphysema, or asthma, a subject with febrile neutropenia, a subject with meningitis, a subject with septic arthritis, a subject with urinary tract infection, a subject with necrotizing fasciitis, a subject with other suspected Group A streptococcus infection, a splenectomized subject, a subject with recurrent or suspected enterococcus infection, other medical and surgical conditions associated with increased risk of infection, Gram positive sepsis, Gram negative sepsis, culture negative sepsis, fungal sepsis, meningococcemia, post-pump syndrome, cardiac stun syndrome, myocardial infarction, stroke, congestive heart failure, hepatitis, epiglotittis, E. coli 0157:H7, malaria, gas gangrene, toxic shock syndrome, pre-eclampsia, eclampsia, HELP syndrome, mycobacterial tuberculosis, Pneumocystic Pneumocystis carinii pneumonia, Leishmaniasis, hemolytic uremic syndrome/thrombotic thrombocytopenic purpura, Dengue hemorrhagic fever, pelvic inflammatory disease, Legionella, Lyme disease, Influenza A, Epstein-Barr virus, encephalitis, inflammatory diseases and autoimmunity including Rheumatoid arthritis, osteoarthritis, progressive systemic sclerosis, systemic lupus erythematosus, inflammatory bowel disease, idiopathic pulmonary fibrosis, sarcoidosis, hypersensitivity pneumonitis, systemic vasculitis, Wegener's granulomatosis, transplants including heart, liver, lung kidney bone marrow, graft-versus-host disease, transplant rejection, sickle cell anemia, nephrotic syndrome, toxicity of agents such as OKT3, cytokine therapy, and cirrhosis.

27. The method of claim 19, wherein the inflammatory condition is systemic inflammatory response syndrome.

28. The method of claim 19, wherein the polymorphic site is selected from one or more of the following: rs1800791; rs3136516; rs253073; rs2227750; rs1361600; rs9332575; rs4656687; rs9332630; rs9332546; rs2774030; rs2026160; rs3211719; rs3093261; rs1799889; rs1050813; rs2069972; rs2069840; rs1800795; rs1800872; rs2243154; rs4149577; rs1413711; rs2069895; rs2069898; rs2069904; rs1799808; rs2069910; rs2069915; rs2069916; rs2069918; rs2069919; rs2069920; rs2069924; rs5937; rs2069931; rs777556; rs1033797; rs1033799; rs2295888; rs867186; or one or more polymorphic sites in LD with any of said sites.

29. The method of claim 19, wherein the improved response polymorphism is rs1800791A; rs3136516G; rs3136516GG; rs253073G; rs253073GG; rs2227750GG; rs1361600GG; rs9332575G; rs4656687T; rs9332630A; rs9332546A; rs2774030AG; rs2026160C; rs3211719G; rs3093261T; rs1799889G; rs1050813A; rs1050813AG; rs2069972TT; rs2069840C; rs1800795G; rs1800872A; rs2243154A; rs2243154AG; rs4149577CT; rs1413711AA; rs2069895AG; rs2069898CT; rs2069904AG; rs1799808CT; rs2069910C; rs2069910CT; rs2069915AG; rs2069916CT; rs2069918A; rs2069918AA; rs2069919AG; rs2069920CT; rs2069924CT; rs5937CT; rs2069931CT; rs777556C; rs1033797C; rs1033799A; rs2295888G; rs867186AG; or rs867186G; or one or more polymorphic sites in LD with any of said sites.

30. The method of claim 19, wherein the one or more polymorphic sites in linkage disequilibrium is selected from the polymorphic sites listed in TABLE 1B.

31. The method of claim 19, wherein the activated protein C or protein C like compound is drotecogin alfa activated.

32. Two or more oligonucleotides or peptide nucleic acids of about 10 to about 400 nucleotides that hybridize specifically to a sequence contained in a human target sequence consisting of a subject's protein C pathway-associated gene sequence, a complementary sequence of the target sequence or RNA equivalent of the target sequence and wherein the oligonucleotides or peptide nucleic acids are operable in determining the presence or absence of two or more improved response polymorphisms in their protein C pathway-associated gene sequence selected from rs1800791; rs3136516; rs253073; rs2227750; rs1361600; rs9332575; rs4656687; rs9332630; rs9332546; rs2774030; rs2026160; rs3211719; rs3093261; rs1799889; rs1050813; rs2069972; rs2069840; rs1800795; rs1800872; rs2243154; rs4149577; rs1413711; rs2069895; rs2069898; rs2069904; rs1799808; rs2069910; rs2069915; rs2069916; rs2069918; rs2069919; rs2069920; rs2069924; rs5937; rs2069931; rs777556; rs1033797; rs1033799; rs2295888; rs867186 or one or more polymorphic sites in linkage disequilibrium with any of said sites.

33. The oligonucleotides or peptide nucleic acids of claim 32, wherein the improved response polymorphism is rs1800791A; rs3136516G; rs3136516GG; rs253073G; rs253073GG; rs2227750GG; rs1361600GG; rs9332575G; rs4656687T; rs9332630A; rs9332546A; rs2774030AG; rs2026160C; rs3211719G; rs3093261T; rs1799889G; rs1050813A; rs1050813AG; rs2069972TT; rs2069840C; rs1800795G; rs1800872A; rs2243154A; rs2243154AG; rs4149577CT; rs1413711AA; rs2069895AG; rs2069898CT; rs2069904AG; rs1799808CT; rs2069910C; rs2069910CT; rs2069915AG; rs2069916CT; rs2069918A; rs2069918AA; rs2069919AG; rs2069920CT; rs2069924CT; rs5937CT; rs2069931CT; rs777556C; rs1033797C; rs1033799A; rs2295888G; rs867186AG; rs867186G; or one or more polymorphic sites in LD with any of said sites.

34. The oligonucleotides or peptide nucleic acids of claim 32, wherein the one or more polymorphic sites in LD is selected from the polymorphic sites listed in TABLE 1B.

35. Two or more oligonucleotides or peptide nucleic acids selected from the group consisting of:
(a) an oligonucleotide or peptide nucleic acid that hybridizes under high stringency conditions to a nucleic acid molecule comprising SEQ ID NO:1 having a G at position 86 but not to a nucleic acid molecule comprising SEQ ID NO:1 having an A at position 86;
(b) an oligonucleotide or peptide nucleic acid that hybridizes under high stringency conditions to a nucleic acid molecule comprising SEQ ID NO:1 having an A at position 86 but not to a nucleic acid molecule comprising SEQ ID NO:1 having a G at position 86;
(c) an oligonucleotide or peptide nucleic acid that hybridizes under high stringency conditions to a nucleic acid molecule comprising SEQ ID NO:2 having a G at position 201 but not to a nucleic acid molecule comprising SEQ ID NO:2 having an A at position 201;
(d) an oligonucleotide or peptide nucleic acid that hybridizes under high stringency conditions to a nucleic acid molecule comprising SEQ ID NO:2 having an A at position 201 but not to a nucleic acid molecule comprising SEQ ID NO:2 having a G at position 201;
(e) an oligonucleotide or peptide nucleic acid that hybridizes under high stringency conditions to a nucleic acid molecule comprising SEQ ID NO:3 having an A at position 201 but not to a nucleic acid molecule comprising SEQ ID NO:3 having a G at position 201;
(f) an oligonucleotide or peptide nucleic acid that hybridizes under high stringency conditions to a nucleic acid molecule comprising SEQ ID NO:3 having a G at position 201 but not to a nucleic acid molecule comprising SEQ ID NO:3 having an A at position 201;
(g) an oligonucleotide or peptide nucleic acid that hybridizes under high stringency conditions to a nucleic acid molecule comprising SEQ ID NO:4 having a G at position 201 but not to a nucleic acid molecule comprising SEQ ID NO:4 having a C at position 201;
(h) an oligonucleotide or peptide nucleic acid that hybridizes under high stringency conditions to a nucleic acid molecule comprising SEQ ID NO:4 having a C at position 201 but not to a nucleic acid molecule comprising SEQ ID NO:4 having a G at position 201;
(i) an oligonucleotide or peptide nucleic acid that hybridizes under high stringency conditions to a nucleic acid molecule comprising SEQ ID NO:5 having an A at position 201 but not to a nucleic acid molecule comprising SEQ ID NO:5 having a G at position 201;
(j) an oligonucleotide or peptide nucleic acid that hybridizes under high stringency conditions to a nucleic acid molecule comprising SEQ ID NO:5 having a G at position 201 but not to a nucleic acid molecule comprising SEQ ID NO:5 having an A at position 201;
(k) an oligonucleotide or peptide nucleic acid that hybridizes under high stringency conditions to a nucleic acid molecule comprising SEQ ID NO:6 having an A at position 201 but not to a nucleic acid molecule comprising SEQ ID NO:6 having a G at position 201;
(l) an oligonucleotide or peptide nucleic acid that hybridizes under high stringency conditions to a nucleic acid molecule comprising SEQ ID NO:6 having a G at position 201 but not to a nucleic acid molecule comprising SEQ ID NO:6 having an A at position 201;
(m) an oligonucleotide or peptide nucleic acid that hybridizes under high stringency conditions to a nucleic acid molecule comprising SEQ ID NO:7 having a C at position 201 but not to a nucleic acid molecule comprising SEQ ID NO:7 having a T at position 201;
(n) an oligonucleotide or peptide nucleic acid that hybridizes under high stringency conditions to a nucleic acid molecule comprising SEQ ID NO:7 having a T at position 201 but not to a nucleic acid molecule comprising SEQ ID NO:7 having a C at position 201;
(o) an oligonucleotide or peptide nucleic acid that hybridizes under high stringency conditions to a nucleic acid molecule comprising SEQ ID NO:8 having an A at position 201 but not to a nucleic acid molecule comprising SEQ ID NO:8 having a G at position 201;
(p) an oligonucleotide or peptide nucleic acid that hybridizes under high stringency conditions to a nucleic acid molecule comprising SEQ ID NO:8 having a G at position 201 but not to a nucleic acid molecule comprising SEQ ID NO:8 having an A at position 201;
(q) an oligonucleotide or peptide nucleic acid that hybridizes under high stringency conditions to a nucleic acid molecule comprising SEQ ID NO:9 having a G at position 201 but not to a nucleic acid molecule comprising SEQ ID NO:9 having an A at position 201;
(r) an oligonucleotide or peptide nucleic acid that hybridizes under high stringency conditions to a nucleic acid molecule comprising SEQ ID NO:9 having an A at position 201 but not to a nucleic acid molecule comprising SEQ ID NO:9 having a G at position 201;
(s) an oligonucleotide or peptide nucleic acid that hybridizes under high stringency conditions to a nucleic acid molecule comprising SEQ ID NO:10 having an A at position 201 but not to a nucleic acid molecule comprising SEQ ID NO:10 having a G at position 201;
(t) an oligonucleotide or peptide nucleic acid that hybridizes under high stringency conditions to a nucleic acid molecule comprising SEQ ID NO:10 having a G at position 201 but not to a nucleic acid molecule comprising SEQ ID NO:10 having an A at position 201;
(u) an oligonucleotide or peptide nucleic acid that hybridizes under high stringency conditions to a nucleic acid molecule comprising SEQ ID NO:11 having an A at position 201 but not to a nucleic acid molecule comprising SEQ ID NO:11 having a C at position 201;
(v) an oligonucleotide or peptide nucleic acid that hybridizes under high stringency conditions to a nucleic acid molecule comprising SEQ ID NO:11 having a C at position 201 but not to a nucleic acid molecule comprising SEQ ID NO:11 having an A at position 201;
(w) an oligonucleotide or peptide nucleic acid that hybridizes under high stringency conditions to a nucleic acid molecule comprising SEQ ID NO:12 having an A at position 201 but not to a nucleic acid molecule comprising SEQ ID NO:12 having a G at position 201;
(x) an oligonucleotide or peptide nucleic acid that hybridizes under high stringency conditions to a nucleic acid molecule comprising SEQ ID NO:12 having a G at position 201 but not to a nucleic acid molecule comprising SEQ ID NO:12 having an A at position 201;
(y) an oligonucleotide or peptide nucleic acid that hybridizes under high stringency conditions to a nucleic acid molecule comprising SEQ ID NO:13 having a T at position 201 but not to a nucleic acid molecule comprising SEQ ID NO:13 having a C at position 201;
(z) an oligonucleotide or peptide nucleic acid that hybridizes under high stringency conditions to a nucleic acid molecule comprising SEQ ID NO:13 having a C at position 201 but not to a nucleic acid molecule comprising SEQ ID NO:13 having a T at position 201
(aa) an oligonucleotide or peptide nucleic acid that hybridizes under high stringency conditions to a nucleic acid molecule comprising SEQ ID NO:14 having a G at position 201 but not to a nucleic acid molecule comprising SEQ ID NO:14 having a deletion at position 201;
(bb) an oligonucleotide or peptide nucleic acid that hybridizes under high stringency conditions to a nucleic acid molecule comprising SEQ ID NO:14 having an deletion at position 201 but not to a nucleic acid molecule comprising SEQ ID NO:14 having a G at position 201;
(cc) an oligonucleotide or peptide nucleic acid that hybridizes under high stringency conditions to a nucleic acid molecule comprising SEQ ID NO:15 having a G at position 201 but not to a nucleic acid molecule comprising SEQ ID NO:15 having an A at position 201
(dd) an oligonucleotide or peptide nucleic acid that hybridizes under high stringency conditions to a nucleic acid molecule comprising SEQ ID NO:15 having an A at position 201 but not to a nucleic acid molecule comprising SEQ ID NO:15 having a G at position 201;
(ee) an oligonucleotide or peptide nucleic acid that hybridizes under high stringency conditions to a nucleic acid molecule comprising SEQ ID NO:16 having a C at position 201 but not to a nucleic acid molecule comprising SEQ ID NO:16 having a T at position 201;
(ff) an oligonucleotide or peptide nucleic acid that hybridizes under high stringency conditions to a nucleic acid molecule comprising SEQ ID NO:16 having a T at position 201 but not to a nucleic acid molecule comprising SEQ ID NO:16 having a C at position 201;
(gg) an oligonucleotide or peptide nucleic acid that hybridizes under high stringency conditions to a nucleic acid molecule comprising SEQ ID NO:17 having a C at position 201 but not to a nucleic acid molecule comprising SEQ ID NO:17 having a G at position 201;
(hh) an oligonucleotide or peptide nucleic acid that hybridizes under high stringency conditions to a nucleic acid molecule comprising SEQ ID NO:17 having a G at position 201 but not to a nucleic acid molecule comprising SEQ ID NO:17 having a C at position 201;
(ii) an oligonucleotide or peptide nucleic acid that hybridizes under high stringency conditions to a nucleic acid molecule comprising SEQ ID NO:18 having a G at position 201 but not to a nucleic acid molecule comprising SEQ ID NO:18 having a C at position 201;
(jj) an oligonucleotide or peptide nucleic acid that hybridizes under high stringency conditions to a nucleic acid molecule comprising SEQ ID NO:18 having a C at position 201 but not to a nucleic acid molecule comprising SEQ ID NO:18 having a G at position 201;
(kk) an oligonucleotide or peptide nucleic acid that hybridizes under high stringency conditions to a nucleic acid molecule comprising SEQ ID NO:19 having a C at position 201 but not to a nucleic acid molecule comprising SEQ ID NO:19 having an A at position 201;
(ll) an oligonucleotide or peptide nucleic acid that hybridizes under high stringency conditions to a nucleic acid molecule comprising SEQ ID NO:19 having an A at position 201 but not to a nucleic acid molecule comprising SEQ ID NO:19 having a C at position 201;
(mm) an oligonucleotide or peptide nucleic acid that hybridizes under high stringency conditions to a nucleic acid molecule comprising SEQ ID NO:20 having a G at position 201 but not to a nucleic acid molecule comprising SEQ ID NO:20 having an A at position 201;
(nn) an oligonucleotide or peptide nucleic acid that hybridizes under high stringency conditions to a nucleic acid molecule comprising SEQ ID NO:20 having an A at position 201 but not to a nucleic acid molecule comprising SEQ ID NO:20 having a G at position 201;
(oo) an oligonucleotide or peptide nucleic acid that hybridizes under high stringency conditions to a nucleic acid molecule comprising SEQ ID NO:21 having a T at position 201 but not to a nucleic acid molecule comprising SEQ ID NO:21 having a C at position 201;
(pp) an oligonucleotide or peptide nucleic acid that hybridizes under high stringency conditions to a nucleic acid molecule comprising SEQ ID NO:21 having a C at position 201 but not to a nucleic acid molecule comprising SEQ ID NO:21 having a T at position 201;
(qq) an oligonucleotide or peptide nucleic acid that hybridizes under high stringency conditions to a nucleic acid molecule comprising SEQ ID NO:22 having an A at position 201 but not to a nucleic acid molecule comprising SEQ ID NO:22 having a G at position 201;
(rr) an oligonucleotide or peptide nucleic acid that hybridizes under high stringency conditions to a nucleic acid molecule comprising SEQ ID NO:22 having a G at position 201 but not to a nucleic acid molecule comprising SEQ ID NO:22 having an A at position 201;
(ss) an oligonucleotide or peptide nucleic acid that hybridizes under high stringency conditions to a nucleic acid molecule comprising SEQ ID NO:23 having an A at position 51 but not to a nucleic acid molecule comprising SEQ ID NO:23 having a G at position 51;
(tt) an oligonucleotide or peptide nucleic acid that hybridizes under high stringency conditions to a nucleic acid molecule comprising SEQ ID NO:23 having a G at position 51 but not to a nucleic acid molecule comprising SEQ ID NO:23 having an A at position 51;
(uu) an oligonucleotide or peptide nucleic acid that hybridizes under high stringency conditions to a nucleic acid molecule comprising SEQ ID NO:24 having a C at position 51 but not to a nucleic acid molecule comprising SEQ ID NO:24 having a T at position 51;
(vv) an oligonucleotide or peptide nucleic acid that hybridizes under high stringency conditions to a nucleic acid molecule comprising SEQ ID NO:24 having a T at position 51 but not to a nucleic acid molecule comprising SEQ ID NO:24 having a C at position 51;
(ww) an oligonucleotide or peptide nucleic acid that hybridizes under high stringency conditions to a nucleic acid molecule comprising SEQ ID NO:25 having an A at position 51 but not to a nucleic acid molecule comprising SEQ ID NO:25 having a G at position 51;
(xx) an oligonucleotide or peptide nucleic acid that hybridizes under high stringency conditions to a nucleic acid molecule comprising SEQ ID NO:25 having a G at position 51 but not to a nucleic acid molecule comprising SEQ ID NO:25 having an A at position 51;
(yy) an oligonucleotide or peptide nucleic acid that hybridizes under high stringency conditions to a nucleic acid molecule comprising SEQ ID NO:26 having a C at position 201 but not to a nucleic acid molecule comprising SEQ ID NO:26 having a T at position 201;
(zz) an oligonucleotide or peptide nucleic acid that hybridizes under high stringency conditions to a nucleic acid molecule comprising SEQ ID NO:26 having a T at position 201 but not to a nucleic acid molecule comprising SEQ ID NO:26 having an C at position 201;
(aaa) an oligonucleotide or peptide nucleic acid that hybridizes under high stringency conditions to a nucleic acid molecule comprising SEQ ID NO:27 having a C at position 201 but not to a nucleic acid molecule comprising SEQ ID NO:27 having a T at position 201;
(bbb) an oligonucleotide or peptide nucleic acid that hybridizes under high stringency conditions to a nucleic acid molecule comprising SEQ ID NO:27 having a T at position 201 but not to a nucleic acid molecule comprising SEQ ID NO:27 having a C at position 201;
(ccc) an oligonucleotide or peptide nucleic acid that hybridizes under high stringency conditions to a nucleic acid molecule comprising SEQ ID NO:28 having an A at position 201 but not to a nucleic acid molecule comprising SEQ ID NO:28 having a G at position 201;
(ddd) an oligonucleotide or peptide nucleic acid that hybridizes under high stringency conditions to a nucleic acid molecule comprising SEQ ID NO:28 having a G at position 201 but not to a nucleic acid molecule comprising SEQ ID NO:28 having an A at position 201;
(eee) an oligonucleotide or peptide nucleic acid that hybridizes under high stringency conditions to a nucleic acid molecule comprising SEQ ID NO:29 having a C at position 201 but not to a nucleic acid molecule comprising SEQ ID NO:29 having a T at position 201;
(fff) an oligonucleotide or peptide nucleic acid that hybridizes under high stringency conditions to a nucleic acid molecule comprising SEQ ID NO:29 having a T at position 201 but not to a nucleic acid molecule comprising SEQ ID NO:29 having a C at position 201;
(ggg) an oligonucleotide or peptide nucleic acid that hybridizes under high stringency conditions to a nucleic acid molecule comprising SEQ ID NO:30 having an A at position 201 but not to a nucleic acid molecule comprising SEQ ID NO:30 having a G at position 201;
(hhh) an oligonucleotide or peptide nucleic acid that hybridizes under high stringency conditions to a nucleic acid molecule comprising SEQ ID NO:30 having a G at position 201 but not to a nucleic acid molecule comprising SEQ ID NO:30 having an A at position 201;
(iii) an oligonucleotide or peptide nucleic acid that hybridizes under high stringency conditions to a nucleic acid molecule comprising SEQ ID NO:31 having an A at position 201 but not to a nucleic acid molecule comprising SEQ ID NO:31 having a G at position 201;
(jjj) an oligonucleotide or peptide nucleic acid that hybridizes under high stringency conditions to a nucleic acid molecule comprising SEQ ID NO:31 having a G at position 201 but not to a nucleic acid molecule comprising SEQ ID NO:31 having an A at position 201;
(kkk) an oligonucleotide or peptide nucleic acid that hybridizes under high stringency conditions to a nucleic acid molecule comprising SEQ ID NO:32 having a C at position 201 but not to a nucleic acid molecule comprising SEQ ID NO:32 having a T at position 201;
(lll) an oligonucleotide or peptide nucleic acid that hybridizes under high stringency conditions to a nucleic acid molecule comprising SEQ ID NO:32 having a T at position 201 but not to a nucleic acid molecule comprising SEQ ID NO:32 having a C at position 201;
(mmm) an oligonucleotide or peptide nucleic acid that hybridizes under high stringency conditions to a nucleic acid molecule comprising SEQ ID NO:33 having a C at position 501 but not to a nucleic acid molecule comprising SEQ ID NO:33 having a T at position 501;
(nnn) an oligonucleotide or peptide nucleic acid that hybridizes under high stringency conditions to a nucleic acid molecule comprising SEQ ID NO:33 having a T at position 501 but not to a nucleic acid molecule comprising SEQ ID NO:33 having a C at position 501;
(ooo) an oligonucleotide or peptide nucleic acid that hybridizes under high stringency conditions to a nucleic acid molecule comprising SEQ ID NO:34 having a C at position 201 but not to a nucleic acid molecule comprising SEQ ID NO:34 having a T at position 201;
(ppp) an oligonucleotide or peptide nucleic acid that hybridizes under high stringency conditions to a nucleic acid molecule comprising SEQ ID NO:34 having a T at position 201 but not to a nucleic acid molecule comprising SEQ ID NO:34 having a C at position 201;
(qqq) an oligonucleotide or peptide nucleic acid that hybridizes under high stringency conditions to a nucleic acid molecule comprising SEQ ID NO:35 having a C at position 201 but not to a nucleic acid molecule comprising SEQ ID NO:35 having a T at position 201;
(rrr) an oligonucleotide or peptide nucleic acid that hybridizes under high stringency conditions to a nucleic acid molecule comprising SEQ ID NO:35 having a T at position 201 but not to a nucleic acid molecule comprising SEQ ID NO:35 having a C at position 201;
(sss) an oligonucleotide or peptide nucleic acid that hybridizes under high stringency conditions to a nucleic acid molecule comprising SEQ ID NO:36 having a C at position 201 but not to a nucleic acid molecule comprising SEQ ID NO:36 having a T at position 201;
(ttt) an oligonucleotide or peptide nucleic acid that hybridizes under high stringency conditions to a nucleic acid molecule comprising SEQ ID NO:36 having a T at position 201 but not to a nucleic acid molecule comprising SEQ ID NO:36 having a C at position 201;
(uuu) an oligonucleotide or peptide nucleic acid that hybridizes under high stringency conditions to a nucleic acid molecule comprising SEQ ID NO:37 having a C at position 201 but not to a nucleic acid molecule comprising SEQ ID NO:37 having a T at position 201;
(vvv) an oligonucleotide or peptide nucleic acid that hybridizes under high stringency conditions to a nucleic acid molecule comprising SEQ ID NO:37 having a T at position 201 but not to a nucleic acid molecule comprising SEQ ID NO:37 having a C at position 201;
(www) an oligonucleotide or peptide nucleic acid that hybridizes under high stringency conditions to a nucleic acid molecule comprising SEQ ID NO:38 having a C at position 201 but not to a nucleic acid molecule comprising SEQ ID NO:38 having an A at position 201;
(xxx) an oligonucleotide or peptide nucleic acid that hybridizes under high stringency conditions to a nucleic acid molecule comprising SEQ ID NO:38 having an A at position 201 but not to a nucleic acid molecule comprising SEQ ID NO:38 having a C at position 201;
(yyy) an oligonucleotide or peptide nucleic acid that hybridizes under high stringency conditions to a nucleic acid molecule comprising SEQ ID NO:39 having an A at position 201 but not to a nucleic acid molecule comprising SEQ ID NO:39 having a G at position 201;
(zzz) an oligonucleotide or peptide nucleic acid that hybridizes under high stringency conditions to a nucleic acid molecule comprising SEQ ID NO:39 having a G at position 201 but not to a nucleic acid molecule comprising SEQ ID NO:39 having an A at position 201;
(aaaa) an oligonucleotide or peptide nucleic acid that hybridizes under high stringency conditions to a nucleic acid molecule comprising SEQ ID NO:40 having an A at position 201 but not to a nucleic acid molecule comprising SEQ ID NO:40 having a G at position 201;
(bbbb) an oligonucleotide or peptide nucleic acid that hybridizes under high stringency conditions to a nucleic acid molecule comprising SEQ ID NO:40 having a G at position 201 but not to a nucleic acid molecule comprising SEQ ID NO:40 having an A at position 201;
(cccc) an oligonucleotide or peptide nucleic acid capable of hybridizing under high stringency conditions to a nucleic acid molecule comprising a first allele for a given polymorphism selected from the polymorphisms listed in TABLE 1D but not capable of hybridizing under high stringency conditions to a nucleic acid molecule comprising a second allele for the given polymorphism selected from the polymorphisms listed in TABLE 1D; and
(dddd) an oligonucleotide or peptide nucleic acid capable of hybridizing under high stringency conditions to a nucleic acid molecule comprising the second allele for a given polymorphism selected from the polymorphisms listed in TABLE 1D but not capable of hybridizing under high stringency conditions to a nucleic acid molecule comprising the first allele for the given polymorphism selected from the polymorphisms listed in TABLE 1D.

36. An array of oligonucleotides or peptide nucleic acids bound to a solid support, the array comprising two or more of the oligonucleotides or peptide nucleic acids according to claim 35.

37. A composition comprising an addressable collection of two or more oligonucleotides or peptide nucleic acids, which oligonucleotides or peptide nucleic acids consist essentially of two or more nucleic acid molecules set out in SEQ ID NO:1-243 or complements, fragments, variants, or analogues thereof.

38. The oligonucleotides or peptide nucleic acids of claim 35, further comprising one or more of the following: a detectable label; a quencher; a mobility modifier; a contiguous non-target sequence situated 5′ or 3′ to the target sequence or 5′ and 3′ to the target sequence.
